(19) United States
(12) Patent Application Publication
(10) Pub. No.: US 2026/0055077 A1
(43) Pub. Date: Feb. 26, 2026

(54) PROTEIN DEGRADATION AGENT

(71) Applicant: SUZHOU KINTOR PHARMACEUTICALS, INC., Suzhou, Jiangsu (CN)

(72) Inventors: Youzhi TONG, Suzhou, Jiangsu (CN); Zhaohui YANG, Suzhou, Jiangsu (CN); Ruo XU, Suzhou, Jiangsu (CN); Liandong MA, Suzhou, Jiangsu (CN); Dong CHEN, Suzhou, Jiangsu (CN)

(73) Assignee: SUZHOU KINTOR PHARMACEUTICALS, INC., Suzhou, Jiangsu (CN)

(21) Appl. No.: 18/995,388

(22) PCT Filed: Aug. 1, 2023
(86) PCT No.: PCT/CN2023/110514

§ 371 Date: Jan. 16, 2025

(30) Foreign Application Priority Data
Aug. 1, 2022 (CN) 202210916308.5
Dec. 20, 2022 (CN) 202211640645.2

(51) Int. Cl. C07D 401/04; A61K 31/454; A61K 31/4545; A61K 31/4709; A61K 31/496; A61P 35/02; C07D 401/14; C07D 471/04

(52) U.S. Cl. CPC C07D 401/04; A61K 31/454; A61K 31/4545; A61K 31/4709; A61K 31/496; A61P 35/02; C07D 401/14; C07D 471/04

(57) ABSTRACT

The present invention relates to a protein degradation agent, a preparation method therefor, and a use thereof. The protein degradation can degrade various proteins including c-Myc protein, and therefore can be used for prevention and treatment of diseases related to disorders of various proteins including c-Myc protein, such as cancer; cardiovascular and cerebrovascular diseases, and viral infection-related diseases.

### CROSS REFERENCES TO RELATED APPLICATION

The present application claims the benefit of Chinese Patent Application No. 2022109163085 filed on Aug. 1, 2022 and No. 2022116406452 filed on Dec. 20, 2022, which is hereby incorporated herein by reference in its entirety.

### TECHNICAL FIELD

The present invention belongs to the field of medicine, and in particular relates to a protein degradation agent, and a preparation method therefor and use thereof.

### BACKGROUND ART

The intrinsically disordered protein c-Myc is a well-known transcription factor that can regulate approximately 15% of human genes. c-Myc can regulate various biological functions, such as cell proliferation, apoptosis, cell cycle progression, cell metabolism and embryonic development, and plays a very important role in the occurrence, development and evolution of diseases.

A large number of studies have shown that c-Myc is closely related to various tumor diseases, including lymphoma, breast cancer, prostate cancer, colon cancer, cervical cancer, multiple myeloma, myeloid leukemia, melanoma, osteosarcoma, malignant glioma, small cell lung cancer and medulloblastoma. c-Myc can promote occurrence and growth of tumors in many aspects (C Yu, et al. Sci Rep 6.1, 1-H).

In addition to cancer, c-Myc is related to some other diseases. For example, with an increased expression of c-Myc in diabetes, insulin-producing β-islet cells dedifferentiate or undergo apoptosis, and the insulin secreted thereby decreases (Magid R et al. J Biol Chem, 2003, 278: 32994). According to the “monoclonal hypothesis” of atherosclerosis, an increased expression of c-Myc in vivo is related to the generation of aortic and carotid plaques. Indeed, it was found in the early coronary artery lesions in Watanabe heritable hyperlipidemic rabbit models and the early lipid accumulation within coronary artery walls of hypercholesterolemic pigs that the c-Myc-dependent signaling pathway was activated, and more importantly, antioxidants down-regulated the overexpression of c-Myc in a manner similar to that observed in tumor cells (Prasad K N et al. Biochem Cell Biol 68, 1250-55).

The c-Myc protein has become one of the most potentially attractive anti-tumor targets. There are currently two main methods reported to inhibit the function of c-Myc protein.

The first method involves directly inhibiting the function of c-Myc protein, comprising inhibiting the binding of Mye/Max dimers and Myc/Max/E-box trimers; and the second method involves eliminating or degrading c-Myc protein.

GSPT1 (G1 to S phase transition 1) mediates the recognition of stop codons and promotes the release of transcribed peptide chains from ribosomes. In addition to playing a role in the transcription termination process, GSPT1 is also closely associated with a variety of important cellular activities, such as cell cycle regulation, cytoskeleton formation and apoptosis. GSPT1 is considered to be an oncogenic factor for various tumors, including breast cancer, liver cancer, gastric cancer and prostate cancer (Cui, Jian, et al. International journal of oncology, 2020, 56(4): 867-878).

Casein kinase 1α (CK1αa) can phosphorylate p53 protein. As a tumor suppressor protein, p53 protein participates in various signal transductions within cells and plays an important role in processes such as cell cycle regulation and apoptosis. Phosphorylated p53 can bind to murine double minute 2 (MDM2) and then be degraded by ubiquitination (iluart, Anne-Sophie, et al. Journal of Biological Chemistry 284.47 (2009): 32384-32394). Therefore, blocking CK1α activity can stabilize p53, thereby exerting its tumor suppressor activity.

The zinc-finger transcription factors IKZF1/2/3 (Aiolos, Helios, Ikaros) belong to the Ikaros zinc-finger (IKZF) family, and are essential for the survival of lymphoid cells. For example, Aiolos can bind to the enhancer of B-cell lymphoma-2 (Bel-2), thereby upregulating the effect of Bel-2 protein on cell survival. Moreover, abnormally activated Helios and Ikaros can upregulate the expression of Bel-XL and drive the occurrence and development of various hematological tumors.

The current methods generally have the disadvantage of poor activity. In order to satisfy this unmet clinical need, the applicant has developed a series of compounds that can efficiently clear c-Myc protein, which can be used to treat diseases related to high expression of various proteins comprising c-Myc.

### SUMMARY OF THE INVENTION

The object of the present invention is to provide a c-Myc protein degradation agent, and a preparation method therefor and use thereof. The compounds of the present invention can be used to degrade various proteins comprising c-Myc protein, such as N-myc, GSPT1, CK1α, IKZF (112/3), AR and AR-V7, thereby preventing, alleviating or treating diseases related to dysregulation of these proteins.

In one aspect, the present invention provides a compound represented by formula (I), and a pharmaceutically acceptable salt, solvent compound, stereoisomer, isotope and prodrug thereof:

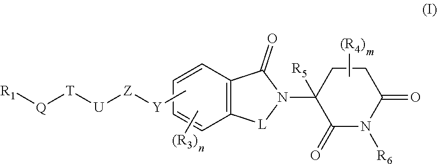

Wherein,
R₁ is selected from: Rₐ(CH₂)ₐ—, Rₐ(CH₂)ₐ—C(═O)—, Rₐ(CH₂)ₐNHC(═O)—, Rₐ(CH₂)OC(═O) or Rₐ(CH₂)ₐS(═O)₂—, wherein the “CH₂” is optionally substituted with one or more R₉, or is optionally substituted with —CH₂CH₂—, and the “NH” is optionally substituted with R₁₄.
Q is selected from: —NR—, —O—

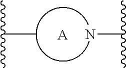

R₂ is selected from: hydrogen, R_{b}—, RC_{b}(═O)—, RS_{b}(═O)₂—, R₁₄;
Ring A represents an optionally substituted heterocycloalkyl containing at least one N atom as a heteroatom, and is linked to T or R₁ through the N atom (that is, when the nitrogen atom in ring A is connected to T, the other connection sites in ring A are connected to R₁; conversely, when it is connected to R₁, the other connection sites in ring A are connected to T), and ring A is alternately substituted with one or more groups selected from R₉;
Rₐ, R_{b} is selected from: C₁-C₈ alkyl, C₃-C₁₀ cycloalkyl, C₃-C₁₀ bridged cyclic group, —NR₁₁(n₁₂, C₃-C₁₀ heterocyclyl optionally containing O, S, SO₂, N or NHC(═O)R₂₂, aryl, heteroaryl, fused arylcycloalkyl, fused alkheterocyclyl, fused heteroarylcycloalkyl, fused heteroarylheterocyclyl, aryl-aryl, aryl-heteroaryl, heteroaryl-aryl, heteroaryl-heteroaryl, aryl-alkyl, heteroary-alkyl, aryl-cycloalkyl, aryl-heterocyclyl, cycloalkyl-heterocyclyl or heterocyclyl-heterocyclyl, wherein a group selected from one of —O—, —S—, —C(═O)—, —S(═O)—, —S(═O)₂—, —NH—, —NHC(═O)—, —NHC(═O)NH— or —NHS(═O)₂— may be inserted between any two C—C of the C₁-C₈ alkyl, and Rₐ may be optionally substituted with one or more R₉, R_{b} may be optionally substituted with one or more R₁₀;
T, U and Z are each independently selected from: chemical bond, N, O, carbonyl, C₁-C₆ alkylene, C₃-C₁₀ cycloalkylene, arylene, heteroarylene or heterocyclylene, wherein the alkylene, cycloalkylene, arylene, heteroarylene or heterocyclylene may be optionally substituted with one or more R₉;
Y is selected from: chemical bond, —C(═O)NH(CH₂)_{b}—, —NHC(═O)_{b}(CH₂)—, —O(CH₂)_{b}—, —NR₂(CH₂)_{b}—, wherein the “CH₂” is optionally substituted with one or more R₉, or is optionally substituted with —CH₂CH₂—, and the “NH” is optionally substituted with R₁₄;
L is selected from —(CR₇R₈)0— or —C(═O)—;
R₃—R₅ and R₇-R₉ are each independently selected from: hydrogen, R₁₃, halogen, cyano, amino, hydroxyl, sulfhydryl, nitro, —R₂₁N(R₂₂)R₂₂, —R₂₂CO(═O)₂₂, —R₂₁NHC(═O)R₂₂, —R₂₃C(═O)NHR₂₂, —R₂₁C(═O)OR₂₂, —R₂₁OC(═O)R₂₂, —R₂₁S(═O)₂R₂₂, —R₂[S(═O)₂NHR₂₂, C₁-C₆ alkyl C₁-C₆ alkoxy, C₁-C₆ alkylthio, C₁-C₆ alkylamino, di(C₁-C₆alkyl)amino, C₃-C₈ cycloalkyl, C₂-C₈ alkenyl, C₂-C₈ alkynyl, aryl, 5- to 6-membered heteroaryl containing 1-3 heteroatoms or 3- to 10f-membered heterocyclyl containing 1-3 heteroatoms, wherein the alkyl, alkoxy, alkylamino, alkylthio, cycloalkyl, aryl, heteroaryl or heterocyclyl is optionally substituted with the groups selected from halogen, cyano, C₁-C₃ alkyl or C₁-C₃ alkoxy; when there are multiple R₃-R₅ and R₇-R₉, any two adjacent ones may be combined to form a ring;
R₁₀ is selected from: hydrogen, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxy, C₁-C₆ alkylthio, C₁-C₆ alkylamino, di(C₁-C₆alkyl)amino, C₃-C₈ cycloalkyl group, C₂-C₈ alkenyl, C₂-C₈ alkynyl, aryl, 5- to 6-membered heteroaryl containing 1-3 heteroatoms or 3- to 10-membered heterocyclyl containing 1-3 heteroatoms, wherein the alkyl, alkoxy, alkylamino, alkylthio, cycloalkyl, aryl, heteroaryl or heterocyclyl is optionally substituted with the groups selected from halogen, cyano, C₁-C₃ alkyl or C₁-C₃ alkoxy; When R₁₀ (is more than one, any two adjacent ones may be combined to form a ring;
R₁₃ is selected from: hydroxyl, sulfhydryl, amino, Rₙ, —ORₙ, —C(═O)Rn, —C(═O)ORₙ, —OC(═O)R₁, —NRₘRₙ, —C(═O)NRₘRₙ, —NRₘ—C(═O)Rₙ, —NRₙ—C(═O)Rₘ, —S(═O)₂Rₙ, —S(═O)NRₘRₙ, —NRₘ—S(═O)₂Rₙ, —NRₙ—S(═O)₂Rₘ;
R₁₄ is selected from: Rₙ, —C(═O)Rₙ, —C(═O)ORₙ, —CC(═O)NRₘRₙ, —S(═O)₂Rₙ, —S(═O)₂NRₘ,Rₙ;
Rₙ is selected from: C₁-C₈ alkyl substituted with hydroxyl, sulfhydryl, or amino group, wherein a group selected from one of —O—, —S—, —C(═O)—, —C(═O)O—, —OC(═O)O—, —S(═O)—, —S(═O)₂—, —NH—, —NHC(═O)—, —NHC(═O)NH—, —NHS(═O)₂— may be inserted between any two C—C of the C₁-C₈ alkyl; C₁-C₈ alkyl is each optionally substituted with 1-3 groups selected from halogen, cyano, amino, nitro or C₁-C₃ alkoxy;
Rₘ is independently selected from: hydrogen, C₁-C₈ alkyl, wherein a group selected from one of —O—, —S—, —C(═O)—, —C(═O)O—, —OC(═O)O—, —S(═O)—, —S(═O)₂—, —NH—, —NHC(═O)—, —NHC(═O)NH—, —NHS(═O)₂— may be inserted between any two C—C of the C₁-C₈ alkyl, C₁-C₈ alkyl is each optionally substituted with 1-3 groups selected from halogen, cyano, amino, nitro or C₁-C₃ alkoxy;
R₆ is selected from: hydrogen, C₁-C₆ alkyl, C₁-C₈ cycloalkyl, C₂-C₈ alkenyl, C₂-C₈ alkynyl, aryl, 5- to 6-membered heteroaryl containing 1-3 heteroatoms or 3- to 10-membered heterocyclyl containing 1-3 heteroatoms, wherein the alkyl, cycloalkyl, aryl, heteroaryl or heterocyclyl is optionally substituted with 1-3 groups each selected from halogen, hydroxyl, Cyano, amino, nitro, —C(═O)OR₂₃, —OC(═O)R₂₃, —NHC(═O)R₂₃, —C(═O)NHR₂, C₁-C₃ alkyl, C₁-C₃ alkoxy or —OP(═O)(OM)₂;
M is independently selected from: hydrogen or C₁-C₄ alkyl;
R₁ and R_{1I} are each independently selected from: hydrogen, C₁-C₄ alkyl, aryl, aryl-alkyl;
R₂₁ is selected from: chemical bond, C₁-C₄ alkylene;
R₂₂ and R₂₃ are each independently selected from: hydrogen, C₁-C₄ alkyl, aryl, aryl-alkyl, and the C₁-C₄ alkyl, aryl, aryl-alkyl are optionally substituted with halogen, hydroxyl, amino;
a is selected from: 0, 1, 2, 3, 4 or 5;
b is selected from: 0, 1, 2, 3, 4 or 5;
ft is selected from: 0, 1, 2 or 3;
m is selected from: 0, 1, 2, 3 or 4;
o is selected from: 1 or 2;

Preferably, the compound of Formula (I) comprises at least one group selected from R₁₃ or R₁₄.

Preferably, not following compounds are in formula (I):

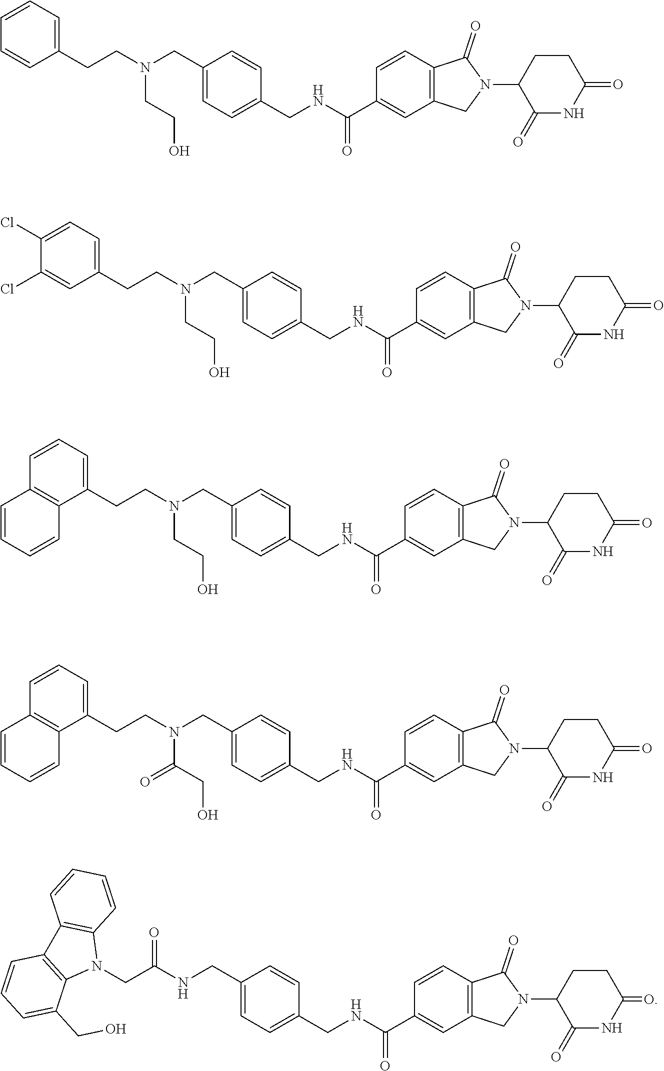 [SMILES: O=C1CCC(N2Cc3cc(C(=O)NCc4ccc(CN(CCO)CCc5ccccc5)cc4)ccc3C2=O)C(=O)N1 ; O=C1CCC(N2Cc3cc(C(=O)NCc4ccc(CN(CCO)CCc5ccc(Cl)c(Cl)c5)cc4)ccc3C2=O)C(=O)N1 ; O=C1CCC(N2Cc3cc(C(=O)NCc4ccc(CN(CCO)CCc5cccc6ccccc56)cc4)ccc3C2=O)C(=O)N1 ; O=C1CCC(N2Cc3cc(C(=O)NCc4ccc(CN(CCc5cccc6ccccc56)C(=O)CO)cc4)ccc3C2=O)C(=O)N1 ; O=C(Cn1c2ccccc2c2cccc(CO)c21)NCc1ccc(CNC(=O)c2ccc3c(c2)CN(C2CCC(=O)NC2=O)C3=O)cc1]

In some embodiments, the present invention provides compounds of Formula (I) and pharmaceutically acceptable salt, solvent compound, stereoisomer, isotope and prodrug thereof:

Wherein,

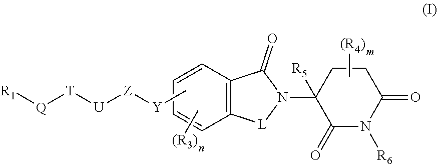

R₁ is selected from: Rₐ(CH₂)ₐ, Rₐ(CH₂)ₐC(═O)—, Rₐ(CH₂)ₐNHC(═O)—, Rₐ(CH₂)ₐOC(═O)—, or Rₐ(CH₂)ₐS(═O)₂—, wherein the “CH₂” is optionally substituted with one or more R₉, or is optionally substituted with CH₂CH₂—, and the “NEH” is optionally substituted with R₁₄.
Q is selected from: —NR₂—, —O—

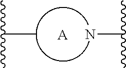

R₂ is selected from: hydrogen, R_{b}—, RC_{b}(═O)—, RS_{b}(═O)₂—, R₁₄;
Ring A represents an optionally substituted heterocycloalkyl containing at least one N atom as a heteroatom, and is linked to T or R₁ through the N atom, and ring A is alternately substituted with one or more groups selected from R₉;
Rₐ, R_{b} is selected from: C₁-C₈ alkyl, C₃-C₁₀ cycloalkyl, C—C bridged cyclic group, —NR₁₁R₁₂, C₃-C₁₀ heterocyclyl optionally containing O, S, SO₂, N or NHC(═O)R₂₂, aryl, heteroaryl, fused arylcycloallyl, fused arylheterocyclyl, fused heteroarylcycloalkyl, fused heteroarylheterocyclyl, ary-alkyl, heteroaryl-alkyl, aryl-cycloalkyl, aryl-heterocyclyl, cycloalkyl-heterocyclyl or heterocyclyl-heterocyclyl, wherein a group selected from one of —O—, —S—, —C(═O)—, —S(═O)—, —S(═O)₂—, —NH—, —NHC(═O)—, —NHC(═O)NH— or —NHS(═O)₂— may be inserted between any two C—C of the C₁-C₈ alkyl, and R, may be optionally substituted with one or more R₉, R_{b} may be optionally substituted with one or more R₁₀;
T, U and Z are each independently selected from: chemical bond, carbonyl, C₁-C₆ alkylene, C₃-C₁₀ cycloalkylene, arylene, heteroarylene or heterocyclylene, wherein the alkylene, cycloalkylene, arylene, heteroarylene or heterocyclylene may be optionally substituted with one or more R₉;
Y is selected from: chemical bond, C(═O)NH(CH₂)_{b}—, —NHC(═O) (CH₂)_{b}—, —O(CH₂)_{b}—, —NR₂(CH₂)_{b}—, wherein the “CH₂” is optionally substituted with one or more R₉, or is optionally substituted with —CH₂CH₂—, and the “NH” is optionally substituted with R₄;
L is selected from —(CR₇R₈), or —C(═O)—;
R₃—R₅ and R₇R₉ are each independently selected from: hydrogen, R₃, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxy, C₁-C₆ alkylthio, C₁-C₆ alkylamino, di(C₁-C₆ alkyl)amino, C₃-C₈ cycloalkyl, C₂-C₈ alkenyl, C₂-C₈ alkynyl, aryl, 5-to 6-membered heteroaryl containing 1-3 heteroatoms or 3- to 10-membered heterocyclyl containing 1-3 heteroatoms, wherein the alkyl, alkoxy, alkylamino, alkylthio, cycloalkyl, aryl, heteroaryl or heterocyclyl is optionally substituted with the group selected from halogen, cyano, C₁-C₃ alkyl or C₁-C₃ alkoxy; when there are multiple R₃-R₅ and R₇-R₉, any two adjacent ones may be combined to form a ring;
R₁₀ is selected from: hydrogen, halogen, cyano, amino, nitro, —R₂₁C(═O)R₂₂, —R₂₁NHC(═O)R₂₂, —R₂₁C(═O)NHR₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxy, C₁-C₆ alkylthio, C₁-C₆ alkylamino, di(C₁-C₈ alkyl)amino, C₃-C₈ cycloalkyl, C₂-C₈ alkenyl, C₂-C₈ alkynyl, aryl, 5- to 6-membered heteroaryl containing 1-3 heteroatoms or 3- to 10-membered heterocyclyl containing 1-3 heteroatoms, wherein the alkyl, alkoxy, alkylamino, alkylthio, cycloalkyl, aryl, heteroaryl or heterocyclyl is optionally substituted with the group selected from halogen, cyano, C₁-C₃ alkyl or C₁-C₃ alkoxy; When R₁₀ is more than one, any two adjacent ones may be combined to form a ring;
R₁₃ is selected from: hydroxyl, Rₙ, —ORₙ, —C(═O)Rₙ, —C(═O)ORₙ, —OC(═O)Rₙ, —NRₘRₙ, C(═O)NRₘRₙ, —NRₘ—C(═O)Rₙ, —NRₙ—C(═O)Rₘ, —S(═O)₂Rₙ—S(═O)₂NRₘRₙ, —NRₘ—S(═O)₂Rₙ, —NRₙ—S(═O)₂Rₙ;
R₁₄ is selected from: Rₙ, —C(═O)Rₙ, —C(C)Rₙ,(═O)NRₘRₙ, —S(═O)₂R, S(═O)₂NRₘRₙ;
Rₙ is selected from: C₁-C₈ alkyl substituted with hydroxyl, wherein a group selected from one of —O—, —S—, —C(═O), —OC(═O)O, —OC(═O)O, —S(═O), —S(═O)₂, —NH, —NHC(═O)—, —NHC(═O)NH—, —NHS(═O)₂— may be inserted between any two C—C of the C₁-C₈ alkyl; C₁-C₈ alkyl is each optionally substituted with 1-3 groups selected from halogen, cyano, amino, nitro or C₁-C₃ alkoxy;
Rₘ; is independently selected from: hydrogen, C₁-C₈ alkyl, wherein a group selected from one of —O, —S—, —C(═O)—, —C(═O)O—, —OC(═O)O—, —S(═O)—, —S(═O)₂,{—NH—, —NHC(═O)—, —NHC(═O)NH, —NHS(═O)₂— may be inserted between any two C—C of the C₁-C₈ alkyl, C₁-C₈ alkyl is each optionally substituted with 1-3 groups selected from halogen, hydroxyl, cyano, amino, nitro or C₁-C₃ alkoxy;
Rₘ is selected from: hydrogen, C₁-C₆ alkyl, C₃-C₈ cycloalkyl, C₂-C₈ alkenyl, C₂-C₅alkynyl, aryl, 5- to 6-membered heteroaryl containing 1-3 heteroatoms or 3- to 10-membered heterocyclyl containing 1-3 heteroatoms, wherein the alkyl, cycloalkyl, aryl, heteroaryl or heterocyclyl is optionally substituted with 1-3 groups each selected from halogen, hydroxyl, cyano, amino, nitro, —C(═O)OR₂₃, —OC(═O)R₂₃, —NHC(═O)R₂₃, —C(═O)NHR₂₃, C₁-C₃ alkyl, C₁-C₃ alkoxy or —OP(═O)(OM)₂;
M is independently selected from: hydrogen or C₁-C₄ alkyl;
R₁₁ and R₁₂ are each independently selected from: hydrogen, C₁-C₄ alkyl, aryl, aryl-alkyl;
R₂₁ is selected from: chemical bond, C₁-C₄ alkylene;
R₂₂ and R₂₃ are each independently selected from: hydrogen, C₁-C₄ alkyl, aryl, aryl-alkyl, and the C₁-C₄ alkyl, aryl, aryl-alkyl are optionally substituted with halogen, hydroxyl, amino:
a is selected from: 0, 1, 2, 3, 4 or 5;
b is selected from: 0, 1, 2, 3, 4 or 5;
n is selected from: 0, 1, 2 or 3;
m is selected from: 0, 1, 2, 3 or 4;
o is selected from: 1 or 2;

Preferably, the compound of Formula (1) comprises at least one group selected from R₁₀ or R₁₄.

Preferably, not following compounds are in formula (I):

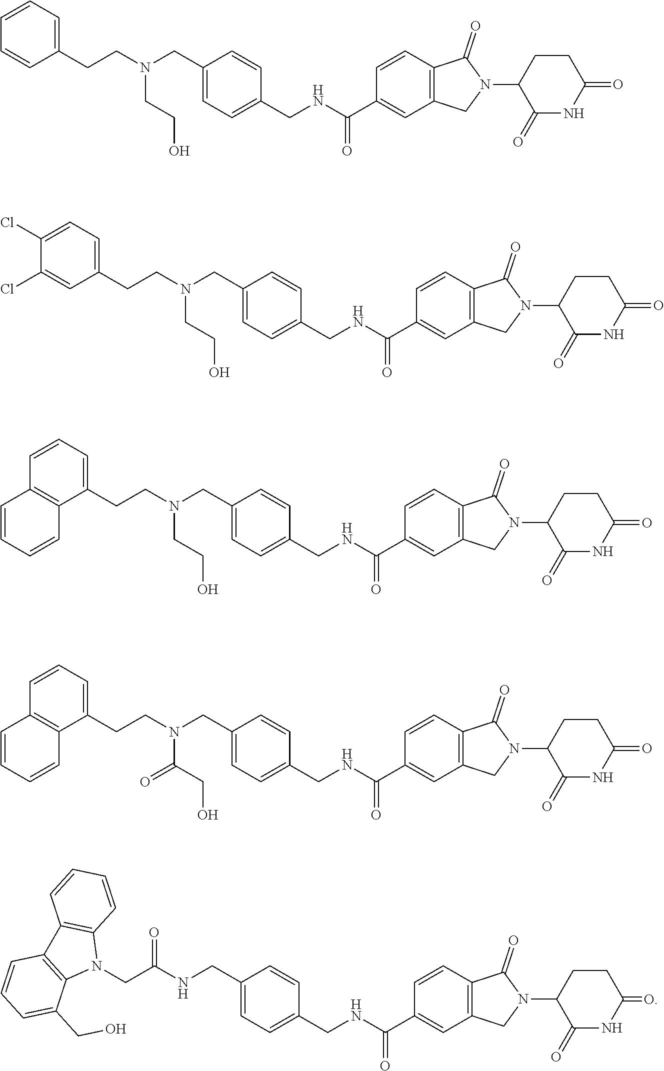 [SMILES: O=C1CCC(N2Cc3cc(C(=O)NCc4ccc(CN(CCO)CCc5ccccc5)cc4)ccc3C2=O)C(=O)N1 ; O=C1CCC(N2Cc3cc(C(=O)NCc4ccc(CN(CCO)CCc5ccc(Cl)c(Cl)c5)cc4)ccc3C2=O)C(=O)N1 ; O=C1CCC(N2Cc3cc(C(=O)NCc4ccc(CN(CCO)CCc5cccc6ccccc56)cc4)ccc3C2=O)C(=O)N1 ; O=C1CCC(N2Cc3cc(C(=O)NCc4ccc(CN(CCc5cccc6ccccc56)C(=O)CO)cc4)ccc3C2=O)C(=O)N1 ; O=C(Cn1c2ccccc2c2cccc(CO)c21)NCc1ccc(CNC(=O)c2ccc3c(c2)CN(C2CCC(=O)NC2=O)C3=O)cc1]

In the present invention, preferably, Rₙ is selected from: C₁-C₈ alkyl substituted with hydroxyl; More preferably, Rₙ is selected from: C₁-C₄ alkyl substituted with hydroxyl; Most preferably, Rₙ is selected from: —CH₂OH, —CH₂CH₂OH, —CH₂CH₂CH₂OH or —CH₂CH₂CH₂CH₂OH. Preferably, Rₘ is selected from: hydrogen, —C₁-C₄ alkyl.

In the present invention, preferably, Rₙ is selected from: C₁-C₄ alkyl substituted with hydroxyl, sulfhydryl or amino; Preferably, Rₙ is selected from: C₁-C₄ alkyl substituted with hydroxyl, sulfhydryl or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH; Rₘ˜is selected from: hydrogen, —C₁-C₄ alkyl.

In some embodiments, R₁ is selected from: RₐC(═O)—, RₐCH₂C(═O)—, RₐCH₂CH₂C(═O)—, Rₐ—, RₐCH₂—, RₐCH₂CH₂—, RₐNHC(═O)—, RₐCH₂NHC(═O)—, RₐCH₂CH₂NHC(═O)—, RₐOC(═O)—, RₐCH₂OC(═O)—, RₐCH₂CH₂OC(═O)—, RₐS(═O)₂—, RₐCH₂S(═O)₂—, RₐCH₂CH₂S(═O)₂—, wherein CH₂ is optionally substituted with one or more R₉, or optionally substituted with —CH₂CH₂—; wherein NH is optionally substituted with R₁₄;

Preferably, R₁ is selected from: RₐC(═O)—, RₐCHR₉C(═O), RₐCH₉R₉CH₂C(═O)—, RₐCHCHR₉C(═O)—, Rₐ—, RₐCHRₐ—, RₐCHR₉CH₂—, RₐCHCHR₉, RₐNR₁₄C(═O)—, RₐCHR₉NHC(═O)—, RₐCHNR₁₄C(═O)—, R₉CHR₉CH₂NHC(═O)—, RₐCH₂CHR₉NHC(═O)—, RₐCH₂CH₂NR₁₄C(═O)—, RₐOC(═O)—, RₐCHR₉OC(═O)—, RₐCHR₉CH₂OC(═O)—, RₐCH₂CHR₉OC(═O)—, RₐCHR₉S(═O)₂—, RₐCHR₉CH₂S(═O)₂, Rₐ CHR₉S(═O).

Preferably, R₉ is selected from: hydrogen, C₁-C₄ alkyl, R₁₃; More preferably, R₉ is selected from: R₁₃.

Preferably, R₁₃ is selected from: hydroxyl, Rₙ, —ORn.

Preferably, R₁₄ is selected from: R₁, —C(═O)R.

Preferably, Rₙis selected from: C₁-C₈ alkyl substituted with hydroxyl; More preferably, Rₙ, is selected from: C₁-C₄ alkyl substituted with hydroxyl; Most preferably, Rₙ is selected from: —CH₂OH, —CH₂CH₂OH, —CH₂CH₂CH₂OH or —CH₂CH₂CH₂CH₂OH.

In some embodiments, R₁ is selected from: RC(O)—, RₐCH₂C(═O)—, RₐCH₂CH₂C(═O)—, Rₐ—, RₐCH₂—, RₐCH₂CH₂—, RₐNHC(═O)—, RₐCH₂NHC(═O)—, RₐCH₂CH₂NHC(═O)—, RₐOC(═O)—RₐCH₂OC(═O)—, RₐCH₂CH₂OC(═O), RₐS(═O)₂—, RₐCH₂S(═O)₂—, RₐCH₂CH₂S(═O)₂—, wherein CH₂ is optionally substituted with one or more R₉, or optionally substituted with —CH₂CH₂—. wherein NH is optionally substituted with R₁₄

Preferably, R₁ is selected from: RₐC(═O)—, RₐCHR₉C(═O)—, RₐCHR₉CH₂C(═O)—RₐCHCHR₉C(═O), Rₐ—, RₐCHR₉—, RₐCHR₉CH₂—, RₐCHCHR₉—, RₐNR₄C(═O)—, Rₐ CHR₉NHC(═O)—, RₐCH NR₁₄C(═O)—, RₐCHR₉CH₂NHC(═O)—, RₐCH₂R₉NHC(═O)—, RₐCH₂CH₂NR₁₄(═O)—, RₐOC(═O), RₐCHR₉OC(═O)—, RₐCHR₉CH₂OC(═O)—RₐCH₂CHR₉OC(═O)—, RₐCHR₉S(═O)₂—, RₐCHR₉CH₂S(═O)₂—, RₐCH₂CHR₉S(═O)₂—. Preferably, R₉ is selected from: hydrogen, C₁-C₄ alkyl, —R₂₁N(R₂₂)R₂₂, —R₂₁C(═O)R₂₂, R₁₃; More preferably, R₉ is selected from: -R₂₁N(R₂₂)R₂₂, —R₂₁C(═O)R₂₂, R₁₃.

Preferably, the R₃ is selected from: hydroxyl, sulfhydryl, amino, Rₙ, —ORn.

Preferably, the R₁. is selected from: Rₙ, —C(═O)Rn, Preferably, Rₙ is selected from: C₁-C₈ alkyl substituted with hydroxyl, sulfydryl or amino; Preferably, Rₙ is selected from: C₁-C₄ alkyl substituted with hydroxyl, sulfhydryl or amino; More preferably, R₁ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂ CH₂CH₂CH₂NH₂, —CH₂SH, —CH₂CH₂SH, —CH₂CH₂CH₂SH, —CH₂CH₂CH₂CH₂SH.

In some embodiments, Rₐ is selected from: phenyl, naphtyl, carbazolyl, 1-aza-carbazolyl, 2-aza-carbazolyl, 1, 8-diaza-carbazolyl, indolyl, 7-aza-indoleyl, 2,3-dihydroindolyl, 2,3-dibydro-7-aza-indole, phenoxazinyl, fluorenyl, quinolinyl, isoquinolinyl, nalididyl, tetrahydronalididyl, tetrahydroquinolinyl, pyrimidinyl, triazolyl, bicyclo[1.1.11penityl, norborneyl, adamantane. Preferably, Rₐ, is optionally substituted with one or more R₉, wherein R) is selected from hydrogen, R₁₃, halogen, cyano, C₁-C₆ alkyl, C₁-C₆ haloalkyl, C₁-C₆ alkoxyl, phenyl, naphlthyl.

Preferably, Rₐ is selected from: phenyl, naphthalen-1-yl, naphthalen-2-yl, carbazole-9-yl, 1-aza-carbazole-9-yl, 2-aza-carbazole-9-yl, 1,8-diaza-carbazole-9-yl, indole-1-yl, 2,3-dihydro-indole-1-yl, 7-aza-indole-1-yl, 2,3-dihydro-7-aza-indole-1-yl, phenoxazine-10-yl, fluorene-9-yl, quinoline-4-yl, quinoline-5 yl, quinoline-8-yl, isoquinoline-1-yl, isoquinoline-4-yl, isoquinoline-5-yl, isoquinoline-8-, 1,2,3,4-tetrahydro-1,8-naphthyridine-1-yl, 1,2,3,4-tetrahydroquinoline-1-yl, pyrimidine-2-yl, pyrimidine-4-yl, pyrimidine-5-yl, 1,2,3-triazol-1-yl, 1,2,4-triazol-1-yl, 1,3,4-triazol-1-yl, bicyclo[1.1.1]pentane-1-yl, norborne-1-yl, adamantane-1-yl. Preferably, Rₙ is optionally substituted with one or more R₉ selected from: R₃, F, Cl, Br, cyano, methyl, ethyl, trifluoromethyl, methoxyl, ethoxyl, phenyl, naphthalen-1-yl, naphthalen-2-yl.

Preferably, R₃ is selected from: hydroxyl, Rₙ, —O, —OC(═O)R, —NRₘRₙ, —NRₘ—C(═O)R, —NR—C(═O)Rₘ, —NRₙ—S(═O)₂Rₙ, —NRₙ—S(═O)₂Rₘ; More preferably, R₁₃ is selected from: hydroxyl, Rₙ, —ORₙ, —NRₘRₙ, —NRₙC(═O)Rₘ, —NRₙ—S(═O)₂Rₘ.

Preferably, Rₙ is selected from: C₁-C₈ alkyl substituted with hydroxyl; More preferably, R₁ is selected from: C₁-C₄ alkyl substituted with hydroxyl; Most preferably, Rₙis selected from: —CH₂OH, —CH₂CH₂OH, —CH₂CH₂CH₂OH or —CH₂CH₂CH₂CH₂OH.

Preferably, Rₘ is selected from: hydrogen, -C₁-C₄ alkyl.

Preferably, R₉ is selected from: F, Cl, Br, cyano, methyl, ethyl, trifluoromethyl, methoxyl, ethoxyl, hydroxyl, —CH₂OH, —CH₂CH₂OH, —CH₂CH₂CH₂OH, —OCH₂OH—OCH₂CH₂OH, —OCH₂CH₂CH₂OH, —NHCH₂OH, —NHCH₂CH₂OH, —NHCH₂CH₂CH₂OH, —N(CH₃)CH₂OH, —N(CH₃)CH₂CH₂OH, —N(CH₃)CH₂CH₂CH₂OH, —N(COCH₃)CH₂OH, —N(COCH₃)CH₂CH₂HOH, —N(COCH₃)CH₂CH₂CH₂OH, —N(SO₂CH₃)CH₂OH, —N(SO₂CH₃)CH₂CH₂OH, —N(SO₂CH₃)CH₂CH₂CH₂OH.

Preferably, Rₙ is selected from the following groups:

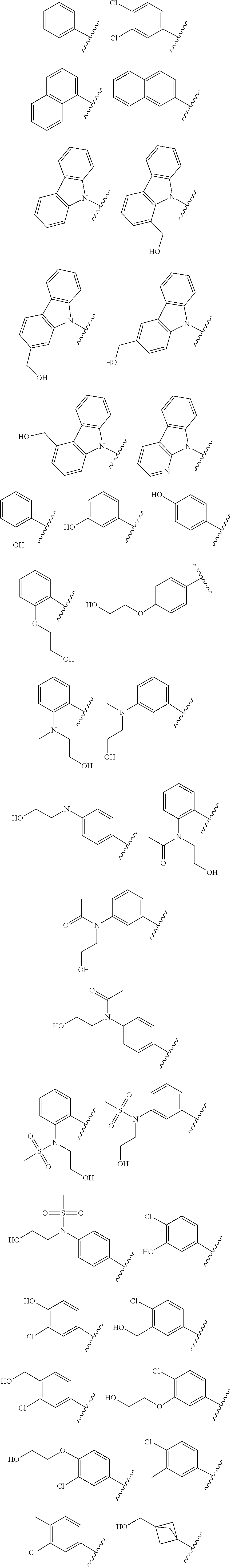

In some embodiments, Rₙ is selected from: phenyl, naphthyl, carbazolyl, 1-aza-carbazolyl, 2-aza-carbazolyl, 1, 8-diaza-carbazolyl, indolyl, 7-aza-indoleyl, 2, 3-dihydroindolyl, 2, 3-dihydro-7-aza-indoleyl, phenoxazinyl, fluorenyl, quinolinyl, isoquinolinyl, nalididyl, tetrahydronalididyl, tetrahydroquinolinyl, pyrimidinyl, pyridinyl, quinoline-pyridinyl, triazolyl, bicyclo[1.1.1]pentyl, norbornyl, adamantyl. Preferably, Rₐ is optionally substituted with one or more R₉. Wherein R₉ is selected from hydrogen, hydroxyl, sulfhydryl, —R₂₁N(R₂₂) R₂, —R₂₁C(═O)RR₂₂, R₁₃, halogen, cyano, C₁-C₆ alkyl, C₁-C₆ haloalkyl, C₁-C₆ alkoxyl, phenyl, naphthyl.

In some specific embodiments, Rₐ is selected from: isoquinoline-pyridyl. Preferably, Rₐ is optionally substituted with one or more Rn Wherein R_{g} is selected from hydrogen, hydroxyl, sulfhydryl, —R₂₁N(R₂₂) R₂₂, —R₂₂C(═O)R₂₂, R₁₃, halogen, cyano, C₁-C₆ alkyl, C₁-C₆ haloalkyl, C₁-C₆ alkoxyl, phenyl, naphthyl.

Preferably, Rₐ is selected from: phenyl, naphthalen-1-yl, naphthalen-2-yl, carbazole-9-yl, 1-aza-carbazole-9-yl, 2-aza-carbazole-9-yl, 1,8-diaza-carbazole-9-yl, indole-1-yl, 2, 3-dihydro-indole-1-yl, 7-aza-indole-1-yl, 2, 3-dihydro-7-aza-indole-1-yl, phenoxazine-10-yl, fluorene-9-yl, quinoline-4-yl, quinoline-5-yl, quinoline-8-yl, isoquinoline-1-yl, isoquinoline-4-yl, isoquinoline-5-yl, isoquinoline-8-yl, 1,2,3,4-tetrahydro-1,8-naphthyridine-l-yl, 1,2,3,4-tertrahydro-quinoline-1-yl, pyrimidine-2-yl, pyrimidine-4-yl, pyrimidine-5-yl, pyridine-2-yl, pyridine-3-yl, pyridine-4-yl, 5-(8-quinoline-yl)-pyridine-2-yl, 1,2,3-triazol-1-yl, 1,2,4-triazol-1-yl, 1,3,4-triazol-1-yl, bicyclol1.1.1pentane-1-yl, norborne-1-yl, adamantane-1-yl; Preferably, Rₐ is optionally substituted with one or more R₉, wherein R₉ is selected from: R₁, F, Cl, Br, cyano, methyl, ethyl, trifluoromethyl, methoxyl, ethoxy, phenyl, naphthalen-1-yl, naphthalen-2-yl.

In some specific embodiments, Rₐ is selected from: 5-(I-isoquinoline-yl)-pyridin-2-yl.

Preferably, R is optionally substituted with one or more R₉. Wherein it R₉ is selected from: R₁₃, F, Cl, Br, cyano, methyl, ethyl, trifluoromethyl, methoxy, ethoxy, phenyl, naphthalen-1-yl, naphthalen-2-yl.

Preferably, R₁₃ is selected from: hydroxyl, sulfhydryl, amino, Rₙ, —ORₙ, —OC(═O)Rₙ, —NRₘRₙ, —NRₘ-CO(═O)R, —NRₙ—C(═O)Rₘ, —NRₘ—S(═O)₂,R, —NRₙ,—S(═O)₂Rₘ; More preferably, R₁₃ is selected from: hydroxyl, sulfhydryl, amino, Rₙ, —ORₙ, —NRₘRₙ, —NRₙ—C(═O)Rₘ, —NRₙ—S(═O)₂Rₘ.

Preferably, Rₙ is selected from: C₁-C₈ alkyl substituted with hydroxyl, sulflhydryl, or amino; Preferably, the Rₙ is selected from: C₁-C₄ alkyl substituted with hydroxyl, sulflhydryl, or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH.

Preferably, Rₘ is selected from: hydrogen, —C₁-C₄ alkyl.

Preferably, R₉ is selected from: F, Cl, Br, cyano, methyl, ethyl, trifluoromethyl, methoxyl, ethoxyl, hydroxyl, sulfhydryl, amino, —C(═O)CH₃, C(═O)CH₂CH₃, C(═O)CH₂CH₂CH₃, —CH₂CH₂N(CH₃)₂, —CH₂OH, —CH₂CH₂OH, —CH₂CH₂CH₂CH₂, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —OCH₂OH, —OCH₂CH₂OHOH₂, —OCH₂O₂NH₂, —OCH₂CH₂NH, —OCH—NH₂, —OCH₂SH, —OCH₂CH₂SH, —OCH₂CH₂CH₂SH, —NHCH₂OH, —NHCH₂CH₂OH, —NHCH₂CH₂CH₂OH, —NHCH₂NH₂, —NHCH₂CH₂NH₂, —NHCH₂CH₂CH₂NH₂, —NHCH₂SH, —NHCH₂CH₂SH, —NHCH₂CH₂CH₂SH, —N(CH₃)CH₂OH, —N(CH₃)CH₂CH₂OH, —N(CH₃)CH₂CH₂CH₂OH, —N(CH₃)CH₂NH₂, —N(CH₃)CH₂CH₂CH₂, —N(CH₃)CH₂CH₂CH₂CH₂, —N(CH₃)CH₂SH, CH₂CH₂SH, CH₂CHSH, —N(CH₃)CH₂CH₂CH₂SH, —N(COCH₃)CH₂OH, —N(COCH₃)CH₂CH₂OH, N(COCH₃)CH₂C—NH₂CH₂OH, N(COCH₃)CH₂NH, —N(COCH₃)CH₂CH₂NH₂, —N(COCH₃)CH₂CH₂CH₂NH₂, —N(COCH₃)CH₂SH, —N(COCH₃)CH₂, CH₂SH, —N(COCH₃)CH₂CH₂CH₂SH, —N(SO₂CH₃)CH₂Off, —N(SO₂CH₃)CH₂CH₂OH, —N(SO₂CH₃)CH₂CH₂CH₂OH, —N(SO₂CH₃)CH₂NH₂, —N(SO₂CH₃)CH₂CH₂NH₂, —N(SO₂CH₃)CH₂CH₂CH₂NH₂, —N(SO₂CH b)CH₂SH, —N(SO₂CH₃)CH₂CH₂SH, —N(SO₂CH₃)CH₂CH₂CH₂SH.

Preferably, Rₐ is selected from the following groups:

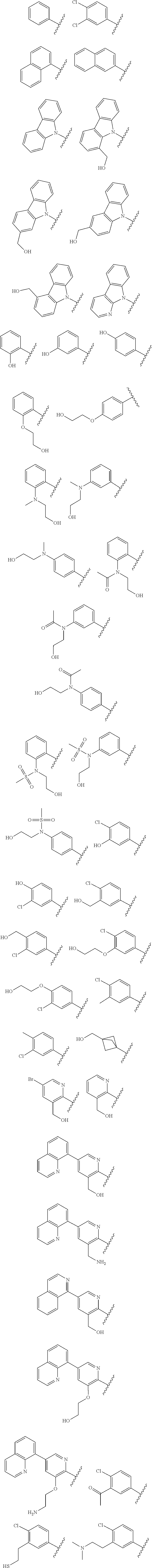

In some particular embodiments, Rₙ is selected from the group:

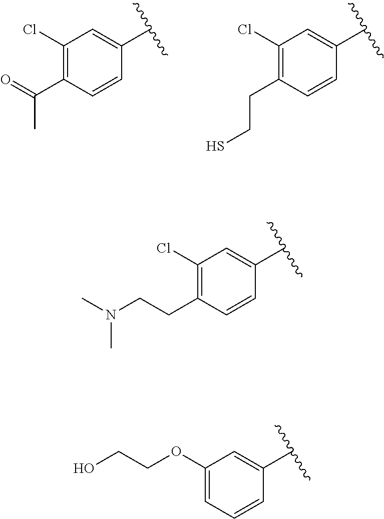 [SMILES: *c1ccc(C(C)=O)c(Cl)c1 ; *c1ccc(CCS)c(Cl)c1 ; *c1ccc(CCN(C)C)c(Cl)c1 ; *c1cccc(OCCO)c1]

In some embodiments, the Q is selected from: —NR₂—

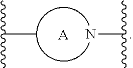

Preferably, R₂ is selected from: hydrogen, R₁₄, C₁-C₈ alky, C₃-C₁₀ cycloalkyl, aryl, —C(═O)C₃-C₁₀ cycloalkyl, —C(═O)C₁-C₈ alkyl or —S(═O)₂C₁-C₈ alkyl, wherein C₁-C₈ alkyl, C₃-C₁₀ cycloalkyl, aryl and the cycloalkyl or alkyl portion of the —C(═O)C₃-C₁₀ cycloalkyl, —C(═O)C₁-C₈ alkyl or —S(═O)₂C₁-C₈ alkyl is optionally substituted with one or more R₉, wherein R₉ is selected from: hydrogen, halogen, amino, cyano, carboxyl, C₁-C₆ alkyl, C₁-C₆ alkoxyl or aryl; Preferably, R₂ is selected from: R₁₄.

Preferably, R₁₄ is selected from: Rₙ, —C(═O)Rₙ, —C(═O)ORₙ, —C(═O)NRₘRₙ, —S(═O)₂Rₙ, —S(═O)₂NRₘRₙ.

Preferably, Rₙ, is selected from: C₁-C₈ alkyl substituted with hydroxyl; More preferably, Rₙ is selected from: C₁-C₄ alkyl substituted with hydroxyl; Most Preferably, Rₙ is selected from: —CH₂OH, —CH₂CH₂OH, —CH₂CH₂CH₂OH or —CH₂CH₂CH₂CH₂OH. In some embodiments, Rₙ is selected from: C₁-C₈ alkyl substituted with hydroxyl, sulfhydryl, or amino; More preferably, Rₙ is selected from: C₁-C₄ alkyl substituted with hydroxyl, sulfhydryl, or amino; Most Preferably, Rₙ, is selected from: —CH₂ OH, —CH₂CH₂OH, —CH₂CH₂CH₂, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SIH.

Preferably, Rₘ is chosen from: hydrogen, —C₁-C₄ alkyl.

Therefore, preferably, R₂ is selected from: hydrogen, methyl, ethyl, —SO₂CH₃, —COCH₃, -CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthyl ethyl, —CH₂OH, —CH₂CH₂OH, —CH₂CH₂CH₂OH, —C(═O)CH₂OH, —C(═O)CH₂CH₂OH, —C(═O)CH₂CH₂CH₂OH, —C(═O)OCH₂OH, —C(═O)OCH₂CH₂OH, —C(═O)OCH₂CH₂CH₂OH, —C(═O)NHCH₂OH, —C(═O)NHCH₂CH₂OH, °—C(═O)NHCH₂CH₂CH₂OH, —S(═O)₂CH₂OH, —S(═O)₂CH₂CHOH, —S(═OC)₂C₂CH₂OH, —S(═O)₂NHCHOH, —S(═O)₂NHCH₂CH₂OH, —S(═O)₂NHCH₂CH₂CH₂OH. In some embodiments, R₂ is further selected from: hydrogen, methyl, ethyl, —SO₂CH₃, —COCH₃, -CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthyl ethyl, —CHOH, —CH₂CH₂OH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —C(═O)CH₂OH, —C(═O)CH₂CH₂OH, —C(═O)CH₂CH₂CH₂OH, —C(═O)CH₂NH₂, —C(═O)CH₂CH₂NH₂, —C(═O)CH₂CH₂CH₂NH₂, —C(═O)CH₂SH, —C(═O)CH₂CH₂SH, —C(═O)CH₂CH₂, —C(═O)OCH₂OH, —C(═O)OCH₂CH₂OH, —C(═O)OCH₂CH₂CH₂OH, —C(═O)OCH₂NH₂, —C(═O)OCH₂CH₂NH₂, —C(═O)OCH₂CH₂CH₂NH₂, —C(═O)OCH₂SH, —C(═O)OCH₂CH₂, —C(═O)OCH₂CH₂CCH₂SH, —C(═O)NHCH₂, —C(═O)NCH₂CH₂OH, —C(═O)NHCH₂CH₂CH₂OH, —C(═O)NHCH₂NH₂, —C(═O)NHCH₂CH₂NH₂, —C(═O)NHCH₂CH₂CH₂NH, —C(═O)NHCH₂SH, —C(═O)NHCH₂CH₂SH, —C(═O)NHCH₂CH₂CH₂SH, —S(═OC)₂CH)₂H, —S(═O)₂CH₂CH₂OH, —S(═O)₂CH₂C—H₂CH₂OH, —S(═O)₂CH₂NH, —S(═O)₂CH₂CH₂NH₂, —S(═O)₂CH₂CH₂CH₂NH₂, —S(═O)₂CH₂SH, —S(═O)₂CH₂CH₂SH, —S(═O)₂CH₂CH₂CH₂SH, —S(═O)₂NHCH₂OH, —S(═O)₂NHCH₂CH₂OH, —S(═O)₂NHCH₂CH₂CH₂OH, —S(═O)₂NHCH₂NH₂, —S(═O)₂NHCH₂CH₂NH₂, —S(═O)₂NHCH₂CH₂CH₂NH, —S(═O)₂NHCH₂SH, —S(═O)₂NHCH₂CH₂SH, —S(═O)₂NHCH₂CH₂CH₂SH.

Preferably,

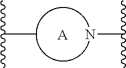

is selected from one of the following structures:

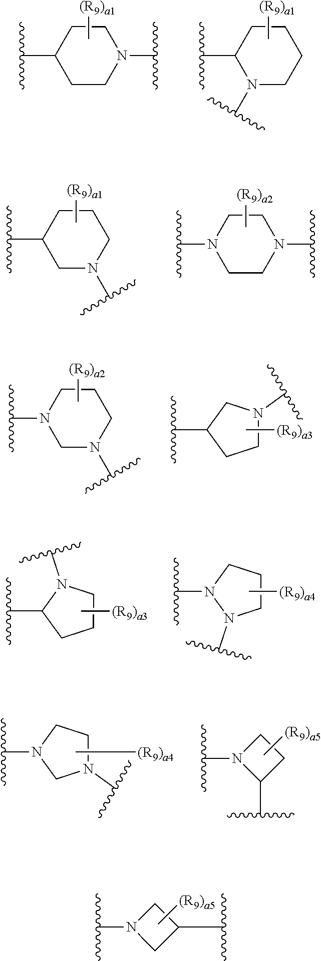

Wherein each R₉ is independently selected from: hydrogen, R₁₃, halogen, cyano, nitro, C₁-C₆ alky, C₁-C₆ haloalkyl, C₁-C₆ alkoxyl; Each at is independently selected from: 0, 1, 2, 3, 4, 5, 6, 7, 8, 9; each a2 is independently selected from: 0, 1, 2, 3, 4, 5, 6, 7, 8; each a3 is independently selected from: 0, 1, 2, 3, 4, 5, 6, 7; each a4 is independently selected from: 0, 1, 2, 3, 4, 5, 6; each a5 is independently selected from: 0, 1, 2, 3, 4, 5.

Preferably, wherein R₁₃ is selected from: hydroxyl, Rₙ, —ORₙ, —OC(═O)Rₙ, —NRₘRₙ, NRₘ—(C═O)Rₙ, —NRₙ,—(C═O)Rₙ, —NRₘ—S(═O)₂Rₙ, —NRₙ—S(═O)₂t; Preferably, R₁₃ is selected from: hydroxyl, Rₙ, —ORₙ.

Preferably, Rₙ, is selected from: C₁-C₈ alkyl substituted with hydroxyl; More preferably, Rₙ is selected from: C₃-C₄ alkyl substituted with hydroxyl; Preferably, R₁₁ is selected from: —CH₂OH, —CH₂CH₂OH, —CH₂CH₂CH₂OH, or —CH₂CH₂CH₂CH₂OH.

In some specific embodiments, R₁₃ is selected from: hydroxyl, sulfhydryl, amino, Rₙ, —OR_{D}, —OC(═O)Rₙ, —NRₘRₙ, —NRₙ—C(═O)Rₙ, —NRₙ—C(═O)Rₘ, —NRₘ—S(═O)₂Rₙ,—NRₙ—S(═O)₂Rₘ;

Wherein Rₙis selected from: C₁-C₈ alkyl substituted with hydroxyl, sulfhydryl, or amino; Preferably, the Rₙ is selected from: C₁-C₄ alkyl substituted with hydroxyl, sulfhydryl, or amino; More preferably, Rₙis selected from: —CH₂OH, —CH₂CH₂H, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH.

Preferably, Rₘ is selected from: hydrogen, —C₁-C₄ alkyl.

Preferably,

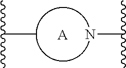

is selected from one of the following structures:

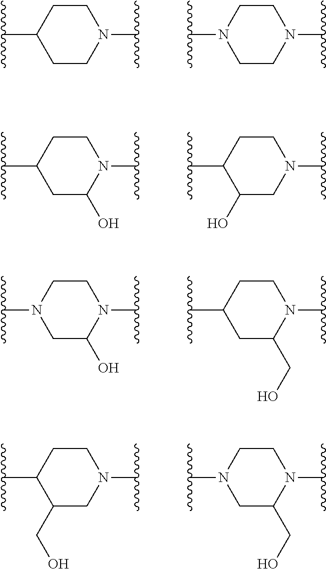

In some embodiments, T, Z are independently selected from: chemical bond, carbonyl, C₁-C₆ alkylene or C₃-C₁₀ cyclohexene, wherein C₁-C₆ alkylene or C₃-C₁₀ cyclohexene is optionally substituted with one or more R₉.

Preferably, T and Z are independently selected from: chemical bond, carbonyl, methylene, 1,2-ethyleneyl, 1,1-cyclopropylene, or 2,2-propylene, wherein methylene, 1,2-ethylidene, 1,1-cyclopropylene is optionally substituted with one or more R₉.

Preferably, wherein R₉ is selected from: hydrogen, R₁₃, C₁-C₆ alkyl or C₃-C₈ cycloalkyl.

More preferably, R₉ is selected from: hydrogen, —R₂₁N(R₂₂)R₂₂, —R_{1I}C(═O)R₂₂, R₁₃, C₁-C₆ alkyl or C₃-C₈ cycloalkyl.

Preferably, R₃ is selected from: hydroxyl, Rₙ, —ORₙ.

Preferably, Rₙis selected from: C₁-C₈ alkyl substituted with hydroxyl; More preferably, Rₙ is selected from: C₁-C₄ alkyl substituted with hydroxyl; Preferably, Rₙ is selected from: —CH₂OH, —CH₂CH₂OH, —CH₂CH₂CH₂OH, or —CH₂CH₂CH₂CH₂OH.

In some specific embodiments, R₉ is selected from: hydrogen, hydroxyl, sulfhydryl, amino, N(R₂₂)R₂₂, —R₂₂C(═O)R₂₂, R₁₃, C₁-C₆ alkyl or C₃-C₈ cycloalkyl; wherein R₁₃ is selected from: hydroxyl, sulfhydryl, amino, Rₙ, —ORₙ; wherein Rₙ is selected from: C₁-C₈ alkyl substituted with hydroxyl, sulfhydryl or amino; Preferably, RH is selected from: C_{f}-C₄ alkyl substituted with hydroxyl, sulfhydryl or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂ SH, —CH₂CH₂ CH₂SH, —CH₂CH₂CH₂CH₂SH.

In some embodiments, U is selected from: C₁-C₆ alkylene, C₃-C₁₁ cycloalkylene, arylene, or heteroarylene, wherein alkylene, cycloalkylene, arylene, or heteroarylene is optionally substituted with one or more R₉.

Preferably, U is selected from: C₂-C₆ alkylene, C₃-C₆ cycloalkylene, C₆-C₁₀ alkylene, 5-6 membered monocyclic heteroarylene, wherein alkylene, cycloalkylene, arylene, or heteroarylene is optionally substituted with one or more R₉.

Preferably, U is selected from: 1,2-ethylidene, 1,3-propylidene, 1,4-butylidene, 1,5-pentylidene, 1,6-hexylidene, 1,3-cyclopentylene, 1,3-cyclohexylidene, 1,4-cyclohexylidene, 1,2-phenylene, 1, 3-phenylene, 1,4-phenylene, 2,5-pyridinylene, 2,5-pyrimidylene, 2,5-thiazolylene or 2,4-oxazolylene, wherein 1,2-ethylidene, 1,3-propylidene, 1,4-butylidene, 1,3-cyclopentylene, 1,3-cyclohexylidene, 1,4-cyclohexylidene, 1,2-phenylene, 1,3-phenylene, 1,4-phenylene, 2,5-pyridinylene, 2,5-pyrimidylene, 2,5-thiazolylene or 2,4-oxazolylene is optionally substituted with one or more R₉.

Preferably, R) is selected from: hydrogen, R₁₃, halogen, C₁-C₆ alkyl, C₁-C₆ haloalkyl, C₁-C₆ alkoxyl.

Preferably, R₁₃ is selected from: hydroxyl, Rₙ—ORₙ, —OC(═O)Rₙ, —NRₘRₙ, —NRₘ—C(═O)Rₙ, —NRₙ—C(═O)Rₘ, —NRₘ—S(═O)₂Rₙ, —NRₐ—S(═O)₂Rₘ; More preferably, R₁₃ is selected from: hydroxyl, Rₙ, —ORₙ, —NRₘRₙ, —NRₙ—C(═O)Rₘ, —NRₙ—S(═O)₂Rₘ.

Preferably, Rₙ is selected from: C₁-C₈ alkyl substituted with hydroxyl; More preferably, Rₙ is selected from: C₁-C₄ alkyl substituted with hydroxyl; Preferably, Rₙ is selected from: —CH₂OH, —CH₂CH₂OH, —CH₂CH₂CH₂OH, or —CH₂CH₂CH₂CH₂OH. In some embodiments, Rn is selected from: C1-C6 alkyl substituted with hydroxyl, sulfitydryl, or amino.

In some specific embodiments, wherein R₉ is selected from: hydrogen, amino, hydroxyl, sulflhydryl, —R₂₁N(R₂₂)R₂, —R₂₁C(═O)R₂₂, R₂₃, halogen, C₁-C₆ alkyl, C₁-C₆ haloalkyl, C₁-C₆ alkoxyl; wherein R₁₃ is selected from: hydroxyl, amino, thiol, Rₙ, —OR. —OC(═O)RC, —NRₘRₙ, —NRₘ—C(═O)Rₙ, —NR—C(═O)Rₘ, —NRₘ—S(═O)₂Rₙ, —NR—S(═O)₂Rₘ; wherein Rₙ is selected from: C₁-C₄ alkyl substituted with hydroxyl, sulfhydryl or amino; More preferably, RP is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, CH₂SH, —CH₂CH₂SH, —CH₂CH₂CH₂SH, —CH₂CH₂CH₂CH₂SH.

Preferably, R₁ is selected from: hydrogen, —C₁-C₄ alkyl.

Thus, preferably, R₉q is selected from: F, Cl, Br, cyano, methyl, ethyl, trifluoromethyl, methoxyl, ethoxyl, hydroxyl, CH₂OH, —CH₂CH₂OH, —CH₂CH₂CH₂OH, —OCH₂OH, —OCH₂CH₂OH, —OCH₂CH₂CH₂OH, —NHCH₂OH, —NHCH₂CH₂OH, —NHCH₂CH₂CH₂OH, —N(CH₃)CH₂OH, —N(CH₃)CH₂CH₂OH, —N(CH₃)CH₂CH₂CH₂OH, —N(COCH₃)CH₂OH, —N(COCH₃)CH₂CH₂OH, —N(COCH₃)CH₂CH₂CH₂,OH, —N(SO₂CH₃)CH₂OH, —N(SO₂CH₃)CH₂CH₂OH, —N(SO₂CH₃)CH₂CH₂CH₂OH. In some embodiments, R₉ is selected from: F, Cl, Br, cyano, methyl, ethyl, trifluoromethyl, methoxyl, ethoxyl, hydroxyl, thiol, amino, —C(═O)CH₃, C(═O)CH₂CH., C(═O)CH₂CH₂CH₃, —CH₂CH₂N(CH₃)₂, —CH₂OH, —CH₂CHOH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —OCH₂OH, —OCH₂CH₂OH, —OCH₂CH₂CH₂OH, —OCH₂NH₂, —OCH₂CH₂NH₂, —OCH₂CH₂CH₂NH₂, —OCH₂SH, —OCH₂CH₂SH, —OCH₂CH₂CH₂SH, —NHCH₂OH, —NHCH₂CH₂OH, —NHCH₂CH₂CH₂OH, —NHCH₂NH₂, —NHCH₂CH₂NH₂, —NHCH₂CH₂CH₂NH₂, —NHCH₂SH, —NHCH₂CH₂SH, —NHCH₂CH₂CH₂SH, —N(CH₃)CH₂OH, —N(CH₃)CH₂CH₂OH, —N(CH₃)CH₂CH₂CH₂OH, —N(CH₃)CH₂NH₂, —N(CH₃)CH₂ CH₂NH₂, —N(CH₃)CH₂CH₂CH₂NH₂, —N(CH₃)CH₂SH, —N(CH₃)CH₂CH₂SH, —N(CH₃)CH₂CH₂CH₂SH, —N(COCH₃)CH₂OH, —N(COCH₃)CH₂CH₂OH, —N(COCH₃)CH₂CH₂OH, —N(COCH₃)CH₂NH₂, —N(COCH₃)CH₂CH₂NH₂, —N(COCH₃)CH₂CH₂CH₂NH₂, —N(COCH₃)CH₂SH, —N(COCH₃)CH₂ CH₂SH, —N(COCH₃)CH₂CH₂CH₂SH—N(SO₂CH₃)CH₂OH, —N(SO₂CH₃)CH₂CH₂OH, —N(SO₂CH₃)CH₂CH₂CH₂OH, —N(SO₂CH₃)CH₂NH₂, —N(SO₂CH₃)CH₂CH₂NH₂, —N(SO₂CH₃)CH CH₂CH₂NH₂, —N(SO₂CH₃)CH₂SH, —N(SO₂CH₃)CH₂CH₂SH, —N(SO₂CH₃)CH₂CH₂CH₂SH.

Preferably, U is selected from the following groups:

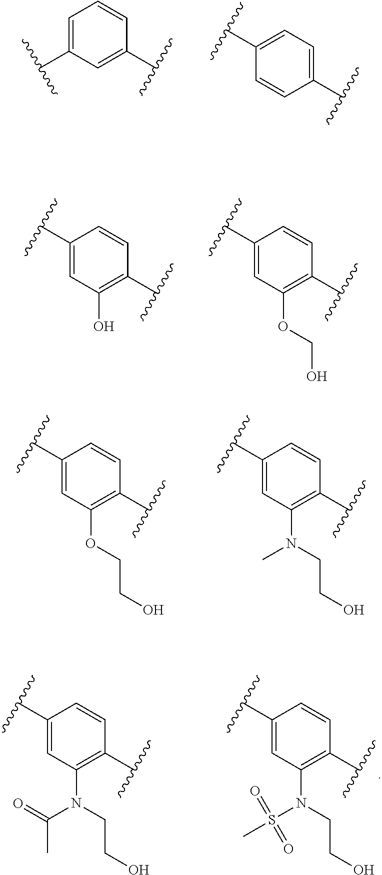

In some embodiments, the -T-U-Z- together forms a group selected from:

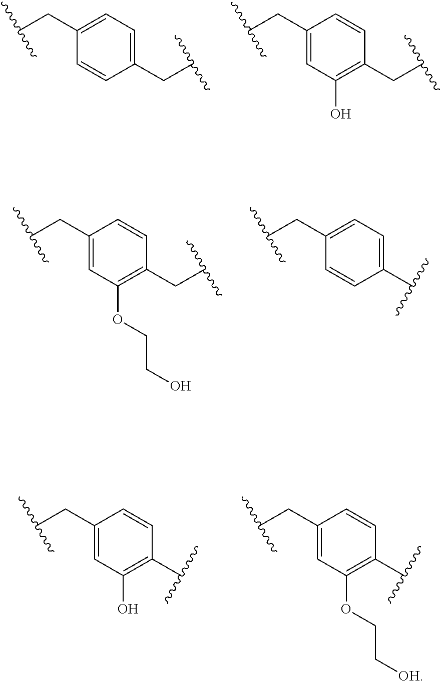 [SMILES: *Cc1ccc(C*)cc1 ; *Cc1ccc(C*)c(O)c1 ; *Cc1ccc(C*)c(OCCO)c1 ; *Cc1ccc(*)cc1 ; *Cc1ccc(*)c(O)c1 ; *Cc1ccc(*)c(OCCO)c1]

In some embodiments, the -T-U-Z- together forms a group selected from: C₁-C₆ alkylene; preferably, the -T-U-Z- together forms a group selected from: CI alkylene, 1,2-ethylidene, 1,3-propylidene, 1,4-butylidene, 1,5-pentylidene, 1,6-hexylidene.

In some embodiments, Y is selected from: chemical bond, —C(═O)NH—, —C(═O)NHCH₂—, —C(═O)NHCH₂CH₂—, —C(═O)NHCH₂,CH₂CH₂—, —NHC(═O)—, —NHC(═O)CH₂—, —NHC(═O)CH₂CH₂—, —NHC(═O)CH₂CH₂CH₂—, —O—, —OCH₂—, —OCH₂CH₂—, —OCH₂CH₂—, —NR₂—, —NR₂CH₂—, —NR₂CH₂CH₂—, —NR₂CH₂CH₂CH₂—.

In some embodiments, Y is selected from: chemical bond, —NR₁₄C(═O)—, —C(═O)NR—, —C(═O)NR₁₄CH₂—, —C(═O)NHCHR₉—, —NR₁₄C(═O)CH—, —NHC(═O)CHR₉—, —O—, —OCHR₉—, —OCH₂CHR₉—, —NR₂-—, —NR₂CH₂—, —NHCHR₉—.

Preferably, R₉ is selected from: hydrogen, C₁-C₄ alkyl, R₁₃; More preferably, R₉ is chosen from: R₁₃.

Preferably, R₁₃ is selected from: hydroxyl, Rₙ, —ORₙ.

Preferably, R₁₁ is selected rom: Rₙ, —C(═O)R.

Preferably, wherein R₂ is selected from: hydrogen, R₁₄, C₁-C₈ alkyl, C₃-C₁₀ cycloalkyl, aryl, —C(═O)C₃-C₁₀ cycloalkyl, —C(═O)C—Cs alkyl or —S(═O)₂C₁-C₈ alkyl, wherein C—Cs alkyl, C₃-C₁₀ cycloalkyl, aryl and the cycloalkyl or alkyl portion of —C(═O)C₃-C₁₀ cycloalkyl, —C(═O)C—Cs alkyl or —S(═O)₂C₁-C₈ alkyl is optionally substituted with one or more R₉, wherein R₉ is selected from: hydrogen, halogen, amino, cyano, carboxyl, C₁-C₆ alkyl, C₁-C₆ alkoxyl or aryl; More preferably, R₂ is selected from: R₁₄; wherein, preferably, R₁₄ is selected from: Rₙ, —C(═O)Rₙ, —C(═O)ORₙ, —C(═O)NRₘRₙ, —S(═O)₂₁R, —S(═O)₂NRₘRₙ.

Preferably, Rₙ is selected from: C₁-C₈ alkyl substituted with hydroxyl; More preferably, Rₙ, is selected from: C₁-C₄ alkyl substituted with hydroxyl; Preferably, Rₙ is selected from: —CH₂OH, —CH₂CH₂OH, —CH₂CH₂CH₂OH, or —CH₂CH₂CH₂CH₂OH.

In some specific embodiments, R₉ is selected from: hydrogen, C₁-C₄ alkyl, —R₂₁N(R₂₂)R₂₂, —R₂₁C(═O)R₂₂, R₁₃; wherein R₁₃ is selected from: hydroxyl, sulfhydryl, amino, Rₙ, —ORₙ; wherein R₁₄ is selected from: Rₙ, —C(═O)Rₙ; wherein R₂ is chosen from: hydrogen, R₁₄, C₁-C₈ alkyl, C₃-C₁₀ cycloalkyl, aryl, —C(═O)C₃-C₁₀ cycloalkyl, —C(═O)C₁-C₈ alkyl or —S(═O) C₁-C₈ alkyl, wherein C₁-C₈ alkyl, C₃-C₁₀ cycloalkyl, aryl and the cycloalkyl or alkyl portion of —C(═O)C₃-C₁₀ cycloalkyl, —C(═O)C₁-C₈ alkyl or —S(═O)₂C₁-C₈ is optionally substituted with one or more R; wherein R₉ is selected from: hydrogen, halogen, amino, cyano, carboxyl, C₁-C₈ alkyl, C₁-C₆ alkoxyl or aryl; wherein R₁₄ selected from: R₁, —C(═O)Rₙ, —C(═O)ORₙ, —C(═O)NRₘRₙ, —S(═O)₂Rₙ, —S(═O)₂NRₘRn; wherein Rₙ is selected from: C₁-C₈ alkyl substituted with hydroxyl, sulfhydryl or amino; Preferably, the Rₙ is selected from: C₁-C₄ alkyl substituted with hydroxyl, sulfhydryl or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH.

Preferably, Rₘ is chosen from: hydrogen, —C₁-C₄ alkyl.

Therefore, preferably, R₂ is selected from: hydrogen, methyl, ethyl, —SO₂CH₃, —COCH₃, -CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthylmethyl, 2-naphthylethyl, —CH₂OH, —CH₂CH₂OH, —CH₂CH₂CH₂OH, —C(═O)CH₂O H, —C(═O)CH₂CH₂OH, —C(═O)CH₂CH₂CH₂OH, —C(═O)OCH₂OH, —C(═O)OCH₂CH₂OH, —C(═O)OCH₂CH₂CH₂OH, —C(═O)NHCH₂OH, —C(═O)NHCHCH₂OH, —C;(═O)NHCH₂CH₂CH₂OH, —S(═O)₂, —S(═O)₂CH₂CH₂OH, —S(═O)₂CH₂CH₂CH₂OH, —S(═O)₂NHCH₂OH, —S(═O)₂NHCH₂CH₂OH, —S(═O)₂NHCH₂CH₂CH₂. In some embodiments, R₂ is further selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenylethyl, 1-naphthylhethyl, 2-naphthylethyl, —CH₂OH, —CH₂CH₂OH, —CH₂CH₂CH₂OH, —CH₂NH₂, —CH₂CH₂NH₂. —CH₂CH₂CH₂NH₂, CH₂SH, —CH₂CH₂SH, —CH₂CH₂CHSH, —CCH₂O CO₂CH₂SH, —C(═O)CH₂OH, —C(═O)CH₂CH₂OH, —C(═O)CH₂CH₂CH₂OH, —C(═O)CH₂NH₂, —C(═O)CH₂CH₂NH₂, —C(═O)CH₂CH₂CH₂NH₂, —C(═O)CH₂SH, —C(═O)CH₂CH₂SH, —C(═O)CH₂CH₂CH₂SH, —C(═O)OCH₂OH, —C(═O)OCH₂CH₂OH, —C(═O)OCH₂CH₂CH₂OH, —C(═O)OCH₂NH₂, —C(═O)OCH₂CH₂NH, —C(═O)OCH₂CH₂NH₂, —C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂CH₂CH₂OH, —C(═O)NHCH₂NH₂, —C(═O)NHCH₂CH₂NH, —C(═O)NHCH₂CH₂CH₂NH₂, —C(═O)NHCH₂SH, —C(═O)NHCH₂CH₂SH, —C(═O)NHCH₂CH₂CH₂SH, —S(═O)₂CH₂OH, —S(═O)₂CH₂CH₂OH, —S(═O)₂CH₂CH₂OH, —S(═O)₂CH₂NH₂, —S(═O)₂CH₂CH₂NH₂, —S(═O)₂CH₂C₁-C₂NH₂, —S(═O)₂CH₂SH, —S(═O)₂CH₂, —S(═O)₂C₂CH₂CH₂SH, —S(═O)₂NHCH₂O OH, —S(═O)₂NHCH₂, —S(═O)₂NHCH₂CH₂CH₂OH, —S(═O)₂NHCH₂NH₂, —S(═O)₂NHCH₂CH₂NH₂, —S(═O)₂NHCH₂CH₂CH₂ NH₂, —S(═O)₂NHCH₂SH, —S(═O)₂NHCH₂CH₂SH, —S(═O)₂NHCH₂CH₂CH₂SHI.

Preferably, R₁₄ is selected from: Rₙ, —C(═O)Rₙ.

Preferably, Rₙis selected from: C₁-C₈ alkyl substituted with hydroxyl; More preferably, Rₙ is selected from: C₁-C₄ alkyl substituted with hydroxyl; Most preferably, Rₙis selected from: —CH₂OH, —CH₂CH₂OH, —CH₂CH₂CH₂, or —CH₂CH₂CH₂CH. In some embodiments, Rₙ is selected from: C₁-C₈ alkyl substituted with hydroxyl, sulfhydryl or amino; More preferably, Rₙis selected from: C₁-C₄ alkyl substituted with hydroxyl, sulfhydryl or amino; Most preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH.

In some embodiments, L is selected from: —CH₂—, —CH₂CH₂—, —C(═O)—,

Preferably, L is selected from: —CH₂—, —C(═O)—, —CH(OH)—.

In some embodiments, wherein R₃—R₅ is independently selected from: hydrogen, R₁₃, halogen, cyano, amino, C₁-C₆ allyl, C₁-C₆ alkoxy, C₁-C₆ alkylthio, C₁-C₆ alkylamino, di(C₁-C₆ alkyl)amino, C₃-C₈ cycloalkyl, wherein the alkyl, alkoxy, alkylamino, alkylthio, or cycloalkyl is optionally substituted with 1-3 groups each selected from halogen, cyano, C₁-C₃ alkyl or C₁-C₃ alkoxy; when there are multiple R₃-Rs, any two adjacent ones may be combined to form a ring.

Preferably, wherein R₃ is selected from: R₁₃.

Preferably, wherein R₁₃ is selected from: hydroxyl, Rₙ, —ORₙ, —OC(═O)Rₙ, —NRₘRₙ, —NHRₘ—C(═O)Rₙ, —NR₁—C(═O)Rₘ, —NRₘ—S(═O)₂Rₙ, —NRₐ—S(═O)₂Rₘ; More preferably, R₁₃ is selected from: hydroxyl, Rₙ, —ORₙ.

Preferably, Rₙ, is selected from: C₁-C₈ alkyl substituted with hydroxyl; More preferably, Rₙ is selected from: C₁-C₄ alkyl substituted with hydroxyl; Most preferably, Rₙ is selected from: —CH₂OH, —CH₂CH₂OH, —CH₂CH₂CH₂OH, or —CH₂CH₂CH₂CH₂OH. In some embodiments, Rₙ, is selected from: C₁-C₈ alkyl substituted with hydroxyl, sulfhydryl or amino; More preferably, Rₙis selected from: C₁-C₄ alkyl substituted with hydroxyl, sulfhydryl or amino; Most preferably, Rₙis selected from: —CH₂H, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH.

Preferably, Rₘ is selected from: hydrogen, -C₁-C₄ alkyl.

Preferably, R₄-R₅ is independently selected from: hydrogen, halogen, cyano, C₁-C₄ alkyl, C₁-C₄ haloalkyl, respectively.

In some embodiments, wherein R₆ is selected from: hydrogen, C₁-C₆ alkyl, C₃- cycloalkyl, wherein the alkyl, cycloalkyl is optionally substituted with 1-3 groups each selected from halogen, hydroxyl, cyano, amino, —C(═O)OR₂₃, —OC(═O)₂₃, —NHC(═O)R₂₃, —C(═O)NHR₂₃ or —OP(═O)(OM)₂;
in some embodiments, wherein R₆ is selected from: hydrogen, C₁-C₄ alkyl, C₁-C₄ haloalkyl, —C₁-C₄ alkyleneOC(═O)R₂₃, —C₁-C₄ alkyleneOP(═O)(OH)₂; wherein R₂₃ is selected from: hydrogen, C₁-C₄ alkyl, and the C₁-C₄ alkyl is optionally substituted with 1-3 groups selected from hydroxyl and amino, respectively.

In some embodiments, wherein R₆ is selected from: hydrogen, C₁-C₄ alkyl, C₁-C₄ haloalkyl, —CH₂OC(═O)R₂₃—CH₂CH(═O)R₂₃, —CH(═O)OR₂₃, —CH₂CH₂C(═O)OR₂₃, —CH₂OP(═O)(OH)₂, —CH₂CH₂OP(═O)(OH)₂; wherein R₂₃ is selected from: hydrogen, C₁-C₄ alkyl, wherein C₁-C₄ alkyl is optionally substituted with 1-3 groups selected from hydroxyl and amino, respectively.

In some embodiments, the Rₙis selected from: hydrogen, C₁-C₄ alkyl, C₁-C₄ haloalkyl, —CH₂OC(═O) C₁-C₄ alkyl, —CH₂OC(═O)CH₂OH, —CH₂OC(═O)CH₂NH₂—CH₂OP(═O)(OH)₂.

In some embodiments, wherein R₆ is selected from: hydrogen, C₁-C₆ alkyl, C₃-C₈ cycloalkyl, wherein alkyl and cycloalkyl are optionally substituted with 1-3 groups selected from a halogen, C₁-C₃ alkyl, or C₁-C₃ alkoxyl, respectively.

Preferably, wherein R₆ is selected from: hydrogen, C₁-C₄ alkyl, C₁-C₄ haloalkyl.

In some embodiments, the compound is a compound represented by formula (1-1) or formula (1-2) and a pharmaceutically acceptable salt, solvent compound, stereoisomer, isotope, and a prodrug thereof:

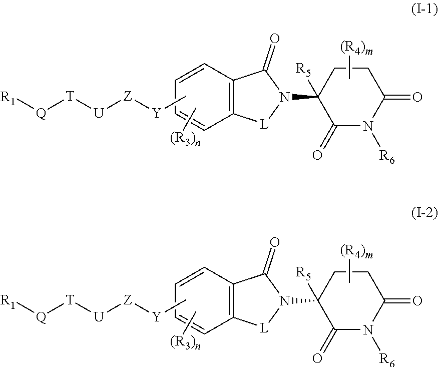

Wherein, R₁, R₃—R₆, Q, T, U, Z, Y, L, n, m are defined as above.

In some embodiments, the compound is a compound represented by Formula (II) or (Ill) and a pharmaceutically acceptable salt, solvent compound, stereoisomer, isotope, and prodrug thereof:

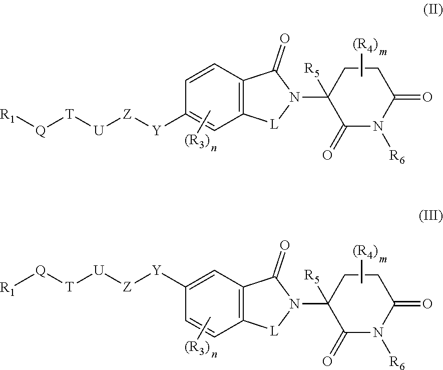

Where R₁, R₃-R₆, Q, T, U, Z, Y, L, n, in are defined as above. in some embodiments, the compound is a compound represented by Formula (IV) or Formula (V) and a pharmaceutically acceptable salt, solvent compound, stereoisomer, isotope and prodrug thereof:

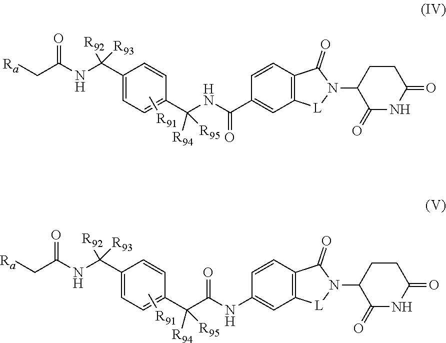

Wherein NH of CH₂C(═O)NH—, —C(R₉₄)(R₉₅)NHC(═O)—, —C(R₉₄)(R₉₅)C(═O)NH—, is optionally substituted with R₁₄. Rₙ, R₁₄ and L are defined as above; R₉₁—R₉₅ is independently selected from: hydrogen, R₁₃, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R₂₁OC(═O)R₂₂, —R₂₃S(═O)₂R₂₂, —R₂₁S(═O)₂NHR₂₂, C₁-C₆ alkyl, C₁-C₆ alkoxyl, C₁-C₆ alkylthio, C₁-C₆ alkylamino, di(C₁-C₆)alkylamino, C₃-C₈ cycloallyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, Cyano, C₁-C₃ alkyl or C₁-C₃ alkoxyl; Any two adjacent ones in R₉₂-R₉₄ may be combined to form a ring.

Preferably, wherein R₉₁ is selected from: hydrogen, R₁₃, halogen, cyano, amino, C₁-C₆ alkyl, C₁-C₆ alkoxyl, C₁-C₆ alkylthio, C₁-C₆ alkylamino, di(C₁-C₆)alkylamino, C₃-C₈ cycloalkyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with 1-3 groups selected from halogen, cyano group, C₁-C₃ alkyl or C₁-C₃ alkoxyl; More preferably, R₉₁ is selected from: hydrogen, halogen, C₁-C₆ alkyl, C₁-C₆ alalkyl, C₁-C₆ alkoxyl, hydroxyl, Rₙ, —ORₙ, —OC(═O)Rₙ, —NRₘ,Rₙ, —NRₘ—C(═O)Rₙ, —NR—C(═O)Rₘ, —NRₘ—S(═O)₂Rₙ, —NR—S(═O)₂Rₘ; Also preferably, is selected from: hydrogen, halogen, C₁-C₆ alkyl, C₁-C₆ haloalkyl, C₁-C₆ alkoxyl, hydroxyl, Rₙ, —ORₙ, —NRₘRₙ, —NR—C(═O)Rₙ, —NRₙ—S(═O)₂Rₘ.

Preferably, the R₉₂-R₉₄ is independently selected from: hydrogen, R₁₃, C₁-C₆ alkyl, or any two adjacent groups in R₉₂-R₉₄ can be combined to form a cycloalkyl group; More preferably, R₉₂—R₉₄ is independently selected from: hydrogen, methyl, ethyl, hydroxyl, Rₙ, —ORₙ, or R₉₂ and a together form —CH₂CH₂—, or R₉₄ and R₉₅ together form —CH₂CH₂—.

In some embodiments, wherein NH of RₐCH₂C(═O)NH—, —C(R₉₄)(R₉)NHC(═O)—, —C(R₉₄)(R₉₅)C(═O)NH— has not been substituted.

In some embodiments, wherein R₉₁ is selected from R₁₃, or one of R₉₁is selected from R₁₃.

R₁₃, Rₘ, Rₙ are defined as above.

In some embodiments, the compound is a compound represented by formula (IV′) or formula (V′) and a pharmaceutically acceptable salt, solvent compound, stereoisomer, isotope and prodrug thereof:

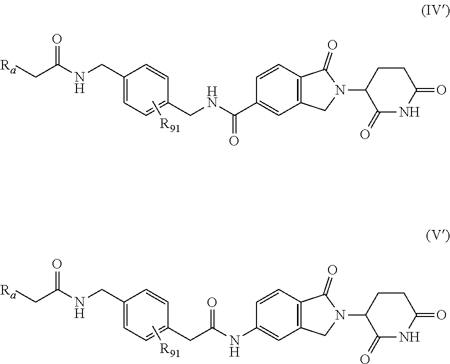

Where Rₐ and R₉₁ are defined as above.

In some embodiments, R₉₁ is selected from: hydroxyl, Rₙ, —ORₙ, —NRₘRₙ, —NRₙ—C(═O)Rₘ, —NRₙ—S(═O)₂Rₘ; wherein Rₙ is selected from: C₁-C₈ alkyl substituted with hydroxyl; Preferably, Rₙ is selected from: C₁-C₄ alkyl substituted with hydroxyl; More preferably, R₁₁ is selected from: —CH₂OH, CH₂CH₂OH, —CH₂CH₂CH₂OH, or —CH₂CH₂CH₂CH₂OH. Wherein is selected from: hydrogen, —C₁-C₄ alkyl. In some embodiments, wherein Rₙ is selected from: C₁-C₈ alkyl substituted with hydroxyl, sulfhydryl or amino; Preferably, wherein Rₙ is selected from: C₁-C₄ alkyl substituted with hydroxyl, sulflhydryl or amino; Most preferably, is selected from: —CH₂OH, —CH₂CH₂OH, —CH₂CH₂CH₂OH, —CH₂CH₂CH₂CH₂OH, —CH₂NH₂, CH₂CH₂NH₂, —CH₂CH₂CH₂NH, —C—NH₂CH₂CH₂CH₂NH₂, —CH₂SH, —CH₂CHCH₂SH, —CH₂CH₂CH₂CH₂SH.

In some embodiments, the compound is a compound represented by formula (VI) or formula (VII) and a pharmaceutically acceptable salt, solvent compound, stereoisomer, isotope and prodrug thereof:

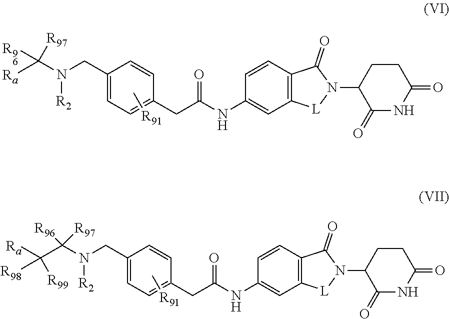

Wherein NH of —CH₂C(═O)NH— is optionally substituted with R₁₄;

Rₐ, R₂, R₉₁, R₁₄, L are defined as above;

R₉₆-R₉₉ are chosen independently from: hydrogen, R₁₃, halogen, cyano, amino, nitro, —R₂₁C(═O)R₁₂₂, —R₂₁NHC(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxyl, C₁-C₆ alkyl, C₁-C₆ alkylamino, di(C₁-C₆)alkylamino, C₃-C₈ cycloallyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, cyano, C₁-C₃ alkyl or C₁-C₃ alkoxyl; Any two adjacent ones in R₉₆R₉₉ may be combined to form a ring.

Preferably, R₉₆—R₉₉ is independently selected from: hydrogen, R₁₃, C₁-C₆ alkyl group, or any two adjacent groups in R₉₆n-1R₉ can be combined to form a cycloalkyl group; More preferably, R₉₆—R₉₉ is independently selected from: hydrogen, methyl, ethyl, hydroxyl, Rₙ, —OR, or R₉₆ and R₉₇ together form —CH₂CH₂—, or 1(₉₈ and R₉₉ together form —CH₂CH₂—, respectively.

Preferably, R₂ is selected from: hydrogen, R₁₄, C₁-C₈ alkyl, C₃-C₁₀ cycloalkyl, —C(═O)C₃-C₁₆ cycloalkyl, —C(═O)C₁-C₈ alkyl or —S(═O)₂C₁-C₈ alkyl, wherein the cycloalkyl or alkyl portion of the C₁-C₈ alkyl, C₃-C₁₀ cycloalkyl, —C(═O)C₃-C₁₀ cycloalkyl, —C(═O)C₁-C₈ alkyl or —S(═O)₂C₁-C₈ alkyl is optionally substituted with one or more 1R₉; wherein R,) is selected from: hydrogen, halogen, amino, cyano, carboxyl, C₁-C₆ alkyl, C₁-C₆ alkoxyl or aryl; More preferably, R₂ is selected from: R₁₄.

Preferably, wherein R₄ is selected from: Rₙ, —C(═O)Rₙ, —C(═O)ORₙ, —C(═O)NRₘ, —S(═O)₂Rₙ a, —S(O)₂NRₘRₙ.

Preferably, wherein Rₙ is selected from: C₁-C₈ alkyl substituted with hydroxyl; More preferably, Rₙ is selected from: C₁-C₄ alkyl substituted with hydroxyl; Most Preferably, Rₙ is selected from: —CH₂OH, —CH₂CH₂OH, —CH₂CH₂CHOH or —CH₂CH₂CH₂CH₂OH. In some embodiments, Rₙ is selected from: C₁-C₈ alkyl substituted with hydroxyl, sulfhydryl, or amino; More preferably, Rₙ is selected from: C₁-C₄ alkyl substituted with hydroxyl, sulfhydryl, or amino; Most Preferably, Rₙ is selected from: —CH₂OH, —CH₂CH₂OH, —CH₂CH₂CH₂Off, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂O₂CH₂SH, —CH₂CH₂CH₂CH₂Si.

Preferably, wherein Rₘ is selected from: hydrogen, —C₁-C₄ alkyl.

In some embodiments, wherein R₂ is selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenylethyl, 1-naphthylmethyl, 2-naphthylethyl, —C₁₂OH, —CH₂CH₂OH, —CH₂CH₂CH₂OH, —C(═O)CH₂OH, —C(═O)CH₂CH₂OH, —C(═O)CH₂CHC₂OH, —C(═O)OCH, —C(═O)OC H₂CH₂OH, —C(═O)OC H₂CH₂₂COH, —C(═O)NHCH₂OH, —C(═O)NHCH₂CH₂OH, —C(═O)NHCH₂CH₂CH₂OH, —S(═O)₂CH₂OH, —S(═O)₂CH₂CH₂OH, —S(═O)₂CH₂CH₂OH, —S(═O)₂NHCH₂OH, —S(═O)₂NHCH₂CH₂OH, —S(═O)₂NHCH₂CH₂CH₂OH. In some embodiments, ₂ is further selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthylethyl, —CH₂OH, —CH₂CH₂OH, —CH₂CH₂CH₂, —CH₂NH₁₂, —CH₂CH₂NH₂, CH₂CH₂CH₂NH₂, —CH₂SH, —CH₂CH₂SH, —CH₂CH₂CH₂SH, —CH₂CH₂CH₂CH₂SH, —C(═O)CH₂OH, —C(═O)CH₂CH₂OH, —C(═O)CH₂CH₂CH₂OH, —C(═O)CH₂NH₂, —C(═O)CH₂CH₂NH₂, —C(═O)CH₂CH₂CH₂NH₂, —C(═O)C H₂SH, —C(═O)CH₂CH₂SH, —C(═O)CH₂CH₂CH₂SH, —C(═O)OCH₂OH, —C(═O)OCH₂CH₂, —C(═O)OCH₂CH₂CHOH, —C(═O)OCH₂NH₂, —C(═O)OCH₂CH₂NH₂, —C(═O)OCH₂CH₂CH₂NH₂, —C(═O)OCH₂SH, —C(═O)OCH₂CHSH, —C(═O)OCH₂CHCH₂SH, —C(═O)NHCH₂OH, —C(═O)NHCH₂CH₂, —C(═O)NHCHC₂CCH₂OH, —C(═O)NHCH₂N H₂, —C(═O)NHCH₂CH₂NH₂, —C(═O)NHCH₂CH₂NH₂, —C(═O)NHCH₂SH, —C(═O)NHCH₂CH₂SH, —C(═O)NCH₂CH₂SH, —S(═C)CH₂OH, —S(═O)₂CH₂CH₂OH, —S(═O)₂C₁₋₂CH₂CH₂OH, —S(═O)₂, —S(═O)₂CH₂CH₂NH₂, —S(═O)₂CH₂CH₂CHNH₂, —S(═O)₂CH₂SH, —S(═O)₂CH₂CH₂SH, —S(═O)₂CH₂CH₂CH₂SH, —S(═O)₂NHCH₂OH, —S(═O)₂NHCH₂CH₂OH, —S(═O)₂NHCH₂CH₂CH₂OH, —S(═O)₂NHCH₂NH₂, —S(═O)₂NHCH₂CH₂NH₂, —S(═O)₂NHC₂CH₂CH₂, —S(═O)₂NHCH₂, —S(═O)₂NCH₂CH₂SH, —S(═O)₂NHCH₂CH₂CH₂SH.

Preferably, wherein R₂ is selected from: hydrogen, methyl, ethyl, isopropyl, cyclopropyl, benzyl, —CH₂OH, —CH₂CH₂OH, .—CH₂CH₂CH₂OH, —C(═O)CH₂OH, —C(═O)CH₂CH₂OH, —C(═O)CH₂CH₂CH₂OH, —C(═O)OCH₂OH, —C(═O)O CH₂OH, —C(═O)OC₂H₂H, —C(═O)NHCH₂OH, —C(═O)NHCH₂CH₂OH, —C(═O)NHCH₂CH₂CH₂OH; In some embodiments, wherein R₂ is selected from: hydrogen, methyl, ethyl, isopropyl, cyclopropyl, benzyl, —CH₂OH, —CH₂CH₂OH, —CH₂CH₂CH₂OH. —CH₂NH₂, —CH₂CH₂NH₂, —CH₂CH₂CH₂NH₂, —CH₂SH, —CH₂CH₂SM, —CH₂CH₂CH₂SH, —C(═O)CH₂OH, —C(═O)CH₂CH₂O, —C(═O)CH₂CH₂CH₂OH, —C(═O)CH₂ NH₂, —C(═O)CH₂CH₂NH₂, —C(═O)CH₂CH₂CH₂NH₂, °—C(═O)CH₂SH, —C(═O)CH₂CH₂S, SH, —C(═O)CH₂CH₂C:2SH, —C(═O)OCH₂OH, —C(═O)OCH₂CH₂OH, —C(═O)OCH₂CH₂CH₂OH, —C(═O)OCH₂NH₂, —C(═O)OCH₂CH₂NH₂, —C(═O)OCH₂CH₂CH₂NH₂, —C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂CH₂CH₂OH, —C(═O)NHCH₂NH₂, —C(═O)HCH₂CH₂, NH₂, —C(═O)NHCH₂CH₂CH₂NH₂, —C(═O)NHCH₂SH, —C(═O)NHCH₂CH₂SH, —C(═O)NHCH₂CH₂CH₂SH.

In some embodiments, NH of —CH₂C(═O)NH— is not substituted.

In some embodiments, wherein R₂ is selected from R₄, or R₉₁ is selected from R₁₃, or one of the R₉-R₉₉ is selected from R₁₃.

Wherein R₁₃, R₁₄, Rₘ, Rₙ are defined as above.

In some embodiments, the compound is a compound represented by formula (Vi′) or (VII′) and a pharmaceutically acceptable salt, solvent compound, stereoisomer, isotope, and prodrug thereof:

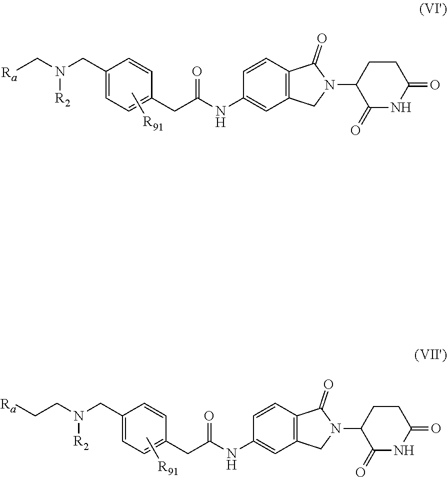

Wherein, Rₙ, R₂, R₉₁ are defined as above.

In some embodiments, wherein R₂ is selected from: Rₙ, —C(═O)Rₙ, —C(═O)ORₙ, —C(═O)NRₘ,R.

In some embodiments, wherein R)i is selected from: hydroxyl, Rₙ, —ORₙ, —NRₘRₙ, —NRₙ—CO(═O)Rₘ, —NRₙ—S(═O)₂Rₘ.

Wherein Rₙis selected from: C₁-C₈ alkyl substituted with hydroxyl; More preferably, Rₙis selected from: C₁-C₄ alkyl substituted with hydroxyl; Most Preferably, R is selected from: —CH₂O, —CH₂CH₂OH, —CH₂CH₂CH₂OH or —CH₂CH₂CH₂OH. Wherein Rₘ is selected from: hydrogen, —C₁-C₄ alkyl.

In some embodiments, the compound is a compound represented by formula (VIII) and a pharmaceutically acceptable salt, solvent compound, stereoisomer, isotope and prodrug thereof:

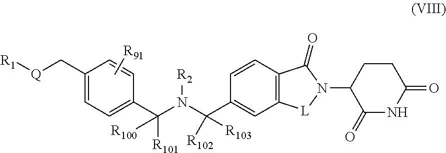

Wherein, R₁, Q, R₂, R₉₁, L are defined as above;

R₁₀₀-R₁₀₃ are selected independently from: hydrogen, R₁₃, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, CH₆ alkyl, C₁-C₆ alkoxyl, C₁-C₆ alkylthio, C₁-C₆ alkylamino, di(C₁-C₆)alkylamino, C₃-C₈ cycloalkyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, cyano, C₁-C₃ alkyl or C₁-C₃ alkoxyl; Any two adjacent ones in R₉₆-R₉₉ may be combined to form a ring.

Preferably, wherein R₁₀₀-R₁₀₃ is independently selected from: hydrogen, R₁₃, C₁-C₆ alkyl group, or any two adjacent groups in R₁₀₀-R₁₀₃ can be combined to form a cycloalkyl group; More preferably, R₁₀₀-R₁₀₃ is independently selected from: hydrogen, methyl, ethyl, hydroxyl, Rₙ, —ORₙ, or R₁₀₀ and R₁₀₁ together form —CH₂CH₂—, or R₁₀₂ and R₁₀₃ together form —CH₂CH₂—, respectively.

Preferably, wherein R₂ is selected from: hydrogen, R₁₄, C₁-C₈ alkyl, C₃-C₁₀ cycloalkyl, —C(═O)cycloalkyl, —C(═O)C₁-C₈ alkyl or —S(═O)₂C—Cs alkyl, wherein the cycloalkyl or alkyl portion of the C₁-C₈ alkyl, C₃-C₁₀ cycloalkyl, —C(═O)C₃-C₁₀ cycloalkyl, —C(═O)C₁-C₈ alkyl or —S(═O)₂C—Cs alkyl is optionally substituted with one or more R₉; wherein R₉ is selected from: hydrogen, halogen, amino, cyano, carboxyl, C₁-C₈ alkyl, C₁-C₆ alkoxyl or aryl; More preferably, R₂ is selected from: R₁₁.

Preferably, wherein R₁₄ selected from: Rₙ, —C(═O)Rₙ, —C(═O)ORₙ, —C(═O)NRₘRₙ, —S(═O)₂Rₙ, —S(═O)₂NRₘRₙ.

Preferably, wherein Rₙis selected from: C₁-C₈ alkyl substituted with hydroxyl; More preferably, R₉ is selected from: C₁-C₄ alkyl substituted with hydroxyl; Most Preferably, Rₙ is selected from: —CH₂OH, —CH₂CH₂OH, —CH₂CH₂CH₂OH or —CH₂CH₂CH₂CH₂OH. In some embodiments, Rₙ is selected from: C₁-C₈ alkyl substituted with hydroxyl, sulfhydryl, or amino; More preferably, Rₙ, is selected from: C_{1I}C₄ alkyl substituted with hydroxyl, sulfhydryl, or amino; Most Preferably, Rₙis selected from: —CH₂H, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH.

Preferably, wherein Rₙ is selected from: hydrogen, —C_{1I}C₄ alkyl.

In some embodiments, wherein R₂ is selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenylethyl, 1-naphthylmethyl, 2-naphthylethyl, —CH₂OH, —CH₂CH₂OH, —CH₂CH₂CH₂OH, —C(═O)CH₂OH, —C(═O)CH₂CH₂OH, —C(═O)CH₂CH₂CH₂OH, —C(═O)OCH₂OH, —C(═O)OCH₂CH₂O H, —C(═O)OCH₂CH₂CH₂O H, —C(═O)N, —C(═O)NHCH₂CH₂OH, —C(═O)NHCH₂CH₂CH₂OH, —S(═O)₂H, —S(═O)₂CH₂CH, —S(═O)₂CH₂CH₂CH₂OH, —S(═O)₂NHCH₂OH, —S(═O)₂NHCH₂CH, —S(═O)₂NHCH₂CH₂CH₂OH. In some embodiments, wherein R₂ is further selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenylethyl, 1-naphthyl methyl, 2-naphthylethyl, —CH₂OH, —CH₂CH₂OH, —CH₂CH₂CH₂OH, —CH₂NH₂, —CH₂CH₂NH₂, —CH₂CH₂CH₂NH₂, ˜—CH₂SH, —CH₂CH₂SH, —CH₂CH₂CH₂SH, —CH₂CH₂CH₂CH₂SH, —C(═O)CH₂OH, —C(═O)CH₂CH₂OH, —C(═O)CH₂CH₂CH₂OH, —C(═O)CH₂NH₂, —C(═O)CH₂CH₂NH₂, —C(═O)CH₂,RₐCH₂NH, —C(═O)C H₂, —C(═O)CH₂CH₂SH, —C(═O)CH₂CH₂CH₂SH, —C(═O)OCH₂OH, —C(═O)OCH₂CH₂OH, —C(═O)OCH₂CH₂CH₂OH, —C(═O)OCH₂NH₂, —C(═O)OCH₂CH₂NH₂, —C(═O)OCH₂CH₂C₂NH₂, —C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂C—₂CH₂OH, —C(═O)NHCH₂NH₂, —C(═O)CH₂Cl₂NH₂, —C(═O)NHCH₂CH₂CH₂NH₂, —C(═O)NHCH₂SH, —C(═O)NHCH₂CH₂SH, —C(═O)NHCH₂CH₂CH₂SH, —S(═O)₂CH₂OH, —S(═OC)₂C₂CH₂OH, —S(═O)₂CH₂CH₂CH₂OH, —S(═O)₂CH₂NH₂, —S(═O)₂CH₂CH₂NH₂, —S(═O)₂CH₂CH₂CH₂NH₂, —S(═O)₂CH₂SH, —S(═O)₂CH₂CH₂CH₂S, —S(═O)₂CH₂CH₂CH₂SH, —S(═O)₂HCH₂OH, —S(═O)₂NHCH₂CH₂O H, —S(═O)₂NHCH₂CH₂CH₂OH, —S(═O)₂NHCH₂NH₂, —S(═O)₂NHCH₂CH₂NH₂, —S(═O)₂NHCHCH₂CH₂NH₂, —S(═O)₂NHCH₂SH, —S(═O)₂NHCH₂CH₂SH, —S(═O)₂NHCH₂CH₂CH₂SH.

Preferably, wherein R₂ is chosen from: hydrogen, methyl, ethyl, isopropyl, cyclopropyl, benzyl, —CH₂OH, —CH₂CH₂OH, —CH₂CH₂CH₂OH, —C(═O)CH₂OH, —C(═O)CH₂CH₂OH, —C(═O)CH₂CH₂CH₂OH, —C(═O)OCH₂OH, —C(═O)OCH₂CH₂OH, —C(═O)OCH₂CH₂CH₂OH, C(O)NHCH₂OH, —C(═O)NHCH₂CH₂OH, —C(═O)NHCH₂CH₂CH₂OH. In some embodiments, wherein R₂ is further selected from: hydrogen, methyl, ethyl, isopropyl, cyclopropyl, benzyl, —CH₂OH, —CH₂CH₂OH, —CH₂CH₂CH₂OH, —CH₂NH₂, —CH₂CH₂NH₂, —CH₂CH₂CH₂NH₂, —CH₂SH, —CH₂CH₂ISH, —CH₂CH₂CH₂SH, —CH₂CH₂CH₂CH₂SH, —C(═O)CH₂OH, —C(═O)CH₂CH₂OH, —C(═O)CH₂CH₂CH₂OH, —C(═O)CH₂ NH₂, —C(═O)CH₂CH₂NH₂, —C(═O)CH₂CH₂CH₂NH₂, —C(═O)CH₂SH, —C(═O)CH₂CH₂SH, —C(═O)CH₂CH₂CH₂SH, —C(═O)OCH₂OH, —C(═O)OCH₂CH₂OH, —C(═O)OCH₂CH₂OH, —C(═O)OCH₂NH₂, —C(=—O)OCH₂C₁₂NH₂, —C(═O)OCH₂C₂CCH₂NH₂, —C(═O)OCH₂SH, —C(═O)OCH₂CH₂SH, —C(═O)OCH₂CH₂CH₁SH, —C(═O)NHCH₂OH, —C(═O)NHCH₂CH₂OH, —C(═O)NHCH₂CH₂CH₂OH, —C(═O)NHCH₂NH₂, —C(═O)NHCH₂CH₂NH₂, -.C(═O)NHCH₂CH₂CH₂NH₂, —C(═O)NHCH₂SH, —C(═O)NHCH₂CH₂SH, —C(═O)NHCH₂CH₂CH₂SH.

In some embodiments, wherein R₂ is selected from R₁₄, or the R₉₁ is selected from R₁₃, or one of the R₁₀₀-R₀₃ is selected from R₁₃.

The R₁₃, R₁₄, Rₙ, Rₙare defined as above.

In some embodiments, the compound is a compound represented by formula (VII′) and a pharmaceutically acceptable salt, solvent compound, stereoisomer, isotope and prodrug thereof:

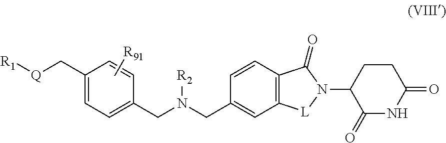

Wherein R₁, Q, R₂, R₉₁ are defined as above.

In some embodiments, wherein R₂ is selected from: Rₙ, —C(═O)Rₙ, —C(═O)ORₙ, —C(═O)NRₘRₙ.

In some embodiments, wherein R₉₁ is selected from: hydroxyl, Rₙ, —ORₙ, —NRₘRₙ, —NRₐ—C(═O)Rₘ, —NRₙ—S(═O)₂Rₘ.

Wherein Rₙ is selected from: C₁-C₈ alkyl substituted with hydroxyl; More preferably, Rₙ is selected from: C₁-C₄ alkyl substituted with hydroxyl; Most Preferably, Rₙ is selected from: —CH₂OH, —CH₂CH₂OH, —CH₂CH₂CH₂OH or —CH₂CH₂CH₂CH₂OH. in some embodiments, Rₙis selected from: C₁-C_{N} alkyl substituted with hydroxyl, sulfhydryl, or amino; More preferably, Rₙ is selected from: C₁-C₄ alkyl substituted with hydroxyl, sulfhydryl, or amino; Most Preferably, Rₙ is selected from: —CH₂OH, —CH₂CH₂₀₁, —CH₂CH₂CH₂OH, —CH₂CH₂CH₂CH₂, —CH₂NH₂, —CH₂CH₂NH₂, —CH₂CH₂CH₂NH₂. —CH₂CH₂CH₂CH₂NH₂, —CH₂SH, —CH₂CH₂SH, —CH₂CH₂CH₂SH, —CH₂CHC₂SH.

In some embodiments, the compound is the compound represented by formula (XI) and pharmaceutically acceptable salt, solvent compound, stereoisomers, isotope, and prodrug thereof:

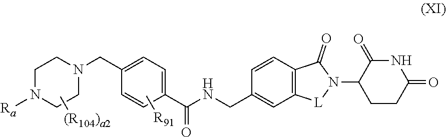

Wherein NH of —C(═O)NHCH₂— is optionally substituted with R₁₄;

Rₐ, R_{b4}, R₉₁, a2 are described as above;

₁₀₄ is chosen from: hydrogen, R₁₃, halogen, cyano, amino, nitro, —R₂C(═O)R₂, —R₂₂NHC(═O)R₂, —R₂₁C(═O)NH R₂₂, —R₂₁C(═O)OR₆₆, —R₂₁OC(═O)Rₙ, —R₂₂S(═O)₂R₂, —R₂₁S(═O)₂NHR₁b, C₁-C₆ alkyl, C₁-C₆ alkoxyl, C₁-C₆ alkylthio, C₁-C₆ alkylamino, di(C₁-C₆)alkylamino, C₃-C₈ cycloalkyl, C₂-C₈ alkenyl, C₂—Cs alkynyl, aryl, 5-6 membered heteroaryl containing 1-3 heteroatoms or 3-10 membered heterocyclyl containing 1-3 heteroatoms, wherein alkyl, alkoxyl, alkylthio, alkylamino, cycloalkyl, aryl, heteroaryl, heterocyclyl is optionally substituted with halogen, cyano, C₁-C₃ alkyl or C₁-C₃ alkoxyl; Preferably, R₁₀₄ is selected from: hydrogen, R₁₃, halogen, C₁-C₆ alkyl, C₁-C₆ haloalkyl; More preferably, the R₁₀,4 is selected from: hydrogen, F, Cl, Br, methyl, ethyl, trifluoromethyl, hydroxyl, Rₙ, —ORₙ.

In some specific embodiments, wherein Rₙ is selected from: C₁-C₈ alkyl substituted with hydroxyl; Preferably, Rₙis selected from: C₁-C₄ alkyl substituted with hydroxyl; More preferably, Rₙis selected from: —CH₂OH, —CH₂CH₂OH, —CH₂CH₂CH₂OH or —CH₂CH₂CH₂CH₂OH.

In some specific embodiments, wherein Rₙ is selected from: C₁-C₈ alkyl substituted with hydroxyl, sulfhydryl, or amino; More preferably, Rₙ is selected from: C₁-C₄ alkyl substituted with hydroxyl, sulfhydryl, or amino; Most preferably, Rₙ is selected from: —CH₂OH, —CH₂CH₂OH, —CH₂CH₂CH₂OH, H₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH.

In some embodiments, NH of —C(═O)NHCH₂— is not substituted.

In some embodiments, wherein R₁ is selected from R₁₃, or one of the R₁₀₄ is selected from R₁₃.

R₁₃, Rₘ, Rₙ are defined as above.

In some embodiments, the compound is a compound represented by formula (XI′) and a pharmaceutically acceptable salt, solvent compound, stereoisomer, isotope and prodrug thereof:

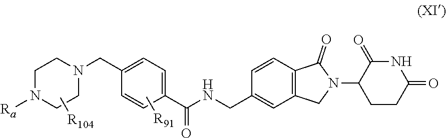

Where Rₐ, R₉₁, R₁₀₄ are defined as above.

In some embodiments, wherein R₉₁ is selected from: hydroxyl, Rₙ, —ORₙ, —NRₘRₙ, —NRₙ—C(═O)Rₘ, —NRₙ—S(═O)₂Rₘ.

In some embodiments, wherein R₁₀₄ is selected from: hydroxyl, Rₙ, —OR₁₁.

Wherein Rₙ, is selected from: C₁-C₈ alkyl substituted with hydroxyl; More preferably, Rₙ is selected from: C₁-C₄ alkyl substituted with hydroxyl; more preferably, Rₙ is selected from: —CH₂OH, —CH₂, —CH₂CH₂CH₂OH or —CH₂CH₂CH₂CH₂. Wherein Rₘ is selected from: hydrogen, —C₁-C₄ alkyl. In some embodiments, Rₙ, is selected from: C₁-C₈ alkyl substituted with hydroxyl, sulfhydryl, or amino; preferably, Rₙ is selected from: C₁-C₄ alkyl substituted with hydroxyl, sulfhydryl, or amino; more preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH.

In one embodiment, the compound represented by formula (I) and a pharmaceutically acceptable salt, solvent compound, stereoisomer, isotope and prodrug thereof: R₁ is selected from: RₐC(═O)—, RₐCHR₉C(═O)—, RₐCHR₉CH₂C(═O), RₐCHCHR₉C(═O), Rₐ, CHR₉—, RₙH is selected from: hydrogen, R₁₃; R₁₃ is selected from: hydroxyl, sulfhydryl, or amino, Rₙ: Rₙ is selected from: C₁-C₄ alkyl substituted with hydroxyl, sulfhydryl, or amino; Rₐ is selected from phenyl, naphthalen-1-yl, naphthalen-2-yl, carbazole-9-yl, 1-aza-carbazole-9-yl, 2-aza-carbazole-9-yl, diaza-carbazole-9-yl, indole-1-yl, 2, 3-dihydro-indole-1-yl, 7-aza-indole-1-yl, 2, 3-dihydro-7-aza-indole-1-yl, phenoxazine-10-yl, fluorene-9-yl, quinoline-4-yl, quinoline-5-yl, quinoline-8-yl, isoquinoline-1-yl, isoquinoline-4-yl, isoquinoline-5-yl, isoquinoline-8-yl, 1,2,3,4-tetrahydro-1,8-naphthyridine-1-yl, 1,2,3,4-tertrahydro-quinoline-1-yl, pyrimidine-2-yl, pyrimidine-4-yl, pyrimidine-5-yl, pyridine-2-yl, pyridine-3-yl, pyridine-4-yl, 5-(8-quinoline-yl)-pyridine-2-yl, 1,2,3-triazol-1-yl, 1,2,4-triazol-1-yl, 1,3,4-triazol-1-yl, bicyclo[1.11]pentane-1-yl, norborne-1-yl, adamantane-1-yl; or Rₙis selected from 5-(1-isoquinoline-yl)-pyridine-2-yl.

Rₐ is optionally substituted with one or more R₉, wherein R₉ is selected from: R₁₃, F, Cl, Br, cyano, methyl, ethyl, trifluoromethyl, methoxyl, ethoxy, phenyl, naphthalen-1-yl, naphthalen-2-yl; wherein R₁₃ is selected from: hydroxyl, sulfydryl, amino, Rₙ, —ORₙ, —OC(═O)Rₙ, —NRₘRₙ, —NRₙ—C(═O)Rₙ, —NRₙ—C(═O)Rₘ, —NRₙ—S(═O)₂Rₙ, —NRₙ—S(═O)₂Rₘ; wherein Rₙ, is selected from: C_{1I}C₄ alkyl substituted with hydroxyl, sulfhydryl or amino; wherein Rₘ is selected from: hydrogen, —C₁-C₄ alkyl;

Q is selected from: —NR₂

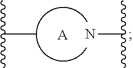

R₂ is selected from: hydrogen, R₁₄, C₁-C₈ alkyl; wherein R₁₄ is selected from: Rₐ, —C(═O)R₁, —C(═O)ORₙ, —C(═O)NRₘRₙ, —S(═O)₂Rₙ, —S(═O)₂NRₘRₙ; wherein Rₙ is selected from: —CH₂OH, —CH₂CH₂OH, —CH₂CH₂CH₂OH, —CH₂CH₂CH₂CH₂OH, —CH₂NH—CH₂CH₂NH₂, —CH₂CH₂CH₂NH₂, —CHC₂CH₂CHO₂NH₂, —CH₂SH, —CH₂CH₂SH, —CH₂CH₂CHCH₂SH, —CH₂CH₂CH₂CH₂SH; wherein Rₘ is selected from: hydrogen, —C₁-C₄ alkyl;

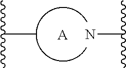

is selected from one of the following structures:

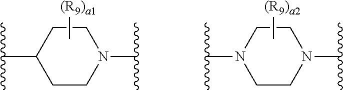

Wherein each R₉ is independently selected from: hydrogen, R₁₃; R₁j is selected from: hydroxyl, sulfydryl, amino, Rₙ; R₁ is selected from: C₁-C₄ alkyl substituted with hydroxyl, sulfhydryl or amino;

Each a t is independently selected from: 0, 1, 2, 3, 4, 5, 6, 7, 8, 9; Each a2 is independently selected from: 0, 1, 2, 3, 4, 5, 6, 7, 8;

T and Z are independently selected from: chemical bond, methylene, 1,2-ethylidene;

U is selected from: C₁-C₆ alkylene, 1,4-phenylene; wherein 1,4-phenylene is optionally substituted with one or more R₉; wherein R₉ is selected from: hydrogen, amino, hydroxyl, sulfhydryl, R₁; wherein R₁₃ is selected from: Rₙ, —ORₙ, —OC(═O)Rₙ, —NRₘRₙ, —NRₘ—C(═O)Rₙ, —NRₙ—CO(═O)Rₘ, —NRₘ—S(═O)₂Rₙ, —NRₙ—S(═O)₂Rₘ; wherein Rₙ is selected from: C₁-C₄ alkyl substituted with hydroxyl, sulfhydryl or amino; wherein Rₘ is selected from: hydrogen, —C₁-C₄ alkyl;

Y is selected from: chemical bond, —C(═O)NH(CH₂)_{b}—, —NHC(═O)(CH₂)_{b}—, —NR₂(CH₂)_{b}—;

L is selected from: —(CR₇R₈)ₒ—, —C(═O)—; wherein R₇-R₅ are each independently selected from: hydrogen;

R₃—R are each independently selected from: hydrogen;

R₆ is selected from: hydrogen;

b is selected from: 0, 1, 2, 3, 4 or 5;

n is selected from: 0, 1, 2 or 3;

m is selected from: 0, 1, 2, 3 or 4;

o is selected from: 1

In one embodiment, the compound represented by formula (I) and a pharmaceutically acceptable salt, solvent compound, stereoisomer, isotope and prodrug thereof:

Wherein R₃ is selected from: Rₐ,C(═O)—, RₐCHR₉C(═O)—, RₐCHR₉CH₂C(═O)—, RₐCHCHR₉C(═O)—, Rₐ—RₐCHR₉—, RₐCHR₉CH₂—, RₐCHCHR₉—; wherein R_{g} is selected from: hydrogen, R₁₃; wherein R₃ is selected from: hydroxyl, sulfhydryl, or amino, Rₙ; wherein Rₙ, is selected from: C₁-C₄ alkyl substituted with hydroxyl, sulfhydryl, or amino;

Rₐ is selected from the following groups:

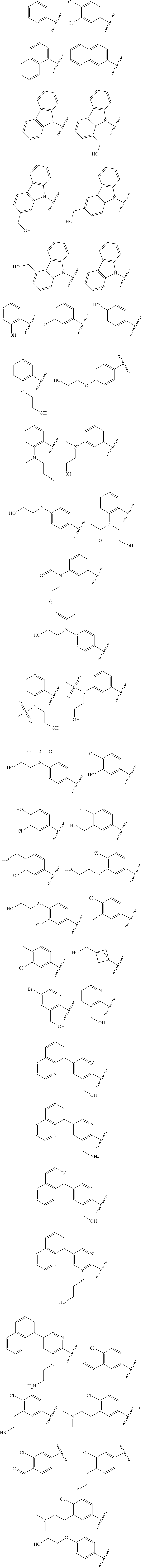

Q is selected from: —NR₂

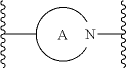

R₂ is selected from: hydrogen, R₁₄, C₁-C₈ alkyl; wherein R₁₄ is selected from: Rₐ, —C(═O)R, —C(═O)ORₙ, —C(═O)NRₘRₙ, —S(═O)₂Rₙ, —S(═O)₂, wherein Rₙ, is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH; wherein Rₙ is selected from: hydrogen, —C₁-C₄ alkyl;

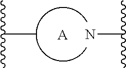

is selected from one of the following structures:

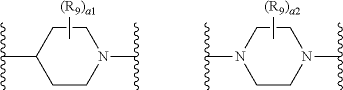

Wherein each R₉ is independently selected from: hydrogen, R₁₃; wherein R₁₃ is selected from: hydroxyl, sulfydryl, amino. R; wherein Rₙ is selected from: C₁-C₄ alkyl substituted with hydroxyl, sulfhydryl or amino;

Each a1 is independently selected from: 0, 1, 2, 3, 4, 5, 6, 7, 8, 9; each a2 is independently selected from: 0, 1, 2, 3, 4, 5, 6, 7, 8;

T and Z are independently selected from: chemical bond, methylene, 1,2-ethylidene;

U is selected from: C₁-C₆ alkylene, 1,4-phenylene; wherein 1,4-phenylene is optionally substituted with one or more R₉; wherein R₉ is selected from: hydrogen, amino, hydroxyl, sulfhydryl, R₁₃; wherein R₁ⱼ is selected from: Ri, —OR, —OC(═O)Rₙ, —NRₘRₙ, —NRₙ—C(═O)Rₙ, —NR—C(═O)Rₘ, NRₘ—SO₂R, NRₙ—S(O)₂Rₘ; wherein Rₙ is selected from: C₁-C₄ alkyl substituted with hydroxyl, sulfhydryl or amino; wherein Rₘ is selected from: hydrogen, —C₁-C₄ alkyl;

Y is selected from: chemical bond, —C(═O)NH(CH₂)_{b}—, (═O)(CH₂)—, —NR₂(CH₂)h-,

L is selected from: —(CR₇R₈)—, —C(═O)—; wherein R₇-R₅ are each independently selected from: hydrogen;
R₃-R₅ are each independently selected from: hydrogen;
R₆ is selected from: hydrogen;
b is selected from: 0, 1, 2, 3, 4 or 5;
n is selected from: 0, 1, 2 or 3;
m is selected from: 0, 1, 2, 3 or 4;
o is selected from: 1;

In some special embodiments, the present invention protect:

Item 1. The compounds and pharmaceutically acceptable salt, solvent compound, stereoisomer, isotope and prodrug thereof according to Formula (I):

Wherein,

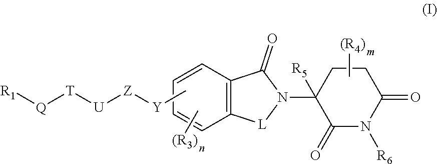

R₁ is selected from: RₐCH₂)ₐ, Rₐ(CH₂)ₐC(═O), Rₐ(CH₂)ₐNHC(═O)—, Rₐ(CH₂)ₐOC(═O)—, or Rₐ(CH₂)ₐS(═O)₂—, wherein the “CH₂” is optionally substituted with one or more R₉, or is optionally substituted with —CH₂CH₂—, and the “NH” is optionally substituted with R₁₄.

Q is selected from: —NR₂—, —O—,

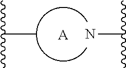

R₂ is selected from: hydrogen, R_{b}—, R_{b}C(═O)—, R_{b}S(═O)₂—, R₁₄;

Ring A represents an optionally substituted hererocycloalkyl containing at least one N atom as a heteroatom, and is linked to T through the N atom, and ring A is alternately substituted with one or more. groups selected from R₉;

Rₐ, R_{b} is selected from: C₁-C₈ alkyl, C₃-C₁₀ cycloallyl, C₃-C₁₀ bridged cyclic group, —NR_{1I}R₁₂, C₃-C₁₀ heterocyclyl optionally containing O, S, SO₂, N or NHC(═O)R₂₂, aryl, heteroaryl, fused arylcycloalkyl, fused arylheterocyclyl, fused heteroarylcycloalkyl, fused heteroarylheterocyclyl, ary-alkyl, heteroaryl-alkyl, aryl-cycloalkyl, aryl-heterocyclyl, cycloalkyl-heterocyclyl or heterocyclyl-heterocyclyl, wherein a group selected from one of —O—, —S—, —CO(═O)—, —S(═O)—, —S(═O)₂—, —NH—, —NH(═O)—, —NHC(═O)NH— or —NHS(═O)₂— may be inserted between any two C—C of the C₁-C₈ alkyl, and R may be optionally substituted with one or more R₉, Rₙ may be optionally substituted with one or more R₁₀;

T, U and Z are each independently selected from: chemical bond, carbonyl, C₁-C₆ alkylene, C₃-C₁₀ cycloalkylene, arylene, heteroarylene or heterocyclylene, wherein the alkylene, cycloalkylene, arylene, heteroarylene or heterocyclylene may be optionally substituted with one or more R₉;

Y is selected from: chemical bond, C(═O)NH(CH₂)s-, —NHC(═O) (CH₂)—, —O(CH₂)—, —NR₂(CH₂)_{b}—, wherein the “CH₂” is optionally substituted with one or more R₉, or is optionally substituted with —CH₂CH₂—, and the “NH” is optionally substituted with R₁₄;

L is selected from —(CR₇R₈)—or —C(═O)—;

R₃-R₅ and R₇-R₉ are each independently selected from: hydrogen, R₁₃, halogen, cyano, amino, nitro, —R₁₁C(═O)R₂₂, —R₂₁NHCO(═O)R₂₂, —R₂₁NR₂₂, —R₂₁C(═O)OR₂₂, —R₂₁OC(═O)R₂₂, —R₂₁S(═O)₂R₂₂, —R₂₁S(═O)₂NHᵢR₂₂, C₁-C₆ alkyl, C₁-C₆ alkoxy, C₁-C₆ allylthio, C₁-C₆ alkylamino, di(C₁-C₆ alkyl)amino, C₃-C₈ cycloalkyl, C₂-C₈ alkenyl, C₂-C₈ alkynyl, aryl, 5-to 6-membered heteroaryl containing 1-3 heteroatoms or 3- to 10-membered heterocyclyl containing 1-3 heteroatoms, wherein the alkyl, alkoxy, alkylamino, alkylthio, cycloalkyl, aryl, heteroaryl or heterocyclyl is optionally substituted with the group selected from halogen, cyano, C₁-C₃ alkyl or C₁-C₃ alkoxy; when there are multiple R - and R₇-9., any two adjacent ones may be combined to form a ring;

R₁₀ is selected from: hydrogen, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R₂₁OC(═O)Rₙ, —R₂₁S(═O)₂R₂₂, —R₂₁S(═O)₂NHR₂₂, C₁-C₆ alkyl, C₁-C₆ alkoxy, C₁-C₆ alkylthio, C₁-C₆ alkylamino, di(C₁-C₆ alkyl)amino, C₃-C₈ cycloalkyl, C₂-C₈ alkenyl, C₂-C₈ alkynyl, aryl, 5- to 6-membered heteroaryl containing 1-3 heteroatoms or 3- to 10-membered heterocyclyl containing 1-3 heteroatoms, wherein the alkyl, alkoxy, alkylamino, alkylthio, cycloalkyl, aryl, heteroaryl or heterocyclyl is optionally substituted with the group selected from halogen, cyano, C₁-C₃ alkyl or C₁-C₃ alkoxy; When R₁₀ is more than one, any two adjacent ones may be combined to form a ring; R₁₃ is selected from: hydroxyl, Rₙ, —ORₙ, —C(═O)Rₙ, —C(═O)ORₙ, —OC(═O)R, —NRₘRₙ, —C(:═O)NRₘRₙ, —NRₘ—C(═O)Rₙ, —NRₙ—C(═O)Rₘ, —S(═O)₂Rₙ, —S(═O)₂NRₘRₙ, —NRₘ—S(═O)Rₙ, —NR—S(═O)₂R₂₁; R₁₄ is selected from: Rₙ,—C(═O)R, —OC(═O)ORₙ, C(═O)NRₘRₙ,S(═O)₂Rₙ,S(═O)₂NRₘRₙ; Rₙ is selected from: C₁-C₈ alkyl substituted with hydroxyl, wherein a group selected from one of —O—, —S—C(═O),@—C(═O)O, —O)C(═O))O, —S(═O)—, —S(═O), —NH— —NHC(═O)—, —NHC(═O)NH— —NHS(═O)—, may be inserted between any two C—C of the C₁-C₈ alkyl; C₁-C₈ alkyl is each optionally substituted with 1-3 groups selected from halogen, cyano, amino, nitro or C₁-C₃ alkoxy; Rₘ is independently selected from: hydrogen, C₁-C₈ alkyl, wherein a group selected from one of —O—, —S—, —C(═O)—, —C(═O)O—, —OC(═O)O—S(═O)—, —S(═O)—, —NH—, —NHC(═O)—, —NHC(═O)NH, —NHS(═O)₂r may be inserted between any two C—C of the C₁-C₈ alkyl, C—Cs alkyl is each optionally substituted with 1-3 groups selected from halogen, hydroxyl, cyano, amino, nitro or C₁-C₃ alkoxy; R₆ is selected from: hydrogen, C₁-C₆ alkyl, C₃-C₈ cycloalkyl, C₁-C₈ alkenyl, C₁-C₈ alkynyl, aryl, 5- to 6-membered heteroaryl containing 1-3 heteroatoms or 3- to 10-membered heterocyclyl containing 1-3 heteroatoms, wherein the alkyl, cycloalkyl, aryl, heteroaryl or heterocyclyl is optionally substituted with 1-3 groups each selected from halogen, hydroxyl, cyano, amino, nitro, —C(═O)OR₂₃, —OC(═O)R₂₃, —NHC(═O)R₂₃, —C(═O)NHR₂₃, C₁-C alkyl, C₁-C₃ alkoxy or —OP(═O)(OM)₂;
M is independently selected from: hydrogen or C₁-C₄ alkyl;
R₁ and R₁₂ are each independently selected from: hydrogen, C₁-C₄ alkyl, aryl, aryl-alkyl;
R₂₁ is selected from: chemical bond, C₁-C₄ alkylene;
R₂₂ and R₂₃ are each independently selected from: hydrogen, C₁-C₄ alkyl, aryl, aryl-alkyl, and the C₁-C₄ alkyl, aryl, aryl-alkyl are optionally substituted with halogen, hydroxyl, amino;
a is selected from: 0, 1, 2, 3, 4 or 5;
b is selected from: 0, 1, 2, 3, 4 or 5;
n is selected from: 0, 1, 2 or 3;
m is selected from: 0, 1, 2, 3 or 4;
o is selected from: 1 or 2;

Preferably, the compound of Formula (1) comprises at least one group selected from R₁₃ or R₁₄.

Preferably, not following compounds are in formula (I):

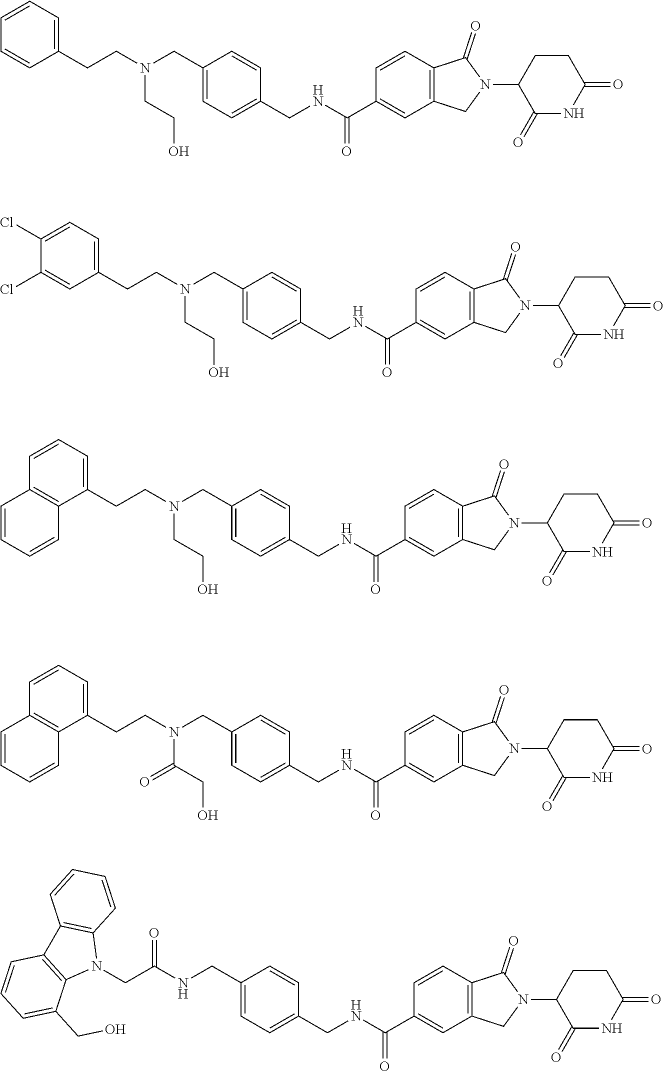 [SMILES: O=C1CCC(N2Cc3cc(C(=O)NCc4ccc(CN(CCO)CCc5ccccc5)cc4)ccc3C2=O)C(=O)N1 ; O=C1CCC(N2Cc3cc(C(=O)NCc4ccc(CN(CCO)CCc5ccc(Cl)c(Cl)c5)cc4)ccc3C2=O)C(=O)N1 ; O=C1CCC(N2Cc3cc(C(=O)NCc4ccc(CN(CCO)CCc5cccc6ccccc56)cc4)ccc3C2=O)C(=O)N1 ; O=C1CCC(N2Cc3cc(C(=O)NCc4ccc(CN(CCc5cccc6ccccc56)C(=O)CO)cc4)ccc3C2=O)C(=O)N1 ; O=C(Cn1c2ccccc2c2cccc(CO)c21)NCc1ccc(CNC(=O)c2ccc3c(c2)CN(C2CCC(=O)NC2=O)C3=O)cc1]

Item 2. The compounds and pharmaceutically acceptable salt, solvent compound, stereoisomer, isotope and prodrug thereof according to Formula (1):

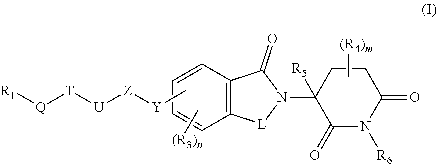

Wherein,

R₁ is selected from: Rₐ(CH₂)a, Rₐ(CH₂)ₐC(═O)—, Rₙ(CH₂)ₐNHC(═O)—, Rₐ(CH₂)ₐOC(═O)—, or Rₐ(CH₂)ₐS(═O)₂—, wherein the “CH₂” is optionally substituted with one or more R₉, or is optionally substituted with —CH₂CH₂—, and the “NH” is optionally substituted with R₁₄.

Q is selected from: —NR₂—, —O—,

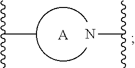

R₂ is selected from: hydrogen, R_{b}—, R_{b}C(═O)—, R_{b}S(═O)₂—, R₁₄;

Ring A represents an optionally substituted hererocycloalkyl containing at least one N atom as a heteroatom, and is linked to T or R₁ through the N atom, and ring A is alternately substituted with one or more groups selected from R₉;

Rₐ, R_{b} is selected from: C₁-C₈ alkyl, C₃-C₁₀ cycloallyl, C₃-C₁₀ bridged cyclic group, —NR₁₁R₁₂, C₃-C₁₀ heterocyclyl optionally containing O, S, SO₂, N or NHC(═O)R₂₂, aryl, heteroaryl, fused arylcycloalkyl, fused arylheterocyclyl, fused heteroarylcycloalkyl, fused heteroarylheterocyclyl, aryl-aryl, aryl-heteroaryl, heteroaryl-aryl, heteroaryl-heteroaryl, aryl-alkyl, heteroaryl-alkyl, aryl-cycloalkyl, aryl-heterocyclyl, cycloalkyl-heterocyclyl or heterocyclyl-heterocyclyl, wherein a group selected from one of —O—, —S—, —C(═O)—, —S(═O)—, —S(═O)₂—, —NH—, —NHC(═O)—, —NHC(═O)NH— or —NHS(═O)₂— may be inserted between any two C—C of the C₁-C₈ alkyl, and Rₐ may be optionally substituted with one or more Rₙ, R_{b} may be optionally substituted with one or more R₁₀;

T, U and Z are each independently selected from: chemical bond, N, O, carbonyl, C₁-C₆ alkylene, C₃-C₁₀ cycloalkylene, arylene, heteroarylene or heterocyclylene, wherein the alkylene, cycloalkylene, arylene, heteroarylene or heterocyclylene may be optionally substituted with one or more R₉;

Y is selected from: chemical bond, —C(═O)NH(CH₂)_{b}—, —NHC(═O)(CH₂)_{b}—, —O(CH₂)˜—, —NR₂(CH₂)_{b}—, wherein the “CH₂” is optionally substituted with one or more R, or is optionally substituted with —CH₂CH₂—, and the “NH” is optionally substituted with R₁₄;

L is selected from —(CR₇R₈)0— or —C(═O)—;

R₃˜R₅ and R₇-R₉ are each independently selected from: hydrogen, R₁₃, halogen, cyano, amino, hydroxyl, sulfhydryl, nitro, —R₂₁N(R_{1I})R₂₂, —R₂₁C(═O)R₂₂, —R₂₁NHCO(═O)R₂₂, —R₂₁C(═O)HR₂₂, —R₂₁C(═O)OR₂₂, —R₂₁OC(═O)R₂₂, —R₂₁S(═O)₂R₂₂, —R₂₁S(═O)₂NHR₂₂, C₁-C₈ alkyl, C₁-C₆ alkoxy, C₁-C₆ alkylthio, C₁-C₆ alkylamino, di(C₁-C₆ alkyl)amino, C₃-C₈ cycloalkyl, C₂-C₈ alkenyl, C₂-C₈ alkynyl, aryl, 5- to 6-membered heteroaryl containing 1-3 heteroatoms or 3- to 10-membered heterocyclyl containing 1-3 heteroatoms, wherein the alkyl, alkoxy, alkylamino, alkylthio, cycloalkyl, aryl, heteroaryl or heterocyclyl is optionally substituted with the group selected from halogen, cyano, C₁-C₃ alkyl or C₁-C₃ alkoxy; when there are multiple R₁-R₅ and 1R₇-R₉, any two adjacent ones may be combined to form a ring;

R₁₀ is selected from: hydrogen, halogen, cyano, amino, nitro, —R₂₁C(═O)R₂₂, —R₂₁NHC(═O)R₂₂, C(═O)R₂, C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxy, C₁-C₆ alkylthio, C₁-C₆ alkylamino, di(C IC_{G} alkyl)amino, C₃-C₈ cycloalkyl group, C₂-C₈ alkenyl, C₂-C₈ alkynyl, aryl, 5- to 6-membered heteroaryl containing 1-3 heteroatoms or 3- to 10-membered heterocyclyl containing 1-3 heteroatoms, wherein the alkyl, alkoxy, alkylamino, alkylthio, cycloalkyl, aryl, heteroaryl or heterocyclyl is optionally substituted with the group selected from halogen, cyano, C₁-C₃ alkyl or C₁-C₃ alkoxy; When R₁₀ is more than one, any two adjacent ones may be combined to form a ring;

R₁₃ is selected from: hydroxyl, sulfhydryl, amino, Rₙ, —ORₙ, —C(═O)Rₙ, —C(═O)OR, —OC(═O)Rₙ, —NRₘRₙ, —C(═O)ₘRₙ, —NRR—C(═O)R, —NRₙ—C(═O)Rₘ, —S(═O)₂R, —S(═O)₂NRₘRₙ, —R—S(═O)₂Rₙ, —NRₙ—S(═O)₂Rₘ; R₁₄ is selected from: Rₙ, —C(═O)Rₙ, —C(═O)ORₙ, —CC(═O)NRₘRₙ, —S(═O)₂Rₙ, —S(═O)₂NRR₁; R₁ is selected from: C₁-C₈ alkyl substituted with hydroxyl, sulfhydryl, or amino group, wherein a group selected from one of —O—, —S—, —C(═O)—, —C(═O)O—, —OC(═O)O—, —S(═O)—, —S(═O)₂, —NH, —NHC(═O)—, —NHC(═O)NH, —NHS(═O)₂— may be inserted between any two C—C of the C₁-C₈ alkyl; C₁-C₈ alkyl is each optionally substituted with 1-3 groups selected from halogen, cyano, amino, nitro or C₁-C₃ alkoxy;

Rₘ is independently selected from: hydrogen, C₁-C₈ alkyl, wherein a group selected from one of —O— —S—, —C(═O)—, —C(═O)O—, —OC(═O)O—, —S(═O)—, —S(═O)₂— —NH—, —NIC(═O), —NHC(═O)NH—, —NHS(═O)₂— may be inserted between any two C—C of the C₁-C₈ alkyl, C₁-C₈ alkyl is each optionally substituted with 1-3 groups selected from halogen, hydroxyl, cyano, amino, nitro or C₁-C₃ alkoxy;

R₆ is selected from: hydrogen, C₁-C₆ alkyl, C₃-C₈ cycloalkyl, C₂-C₈ alkenyl, C₂-C₈ alkynyl, aryl, 5- to 6-membered heteroaryl containing 1-3 heteroatoms or 3- to 10-membered heterocyclyl containing 1-3 heteroatoms, wherein the alkyl, cycloalkyl, aryl, heteroaryl or heterocyclyl is optionally substituted with 1-3 groups each selected from halogen, hydroxyl, cyano, amino, nitro, —C(═O)OR₂₃, —OC(═O)R₂₃, —NHC(═O)R₂₃, —C(═O)NHR₂₃, C₁-C₃ alkyl, C₁-C₃ alkoxy or —OP(═O)(OM)₂;
M is independently selected from: hydrogen or C₁-C₄ alkyl;
R₂₁ and R₁₂ are each independently selected from: hydrogen, C₁-C₄ alkyl, aryl, aryl-alkyl;
R₂₁ is selected from: chemical bond, C₁-C₄ alkylene;
R₂₂ and R₂₃ are each independently selected from: hydrogen, C₁-C₄ alkyl, aryl, aryl-alkyl, and the C₁-C₄ alkyl, aryl, aryl-alkyl are optionally substituted with halogen, hydroxyl, amino;
a is selected from: 0, 1, 2, 3, 4 or 5;
b is selected from: 0, 1, 2, 3, 4 or 5;
n is selected from: 0, 1, 2 or 3;
in is selected from: 0, 1, 2, 3 or 4;
is selected from: 1 or 2;
Not following compounds are in formula (I)

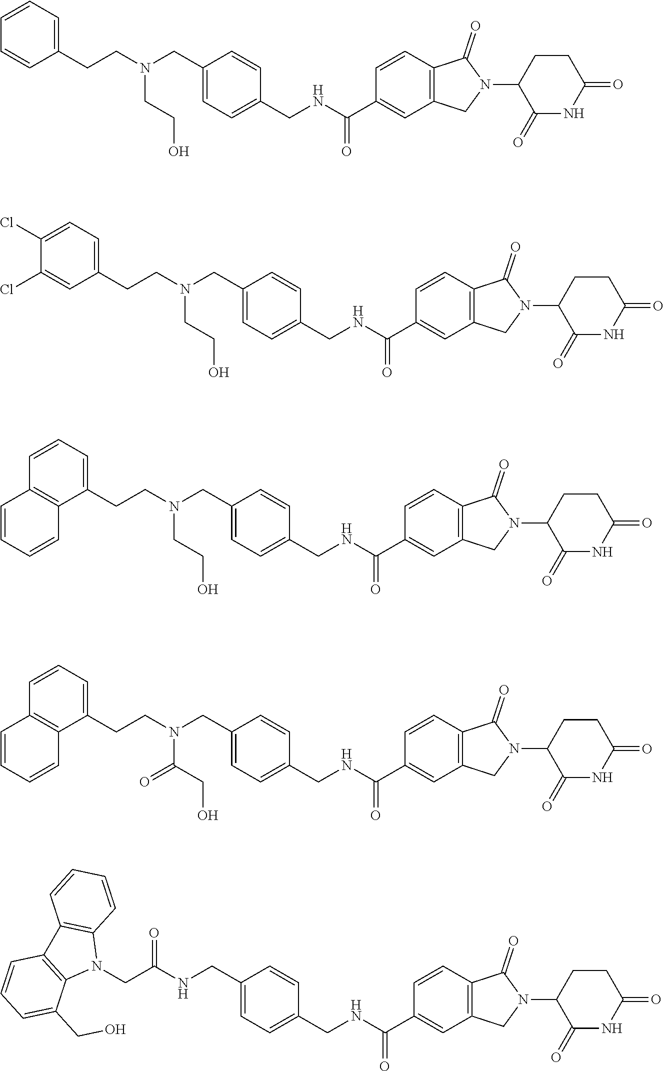 [SMILES: O=C1CCC(N2Cc3cc(C(=O)NCc4ccc(CN(CCO)CCc5ccccc5)cc4)ccc3C2=O)C(=O)N1 ; O=C1CCC(N2Cc3cc(C(=O)NCc4ccc(CN(CCO)CCc5ccc(Cl)c(Cl)c5)cc4)ccc3C2=O)C(=O)N1 ; O=C1CCC(N2Cc3cc(C(=O)NCc4ccc(CN(CCO)CCc5cccc6ccccc56)cc4)ccc3C2=O)C(=O)N1 ; O=C1CCC(N2Cc3cc(C(=O)NCc4ccc(CN(CCc5cccc6ccccc56)C(=O)CO)cc4)ccc3C2=O)C(=O)N1 ; O=C(Cn1c2ccccc2c2cccc(CO)c21)NCc1ccc(CNC(=O)c2ccc3c(c2)CN(C2CCC(=O)NC2=O)C3=O)cc1]

Preferably, the compound shown in formula (I) comprises at least one group selected from R₁₃ or R₁₄.

Item 3. The compounds and pharmaceutically acceptable salt, solvent compound, stereoisomer, isotope and prodrug thereof according to item 1 or item 2, wherein R₁ is selected from: RₐC(═O)—1 C)—, RH₂CH₂(═O), RₐCHC(═O)—, Rₐ—, RₐCH₂—, ₐCH₂CH₂—, RₐNHC(═O)—, RₐCH₂NHC(═O)—, RₐCH₂CH₂NHC(═O)—, RₐOC(═O)—, RₐCH₂OC(═O)—, RₐCH₂CH₂OC(═O)—, RₐS(═O)₂—CH₂S(═O)₂—, RₐCH₂CH₂S(═O)₂—, wherein “CH₂” is optionally substituted with one or more R₉, or optionally substituted with —CH₂CH₂—; and the “NH” is optionally substituted with R₁₄;

Item 4. The compounds and pharmaceutically acceptable salt, solvent compound, stereoisomer, isotope and prodrug thereof according to item 3, wherein R₁ is selected from: RₐC(═O)—, RₙCHR₉C(═O)—, RₐCHR₉CH₂C(═O)—, RₐCHCHR₉C(═O)—, Rₐ—, RₐCHR₉—, RₐCHR₉CH₂—, RCHCHR₉—, RₐNR₄C(═O)—, RₐCHRNHC(═O)—, RₐCINR₁₄C(═O)—, RₐCHRₐCH₂NHC(═O)—, RₐCH₂CHR₉NHC(═O)—, RₐCH₂CH₂NR₄C(═O)—, RₙOC(═—, (═O), Rₙ,CHRC₂OC(═O), RₐCH₂CHR₉OC(═O)—, RₐCHR₉S(═O)₂—, RₐCHR₉CH₂S(═O)₂—, RₐCH₂CHRS(═O)₂—.

Item 5. The compound and the pharmaceutically acceptable salt, solvent compound, stereoisomer, isotope, and prodrag thereof according to item 3 or 4, wherein,
R₉ is selected from: hydrogen, C₁-C₄ alkyl, R₃;
R₁₃ is selected from: hydroxyl, Rₙ, —OR;
R₁₄ is selected from: Rₙ, —C(═O)Rₙ;
Rₙ is selected from: C₁-C₈ alkyl substituted with hydroxyl; Preferably, Rₙis selected from: C₁-C₄ alkyl substituted with hydroxyl; More preferably, R₁ is selected from: —OH, —CH₂CH₂OH, —CH₂CH₂CH₂OH, —CH₂CH₂CH₂CH₂OH.

Item 6. The compounds and pharmaceutically acceptable salt, solvent compound, stereoisomer, isotope and prodrug thereof according to item 3 or item 4, wherein,
R₉ is selected from: hydrogen, C₁-C₄ alkyl, —R₂IN(R₂₂)R₂₂, —R₂₁C(═O)R₂₂, R₁₃;
R₁₃ is selected from: hydroxyl, sulfhydryl, amino, Rₙ, —OR;
R₁₄ is selected from: Rₙ, —C(═O)Rₙ;
Rₙ is selected from: C₁-C₈ alkyl substituted with hydroxyl, sulfhydryl or amino; Preferably, the R₉ is selected from: C₁-C₄ alkyl substituted with hydroxyl, sulfhydryl or amino; More preferably, Rₙis selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂H₂₂CH₂SH, —CH₂CH₂CH₂CH₂SH.

Item 7. The compounds and pharmaceutically acceptable salt, solvent compound, stereoisomer, isotope and prodrug thereof according to item 1 or item 2, wherein, Rₐ is selected from: phenyl, naphthyl, carbazolyl, 1-aza-carbazolyl, 2-aza-carbazolyl, 1,8-diaza-carbazolyl, indolyl, 7-aza-indole, 2,3-dihydroindolyl, 2,3-dihydro-7-aza-indole, phenoxazinyl, fluorenyl, quinolinyl, isoquinolinyl, nalididinyl, tetrahydronalididinyl, tetrahydroquinolinyl, pyrimidinyl, triazolyl, bicyclo[1.1.1]pentyl, norborneyl, adamantane; Preferably, the is optionally substituted with one or more R, wherein R₉ is selected from hydrogen, R₁₃, halogen, cyano, C₁-C₆ alkyl, C₁-C₆ haloalkyl, C₁-C₆ alkoxyl, phenyl, naphthyl.

Item 8. The compounds and pharmaceutically acceptable salt, solvent compound, stereoisomer, isotope and prodrug thereof according to item 1 or item 2, wherein, Rₙ is selected from: phenyl, naphthyl, carbazolyl, 1-aza-carbazolyl, 2-aza-carbazolyl, 1,8-diaza-carbazolyl, indolyl, 7-aza-indole, 2,3-dihydroindolyl, 2,3-dihydro-7-aza-indole, phenoxazinyl, fluorenyl, quinolinyl, isoquinolinyl, nalididinyl, tetrahydronalididinyl, tetrahydroquinolinyl, pyrimidinyl, pyridinyl, quinolinyl-pyridyl, triazolyl, bicyclo[1.1.11pentyl, norborneyl, adamantane; Preferably, the Rₐ is optionally substituted with one or more R₉, wherein R₉ is selected from hydrogen, R₁₃, halogen, cyano, C₁-C₆ alkyl, C₁-C₆ haloalkyl, C₁-C₆ alkoxyl, phenyl, naphthyl.

Item 9. The compounds and pharmaceutically acceptable salt, solvent compound, stereoisomer, isotope and prodrug thereof according to item 7 or item 8, wherein, Rₐ is selected from: phenyl, naphthalen-1-yl, naphthalen-2-yl, carbazole-9-yl, 1-aza-carbazole-9-yl, 2-aza-carbazole-9-yl, 1,8-diaza-carbazole-9-yl, indole-1-yl, 2, 3-dihydro-indole-1-yl, 7-aza-indole-1-yl, 2, 3-dihydro-7-aza-indole-1-yl, phenoxazine-10-yl, fluorene-9-yl, quinoline-4-yl, quinoline-5-yl, quinoline-8-yl, isoquinoline-1-yl, isoquinoline-4-yl, isoquinoline-5S-yl, isoquinoline-8-yl, 1,2,3,4-tetrahydro-1,8-naphthyridine-1-yl, 1,2,3,4-tertrahydro-quinoline-1-yl, pyrimidine-2-yl, pyrimidine-4-yl, pyrimidine-5-yl, 1,2,3-triazol-1-yl, 1,2,4-triazol-1-yl, 1,3,4-triazol-1-yl, bicyclol1.1.11pentane-1-yl, norborne-1-yl, adamantane-1-yl; Preferably, Rₐ is optionally substituted with one or more R₉, wherein R₉ is selected from: R₁₃, F, Cl, Br, cyano, methyl, ethyl, trifluoromethyl, methoxyl, ethoxy, phenyl, naphthalen-1-yl, naphthalen-2-yl.

Item 10. The compounds and pharmaceutically acceptable salt, solvent compound, stereoisomer, isotope and prodrug thereof according to item 7 or item 8, wherein, Rₐ is selected from: phenyl, naphthalen-1-yl, naphthalen-2-yl, carbazole-9-yl, 1-aza-carbazole-9-yl, 2-aza-carbazole-9-yl, 1,8-diaza-carbazole-9-yl, indole-1-yl, 2, 3-dihydro-indole-1-yl, 7-aza-indole-1-yl, 2, 3-dihydro-7-aza-indole-1-yl, phenoxazine-10-yl, fluorene-9-yl, quinoline-4-yl, quinoline-5-yl, quinoline-8-yl, isoquinoline-1-yl, isoquinoline-4-yl, isoquinoline-5-yl, isoquinoline-8-yl, 1,2,3,4-tetrahydro-1,8-naphthyridine-1-yl, 1,2,3,4-tertrahydro-quinoline-1-yl, pyrimidine-2-yl, pyrimidine-4-yl, pyrimidine-5-yl, pyridine-2-yl, pyridine-3-yl, pyridine-4-yl, 5-(8-quinoline-yl)-pyridine-2-yl, 1,2,3-triazol-1-yl, 1,2,4-triazol-yl, 1,3,4-triazol-1-yl, bicyclol1.1.11pentane-1-yl, norborne-yl, adamantane-1-yl; Preferably, Rₙis optionally substituted with one or more R₉, wherein R₉ is selected from: R_{J}, F, Cl, Br, cyano, methyl, ethyl, trifluoromethyl, methoxyl, ethoxy, phenyl, naphthalen-1-yl, naphthalen-2-yl.

Item 11. The compounds and pharmaceutically acceptable salt, solvent compound, stereoisomer, isotope and prodrug thereof according to item 7 to item 10, wherein,

R₁₃ is selected from: hydroxyl, Rₙ, —ORₙ, —OC(═O)Rₙ, —NRₘRₙ, —NRₘ—C(═O)Rₐ, —NRₙ—C(═O)Rₙ, —NRₘ—S(═O)₂Rₙ, —NRₙ—S(═O)₂Rₘ; Preferably, R₁₃ is selected from: hydroxyl, R₁, —ORₙ, —NRₘRₙ, —NRₙ—C(═O)Rₘ, —NRₙ—S(═O)₂Rₘ;

Rₙis selected from: C₁-C₈ alkyl substituted with hydroxyl; More preferably, Rₙis selected from: C₁-C₄ alkyl substituted with hydroxyl; Preferably, Rₙ is selected from: —CH₂OH, —CH₂CH₂OH, —CH₂CH₂CH₂OH, —CH₂CH₂CH₂CH₂;

Rₘ is selected from: hydrogen, —C₁-C₄ alkyl.

Item 12. The compounds and pharmaceutically acceptable salt, solvent compound, stereoisomer, isotope and prodrug thereof according to item 7 to item 10, wherein,

R₁₃ is selected from: hydroxyl, sulfydryl, amino, Rₙ, —ORₐ, —OC(═O)Rₙ, —NRₘRₙ, —NRₘ,—C(═O)Rₙ, —NRₙ—C(═O)Rₘ, —NRₘ=R—S(═O)₁Rₙ, —NRₙ,—S(═O)₂Rₘ; Preferably, R₁₃ is selected from: hydroxyl, Rₙ, —ORₙ, —NRₘRₙ, —NRₙ—C(═O)Rₘ, —NRₙ—S(O)₂Rₘ;

Rₙ is selected from: C₁-C₈ alkyl substituted with hydroxyl, sulfhydryl or amino; More preferably, Rₙ, is selected from: C₁-C₄ alkyl substituted with hydroxyl, sulfydryl or amino; Preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SIH, —CH₂CH₂SH, —CH₂CH₂CH₂SH-1, —CH₂CH₂CH₂CH₂SH Rₘ is selected from: hydrogen, —C₁-C₄ alkyl.

Item 13. The compounds and pharmaceutically acceptable salt, solvent compound, stereoisomer; isotope and prodrug thereof according to item 11 to item 12, wherein, R₉ is selected from: F, Cl, Br, cyano, methyl, ethyl, trifluoromethyl, methoxyl, ethoxyl, hydroxyl, —CH₂OH, —CH₂CH₂OH, —CH₂CH₂CH₂OH, —OCH₂OH, —OCH₂CH₂OH, —OCH₂CH₂CH₂OH, —NHCH, —NHCH₂CH₂OH, —NHCH₂CH₂CH₂OH, —N(CH₃)CH₂OH, —N(CH₃)CH₂CH₂OH, —N(CH₃)CH₂CH₂CH₂OH, —N(COCH₃)CH₂OH, —N(COCH₃)CH₂CH₂OH, —N(COCH₃)CH₂CH₂CH, —N(SO₂CH₃)CH₂OH, —N(SO₂CH₃)CH₂C₂OH, —N(SO₂CH₃)CH₂CH₂CH₂OH.

Item 14. The compounds and pharmaceutically acceptable salt, solvent compound, stereoisomer; isotope and prodrug thereof according to item 11 to item 12, wherein, R₉ is selected from: F, Cl, Br, cyano, methyl, ethyl, trifluoromethyl, methoxyl, ethoxyl, hydroxyl, sulfhydryl, amino, —C(═O)CH₃, C(═O)CH₂CH₃, C(═O)CH₂CH₂CH₃, —CH₂CH₂N(CH₃)₂, —CH₂OH, —CH₂CH₂OH, —CH₂CH₂CH₂₀₁, —CH₂NH₂, —CH₂CH₂NH, —CH₂CH₂CH₂NH₂, —CH₂SH, —CH₂CH₂SH, —CH₂CH₂CH₂SH, —CH₂CH₂CH₂CH₂SH, —OCH₂OH, —OCH₂CH₂OH, —OCH₂CH₂CH₂OH, —OCH₂ NH₂, —OCH₂CH₂NH₂, —OCH₂CH₂CH₂NH₂, —OCH₂SH, —OCH₂CH₂SH, —OCH₂CH₂CH₂SH, —NHCH₂OH, —NHCH₂CH₂OH, —NHCH₂CH₂CH₂OH, —NHCH₂NH₂, —NHCH₂CH₂NH₂, —NHCH₂CH₂CH₂NH₂, —NHCH₂SH, —NHCH₂CH₂SH, —NHCH₂CH₂CH₂SH, —N(CH₃)CH₂OH, —N(CH₃)CH₂CH₂OH, —N(CH₃)CH₂CH₂CH₂OH, —N(CH₃)CH₂NH₂, —N(CH₃)CH₂CH₂NH₂, —N(CH₃)CH₂CH₂CH₂NH₂, —N(CH₃)CH₂SH, —N(CH₃)CH₂CH₂SH, —N(CH₃)CH₂CH₂CH₂SH, —N(COCH₃)CH₂OH, —N(COCH₃)CH₂CH₂OH, —N(COCH₃)CH₂CH₂CH₂OH, —N(COCH₃)CH₂NH₂, —N(COCH₃)CH₂CH₂NH₂, —N(COCH₃)CH₂CH₂CH₂NH₂, —N(COCH₃)CH₂SH, —N(COCH₃)CH₂CH₂SH, —N(COCH₃)CH₂CH₂CH₂SH, —N(SO₂CH₃)CH₂OH, —N(SO₂CH₃)CH₂CH₂OH, —N(SO₂CH₃)CH₂CH₂CH₂OH, —N(SO₂CH₃)CH₂NH₂, —N(SO₂CH₃)CH₂CH₂NH₂, —N(SO₂CH₃)CH₂CH₂CH₂NH₂, ˜—N(SO₂CH₃)CH₂SH, ˜—N(SO₂CH₃)CH₂CH₂SH, —N(SO₂CH₃)CH₂CH₂CH₂SH.

Item 15. The compounds and pharmaceutically acceptable salt, solvent compound, stereoisomer, isotope and prodrug thereof according to item 1 or item 2, wherein, Rₐ is selected from:

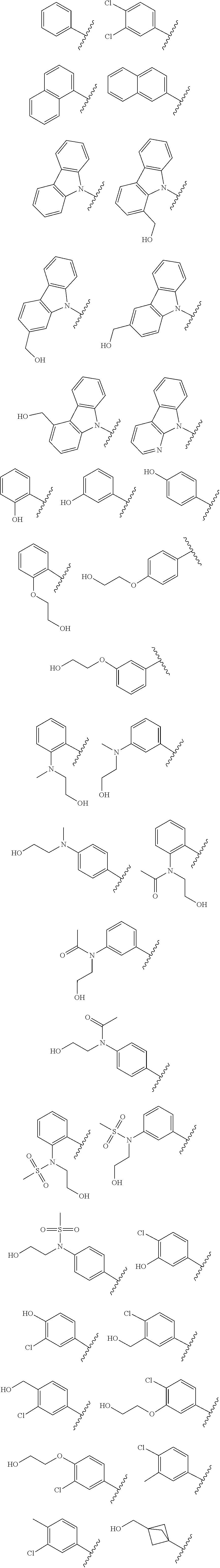

Item 16. The compounds and pharmaceutically acceptable salt, solvent compound, stereoisomer, isotope and prodrug thereof according to item 1 or item 2, wherein, Rₐ is selected from:

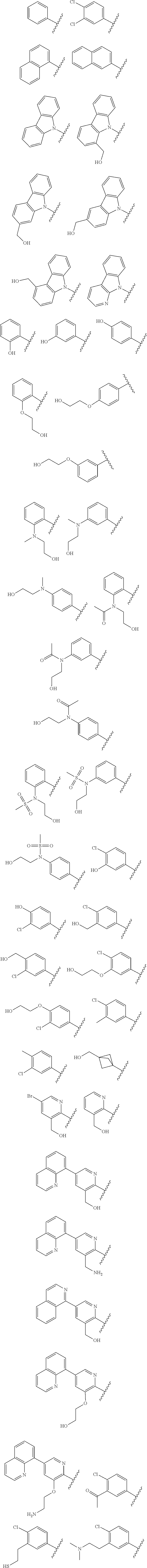

Item 17. The compounds and pharmaceutically acceptable salt, solvent compound, stereoisomer, isotope and prodrug thereof according to item 1 or item 2, wherein, R₂ is selected from: hydrogen, R₁₄, C₁-C₈ alkyl, C₃-C₁₀ cycloalkyl, aryl, —C(═O)C₃-C₀cycloalkyl, —C(═O)C₁-C₈ alkyl or —S(═O)₂C₁-C₈ alkyl, wherein C₁-C₈ alkyl, C₃-C₁₀ cycloalkyl, aryl and the cycloalkyl or alkyl portion of —C(═O)C₃-C₁₀ cycloalkyl, —C(═O)C₁-C₈ alkyl or —S(═O)₂C₁-C₈ alkyl is optionally substituted with one or more R₉, wherein R₉ is selected from: hydrogen, halogen, amino, cyano, carboxyl, C₁-C₆ alkyl, C₁-C₆ alkoxyl or aryl; Preferably, R₂ is selected from: R₁₄.

Item 18. The compounds and pharmaceutically acceptable salt, solvent compound, stereoisomer, isotope and prodrug thereof according to item 17, wherein, R₁₄ is selected from: Rₙ, —C(═O)Rₐ, —C(═O)ORₙ, —C(═O)NRₘRₙ, —S(═O)₂Rₙ, —S(═O)₂NRₘRₙ.

Rᵣ, is selected from: C₁-C₈ alkyl substituted with hydroxyl; Preferably, Rₙ is selected from: C₁-C₄ alkyl substituted with hydroxyl; More preferably, Rₙ is selected from: —CH₂OH, —CH_{2:1}, —CH₂CH₂CH₂OH, —CH₂CH₂CH₂CH₂OH; Rₘ is selected from: hydrogen, —C₁-C₄ alkyl.

Item 19. The compounds and pharmaceutically acceptable salt, solvent compound, stereoisomer, isotope and prodrug thereof according to item 17, wherein,

R₁₄ is selected from: Rₙ, —C(═O)Rₙ, —C(═O)ORₙ, —C(═O)NRₘRₙ, —S(═O)₂Rₐ, —S(═O)₂NRₘRₙ.

Rₙ is selected from: C₁-C₈ alkyl substituted with hydroxyl, sulfhydryl or amino; Preferably, Rₙ is selected from: C₁-C₄ alkyl substituted with hydroxyl, sulfhydryl or amino; More preferably, R₁ is selected from: —CH₂OH, —CH₂CH₂OH, —CH₂CH₂CH₂OH, —CH₂CH₂CH₂CH₂OH, —CH₂NH₂, —CCH₂CH₂NH₂, —CH₂CH₂CH₂, —CH₂CH₂CH₂CH₂NH₂, —CH₂SH, —CH₂CH₂SH, —CH₂CH₂CH₂SH, —CH₂CH₂CH₂CH₂SH; Rₘ is selected from: hydrogen, —C₁-C₄ alkyl.

Item 20. The compounds and pharmaceutically acceptable salt, solvent compound, stereoisomer, isotope and prodrug thereof according to item 18 or item 19, wherein, R₂ is selected from hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthyl ethyl, —C₂OH, —CH₂CH₂OH, —CH₂CH₂CH₂OH, —C(═O)CH₂OH, —C(═O)CH₂CH₂OH, —C(═O)CH₂CH₂CH₂OH, —C(═O)OCH₂OH, —C(═O)OCH₂CH₂OH, —C(═O)OCH₂CH₂CH₂OH, —C(═O)NHCH₂OH, —C(═O)NHCH₂CH₂OH, —C(═O)NHCH₂CH₂CH₂OH, —S(═O)₂CH₂OH, —S(═O)₂CH₂CH₂OH, —S(═O)₂CH₂CH₂, —S(═O)₂NHCH₂OH, —S(═O)₂NHCH₂CH₂OH, —S(═O)₂NHCH₂CH₂CH₂OH.

Item 21. The compounds and pharmaceutically acceptable salt, solvent compound, stereoisomer, isotope and prodrug thereof according to item 18 or item 19, wherein, R₂ is selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthyl ethyl, —CH₂OH, —CH₂CH₂OH, —CH₂CH₂CH₂, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —C(═O)CH₂OH, —C(═O)CH₂CH₂OH, —C(═O)CH₂CH₂CH₂OH, —C(═O)CH₂NH₂, —C(═O)CH₂CH₂NH₂, —C(═O)CH₂CH₂CH₂NH₂, —C(═O)CH₂SH, —C(═O)CH₂CH₂SH, —C(═O)CH₂CH₂CH₂SH, —C(═O)OCH₂OH, —C(═O)OCH₂CH₂OH, —C(═O)OCH₂CH₂CH₂OH, —COC₁NH, —C(═O)OCH₂CH₂NH₂, C(═O)OC—CH₂CH₂H₂, —C(═O)OCH₂SH, —C(═O)OCH₂CH₂SH, —C(═O)OCHCH₂CH₂SH, —C(═O)NH₂OH, —C(═O)NHCH₂CH₂OH, —C(═O)NHCH₂CH₂CH₂OH, —C(═O)N HCH₂NH₂, —C(═O)NHCH₂CH₂CH₂, -.C(═O)NHCH₂CH₂CH₂CH₂, —C(═O)NHCH₂SH, —C(═O)NHCH₂CH₂SH, —C(═O)NHCH₂CH₂CH₂SH, —S(═O)₂CH₂—S(═O)₂CH₂CH₂OH, —S(═O)₂CH₂CH₂CH₂OH, —S(═O)₂CH₂, —S(═O)C: ₂CH₂NH₂, —S(═O)CH₂CH₂CH₂NH₂, —S(═O)₂CH₂SH, —S(═O)₂CH₂CH₂SH, —S(═O)₂CH₂CH₂CH₂SH, —S(═O)₂NHCH₂, —S(═O)₂NHCH₂CH₂OH, —S(═O)₂NHCH₂CH₂CH₂OH, —S(═O)₂NHCH₂NH₂, —S(═O)₂NHCH₂CH₂NH₂,
S(═O)₂NH₆CH₂CH₂CH₂NH₂, —S(═O)₂NHCH₂SH, —S(═O)₂NHCH₂CH₂SH, —S(═O)₂NHCH₂CH₂CH₂SH.

Item 22. The compounds and pharmaceutically acceptable salt, solvent compound, stereoisomer, isotope and prodrug thereof according to item 1 or item 2, wherein,

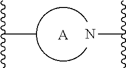

is selected from one of the following structures:

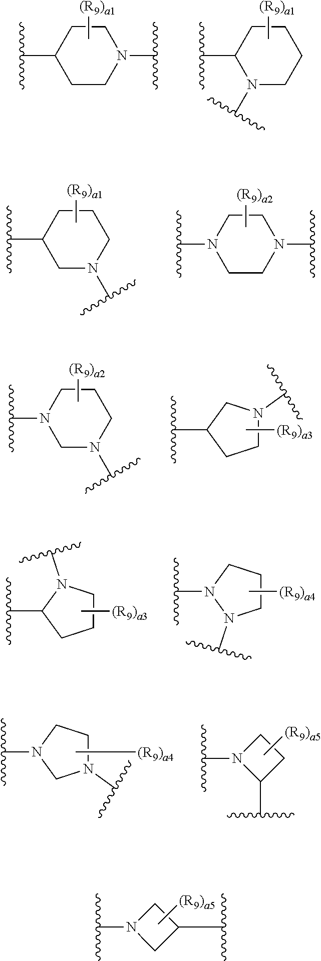

Wherein each R₉ is independently selected from: hydrogen, R₁₃, halogen, cyano, nitro, C₁-C₆ C₁-C₆ haloalkyl, C₁-C₆ alkoxyl;

Each a1 is independently selected from: 0, 1, 2, 3, 4, 5, 6, 7, 8, 9; each a2 is independently selected from: 0, 1, 2, 3, 4, 5, 6, 7, 8; each a3 is independently selected from: 0, 1, 2, 3, 4, 5, 6, 7; each a4 is independently selected from: 0, 1, 2, 3, 4, S, 6; each a5 is independently selected from: 0, 1,2,3,4,5.

Item 23. The compounds and pharmaceutically acceptable salt, solvent compound, stereoisomer, isotope and prodrug thereof according to item 22, wherein, R₁₃ is selected from: hydroxyl, Rₙ, —ORₙ, —OC(═O)Rₐ, —NRₘRₙ, —NRₘ—(C═O)Rₙ, —NRₙ—(═O)Rₘ, —NRₘ—S(═O)₂Rₙ, —NRₐ—S(═O)₂Rₘ; Rₙis selected from: C₁-C₈ alkyl substituted with hydroxyl; Preferably, Rₙ is selected from: C₁-C₄ alkyl substituted with hydroxyl; More preferably, R is selected from: —CH₂OH, —CH₂CH₂OH, —CH₂CH₂CH₂OH, or —CH₂CH₂CH₂CH₂OH.

Rₘ is selected from: hydrogen, —C₁-C₄ alkyl.

Item 24. The compounds and pharmaceutically acceptable salt, solvent compound, stereoisomer, isotope and prodrug thereof according to item 22, wherein,

R₁₃ is selected from: hydroxyl, sulfhydryl, amino, Rₙ, —ORₙ, —OC(═O)R₁, —NRₘRₙ, —NRₘ,—C(═O)Rₙ, —NRₙ—C(═O)Rₘ, —NRₘ—S(═O)₂Rₙ, —NRₙ—S(═O)₂Rₘ

Rₙ is selected from: C₁-C₈ alkyl substituted with hydroxyl, sulfhydryl or amino; Preferably, Rₙ is selected from: C₁-C₄ alkyl substituted with hydroxyl, sulfhydryl or amino; More preferably, R₁ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂ CH₂SH, —CH₂CH₂CH₂SH, —CH₂CH₂CH₂CH₂SH;

Rₘ is selected from: hydrogen, —C₁-C₄ alkyl.

Item 25. The compounds and pharmaceutically acceptable salt, solvent compound, stereoisomer, isotope and prodrug thereof according to item 22, wherein,

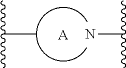

is selected from one of the following structures:

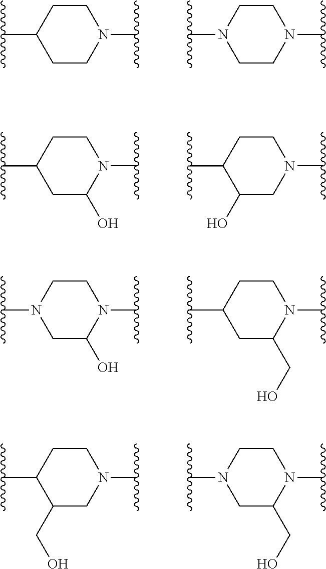

Item 26. The compounds and pharmaceutically acceptable salt, solvent compound, stereoisomer, isotope and prodrug thereof according to item 1 or item 2, wherein T, Z is independently selected from: chemical bond, carbonyl, C₁-C₆ alkylene or C₃-C₁₀ cycloalkylene, wherein the C₁-C₆ alkylene or C₃-C₁₀ cycloalkylene may be optionally substituted with one or more R₉.

Item 27. The compounds and pharmaceutically acceptable salt, solvent compound, stereoisomer, isotope and prodrug thereof according to item 26, wherein T, Z is independently selected from: chemical bond, carbonyl, methylene, 1,2-ethyleneyl, 1,1-cyclopropylene, or 2,2-propylene, wherein methylene, 1,2-ethylidene, 1,l-cyclopropylene is optionally substituted with one or more R₉.

Item 28. The compounds and pharmaceutically acceptable salt, solvent compound, stereoisomer, isotope and prodrug thereof according to item 26 or item 27, wherein
R₉ is selected from: hydrogen, R₃, C₁-C₆ alkyl or C₃-C₈ cycloalkyl;
R₁₃ is selected from: hydroxyl, Rₙ—OR;
Rₙ is selected from: C₁-Cs alkyl substituted with hydroxyl; Preferably, Rₙ is selected from: C₁-C₄ alkyl substituted with hydroxyl; More preferably, Rₙ is selected from: —CH₂OH, —CH₂CH₂OH, —CH₂CH₂CH₂OH, or —CH₂CH₂CH₂CH₂.

Item 29. The compounds and pharmaceutically acceptable salt, solvent compound, stereoisomer, isotope and prodrug thereof according to item 26 or item 27, wherein
R₉ is selected from: hydrogen, hydroxyl, sulfhydryl, amino, —R₂₁N(R₂)R₂, -n₂₁C(═O)R₂₂,
R₁₃, C₁-C₆ alkyl or C₃-C₈ cycloalkyl;
R₁₃ is selected from: hydroxyl, sulfhydryl, amino, R₁, —ORₙ; Rₙis selected from: C₁-C₈ alkyl substituted with hydroxyl, sulfhydryl or amino; Preferably, Rₙ, is selected from: C₁-C₄ alkyl substituted with hydroxyl, solidary or amino; More preferably, Rₙis selected from: —CH₂, —CH₂CH₂, —CH₂C₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SHI.

Item 30. The compounds and pharmaceutically acceptable salt, solvent compound, stereoisomer, isotope and prodrug thereof according to item 1 or item 2, wherein U is selected from: C₁-C₆ alkylene, C₃-C₁₀ cycloalkylene, arylene, or heteroarylene, wherein alkylene, cycloalkylene, arylene, or heteroarylene is optionally substituted with one or more R₉. Preferably, U is selected from: C₂-C₆ alkylene, C₅-C₆ cycloalkylene, C₆-C₁₀ alkylene, 5-6 membered monocyclic heteroarylene, wherein alkylene, cycloalkylene, arylene, or heteroarylene is optionally substituted with one or more R₉.

Item 31. The compounds and pharmaceutically acceptable salt, solvent compound, stereoisomer, isotope and prodrug thereof according to item 1 or item 2, wherein U is selected from: 1,2-ethylidene, 1,3-propylidene, 1,4-butylidene, 1,5-pentylidene, 1,6-hexylidene, 1,3-cyclopentylene, 1,3-cyclohexylidene, 1,4-cyclohexylidene, 1,2-phenylene, 1, 3-phenylene, 1,4-phenylene, 2,5-pyridinylene, 2,5-pyrimidinone, 2,5-thiazolylene or 2,4-oxazolylene, wherein 1,2-ethylidene, 1,3-propylidene, 1,4-butylidene, 1,3-cyclopentylene, 1,3-cyclohexylidene, 1,4-cyclohexylidene, 1,2-phenylene, 1,3-phenylene, 1,4-phenylene, 2,5-pyridinylene, 2,5-pyrimidylene, 2,5-thiazolylene or 2,4-oxazolylene is optionally substituted with one or more R₉.

Item 32. The compounds and pharmaceutically acceptable salt, solvent compound, stereoisomer, isotope and prodrug thereof according to item 30 or item 31, wherein

R₉ is selected from: hydrogen, Rₙ, halogen, C₁-C₆ alkyl, C₁-C₆ haloalkyl, C₁-C₆ alkoxyl.

R₁₃ is selected from: hydroxyl, Rₙ, —ORₙ, —OC(═O)Rₙ, —NRₘRₙ, —NRₘ—C(═O)Ri, —NR—C(═O)Rₘ, —NRₘ˜S(═O)₂Rₙ, —NRₙ—S(═O)₂Rₘ; R is selected from: C₁-C₈ alkyl substituted with hydroxyl; Preferably, Rₙ is selected from: C₁-C₄ alkyl substituted with hydroxyl; More preferably, Rₙ is selected from: —CH₂OH, —CH₂CH₂OH, —CH₂CH₂CH₂OH, or —CH₂CH₂CH₂CH₂OH.

Rₘ is selected from: hydrogen, —C₁-C₄ alkyl.

Item 33. The compounds and pharmaceutically acceptable salt, solvent compound, stereoisomer, isotope and prodrug thereof according to item 30 or item 31, wherein
R₉ is selected from: hydrogen, amino, hydroxyl, sulfhydryl, —R₂₁N(R₂₂)R₂₂, —R₂₁C(═O)R₂₂, Rₙ, halogen, C₁-C₆ alkyl, C₁-C₆ haloalkyl, C₁-C₆ alkoxyl;
R₁₃ is selected from: hydroxyl, amino, sulfhydryl, R₁, —ORₙ, —OC(═O)Rₙ, —NRₘ,Rₙ, —NRₙ—C(═O)Rₙ, —NRₙ—C(═O)Rₘ, —NRₘ—S(═O)₂Rₙ, —NRₙ—S(═O)₂Rₘ RC is selected from: C₁-C₈ alkyl substituted with hydroxyl, sulfhydryl or amino; Preferably,
Rₙ is selected from: C₁-C₄ alkyl substituted with hydroxyl, sulfhydryl or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂SH, —CH₂CH₂CH₂SH, —CH₂CH₂CH₂CH₂SH. Rₘ is selected from: hydrogen, —C₁-C₄ alkyl.

Item 34. The compounds and pharmaceutically acceptable salt, solvent compound, stereoisomer, isotope and prodrug thereof according to item 32 or item 33, wherein R₉ is selected from: F, Cl, Br, cyano, methyl, ethyl, trifluoromethyl, methoxyl, ethoxyl, hydroxyl, CH₂OH, —CH₂CH, —CH₂CH₂CH₂OH, —OCH₂OH, —OCH₂CH₂, —OCH₂CH₂CH₂OH, —NHCH₂OH, —NHCH₂CH₂OH, —NHCH₂CH₂CH₂OH, —N(CH₃)CH₂OH, —N(CH₃)CH₂CH₂OH, —N(CH₃)CH₂CH₂CH₂OH, —N(COCH₃)CH₂OH, —N(COCH₃)CH₂CH₂OH, —N(COC₃)CH₂CH₂CH₂OH, —N(SO₂CH₃)CH₂₀₁, —N(SO₂CH₃)CH₂CH₂OH, —N(SO₂CH₃)CHCH₂CCH₂OH.

Item 35. The compounds and pharmaceutically acceptable salt, solvent compound, stereoisomer, isotope and prodrug thereof according to item 32 or item 33, wherein R₉ is selected from: F, Cl, Br, cyano, methyl, ethyl, trifluoromethyl, methoxyl, ethoxyl, hydroxyl, thiol, amino, —COCH₃, COCH₂CH₃, COCH₂CH₂CH₃, —CH₂CH₂N(CH₃)₂, —CH₂OH, —CH₂CH₂OH, —CH₂CH₂, —C—CH₂, —CH₂CH₂CH₂NH₂, —CH₂SIH, —CH₂CH₂SIH, —CH₂CH₂CH₂SH, —CH₂CH₂CH₂CH₂SH, —OCH₂OH, —OCH₂CH₂OH, —OCH₂CH₂CH₂OH, —OCH₂NH₂, —OCH₂CH₂NH₂, —OCH₂CH₂CH₂NH₂, —OCH₂SIH, —OCH₂CH₂SH, —OCH₂CH₂CH₂SH, —NHCH₂OH, —NHCH₂CH₂OH, —NHCH₂CH₂CH₂OH, —NHCH₂NH₂, —NHCH₂CH₂NH₂, —NHCH₂O₂CH₂NH₂, NHCH₂SH, —NHCH₂CH₂SH, —NHCH₂CH₂CH₂SH, —N(CH₃)CH₂OH, —N(CH₃)CH₂CH₂OH, —N(CH₃)CH₂CH₂CH₂OH, —N(CH₃)CH₂NH₂, —N(CH₃)CH₂CH₂NH₂, —N(CH₃)CH₂CH₂CH₂NH₂, —N(CH₃)CH₂SH, —N(CH₃)CH₂CH₂SH, —N(CH₃)CH₂CH₂CH₂SH, —N(COCH₃)CH₂OH, —N(COCH₃)CH₂CH₂OH, —N(COCH₃)CH₂CH₂CH₂OH, —N(COCH₃)CH₂NH, —N(COCH₃)CH₂C:2NH₂, —N(COCH₃)CH₂—₂CH₂N₂, —N(COCH)CH₂SH, —N(COCH)CH₂CH₂SH, —N(COCH₃)CH₂CH₂CH₂SH—N(SO₂CH₃)CH₂OH, —N(SO₂CH₃)CH₂CH₂, —N(SO₂CH₃)CH₂CH₂CH₂OH, —N(SO₂CH₃)CH₂NH₂, —N(SO₂CH₃)CH₂CH₂NH₂, —N(SO₂CH₃)CH₂CH₂CH₂NH₂, —N(SO₂CH₃)CH₂SH, —N(SO₂CH₃)CH₂CH₂SH, —N(SO₂CH₃)CH₂—CH₂CH₂SH.

Item 36. The compounds and pharmaceutically acceptable salt, solvent compound, stereoisomer, isotope and prodrug thereof according to item 1 or item 2, wherein U is selected from the following groups:

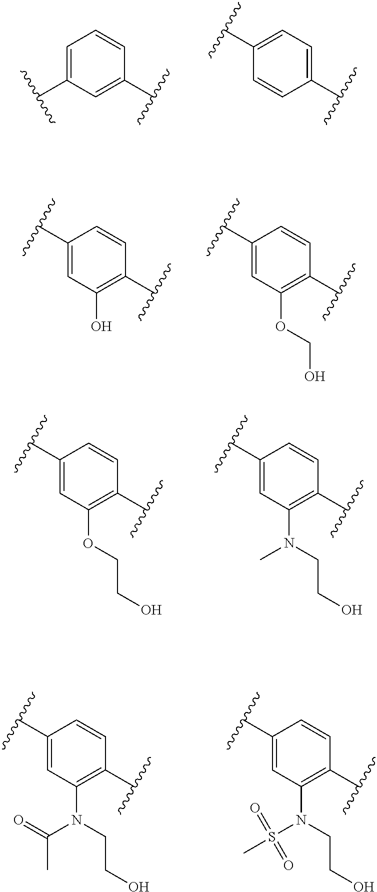

Item 37. The compounds and pharmaceutically acceptable salt, solvent compound, stereoisomer, isotope and prodrug thereof according to item 1 or item 2, wherein -T-UJ-Z-together forms a group selected from:

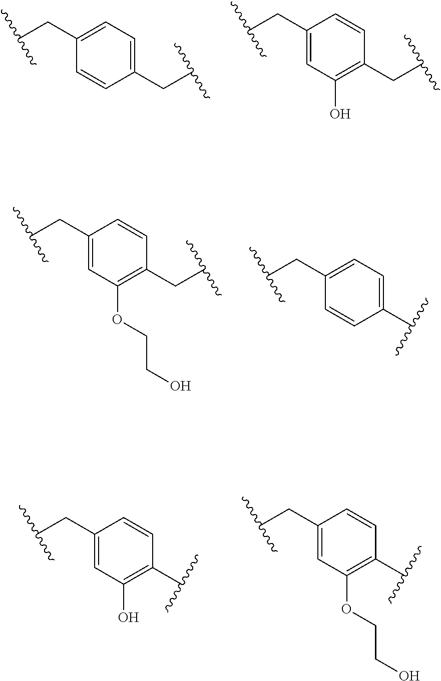 [SMILES: *Cc1ccc(C*)cc1 ; *Cc1ccc(C*)c(O)c1 ; *Cc1ccc(C*)c(OCCO)c1 ; *Cc1ccc(*)cc1 ; *Cc1ccc(*)c(O)c1 ; *Cc1ccc(*)c(OCCO)c1]

Item 38. The compounds and pharmaceutically acceptable salt, solvent compound, stereoisomer, isotope and prodrug thereof according to item 1 or item 2, wherein Y is selected from: chemical bond, —NR₁₄C(═O)—, —C(═O)NR₁—, —C(═O)NR₁₄CH₂—, —C(═O)NHCHR₉—, —NR₁₄C(═O)CH₂—, —NHC(═O)CHR₉—, —OCHR₉—, —OCH₂13 NR, —NR₂CH₂—, —NHCHR₉—;
wherein R₉ is selected from: hydrogen, C₁-C₄ alkyl, R₁₃;
wherein R₁₃ is selected from: hydroxyl, Rₙ, —ORₙ.
wherein R₁₄ is selected rom: Rₙ, —C(═O)R.;
wherein R₂ is selected from: hydrogen, R₁, C₁-C₈ alkyl, C₃-C₁₀ cycloalkyl, aryl, —C(═O)C₃-C₁₀ cycloalkyl, —C(═O)C₁-C₈ alkyl or —S(═O)₂C₁-C₈ alkyl, wherein C₁-Cₐ alkyl, C₃-C₁₀ cycloalkyl, aryl and the cycloalkyl or alkyl portion of —C(═O)C₃-Co cycloalkyl, —C(═O)C₁-C₈ alkyl or —S(═O)₂C₁-C₈ alkyl is optionally substituted with one or more R₉, wherein R₉ is selected from: hydrogen, halogen, amino, cyano, carboxyl, C₁-C₆ alkyl, C₁-C₆ alkoxyl or aryl; wherein R₁₄ is selected from: R₁, —C(═O)Rₙ, —C(═O)ORₙ, —C(═O)NRₘRₙ, —S(═O)₂R, —S(═O)₂NRₘR;

wherein Rₙ is selected from: C₁-C₈ alkyl substituted with hydroxyl; Preferably, Rₙ is selected from: C₁-C₄ alkyl substituted with hydroxyl; More preferably, Rₙ, is selected from: —CH₂OH, —CH₂ CH₂OH, —CH₂CH₂CH₂OH, or —CH₂CH₂CH₂CH₂OHI.

Rₘ, is selected from: hydrogen, —C₁-C₄ alkyl.

Item 39. The compounds and pharmaceutically acceptable salt, solvent compound, stereoisomer, isotope and prodrug thereof according to item 1 or item 2, wherein Y is selected from: chemical bond, —NR₄C(═O)—, —C(═O)NR₁₄—, —C(═O)NR₁₄CH, —C(═O)NHCHR₉—, —NR₁₄C(═O)CH—, —NHC(═O)CHR, —O—, —OCHR₉—, —OCHR₉CH₂r, —OCH₂CHR₉—, —NR, —NR₂CH₂—, —NHCHR₉—;
wherein R₉ is selected from: hydrogen, C₁-C₄ alkyl, —R₂₂N(R₂₂)R(ss, —R₂₁C(═O)R₂₂, R₁₃;
wherein R₁₃ is selected from: hydroxyl, sulfhydryl, amino, Rₙ, —OR;
wherein R₁₄ is selected from: Rₙ, —C(═O)Rₙ;
wherein R₂ is selected from: hydrogen, R₄, C₁-C₈ alkyl, C₁-C₁₀ cycloalkyl, aryl, —C(═O)C₃CH₂ cycloalkyl, —C(═O)C Cs alkyl or —S(═O)₂alkyl, wherein C₁-C₈ alkyl, cycloalkyl, aryl and the cycloalkyl or alkyl portion of —C(═O)C₁₀ cycloalkyl, —C(═O)C₁-C₈ alkyl or —S(═O)₂C₁-C₈ alkyl is optionally substituted with one or more R₉, wherein R₉ is selected from: hydrogen, halogen, amino, cyano, carboxyl, C₁-C₆ alkyl, C₁-C₆ alkoxyl or aryl; wherein R₁₄ is selected from: Rₙ, —C(═O)Rₙ, —C(═O)ORₙ, —C(═O)NRₘRₙ, —S(═O)₂Rₙ, —S(═O)₂NRₘRₙ.

wherein Rₙ is selected from: C₁-C₈ alkyl substituted with hydroxyl, sulflhydryl or amino; Preferably, the Rₙ is selected from: C₁-C₄ alkyl substituted with hydroxyl, sulfhydryl or amino; More preferably, Rₙ is selected from: —CH₂OH, —CH₂CH₂OH, —CH₂CH 2CH₂OH, —CH₂CH₂CH₂CH₂OH, —CH₂NH₂, —CH₂CH₂NH₂, —CH₂CH₂N₂I, —CH₂CH₂CH₂CH₂NH₂, —CH₂SH, —CH₂CH₂SH, —CH₂CH₂CH₂SH, —CH₂CH₂CH₂CH₂SH;

Rₘ is selected from: hydrogen, —C₁-C₄ alkyl.

Item 40. The compounds and pharmaceutically acceptable salt, solvent compound, stereoisomer, isotope and prodrug thereof according to item 38 or item 39, wherein R₂ is selected from hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenylethyl, 1-naphthylmethyl, 2-naphthylethyl, —CH₂OH. —CH₂CH₂OH, —CH₂CH₂CH₂OH, —C(═O)CH₂OH, —C(═O)CH₂CH₂OH, —C(═O)CH₂CH₂CH₂OH, —C(═O)OCH₂O H, —C(═O)OCH₂CH₂OH, —C(═O)OCH₂CH₂CH₂OH, —C(═O)NHCH₂OH, —C(═O)NHCH₂CH₂OH, —C(═O)NHCH₂CH₂CH₂OH, —S(═O)₂CH₂OH, —S(═O)₂CH₂CH₂OH, —S(═O)₂CH₂CH₂ —S(═O)₂NHCH₂OH, —S(═O)₂NHCH₂CH₂OH, —S(═O)₂N HCH₂CH₂CH₂OH.

Item 41. The compounds and pharmaceutically acceptable salt, solvent compound, stereoisomer, isotope and prodrug thereof according to item 38 or item 39, wherein R₂ is further selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthylethyl, —CH₂OH, —CH₂CH₂OH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —C(═O)CH₂OH, —C(═O)CH₂CH₂OH, —C(═O)CH₂CH₂C₂Off, —C(═O)CH₂NH₂, —C(═O)CH₂CH₂NH₂, —C(═O)CH₂CH₂CH₂NH₂, —C(═O)OCH₂OH, —C(═O)OCH₂CH₂OH, —C(═O)OCH₂CH₂CH₂OH, —C(═O)OCH₂NH₂, —C(═O)OCH₂CH₂NH₂, —C(:O)OCH₂CH₂CH₂NH₂, —C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂CH₂CH₂OH, —C(═O)NH, —C(═O)NHCH₂CH₂NH₂, —C(═O)NHCH₂CH₂CH₂NH₂, —C(═O)NHCH₂SH, —C(═O)NHCH₂CH₂SH, —C(═O)NHCH₂ CH₂CH₂SH, —S(═O)₂CH₂OH, —S(═O)₂CH₂CH₂OH, —S(═O)₂CH₂CH₂CH₂ OH, —S(═O)₂CH₂NH₂, —S(═O)₂CH₂ CH₂NH₂, —S(═O)₂CH₂ CH₂CH₂NH₂, —S(═O)₂CH₂SH-, —S(═O)₂CH₂CH₂SH, —S(═O)₂CH₂CH₂CH₂SH, —S(═O)₂NHCH₂OH, —S(═O)₂NHCH₂CH₂OH, —S(═O)₂NHCH₂CH₂CH₂OH, —S(═O)₂NHCH₂NH₂y, —S(═O)₂NHCH₂CH₂NH, —S(═O)₂NHCH₂CH₂CH₂NH₂, —S(═O)₂NHCH₂SH, —S(═O)₂NHCH₂CH₂SH, —S(═O)₂NHCH₂CH₂CH₂SH.

Item 42. The compounds and pharmaceutically acceptable salt, solvent compound, stereoisomer, isotope and prodrug thereof according to item 1 or item 2, wherein L is selected from: —CH₂—, —CH₂CH₂—, C(═O)—, —C(OH)—.

Item 43. The compounds and pharmaceutically acceptable salt, solvent compound, stereoisomer, isotope and prodrug thereof according to item 1 or item 2, wherein R₃—R is independently selected from: hydrogen, R₁₃, halogen, cyano, amino, C₁-C₆ alkyl, C₁-C₆ alkoxy, C₁-C₆ alkylthio, C₁-C₆ alkylamino, di(C₁-C₆ alkyl)amino, C₃-C₈ cycloalkyl, wherein the alkyl, alkoxy, alkylamino, alkylthio, or cycloalkyl is optionally substituted with 1-3 groups each selected from halogen, C₁-C₃ alkyl or C₁-C₆ alkoxy; when there are multiple R₃-Rs, any two adjacent ones may be combined to form a ring.

Item 44. The compounds and pharmaceutically acceptable salt, solvent compound, stereoisomer, isotope and prodrug thereof according to item 1 or item 2, wherein R₆ is selected from: hydrogen, C₁-C₆ alkyl, C₃-C₈ cycloalkyl, wherein alkyl or cycloalkyl is optionally 20 substituted with 1-3 groups each selected from halogen, hydroxyl, cyano, amino, —C(═O)OR₂₃, —OC(═O)R₂₃, —NHC(═O)R₂₃, —C(═O)NHR₂₃ or —OP(═O)(OM)₂; Preferably, R₆ is selected from: hydrogen, C₁-C₄ alkyl, C₁-C₄ haloalkyl, —C₁-C₄ alkyleneOC(═O)R₂₃, —C₁-C₄ alkylene C(═O)OR₂₃, —C₁-C₄ alkyleneOP(═O)(OH)₂; wherein R₂₃ is selected from: hydrogen, C₁-C₄ alkyl, and the C₁-C₄ alkyl is optionally substituted with 1-3 groups selected from hydroxyl and amino, respectively.

Item 45. The compounds and pharmaceutically acceptable salt, solvent compound, stereoisomer, isotope and prodrug thereof according to item 1 or item 2, wherein, the compound is a compound represented by formula (I-1) or formula (1-2):

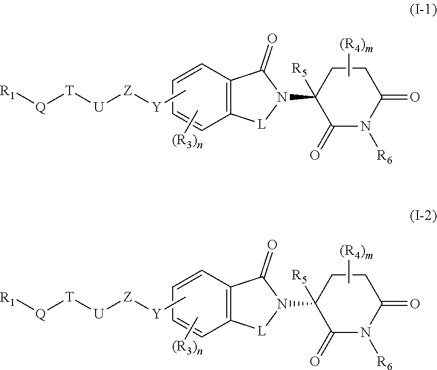

Wherein, R₁, R₃—R₆, Q, T, U, Z, Y, L, ii, m are defined as above.

Item 46. The compounds and pharmaceutically acceptable salt, solvent compound, stereoisomer, isotope and prodrug thereof according to item 1 or item 2, wherein, the compound is a compound represented by formula (H) or (III):

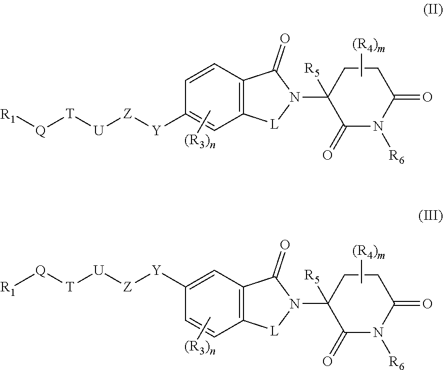

Wherein R₁, R₃-R₆, Q, T, U, Z, Y, L, n, m are defined as above.

Item 47. The compounds and pharmaceutically acceptable salt, solvent compound, stereoisomer, isotope and prodrug thereof according to item 1 or item 2, wherein, the compound is a compound represented by formula (IV) or Formula (V):

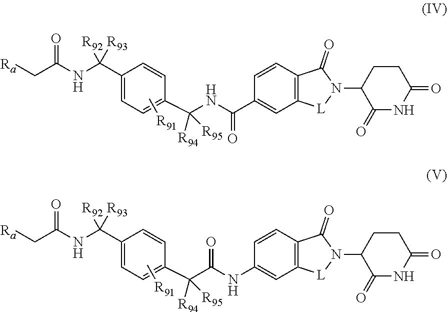

Wherein NH of CH₂C(═O)NH—, —C(R₉₄)(R₉₅)NHC(═O)—, —C(R₉₄)(R₉₅)C(═O)NH—, is optionally substituted with R₁₄; 5Rₐ, R_{b4} and L are defined as above.

R₉₁-R₉₅ is independently selected from: hydrogen, R₁₃, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R_{2I}OC(═O)R₂₂, —R₂₁S(═O)₂R₂₂, —R₂₁S(═O)₂NHR₂₂, C₁-C₆ alkyl, C₁-C₆ alkoxyl, C₁-C₆ alkylthio, C₁-C₆ alkylamino, di(C₁-C₆)alkyl)amino, C₃-C₈ cycloalkyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, cyano group, C₁-C₃ alkyl or C₁-C₃ alkoxyl; Any two adjacent ones in R₉₂R₉₄ may be combined to form a ring.

Item 48. The compounds and pharmaceutically acceptable salt, solvent compound, stereoisomer, isotope and prodrug thereof according to item 47, wherein R₉₁ is selected from: hydrogen, R₂₃, halogen, cyano, amino, C₁-C₆ alkyl, C₁-C₆ alkoxyl, C₁-C₆ alkylthio, C₁-C₆ alkylamino, di(C₁-C₆ alkyl)amino, C₃-C₈ cycloalkyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with 1-3 groups selected from halogen, cyano group, C₁-C₃ alkyl or C₁-C₃ alkoxyl; Preferably, R₉₁ is selected from: hydrogen, halogen, C₁-C₆ alkyl, C₁-C₆ haloalkyl, C₁-C₆ alkoxyl, hydroxyl, R₁, —ORₙ, —OC(═O)Rₙ, —NRₘRₙ, —NRₘ—C(═O)Rₙ, —NRₙ,—CO(═O)Rₘ, —NRₙ—S(═O)₂Rₙ, —NRₙ—S(═O)₂Rₘ; More preferably, is selected from: hydrogen, halogen, C₁-C₆ alkyl, C₁-C₆ haloalkyl, C₁-C₆ alkoxyl, hydroxyl, Rₐ, —ORₙ, —NR—C(═O)Rₘ, —NRₙ—S(═O)₂Rₘ; Wherein R₉₂1R₉₄ is independently selected from: hydrogen, R₁₃, C₁-C₆ alkyl, or any two adjacent groups in R₉₂-R₉₄ can be combined to form a cycloalkyl group; Preferably, R₉₂—R₉₄ is independently selected from: hydrogen, methyl, ethyl, hydroxyl, Rₙ, —ORₙ, or R₉₂ and R₉₃ together form —CH₂CH₂—, or R₉₄ and R₉₅ together form —CH₂CH₂—.

Item 49. The compounds and pharmaceutically acceptable salt, solvent compound, stereoisomer, isotope and prodrug thereof according to item 47 or item 48, wherein R₉₁ is selected from R₁₃, or one of R₉₁-R₉₅ is selected from R₁₃.

Item 50. The compounds and pharmaceutically acceptable salt, solvent compound, stereoisomer, isotope and prodrug thereof according to item 1 or item 2, wherein the compound is a compound represented by formula (IV′) or formula (V′):

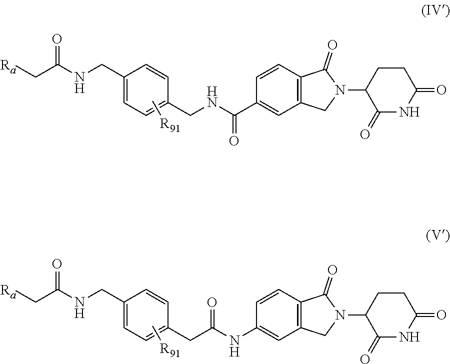

Wherein, Rₐ and R)1 are defined as above; Preferably, R₉₁ is selected from: hydroxyl, Rₙ, —ORₙ, —NRₘRₙ, —NRₐC(═O)Rₘ, —NRₙ,—S(═O)₂Rₘ.

Item 51. The compounds and pharmaceutically acceptable salt, solvent compound, stereoisomer, isotope and prodrug thereof according to item 1 or item 2, wherein the compound is a compound represented by formula (VI) or formula (VII):

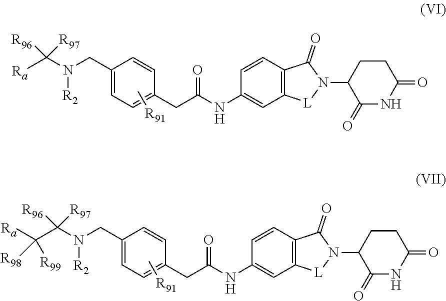

Wherein each NH of —CH₂C(═O)NH— is substituted with R₁₄;

Rₙ, R₂, R₉₁, R₁₄, L are defined as above;

R₉₆-R₉₉ is independently selected from: hydrogen, R₁₃, halogen, cyano, amino, nitro, —R₂₁C(═O)R₂₂, —R₂₁NHC(═O)R₂₂, —R₂₁C(═O)NHR₂₂, —R₂₁C(═O)OR₂₂, ˜R₂₁₀(═O)R₂, —(═O)₂R₂₂, —R₂₁S(═O)₂NHR₂₂, C_{1-C} alkyl, C₁=_{C} alkoxyl, C₁-C alkylthio, C₁-C alkylamino, di(C₁-C₆ alkyl)amino, C₃—Cs cycloalkyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, cyano group, C₁-C₃ alkyl or C₁-C₆ alkoxyl; Any two adjacent ones in R₉₆-R₉₉ may be combined to form a ring.

Preferably, R₉₆-R₉₉ is independently selected from: hydrogen, Rₙ, C—C, alkyl group, or any two adjacent groups in R₉₆₃-R₉₉ can be combined to form a cycloalkyl group; More preferably, R₉₆—R₉₉ is independently selected from: hydrogen, methyl, ethyl, hydroxyl, Rₙ, —ORₙ, or R₉₆ and R₉₇ together form —CH₂CH₂—, or R)ₛ and R₉₉ together form —CH₂CH₂—, respectively.

Item 52. The compounds and pharmaceutically acceptable salt, solvent compound, stereoisomer, isotope and prodrug thereof according to item 51, wherein R₂ is selected from: hydrogen, R₁₄, C₁-C₈ alkyl, C₃-C₁₀ cycloalkyl, —C(═O)C₃-C₁₀ cycloalkyl, —C(═O)C₁-C₈ alkyl or —S(═O)₂C₁-C₈alkyl, wherein Ct-C₈ alkyl, C₁-C₃₀ cycloalkyl and the cycloalkyl or alkyl portion of —C(═O)C₃-C-1 cycloalkyl, —C(═O)alkyl or —S(═O)₂C₁-C₈ alkyl is optionally substituted with one or more R₉, wherein R₉ is selected from: hydrogen, halogen, amino, cyano, carboxyl, C₁-C₆ alkyl, C₁-C₆ alkoxyl or aryl; Wherein R₁₄ is selected from: R, —C(═O)Rₙ, —C(═O)ORₙ, —C(═O)NRₘRₙ, —S(═O)₂Rₐ, Wherein Rₙ is selected from: C₁-C₈ alkyl substituted with hydroxyl; Preferably, Rₙ is selected from: C₁-C₄ alkyl substituted with hydroxyl; More preferably, Rₙ is selected from: —CH₂OH, —CH₂CH₂OH, —CH₂CH₂CH₂OH, —CH₂CH₂CH₂CH₂OH; Wherein Rₘ is selected from: hydrogen, —C₁-C₄ alkyl.

Item 53. The compounds and pharmaceutically acceptable salt, solvent compound, stereoisomer, isotope and prodrug thereof according to item 51, wherein R₂ is selected from: hydrogen, R₁₄ C₁-C₈ alkyl, C₃-C₁₀ cycloalkyl, —C(═O)C₃-C₁₀ cycloalkyl, —C(═O)C₁-C₈ alkyl or —S(═O)₂C₁-C₈ alkyl, wherein C₁-C₈ alkyl, C₃-C₁₀ cycloalkyl and the cycloalkyl or alkyl portion of —C(═O)C₃-C₁₀ cycloalkyl, —C(═O)C_{1I}C₈ alkyl or —S(═O)₂C₁-C₈ alkyl is optionally substituted with one or more R₉, wherein R₉ is selected from: hydrogen, halogen, amino, cyano, carboxyl, C₁-C₆ alkyl, C₁-C₆ alkoxyl or aryl;

Wherein R₁₄ is selected from: Rₙ, —C(═O)Rₙ, —C(═O)ORₙ, —C(═O)NRₘRₙ, —S(═O)₂Rₙ, —S(═O)₂NRₘRₙ.;

Wherein Rₙ is selected from: C₁-C₈ alkyl substituted with hydroxyl, sulfhydryl or amino; Preferably, Rₙ is selected from: C₁-C₄ alkyl substituted with hydroxyl, sulfhydryl or amino; More preferably, Rₙ is selected from: —CHOH, —CH₂CH₂, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —, —CH₂CH₂SH, —CH₂CH₂CH₂SH, —CH₂CH₂CH₂CH₂SH;

Wherein Rₘ is selected from: hydrogen, —C₁-C₄ alkyl.

Item 54. The compounds and pharmaceutically acceptable salt, solvent compound, stereoisomer, isotope and prodrug thereof according to item 52 or item 53, wherein R₂ is selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthylmethyl, 2-naphthylethyl, —CH₂OH, —CH₂CH₂OH, —CH₂CH₂CH₂OH, —C(═O)CH₂OH, —C(═O)CH₂CH₂OH, —C(═O)CH₂CH₂CH₂OH, —C(═O)OCH₂OH, —C(═O)OCH₂CH₂OH, —C(═O)OCH₂CH₂CH₂OH, —C(═O)NHCH₂OH, —C(═O)NHCH₂CH₂OH, —C(═O)NHCH₂CH₂CH₂OH, —S(═O)₂CH₂OH, —S(═O)₂CH₂CH₂OH, —S(═O)₂CH₂CH₂CH₂OH. —S(═O)₂NHCH₂OH, —S(═O)₂NCH₂CH₂Of, —S(═O)₂NHCH₂CH₂Preferably, R₂ is selected from: hydrogen, methyl, ethyl, isopropyl, cyclopropyl, benzyl, —H₂O, —CH₂CH₂OH, —CH₂CH₂, —C(═O)CH₂, —C(═O)CH₂CH₂OH, —C(═O)CH₂CH₂, —C(═O)OCH₂OH, —C(═O)OCH₂CH₂OH, —C(═O)OCH₂CH₂CH₂OH, —C(═O)NHCH₂OH, —C(═O)NHCH₂CH₂OH, —C(═O)NHCH₂CH₂CH₂OH.

Item 55. The compounds and pharmaceutically acceptable salt, solvent compound, stereoisomer, isotope and prodrug thereof according to item 52 or item 53, R₂ is selected from: hydrogen, methyl, ethyl, —SO₂CHs, —COCH₃, —CO-isopropyl, —CO-cyclopropyl, isopropyl, cyclopropyl, 2-methoxyethyl, 2-cyanoethyl, phenyl, naphthyl, benzyl, 2-phenyl ethyl, t-naphthyl methyl, 2-naphthylethyl, —CH₂OH, —CH₂CH₂O-1, —CH₂)CH₂E, —CH₂NH₂, —CH₂C₂NH₂, —CH₂CH₂CH₂NH₂, —CH₂SH, —CH₂CH₂SH, —CH₂CH₂CH₂SH, —CH₂CH₂CH₂CH₂SH, —C(═O)CH₂OH, —C(═O)CHCH₂OH, —C(═O)CH₂CH₂, —C(═O)CH₂, —C(═O)CH₂CH₂NH₂, —C(═O)CH₂CH₂CH₂NH₂, —C(═O)OCH₂OH, —C(═O)OCH₂CH₂OH, —C(═O)OCH₂CH₂CH₂OH, —C(═O)OCH₂NH₂, —C(═O)OCH₂CH₂,—C(═O)OCH₂CH₂NH, °—C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂CH₂CH₂OH, —C(═O)NHCH₂NH₂, —C(═O)NHCH₂CH₂NH₂, —C(═O)NHCH₂CH₂CH₂NH₂, —C(═O)NHCH₂SH, —C(═O)NHCH₂CH₂SH, —C(═O)NHCH₂CH₂CH₂SH, —S(═O)₂CH₂₂₀I, —S(═O)₂CH₂, —S(═O)₂CH₂CH₂CH₂OH, —S(═O)CH₂NH₂, —S(═O)₂CH₂CH₂NH₂, —S(═O)₂CH₂CH₂CH₂NH₂, —S(═O)₂CH₂SH, —S(═O)₂CH₂CH₂SH, —S(═O)₂CH₂CH₂C₂S, —S(═O)₂NHCHOH, —S(═O)₂NHCH₂CH₂₉OH, —S(═O)₂NHCH₂CH₂CH₂OH, —S(═O)₂NHCH₂NH₂, —S(═O)₂NHCH₂CH₂NH₂, —S(═O)₂NHCH₂CH₂CH₂NH₂, —S(═O)₂NHCH₂SH, —S(═O)₂NH₂SH, —S(═O)₂NHCH₂CH₂CH₂SH.

Item 56. The compounds and pharmaceutically acceptable salt, solvent compound, stereoisomer, isotope and prodrug thereof according to item 51 to item 55, wherein R₂ is selected from R₁₄, or R₉₃ is selected from R₁₃, or one of the R₉₆-R₉₉ is selected from R₁₃.

Item 57. The compounds and pharmaceutically acceptable salt, solvent compound, stereoisomer, isotope and prodrug thereof according to item 1 to item 2, wherein the compound is a compound represented by formula (VI) or (VII′):

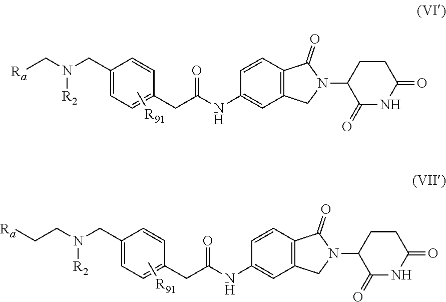

wherein, Rₐ, R₂, R₉₁ are defined as above.

wherein R₂ is selected from: Rₙ, —C(═O)Rₙ, —C(═O)ORₙ, —C(═O)NRₘRₙ.

wherein R₁₁ is selected from: hydroxyl, Rₙ, —ORₙ, —NRₘRₙ, —NRₙ—C(═O), —NRₙ—S(═O)Rₘ.

Item 58. The compounds and pharmaceutically acceptable salt, solvent compound, stereoisomer, isotope and prodrug thereof according to item 1 to item 2, wherein the compound is a compound represented by formula (VII):

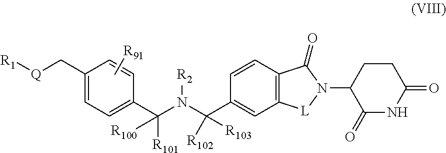

Wherein, R₁, Q, R₂, R₉₁, L are defined as above; R₁₀₀-R₁₀₃ are selected independently from: hydrogen, R₁₃, halogen, cyano, amino, nitro, —R₂₁C(═O)R₂₂, —R₂₁(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxyl, C₁-C₆ alkylthio, Ct-C₆ alkylamino, di(C₁-C₆)alkylamino, C₃-C₈ cycloalkyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, cyano, C₁-C₃ alkyl or C₁-C₃ alkoxyl; Any two adjacent ones in R₉₆-R₉₉ may be combined to form a ring.

Preferably, wherein R₁₀₀-R₁₀₃ is independently selected from: hydrogen, R₁₃, C₁-C₆ alkyl group, or any two adjacent groups in R₁₀₀-R₁₀₃ can be combined to form a cycloalkyl group: More preferably, R₁₀₀ is independently selected from: hydrogen, methyl, ethyl, hydroxyl, Rₙ, —ORₙ, or R₁₀₀ and R₁₀₃ together form —CH₂CH₂—, or R₁₀₀ and R₁₀₃ together form —CH₂CH₂—, respectively.

Item 59. The compounds and pharmaceutically acceptable salt, solvent compound, stereoisomer; isotope and prodrug thereof according to item 58, wherein R₂ is selected from: hydrogen, R₁₄, C₁-C₈ alkyl, C₃-C₁₀ cycloalkyl, —C(═O)C₃-C₁₀ cycloalkyl, —C(═O)C—Cs alkyl or —S(═O)₂C₁-C₈ alkyl, wherein C₁-C₈ alkyl, C₃-C₁₀ cycloalkyl and the cycloalkyl or alkyl portion of —C(═O)C₃-C₁₀ cycloalkyl, —C(═O)C₁-C₈ alkyl or —S(═O)₂C₁-C₈ alkyl is optionally substituted with one or more R₉, wherein R₉ is selected from: hydrogen, halogen, amino, cyano, carboxyl, C₁-C₆ alkyl, C₁-C₆ alkoxyl or aryl;

Wherein R₁₄ is selected from: Rₙ, —C(═O)Rₙ, —C(═O)ORₙ, —C(═O)NRₘRₙ, —S(═O)₂Rₙ, —S(═O)₂NRₘRₙ;

Wherein Rₙ is selected from: C₁-C₈ alkyl substituted with hydroxyl; Preferably, Rₙ is selected from: C₁-C₄ alkyl substituted with hydroxyl; More preferably, Rₙis selected from: —CH₂OH, —CH₂ CH₂OH, —CH₂CH₂CH₂OH, —CH₂CH₂CH₂CH₂OHI;

Wherein Rₘ is selected from: hydrogen, —CH₄ alkyl.

Item 60. The compounds and pharmaceutically acceptable salt, solvent compound, stereoisomer, isotope and prodrug thereof according to item 58, wherein R₂ is selected from: hydrogen, R₁₄, C₁-C₈ alkyl, C₃-C₁₀ cycloalkyl, —C(═O)C₃-C₁₀ cycloalkyl, —C(═O)C₁-C₈ alkyl or —S(═O)₂C₁-C₈ alkyl, wherein C₁-C₈ alkyl, C₃-C₁₀ cycloalkyl and the cycloalkyl or alkyl portion of —C(═O)C₃-C₁₀ cycloalkyl, —C(═O)C—Cs alkyl or —S(═O)₂alkyl is optionally substituted with one or more R₉, wherein R₉ is selected from: hydrogen, halogen, amino, cyano. carboxyl, C₁-C₆ alkyl, C₁-C₆ alkoxyl or aryl;

Wherein R₂₄ is selected from: Rₙ, —C(═O)Rₙ, —C(═O)ORₙ, —C(═O)NRₘRₙ, —S(═O)₂Rₙ, —S(═O)₂NRₘRₙ;

Wherein Rₙ is selected from: C₁-C₈ alky substituted with hydroxyl, sulfhydryl or amino; Preferably, Rₙ is selected from: C₁-C₄ alkyl substituted with hydroxyl, sulfhydryl or amino; More preferably, R₁ is selected from: —CH₂OH, —CH₂CH₂OH, —CH₂CH₂CH₂OH, —CH₂CH₂CH₂CH₂ OH, —CH₂NH₂, —CH₂CH₂ NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH; Wherein Rₘ is selected from: hydrogen, —C₁-C₄ alkyl.

Item 61. The compounds and pharmaceutically acceptable salt, solvent compound, stereoisomer, isotope and prodrug thereof according to item 59 or item 60, wherein R₂ is selected from: hydrogen, methyl, ethyl, —SO₂CH₃, —CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthylmethyl, 2-naphthylethyl, —CH₂OH. —CH₂CH₂OH, —CH₂CH₂CH₂OH, —C(═O)CH₂OH, —C(═O)CH₂OH, —C(═O)CH₂CH₂CH₂OH, —C(═O)OCH₂OH, —C(═O)OCH₂CH₂OH, —C(═O)OCH₂CH₂CH₂OH, —C(═O)NHCH₂OH, —C(═O)NHCH₂CH₂OH, —C(═O)NHCH₂CH₂CH₂OH, —S(═O)₂CH₂OH, —S(═O)₂CH₂CH₂OH, —S(═O)₂CH₂CH₂CH₂OH, —S(═O)₂NHCHOH, —S(═O)₂NCH₂CH₂OH, —S(═O)₂NHCH₂. Preferably, R₂ is selected from: hydrogen, methyl, ethyl, isopropyl, cyclopropyl, benzyl, —CH₂OH, —CH₂CH₂OH, —CH₂CH₂CH₂, —C(═O)CH₂, —C(═O)CH₂CH₂, —C(═O)CHCH₂CH₂OH, °—C(═O)OCH₂OH, —C(═O)OCH₂CH₂OH, —C(═O)OCH₂CH₂CHOH. —C(═O)NHCH₂OH, —C(═O)HCH₂CH₂OH, —C(═O)NHCH₂CH₂CH₂OH.

Item 62. The compounds and pharmaceutically acceptable salt, solvent compound, stereoisomer, isotope and prodrug thereof according to item 59 or item 60, wherein R₂ is selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthyl ethyl, —CH₂OH, —CH₂CH₂OH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —C(═O)CH₂OH, —C(═O)CH₂CH₂OH, —C(═O)CH₂CH₂CH₂OH, —C(═O)CH₂NH₂, —C(═O)CH₂NH₂, —C(═O)CH₂CH₂CH₂NH₂, —C(═O)CH₂, —C(═O)CH₂CH₂SH, —C(═O)CH₂C₂CH₂SH, —C(═O)OCH₂OH, —C(═O)OCH₂CH₂OH, —C(O)CH₂CH₂CH, CH₂NH₂, —C(═O)OCH₂CH₂CH₂, —C(═O)OCH₂CH₂CH₂NH₂, —C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂CH₂CH₂OH, —C(═O)NHCH₂NH₂, —C(═O)NHCH₂CH₂NH₂, —C(═O)NHCH₂CH₂CH₂NH₂, —C(═O)NHCH₂SH, —C(═O)NHCH₂CH₂SH, —C(═O)NHCH₂CH₂CH₂SH, —S(═O)₂CH₂OH, —S(═O)₂CH₂CH₂, —S(═O)₂CH₂CH₂CH₂OH, —S(═O)₂CH₂NH₂, —S(═O)₂CH₂CH₂NH₂, —S(═O)₂CH₂CH₂CH₂NH₂, —S(═O)₂CH₂SH, —S(═O)₂CH₂SH, —S(═O)₂C₂CH₂CH₂SH, —S(═O)₂NHCH₂O OH, —S(═O)₂CH₂OH, —S(═O)₂NHCH₂CH₂CH₂OH, —S(═O)₂NHCH₂NH₂, —S(═O)₂NHCH₂CH₂NH₂, —S(═O)₂NHCH₂CH₂CH₂NH₂, —S(═O)₂NHCH₂SH, —S(═O)₂NHCH₂CH₂SH, —S(:═O)₂NHCH₂CH₂CH₂; Preferably R₂ is selected from hydrogen, methyl, ethyl, isopropyl, cyclopropyl, benzyl, —CH₂OH, —CH₂CH₂OH, —CH₂CH₂, —CH₂NH₂, —CH₂CH₂NH₂, —CH₂CH₂CH, NH₂, —CH₂SH, —CH₂CH₂SH, —CH₂CH₂CHSH, —CH₂CH₂CH₂CH₂SH, —C(═O)CH₂OH, —C(═O)CH₂CH₂OH, —C(═O)CH₂CCH₂OH, —C(═O)CH₂NH₂, C(═O)CH₂CH₂NH₂, —C(═O)CH₂CH₂CH₂NH, —C(═O)CH₂, —C-(═O)CH₂,CH₂, —C(═O)CH₂CH₂CH₂SH, —C(═O)OCH₂OHC(═O)OCH₂CH₂OH, —C(═O)OCH₂CH₂CH₂OH, —C(═O)OCH₂NH₂, —C(═O)OCH₂CH₂H₂, —C(═O)OCH₂CH₂CH₂NH₂, —C(═O)OCH₂SIH, —C(═O)OCH₂CH₂SH, —C(═O)OCH₂CH₂CH₂SH. C(═O)NHCH₂OH, —C(═O)NHCH₂CH₂OH, —C(═O)NH CH, CH₂CH₂OH, —C(═O)NHCH₂, C(═O)NHCH₂NH₂, —C(═O)NHCH₂CH₂CH₂NH₂, —C(═O)NHCH₂SH, —C(═O)NHCH₂CH₂SH, —C(═O)NHCH₂CH₂CH₂SH.

Item 63. The compounds and pharmaceutically acceptable salt, solvent compound, stereoisomer, isotope and prodrug thereof according to any one of items 58-62, wherein R₂ is selected from R₁₄, or R₉₁ is selected from R₁₃, or one of the R₁₀₀-R₀ is selected from R₁₃.

Item 64. The compounds and pharmaceutically acceptable salt, solvent compound, stereoisomer, isotope and prodrug thereof according to item 1 to item 2, wherein the compound is a compound represented by formula (VII′):

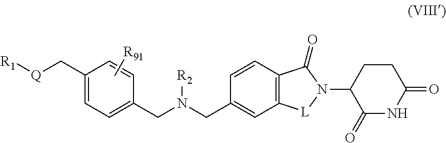

Wherein R₁, Q, R₂, R₉₁ are defined as above.

Preferably, R₂ is selected from: Rₙ, —C(═O)Rₙ, —C═O)ORₙ, —CO(═O)NRₘRₙ;

Preferably, R₉₁ is selected from: hydroxyl, Rₙ, —ORₙ, —NRₘRₙ, —NRₐC(═O)Rₘ, —NRₙ—S(═O)₂Rₘ.

Item 65. The compounds and pharmaceutically acceptable salt, solvent compound, stereoisomer, isotope and prodrug thereof according to item 1 to item 2, wherein the compound is a compound represented by formula (X):

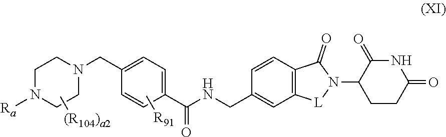

Wherein NH of —C(═O)NHCH₂— is optionally substituted with R₁₄;

Rₐ, R₁₄, R₉₁, a2 is defined as above;

R₁₀₄ is selected from: hydrogen, R₁₃, halogen, cyano, amino, nitro, —R₂₂C(═O)R₂₂, —R₂₂NHC(═O)R₂₂, —R₂₁C(═O)NHR₂₂, —R₂₁C(═O)OR₂₂, —Rₙ1OC(═O)Rₙ, —R₂₂S(═O)₂, —R(═O)₂NHR₂n, C₁-C₆ alkyl, C₁-C₆ alkoxyl, C₁-C₆ alkylthio, C₁-C₆ alkylamino, di(C₁-C₆alkyl)amino, C₃-C₈ cycloalkyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, cyano group, C₁-C₃ alkyl or C₁-C₃ alkoxyl;

Preferably, R₁₀₄ is selected from: hydrogen, R₁₃, halogen, C₁-C₆ alkyl, C₁-C₆ haloalkyl; More preferably, the R₁₀₄ is selected from: hydrogen, F, Cl, Br, methyl, ethyl, trifluoromethyl, hydroxyl, Rₙ, —ORₙ.

Item 66. The compounds and pharmaceutically acceptable salt, solvent compound, stereoisomer, isotope and prodrug thereof according to item 65, wherein Rₙ is selected from: C₁-C₈ alkyl substituted with hydroxyl; Preferably, Rₙ is selected from: C₁-C₄ alkyl substituted with hydroxyl; More preferably, Rₙ is selected from: —CH₂H, —CH₂CH₂OH, —CH₂CH₂CH₂, —CH₂CH₂CH₂CH₂OH.

Item 67. The compounds and pharmaceutically acceptable salt, solvent compound, stereoisomer, isotope and prodrug thereof according to item 65, wherein Rₙis selected from: C₁-C₈ alkyl substituted with hydroxyl, sulfhydryl or amino; Preferably, Rₙis selected from: C₁-C₄ alkyl substituted with hydroxyl, sulfhydryl or amino; More preferably, Rₙ is selected from: —C₂OH, —CH₂CH₂OH, —CH₂CH₂CH₂OH, —CH₂CH₂CH₂, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CCH₂CH₂SH.

Item 68. The compounds and pharmaceutically acceptable salt, solvent compound, stereoisomer, isotope and prodrug thereof according to any one of items 65-67, wherein, R₉₁ is selected from R₁₃, or one of R₁₀₄ is selected from R₁₃.

Item 69. The compounds and pharmaceutically acceptable salt, solvent compound, stereoisomer, isotope and prodrug thereof according to item 1 to item 2, wherein the compound is a compound represented by formula (XI t):

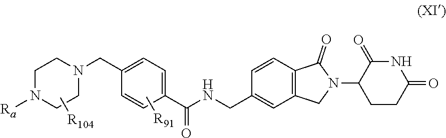

Wherein, Rₙ, Rₙ, R₁₀₄ is defined as above;

Preferably, R₉₁ is selected from: hydroxyl, Rₙ, —ORₙ, —NRₘRₙ, —NRₐC(═O)Rₘ, —NRₙ—S(═O)₂Rₘ,

Preferably, R₄ is selected from: hydroxyl, Rₙ, —ORₙ.

The term “pharmaceutically acceptable salt” is used to describe a salt form of one or more compounds described herein, which is provided to increase the solubility of the compounds in the gastric juices of the gastrointestinal tract of a patient, thereby facilitating dissolution and bioavailability of the compounds. Pharmaceutically acceptable salts include, where applicable, salts derived from pharmaceutically acceptable inorganic or organic bases and acids. Suitable salts include those derived from alkali metals (such as potassium and sodium) and alkaline earth metals (such as calcium, magnesium and ammonium salts), as well as salts of many other acids and bases well known in the pharmaceutical field.

Pharmaceutically acceptable salts according to the present invention include hydrochloride, phosphate, hydrogen phosphate, dihydrogen phosphate, sulfate, nitrate, bicarbonate, carbonate, glutarate, hydrobromide, acetate, citrate, lactate, maleate, benzoate, mesylate, oxalate, benzenesulfonate, p-toluenesulfonate, tartrate, nalate, succinate, ascorbate, gluconate, lactate, etc.

The term “solvent compound” is selected from hemihydrate, monohydrate, dihydrate, etc.

The term “stereoisomer” is selected from enantiomers, diastereomers, etc. The term “prodrug” is a derivative of a compound and contains an additional moiety that is readily removed in the body to yield a parent molecule as a pharmacologically active substance. An example of prodrugs is an ester, which is cleaved in the body to produce a compound of interest. Another example is an N-methyl derivative of a compound, which is susceptible to oxidative metabolic mechanisms leading to N-demethylation. Prodrugs of various compounds as well as materials and methods for producing prodrugs by derivatizing parent compounds am, known and are suitable for use in the present invention.

In particular, the present invention provides the following compounds.

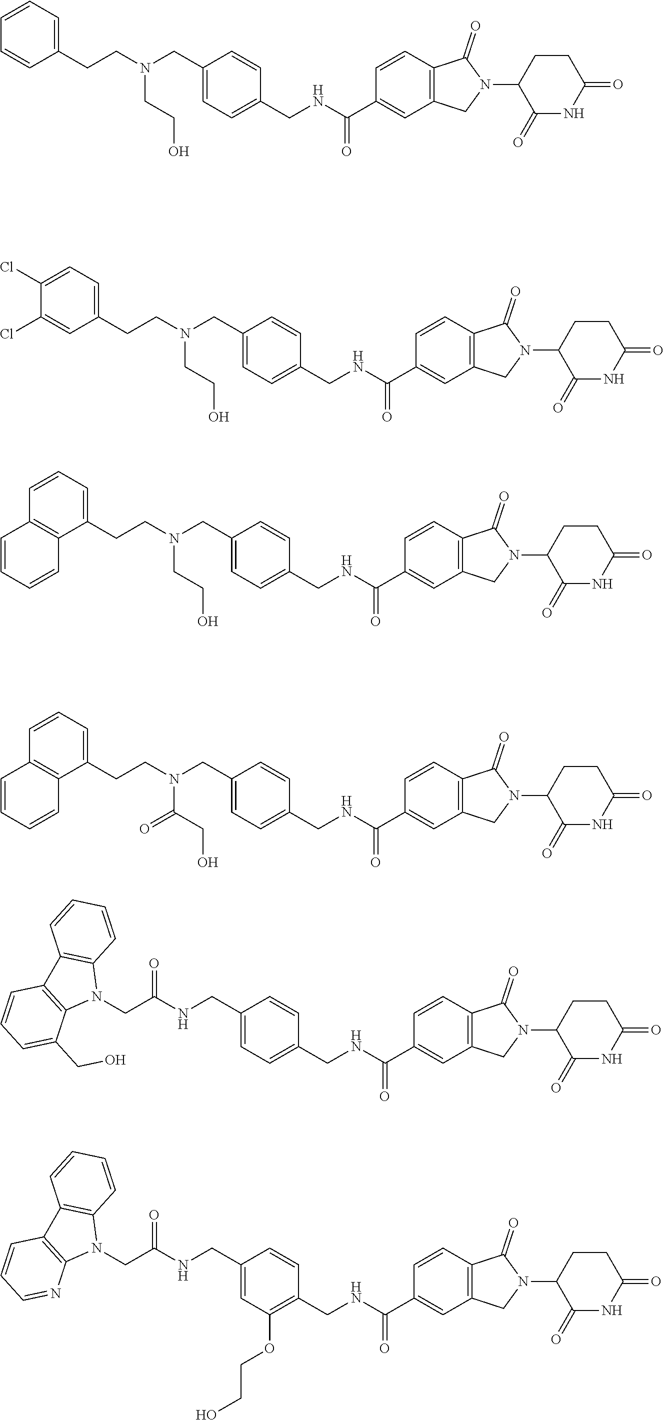 [SMILES: O=C1CCC(N2Cc3cc(C(=O)NCc4ccc(CN(CCO)CCc5ccccc5)cc4)ccc3C2=O)C(=O)N1 ; O=C1CCC(N2Cc3cc(C(=O)NCc4ccc(CN(CCO)CCc5ccc(Cl)c(Cl)c5)cc4)ccc3C2=O)C(=O)N1 ; O=C1CCC(N2Cc3cc(C(=O)NCc4ccc(CN(CCO)CCc5cccc6ccccc56)cc4)ccc3C2=O)C(=O)N1 ; O=C1CCC(N2Cc3cc(C(=O)NCc4ccc(CN(CCc5cccc6ccccc56)C(=O)CO)cc4)ccc3C2=O)C(=O)N1 ; O=C(Cn1c2ccccc2c2cccc(CO)c21)NCc1ccc(CNC(=O)c2ccc3c(c2)CN(C2CCC(=O)NC2=O)C3=O)cc1 ; O=C(Cn1c2ccccc2c2cccnc21)NCc1ccc(CNC(=O)c2ccc3c(c2)CN(C2CCC(=O)NC2=O)C3=O)c(OCCO)c1]

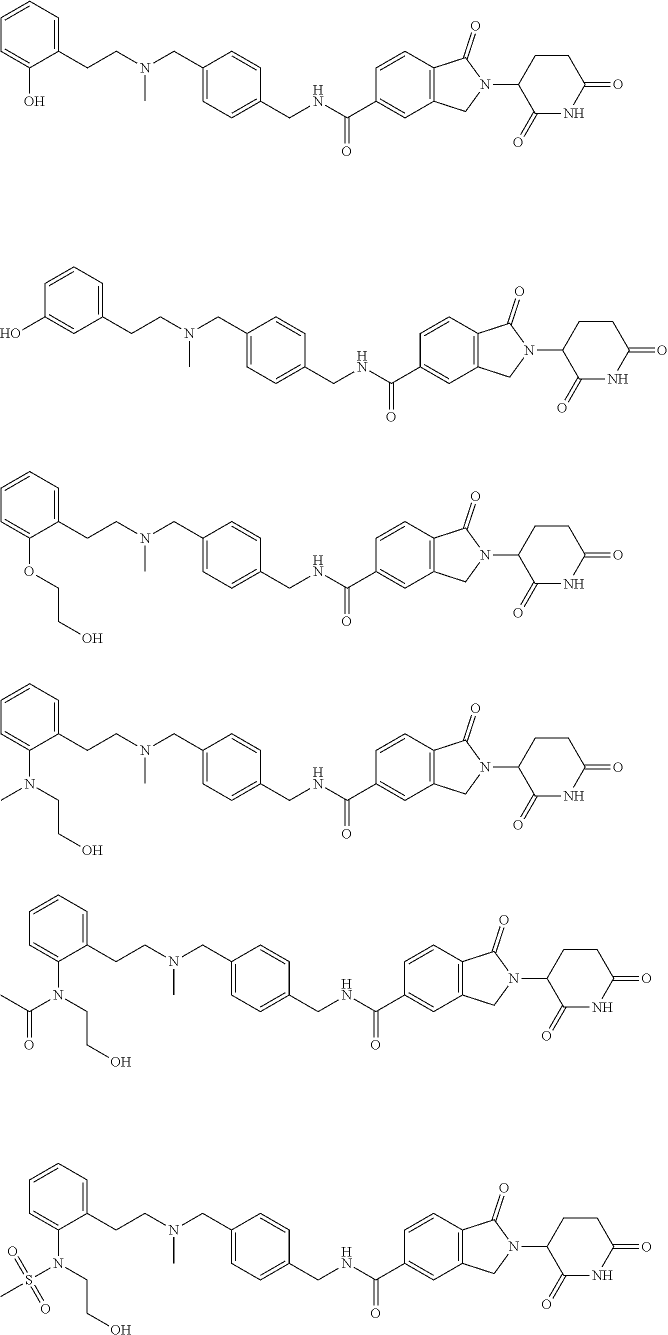 [SMILES: CN(CCc1ccccc1O)Cc1ccc(CNC(=O)c2ccc3c(c2)CN(C2CCC(=O)NC2=O)C3=O)cc1 ; CN(CCc1cccc(O)c1)Cc1ccc(CNC(=O)c2ccc3c(c2)CN(C2CCC(=O)NC2=O)C3=O)cc1 ; CN(CCc1ccccc1OCCO)Cc1ccc(CNC(=O)c2ccc3c(c2)CN(C2CCC(=O)NC2=O)C3=O)cc1 ; CN(CCc1ccccc1N(C)CCO)Cc1ccc(CNC(=O)c2ccc3c(c2)CN(C2CCC(=O)NC2=O)C3=O)cc1 ; CC(=O)N(CCO)c1ccccc1CCN(C)Cc1ccc(CNC(=O)c2ccc3c(c2)CN(C2CCC(=O)NC2=O)C3=O)cc1 ; CN(CCc1ccccc1N(CCO)S(C)(=O)=O)Cc1ccc(CNC(=O)c2ccc3c(c2)CN(C2CCC(=O)NC2=O)C3=O)cc1]

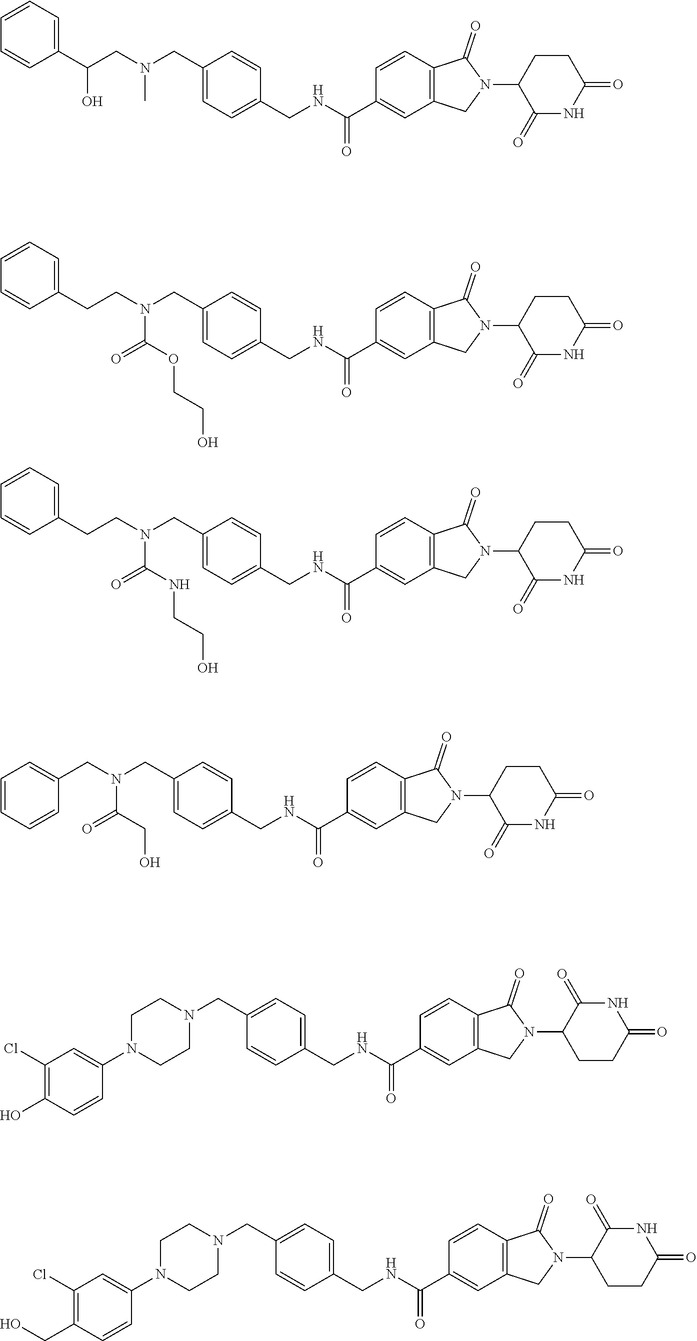 [SMILES: CN(Cc1ccc(CNC(=O)c2ccc3c(c2)CN(C2CCC(=O)NC2=O)C3=O)cc1)CC(O)c1ccccc1 ; O=C1CCC(N2Cc3cc(C(=O)NCc4ccc(CN(CCc5ccccc5)C(=O)OCCO)cc4)ccc3C2=O)C(=O)N1 ; O=C1CCC(N2Cc3cc(C(=O)NCc4ccc(CN(CCc5ccccc5)C(=O)NCCO)cc4)ccc3C2=O)C(=O)N1 ; O=C1CCC(N2Cc3cc(C(=O)NCc4ccc(CN(Cc5ccccc5)C(=O)CO)cc4)ccc3C2=O)C(=O)N1 ; O=C1CCC(N2Cc3cc(C(=O)NCc4ccc(CN5CCN(c6ccc(O)c(Cl)c6)CC5)cc4)ccc3C2=O)C(=O)N1 ; O=C1CCC(N2Cc3cc(C(=O)NCc4ccc(CN5CCN(c6ccc(CO)c(Cl)c6)CC5)cc4)ccc3C2=O)C(=O)N1]

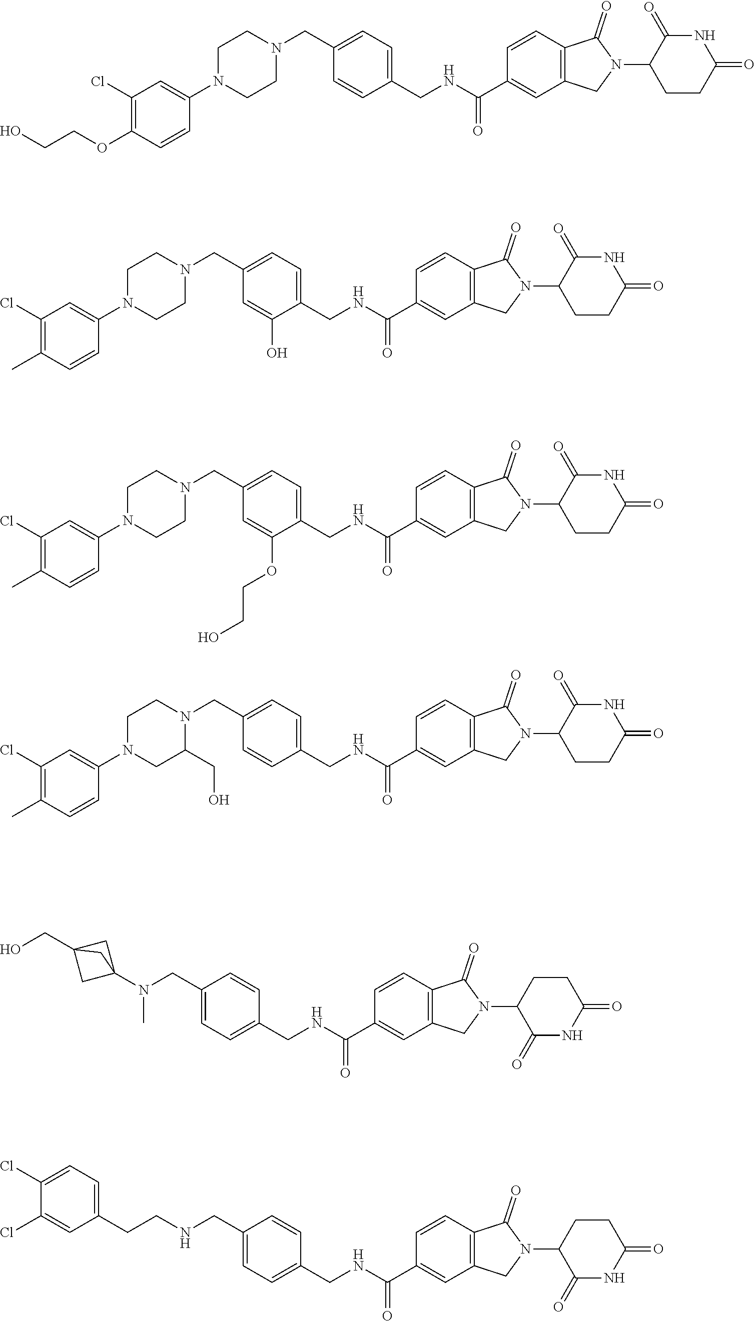 [SMILES: O=C1CCC(N2Cc3cc(C(=O)NCc4ccc(CN5CCN(c6ccc(OCCO)c(Cl)c6)CC5)cc4)ccc3C2=O)C(=O)N1 ; Cc1ccc(N2CCN(Cc3ccc(CNC(=O)c4ccc5c(c4)CN(C4CCC(=O)NC4=O)C5=O)c(O)c3)CC2)cc1Cl ; Cc1ccc(N2CCN(Cc3ccc(CNC(=O)c4ccc5c(c4)CN(C4CCC(=O)NC4=O)C5=O)c(OCCO)c3)CC2)cc1Cl ; Cc1ccc(N2CCN(Cc3ccc(CNC(=O)c4ccc5c(c4)CN(C4CCC(=O)NC4=O)C5=O)cc3)C(CO)C2)cc1Cl ; CN(Cc1ccc(CNC(=O)c2ccc3c(c2)CN(C2CCC(=O)NC2=O)C3=O)cc1)C12CC(CO)(C1)C2 ; O=C1CCC(N2Cc3cc(C(=O)NCc4ccc(CNCCc5ccc(Cl)c(Cl)c5)cc4)ccc3C2=O)C(=O)N1]

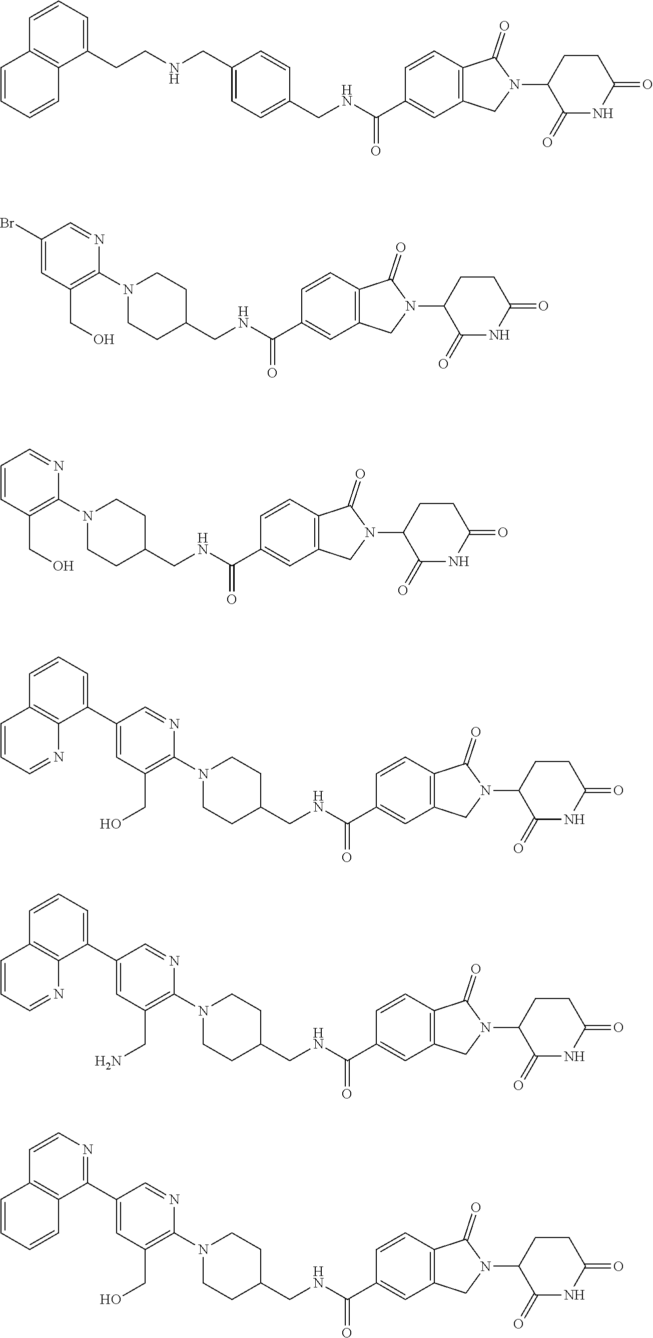 [SMILES: O=C1CCC(N2Cc3cc(C(=O)NCc4ccc(CNCCc5cccc6ccccc56)cc4)ccc3C2=O)C(=O)N1 ; O=C1CCC(N2Cc3cc(C(=O)NCC4CCN(c5ncc(Br)cc5CO)CC4)ccc3C2=O)C(=O)N1 ; O=C1CCC(N2Cc3cc(C(=O)NCC4CCN(c5ncccc5CO)CC4)ccc3C2=O)C(=O)N1 ; O=C1CCC(N2Cc3cc(C(=O)NCC4CCN(c5ncc(-c6cccc7cccnc67)cc5CO)CC4)ccc3C2=O)C(=O)N1 ; NCc1cc(-c2cccc3cccnc23)cnc1N1CCC(CNC(=O)c2ccc3c(c2)CN(C2CCC(=O)NC2=O)C3=O)CC1 ; O=C1CCC(N2Cc3cc(C(=O)NCC4CCN(c5ncc(-c6nccc7ccccc67)cc5CO)CC4)ccc3C2=O)C(=O)N1]

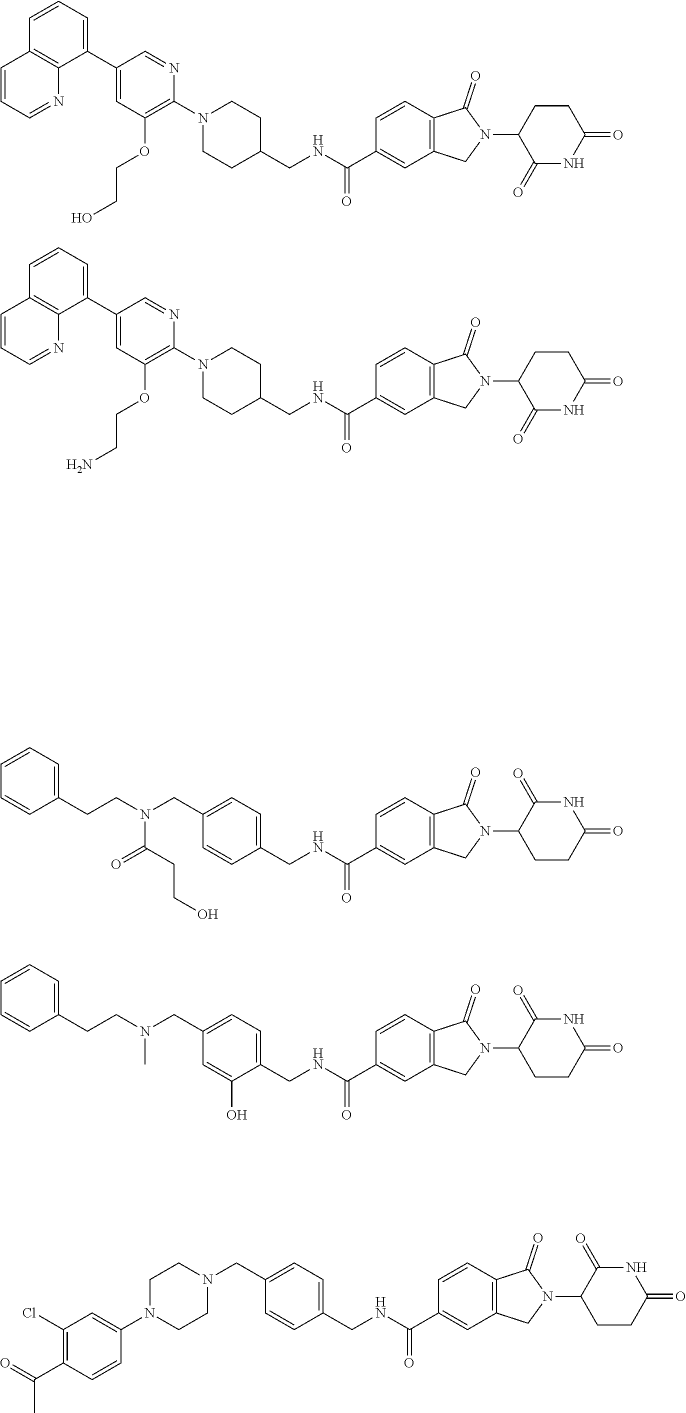

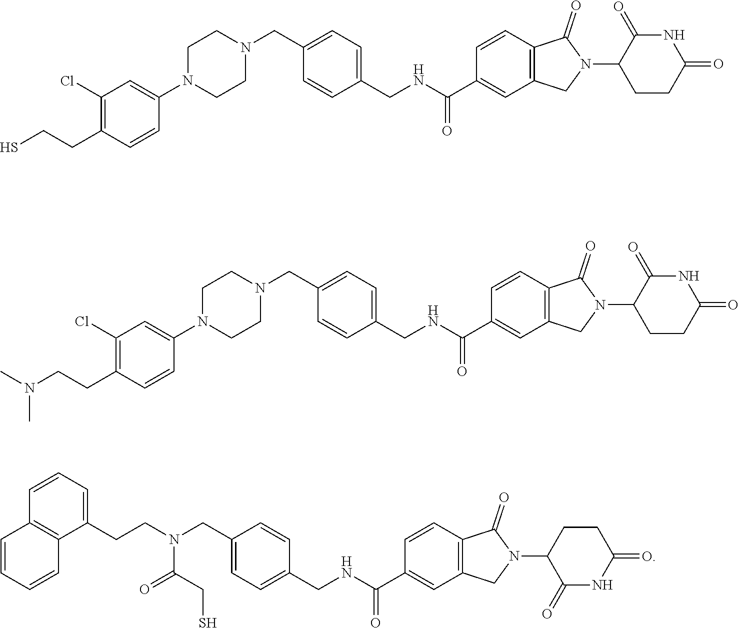 [SMILES: O=C1CCC(N2Cc3cc(C(=O)NCc4ccc(CN5CCN(c6ccc(CCS)c(Cl)c6)CC5)cc4)ccc3C2=O)C(=O)N1 ; CN(C)CCc1ccc(N2CCN(Cc3ccc(CNC(=O)c4ccc5c(c4)CN(C4CCC(=O)NC4=O)C5=O)cc3)CC2)cc1Cl ; O=C1CCC(N2Cc3cc(C(=O)NCc4ccc(CN(CCc5cccc6ccccc56)C(=O)CS)cc4)ccc3C2=O)C(=O)N1]

Preferably, the present invention provides the following compounds:

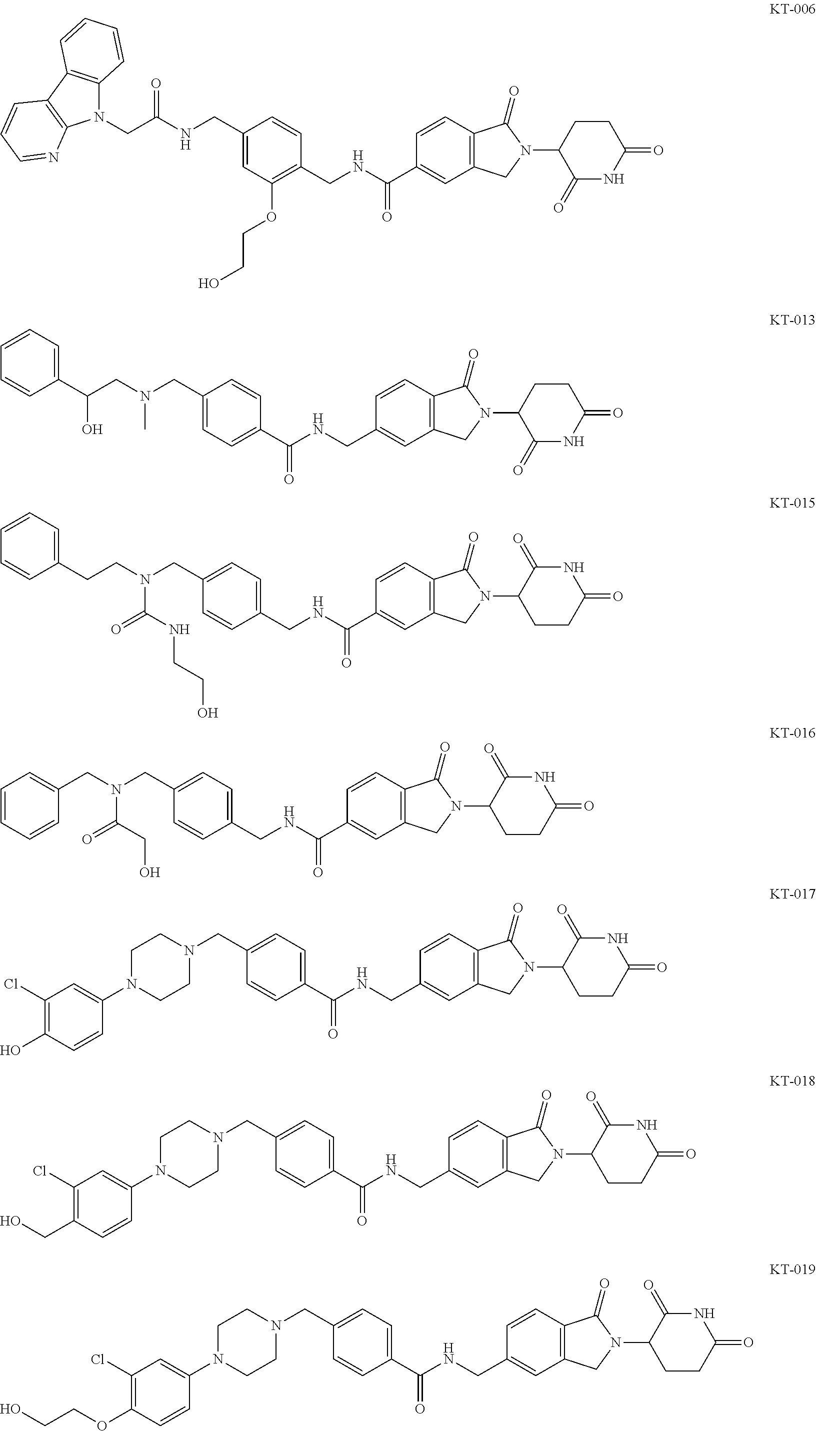

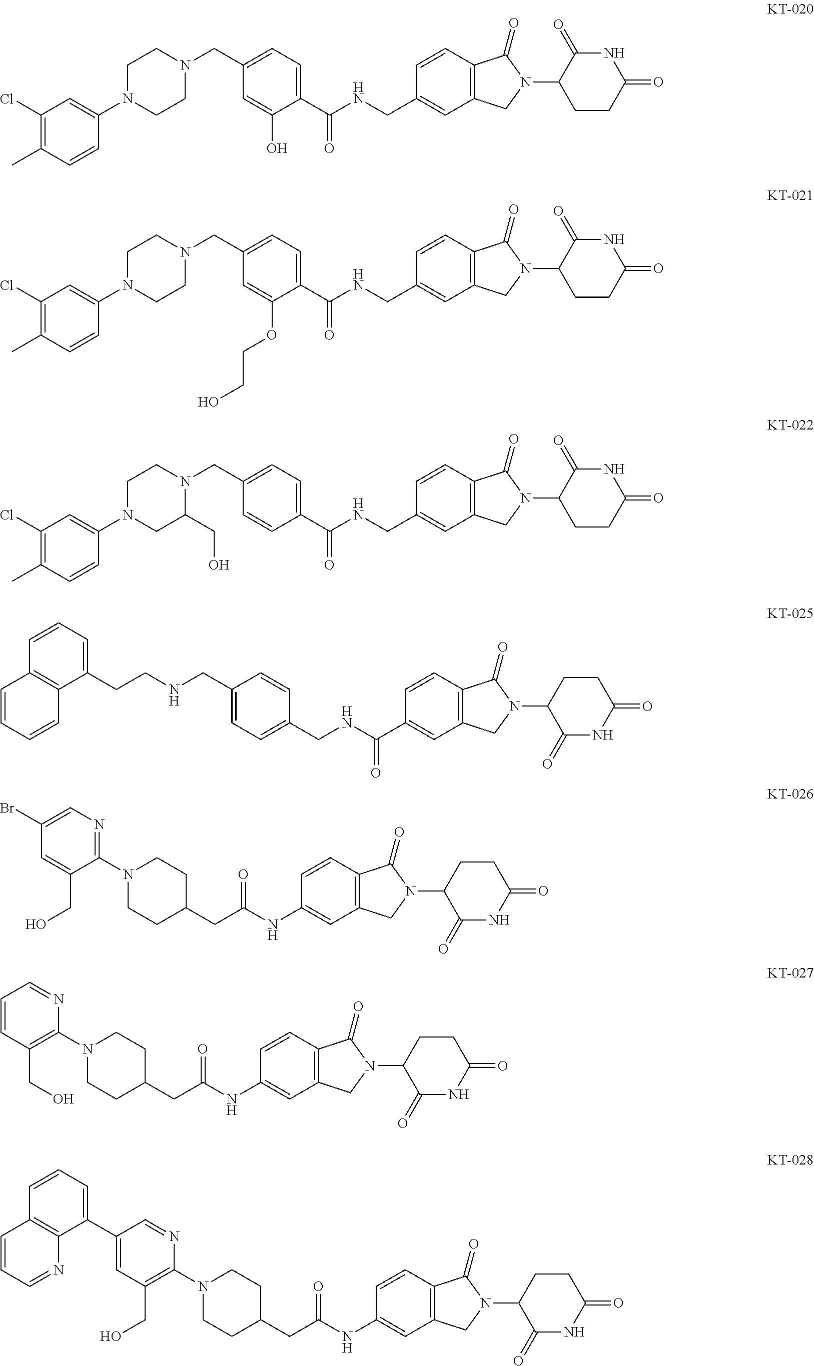

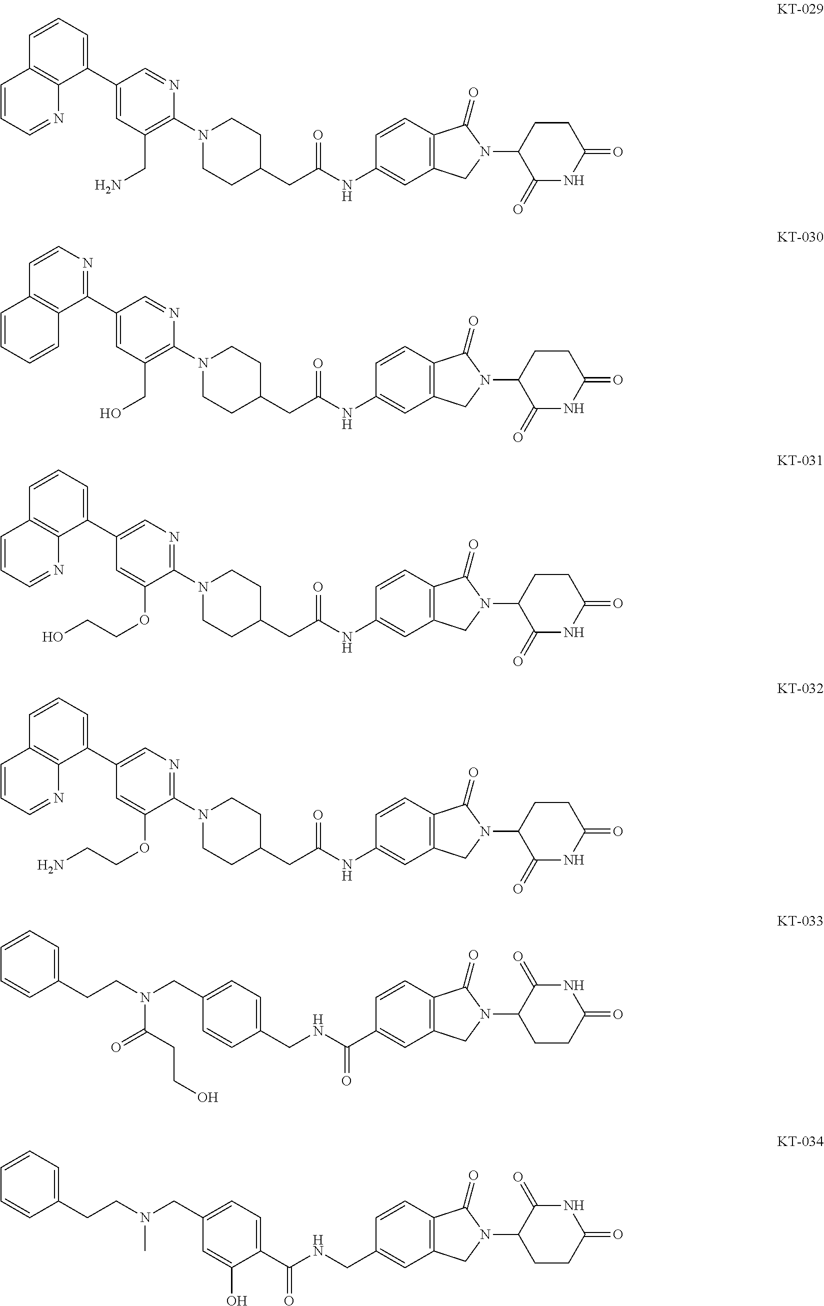 [SMILES: NCc1cc(-c2cccc3cccnc23)cnc1N1CCC(CC(=O)Nc2ccc3c(c2)CN(C2CCC(=O)NC2=O)C3=O)CC1 ; O=C1CCC(N2Cc3cc(NC(=O)CC4CCN(c5ncc(-c6nccc7ccccc67)cc5CO)CC4)ccc3C2=O)C(=O)N1 ; O=C1CCC(N2Cc3cc(NC(=O)CC4CCN(c5ncc(-c6cccc7cccnc67)cc5OCCO)CC4)ccc3C2=O)C(=O)N1 ; NCCOc1cc(-c2cccc3cccnc23)cnc1N1CCC(CC(=O)Nc2ccc3c(c2)CN(C2CCC(=O)NC2=O)C3=O)CC1 ; O=C1CCC(N2Cc3cc(C(=O)NCc4ccc(CN(CCc5ccccc5)C(=O)CCO)cc4)ccc3C2=O)C(=O)N1 ; CN(CCc1ccccc1)Cc1ccc(C(=O)NCc2ccc3c(c2)CN(C2CCC(=O)NC2=O)C3=O)c(O)c1]

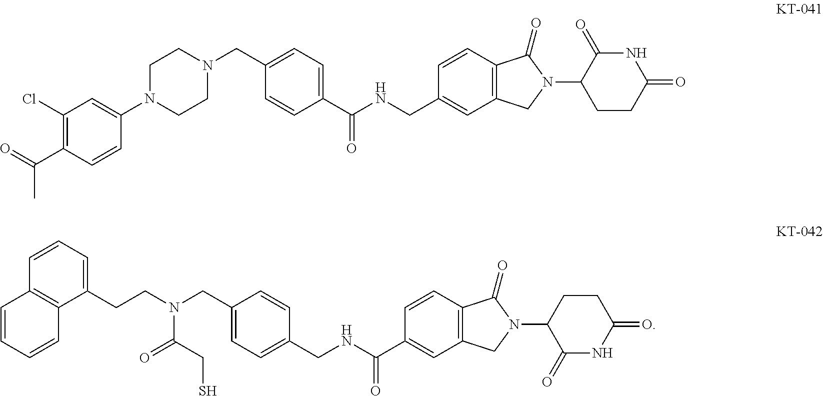 [SMILES: CC(=O)c1ccc(N2CCN(Cc3ccc(C(=O)NCc4ccc5c(c4)CN(C4CCC(=O)NC4=O)C5=O)cc3)CC2)cc1Cl ; O=C1CCC(N2Cc3cc(C(=O)NCc4ccc(CN(CCc5cccc6ccccc56)C(=O)CS)cc4)ccc3C2=O)C(=O)N1]

any one or more of 5-azacytidine, RG108, SGI-1027, GSK3685032, CM272, Bobcat339 hydrochloride, Decitabine (NSC 127716), Thioguanine (NSC 752), 2Deoxy-5-Fluorocytidine, Procainamnide HCl or Zebularine (NSC 309132); the Bel-2 selective inhibitor is any one or more of Venetoclax (ABT-199), S55746, BDA-366, Obatoclax Mesylate (CX15-070), HA14-1 or APG-2575 (CAS No. 2180923-05-9). Further preferably, the pharmaceutical composition comprises a Bel-2 selective inhibitor, and the Bel-2 selective inhibitor is Venetoclax (ABT-199), Obatoclax Mesylate (GX15—O⁷0) or APG-2575 (CAS No. 2180923-05-9).

Yet another object of the present invention is to provide the use of the compound and the pharmaceutically acceptable salt, solvent compound, stereoisomer, isotope and prodrug thereof and the pharmaceutical composition according to the present invention in the preparation of a protein degradation agent for any one of or at least two of c-Myc, N-myc, GSPT1, CK1W, IKZF (1/2/3), AR and AR-V7.

Yet another object of the present invention is to provide the use of the compound and the pharmaceutically acceptable salt, solvent compound, stereoisomer, isotope and prodrug thereof and the pharmaceutical composition according to the present invention in the preparation of a drug for treating a disease related to dysregulation of proteins comprising any one of or at least two of c-Myc, N-myc, GSPT1, CK1α, IKZF (1/213), AR and AR-V7.

Yet another object of the present invention is to provide the use of the compound and the pharmaceutically acceptable salt, solvent compound, stereoisomer, isotope and prodrug thereof and the pharmaceutical composition according to the present invention in the treatment of a disease related to dysregulation of proteins comprising any one of or at least two of c-Myc, N-myc, GSPT1, CK1α, IKZF (1/2/3), AR and AR-V7.

Yet another object of the present invention is to provide a method for treating diseases related to protein disorder comprising any one or at least two or more of c-Myc, N-mye, GSPT1, CK1α, IKZF (112/3), AR and AR-V7, which comprises administering to a subject in need thereof a therapeutically effective amount of the compound and the pharmaceutically acceptable salt, solvent compound, stereoisomer, isotope and prodrug thereof and the pharmaceutical composition according to the present invention.

Yet another object of the present invention is that the compound and the pharmaceutically acceptable salt, solvent compound, stereoisomer, isotope and prodrug thereof and the pharmaceutical composition according to the present invention are used to treat diseases related to protein disorder comprising any one or at least two or more of c-Myc, N-myc, GSPT1, CK1α, IKZ (1/2/3), AR and AR-V7.

Preferably, the dysregulation of proteins is selected from an overexpression of protein.

Preferably, the disease related to dysregulation of proteins is selected from cancer, a cardiovascular and cerebrovascular disease, a viral infection-related disease, etc.

Preferably, the cancer is selected from leukemia, lymphoma, malignant glioma, medulloblastoma, melanoma, multiple myeloma, myelodysplastic syndrome, liver cancer, lung cancer, kidney cancer, pancreatic cancer, oral cancer, gastric cancer, esophageal cancer, laryngeal cancer, nasopharyngeal cancer, skin cancer, breast cancer, colon cancer, rectal cancer, cervical cancer, ovarian cancer, prostate cancer, rhabdomyosarcoma, osteoblastic sarcoma or chondrosarcoma; the viral infection-related disease is selected from HIV, hepatitis B, hepatitis C, hepatitis A, influenza, epidemic encephalitis B, herpes, etc.; the leukemia includes chronic lymphocytic leukemia (CLL), chronic granulocytic leukemia (CML), acute myeloid leukemia (AML), acute non-lymphocytic leukemia (ANLL), etc.

Preferably, the present invention provides the use of the compound and the pharmaceutically acceptable salt, solvent compound, stereoisomer, isotope and prodrug thereof and the pharmaceutical composition according to the present invention in the preparation of a drug for treating acute myeloid leukemia (AML). Further preferably, the pharmaceutical composition comprises a Bel-2 selective inhibitor, and the Bel-2 selective inhibitor is Venetoclax (ABT-199), Obatoclax Mesylate (GX15-070) or APC-2575 (CAS No. 2180923-05-9). The compound and the pharmaceutically acceptable salt, solvent compound, stereoisomer, isotope and prodrug thereof according to the present invention are used in combination or in connection with a Bel-2 selective inhibitor. For example, using compounds of the present invention in combination or in connection with the Bel-2 selective inhibitor Venetoelax (ABT-199) can solve the drug resistance problem of using the Bcl-2 selective inhibitor Venetoclax (ABT-199) alone to treat acute myeloid leukemia (AML).

Yet another object of the present invention is to provide the use of the compound and the pharmaceutically acceptable salt, solvent compound, stereoisomer, isotope and prodrug thereof according to the present invention in the preparation of proteolysis targeting chimera, PROTAC), antibody-drug conjugate (ADC), peptide-drug conjugate (PDC) or smallmolecular-drug conjugate (PDC), SMDC).

Beneficial effects of the invention The compounds provided by the present invention have excellent effects on degrading any one or more of proteins comprising c-Myc, N-myc, GSPT1, CK1α, IKZF (1/213), AR, AR-V⁷, etc., and therefore can be used to prevent, alleviate or treat diseases related to high expression of any one or more of the above proteins (e.g., c-Myc), such as cancer, cardiovascular and cerebrovascular diseases and viral infection-related diseases. The methods for synthesizing the compounds are simple and convenient, and the compounds have precise and significant effects on degrading c-Myc, N-myc, GSPT1, CKlW, IKZF (1/2/3), AR and AR-V⁷ proteins.

### Definition

Unless defined otherwise, the terms used herein have the same meanings as commonly understood by those of ordinary skill in the art to which the present invention pertains. The terms used in the specification are merely used to describe specific embodiments, and are not intended to limit the present invention.

In the present invention, “ [symbol] ” indicates a connection point for connecting moieties.

The term “unsubstituted” shall mean substitution only with a hydrogen atom.

The term “substituted” or “optionally substituted” means the independent presence of one or more substituents, preferably 1-5 substituents, and most preferably 1-3 substituents at any carbon (or nitrogen) position in a molecule, wherein the substituents may be selected from hydroxyl, thiol, carboxyl, cyano, nitro, halogen (preferably, 1, 2 or 3 halogen, especially on alkyl, especially methyl, such as trifluoromethyl), alkyl (preferably C₁-C₁₀ alkyl, and more preferably C₁-C₆ alkyl), haloalkyl, alkenyl, alkynyl, cycloalkyl, aryl (especially phenyl), heteroaryl, heterocyclyl, alkoxy (preferably C₁-C₆ alkoxy), aryloxy (preferably phenoxy), thioether (C₁-C₆ alkylthio or arylthio such as phenylthio), acyl (preferably C₁-C₆ acyl, or arylcarbonyl such as benzoyl), C₁-C₆ alkylamino, bis(C₁-C₆ alkyl)amido, aminoacyl substituted with one or two C₁-C₆ alkyl (including carbamoyl optionally substituted with one or two C₁-C₆ alkyl), a C₁-C₆ ester group, etc.

The term “alkyl” refers to a linear or branched fully saturated hydrocarbon group or alkyl group which may be optionally substituted, preferably C₁-C₁₀ alkyl, more preferably C₁-C₆ alkyl, or C₁-C₄ alkyl. Examples of alkyl are methyl, ethyl, propyl, isopropyl, n-butyl, sec-butyl, isobutyl, tert-butyl, n-pentyl, n-hexyl, n-heptyl, n-octyl, n-nonyl, n-decyl, etc.

The term “alkylene” refers to a divalent group formed by further removing one hydrogen atom from alkyl.

The term “cycloalkyl” refers to a cyclic hydrocarbon group or alkyl group which may be optionally substituted, preferably C₃-C₂₀ alkyl, more preferably C₃-C₁₅ alkyl, or C₃-C₈ alkyl.

Examples of cycloalkyl are cyclopropyl, cyclobutyl, cyclopentyl, cyclohexyl, cyclohexyl, cycloheptyl, etc.

The term “cycloalkylene” refers to a divalent group formed by further removing one hydrogen atom from cycloalkyl.

The term “bridged cyclic group” refers to a cyclic structure formed by two or more cyclic structures which may be optionally substituted and share two non-adjacent carbon atoms with each other, preferably a C-CH₅ bridged cyclic group, more preferably a C₆-C₁₂ bridged cyclic group, or a C₇-C₁₀ bridged cyclic group. Examples of bridged cyclic groups are bicyclic [1.1.11 pentyl, norbornyl, adamantyl, etc The term “alkenyl” refers to a linear chain, branched chain or cyclic C₂-C₁₀ (preferably C₂-C₅) hydrocarbon group containing at least one C═C bond.

The term “aryl” refers to a C₆-C₁₆ aromatic group, preferably a C₆-C₁₀ aromatic group, which may be optionally substituted and has a single ring (such as phenyl) or a fused ring (such as naphthyl, anthryl, phenanthryl and fluorenyl).

The term “arylene” refers to a divalent group formed by further removing one hydrogen atom from aryl.

The term “heteroaryl” refers to a 5- to 16-membered aromatic group, preferably a 5- to 10-membered aromatic group, which may be optionally substituted and contains one or more heteroatoms selected from N, O, S or P. Examples of heteroaryl include imidazolyl, pyrazolyl, triazolyl, tetrazolyl, pyrrolyl, furyl, thienyl, oxazolyl, isoxazolyl, thiazolyl, isothiazolyl, pyridyl, pyrannyl, pyrimidyl, pyridazinyl, indolyl, azaindolyl (such as 7-aza-indolyl), benzoimidazolyl, benzopyrazolyl, benzofuryl, benzothienyl, benzothiazolyl, dibenzofuryl, dibenzothienyl, quinolyl, isoquinolyl, naphthyridinyl, carbazolyl, azacarbazolyl (such as 1-azacarbazolyl, 2-azacarbazolyl and 1,8-diazacarbazolyl), indolazinyl, azaindolizinyl, phenoxazinyl, phenothiazinyl, etc.

The term “heteroarylene” refers to a divalent group formed by further removing one hydrogen atom from heteroaryl.

The term “heterocyclyl” refers to partially unsaturated or fully unsaturated 3- to 20-membered cyclic group, preferably 3- to 10-membered cyclic group, more preferably 3- to 6-membered cyclic group, which may be optionally substituted and contain one or more heteroatoms selected from N, O, S, SO, SO₂ or P; and the heterocyclyl contains 1-19 carbon atoms, preferably 2-10 carbon atoms, more preferably 3-5 carbon atoms. Examples of heterocyclyl include: azacyclopropanyl, oxacyclopropanyl, azetidinyl, oxetanyl, 1,4-benzodioxanyl, 1,3-benzodioxolyl, dihydroimidazolyl, dihydropyranyl, dihydrofuryl, dioxanyl, ethyleneureido, 1,3-dioxacyclopropanyl, 1,3-dioxanyl, 1,4-dioxanyl, imidazolinyl, indolinyl, morpholinyl, pyridone, 2-pyrrolidone, piperazinyl, homopiperazinyl, piperidyl, homopiperidinyl, phthalimidyl, succimidyl, pyrazinyl, pyrazolinyl, pyrrolinyl, tetrahydrofuryl, tetrahydropyranyl, tetrahydroquinolyl, tetrahydrothienyl, oxanyl, oxathiolanyl, thianyl, etc.

The term “heterocyclylene” refers to a divalent group formed by further removing one hydrogen atom from heterocyclyl.

The term “heterocycloalkyl” refers to a fully saturated heterocyclyl group.

The term “fused arylcycloalkyl” refers to a group in which aryl is fused to cycloalkyl as described above.

The tern “fused arylheterocyclyl” refers to a group in which aryl is fused to heterocyclyl as described above.

The term “fused heteroarylcycloalkyl” refers to a group in which heteroaryl is fused to cycloalkyl as described above.

The term “fused heteroarylheterocyclyl” refers to a group in which heteroaryl is fused to heterocyclyl as described above.

The term “aryl-aryl” refers to the aryl and aryl group are connected by a single bond, and other sites are connected by the aryl group. The terms “aryl-heteroaryl,” “heteroaryl-aryl,” “heteroaryl-heteroaryl,” etc.

The term “aryl-alkyl” refers to that the aryl and alkyl group are connected by a single bond, and other sites are connected by an alkyl group. The terms “heteroaryl-alkyl,” “aryl-cycloalkyl,” “aryl-heterocyclic,” “cycloalkyl-heterocyclic,” “heterocyte-heterocyclic,” etc.

The term “halogen” refers to F, Cl, Br, I.

The term “pharmaceutically acceptable carrier” may mean any and all solvents, dispersion media, coatings, antibacterial agents, etc. that are compatible with drug administration.

Unless otherwise indicated, the ten “compound” refers to any specific compound disclosed herein and includes tautomers, regioisonmers, geometric isomers, and, where applicable, stereoisomers, including optical isomers (enantiomers) and other stereoisomers (diastereomners), as well as pharmaceutically acceptable salts and derivatives (including prodrug forms) thereof. When used in the context, the term “compound” generally refers not only to a single compound, but may include other compounds, such as stereoisomers, regioisomers and/or optical isomers (including racemic mixtures), as well as specific enantiomers or enantionmer-enriched mixtures of the disclosed compounds. The term in the context also refers to a prodrug form of the compound, which has been modified to facilitate administration and delivery of the compound to an active site.

The term “proteolysis targeting chimera (PROTAC)” is a hybrid bifunctional small molecule compound (Sakamoto KM. et al. Proc Natl Acad Sci USA, 2001, 98:8554-8559), including a small molecule compound that can bind to a protein of interest (POI) connected via a linking group at an appropriate position thereof to a small molecule compound that can bind to an E3 ubiquitin ligase.

The term “antibody-drug conjugate (ADC)” refers to a product in which a biologically active small molecule drug is connected to a monoclonal antibody via a chemical linker, wherein the monoclonal antibody serves as a carrier to deliver the small molecule drug to the cells of interest.

The term “peptide-drug conjugate (PDC)”, also known as peptide-drug conjugate, is a new type of molecular drug delivery system, which is generally composed of a polypeptide, a covalent linker, and a payload. Usually, the combination of a drug (compound) and a polypeptide through a covalent linker endods the bidirectional function of the peptide and the drug (compound), which can promote the killing or targeting effect of the drug.

The term “smalhnolecular-drug conjugate (SMDC)” is a new type of molecular drug delivery system, which is generally composed of a small molecule targeting ligand, a linker, and a payload. Usually, a drug (compound) can be combined with a small molecule targeting ligand through a linker to give the targeting function, which can promote the killing or targeting effect of the drug.

In addition, the full text of international application number PCT/CN2022/100078 is also incorporated herein by reference.

### BRIEF DESCRIPTION OF DRAWINGS

FIG. 1 shows the degradation diagram of c-Myc and GSPT1 proteins in HL60 cells by the compounds of the invention.

### DETAILED DESCRIPTION OF EMBODIMENTS

The present invention will be exemplified below in connection with further detailed description. It should be noted that the following description is to exemplify rather than limit the technical solutions of the present invention. The scope of protection of the present invention shall be in accordance with the appended claims.

Bruker 400 MHz NMR instrument was used for ¹H NMR measurement; The test solvents were deuterated methanol (CD₃OD), deuterated chloroform (CDCl₃) or hexadeuterated dimethyl sulfoxide (DMSO-d). Tetramethylsilane (TMS) was used as internal standard.

The abbreviations in the nuclear magnetic resonance (NMR) profiles used in the examples are shown below. S: unimodal (singlet), d: double peak (doublet), t: triple peak (triplet), q: quad peak (quartet), dd: double double peak (double doublet), qd: four double peak (quartet doublet), ddd: double double doublet, ddt: double double triplet, dddd: double double double doublet, m: multiple peaks (multiplet), br: broad peak (broad), J1: coupling constant, Hz: Hertz, DMSO-d₆: deuteride di methyl sufoxideo 6 values are expressed as ppm values.

### Abbreviations of Reagents in Examples

| | | | |
|---|---|---|---|
| HCl | hydrochloric acid | DMF | N,N-dimethylformamide |
| DCM | dichloromethane | MeOH | methanol |
| NBS | N-Bromosuccinimide | AIBN | 2,2′-Azobis(2-methylpropionitrile) |
| MTBE | methyl tert-butyl ether | DCC | dicyclohexyl carbodiimide |
| Dioxane | 1,4-dioxane | DIPEA | N,N-diisopropylethylamine |
| HATU | 2-(7-aza-benzotriazole)-N,N,N′,N′- | Xantphos | 4,5-bis(diphenylphosphino)-9,9- |
| | tetramethylurea hexafluorophosphate | | dimethylxanthene |
| NaBH₃CN | sodium cyanoborohydride | NaHCO₃ | sodium bicarbonate |
| AcOH | acetic acid | TEA | triethylamine |
| THF | tetrahydrofuran | NaH | sodium hydride |
| DPPF | 1,1′-Bis(diphenylphosphino)ferrocene | FmocCl | chloroformate-9-fluorenylmethyl |
| | | | ester |
| DMSO | dimethyl sulfoxide | FBS | fetal calf serum |
| PB/PBS | phosphate buffer | Dess-Martin | Dess-Martin reagent |

### Synthetic embodiments

### Example 1

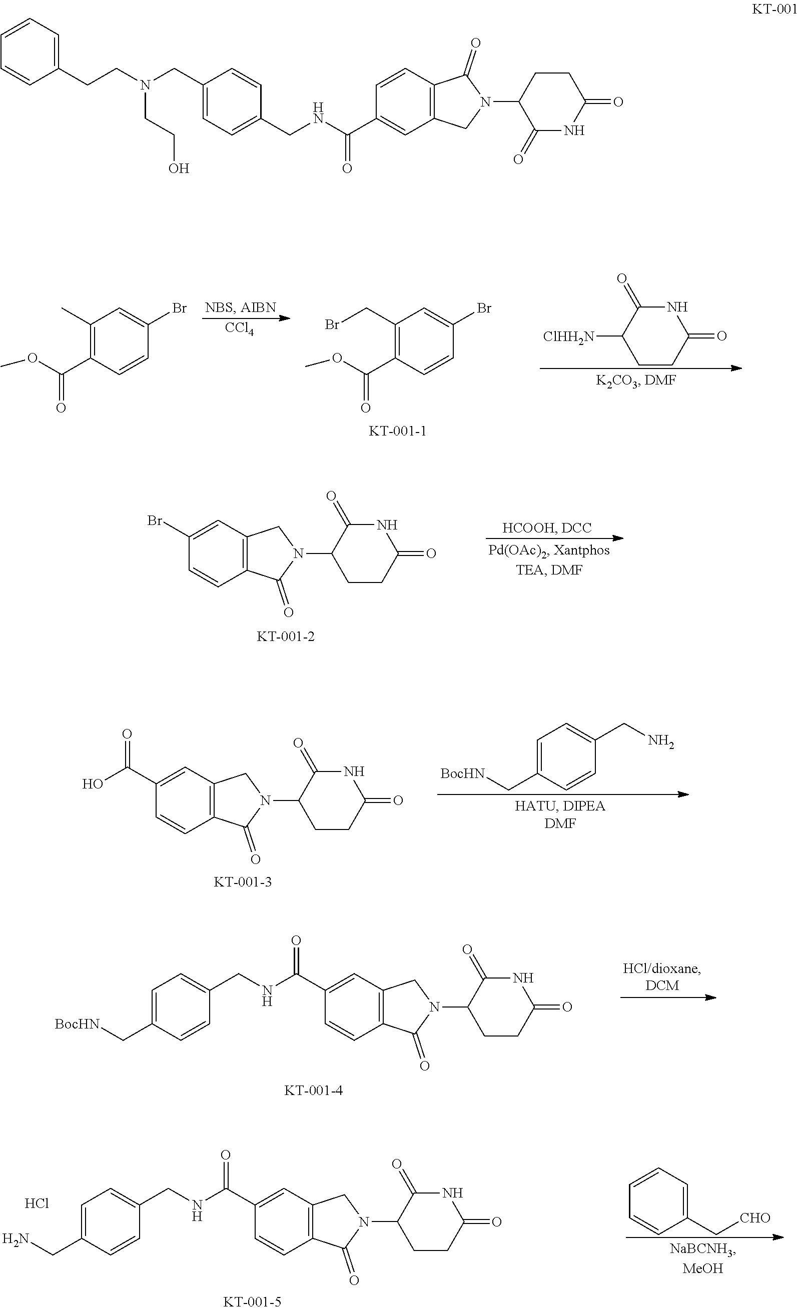

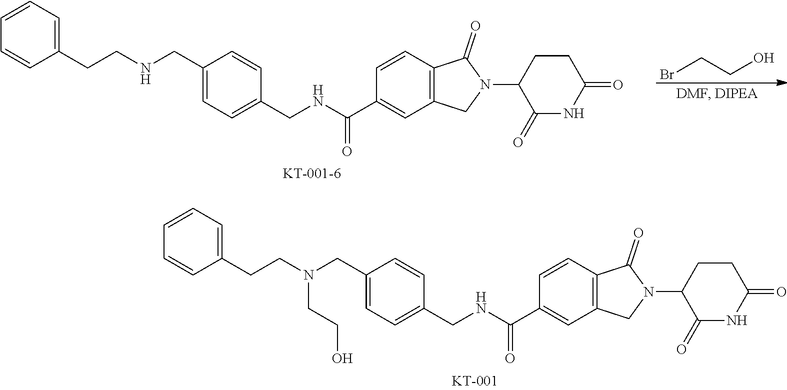

### Synthesis of compound KT-001-1

Methyl-4-bromo-2-methylbenzoate (30.0 g, 131 mmol) was dissolved in 300 mL of carbon tetrachloride to which NBS (28 g, 131 mmol) and AIBN (6.4 g, 40 mmol) were added. After reacted at 80° C. for 1 h, TLC showed that the reaction was complete. After dilution by adding 150 mL of MTRE, the reaction was filtered. The filtrate was washed with saturated aqueous sodium bicarbonate (30 mL*3), The organic phase was dried and dried by rotary evaporation to yield compound KT-001-1 (48 g, crude).

### Synthesis of compound KT-001-2

Compound KT-001-1 (48 g, 131 mmol) was dissolved in 450 nL DM1F, to which 3-aminopperidine-2, 6-dione hydrochloride (28 g, 172 mmol) and potassium carbonate (65 g, 469 mmol) were added. After reacted at 70° C. for 1 h, TLC showed that the reaction was complete. The reaction solution was filtered and the filtrate was evaporated most of the solvent. After slurried in 200 mL dichloromethane/water (1: 1), the precipitate was filtered. The filted cake was dried to yield compound KT-001-2 (19 g, 38% yield).

### Synthesis of compound KT-001-3

Compound KT-001-2 (10 g, 31 mmol) was dissolved in 80 mL of DMF, to which formic acid (5 g, 109 mmol), palladium acetate (209 mg, 0.93 mmol), DCC (1,3 g, 6.2 mmol), triethylamine (6.3 g, 62 mmol) and Xantphos (539 mg, 0.93 mmol) were added in sequence. After reacted at 100° C. for 3 h tinder nitrogen protection, TLC showed that the reaction was complete. The reaction solution was filtered and the filtrate was evaporated most of the solvent. After slurried in 50 mL methylene chloride, the precipitate was filtered. The filted cake was dried to yield compound KT-001-3 (7.8 g, 88% yield). ¹H NMR (400 MHz, DMSO) δ 11.02 (s, 1H), 8.17 (s, 1H), 8.00 (d, J=7.9 Hz, 1H), 7.96 (s, 1H), 7.84 (d, J=7.9 Hz, 1H), 5.14 (dd, J=13.3, 5.1 Hz, 1H), 4,55-4.37 (m, 2H), 2.99-2.84 (m, 1H), 2.69-2.58 (m, 1H), 2.46-2.38 (m, 1H), 2,07-1.95 (m, 1H).

### Synthesis of compound KT-001-4

Compounds KT-001-3 (730 mg, 2.53 mmol), 1-(N-Boc-aminomethyl)-4-(aminomethyl)benzene (600 mg, 2.53 mmol) and DIPEA (1.96 g, 15.18 mmol) were successively added to 20 mL DMF. ATU (1.15 g, 3.04 mmol) was added to this solution in ice bath. After reacted at room temperature for 1 h, TLC showed that the reaction was complete. After slurried in 120 mL of water/ethyl acetate (1: 1), the precipitate was filtered. The filted cake was washed with ethyl acetate and dried to yield compound KT-001-4 (710 mg, 55% yield). ¹H NMR (400 MHz, DMSO) δ 11.01 (s, 1H), 9.21 (t, J=5.8 Hz, 1H), 8.09 (s, 1H), 8.01 (d, J=8.3 Hz, 1H), 7.82 (d, J=8.0 Hz, 1H), 7.36 (s, 1H), 7.28 (d, J=7.8 Hz, 2H), 7.19 (d, J=7.9 Hz, 2H), 5.14 (dd, J=13.3, 5.0 Hz, 1H), 4.56 4.36 (m, 4 H1), 4.09 (d, J=5.9 Hz, 2H), 2.97 2.85 (m, 1H), 2.69-2.57 (m, 1H), 2.46 2.37 (m, 1H), 2.08-1.98 (m, 1H), 1.38 (s, 9H).

### Synthesis of compound KT-001-5

Compound KT-001-4 (710 mg, 1.4 mmol) was dissolved in dichloromethane (20 mL) to which HC/Dioxane (10 mL, 4 M) was added. After reacted at room temperature for 1 h, TLC showed that the reaction was complete. The reaction was dried by rotary evaporation to yield compound KT-001-5 (700 mg, crude), which was directly used for next step.

Synthesis of compound KT-001-6 Compound KT-001-5 (700 mg, 1.4 mmol) and phenylacetaldehyde (185 ng, 1.5 mmol) were dissolved in methanol (10 mL). After stirring for 5 min at room temperature. NaBH₃CN (132 mg, 2.1 mmol) was added. After reacted at room temperature for another 2 h, TLC showed the reaction was complete. The reaction mixture was added with aqueous NaHCO₃ (30 ml). The mixture was extracted with dichloromethane (35 ml*3). The organic phases were combined and dried by rotary evaporation. The crude product was purified by column chromatography (dichloromethane: methanol=15: 1 as eluent) to yield compound KT1-001-6 (600 mg, 84% yield).

ES1-MS(M+H): 511.2.

### Synthesis of compound KT-001

Compound KT-001-6 (600 mg, 1.17 mmol), 2-bromoethanol (293 mg, 3.51 mmol), and DIPEA (452 mug, 3.5 mmol) were dissolved in DMF (15 mL). After stirring at 80° C. overnight, TLC showed that the reaction was complete. The reaction was quenched by adding water (50 mL). The aqueous phase was extracted with ethyl acetate (5 mL*3) and the organic phases were combined and washed with saturated brine (30 mL*3). The organic phase was dried with anhydrous sodium sulfate, filtered, and the solvent was removed by rotary evaporation. The crude product was purified by column chromatography (dichloromethane: methanol=15: 1 as eluent) to yield compound KT-001 (200 mug, 31% yield). ¹H NMR (400 MHz, DMS) 6 11.01 (s, 1H), 9.22 (s, H), 8.10 (s, 1H), 8.01 (d, J=8.0 Hz, 1H), 7.82 (d, J=7.9 Hz, H), 7.39-7.10 (m, 9H), 5.13 (dd, J=13.2, 5.1 Hz, 1H), 4.60-4.32 (m, 4H), 3,78-3.48 (m, 6H), 2.98-2.86 (m, 1H), 2.83-2.53 (m, 5H), 2.46-2.31 (m, 1H), 2.09-1.96 (m, 1H). ES1-MS(M+H)⁺: 555.3. KT-002 and KT1-003 were synthesized according to KT-001

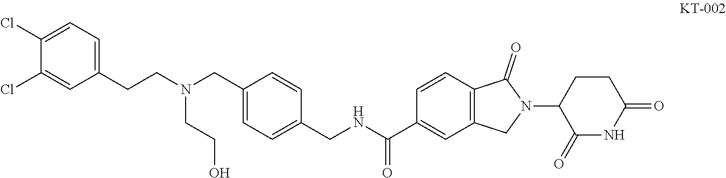 [SMILES: O=C1CCC(N2Cc3cc(C(=O)NCc4ccc(CN(CCO)CCc5ccc(Cl)c(Cl)c5)cc4)ccc3C2=O)C(=O)N1]

¹H NMR (400 MHz, DMSO) δ 11.01 (s, 1H), 9.20 (t, J=6.1 Hz, 1H-), 8.10 (s, 1H), 8.01 (d, J=7.7 Hz, 1H), 7.82 (d, J=7.9 Hz, 1H), 7.49-7.46 (m, 2H), 725-7.14 (m, 5H), 5.14 (dd, J=13.3, 5.1 Hz, 1H), 4.55-4.28 (m, 5H), 3.62 (s, 2H), 3.46-3.39 (m, 2H), 2.98-2.85 (m, 1H), 2.77-2.65 (m, 4H), 2.63-2.51 (m, 3H), 242 (dd, J=12.9, 4.5 Hz, 1H), 2.06-1.98 (m, H). ES1-MS(M+H)⁺: 623.2.

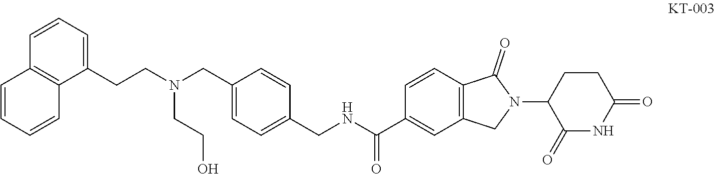 [SMILES: O=C1CCC(N2Cc3cc(C(=O)NCc4ccc(CN(CCO)CCc5cccc6ccccc56)cc4)ccc3C2=O)C(=O)N1]

¹H NMR (400 MHz, DMSO) δ 11.01 (s, 11H), 9.22 (t, J=6.0 Hz, 1H), 8,11 f(s, 1H), 8.03 (d, J=8.1 Hz, 1H), 7.89-7.80 (m, 3H), 7.73 (d, J=7.9 Hz, 1H), 7.45-7.36 (m, 3H), 7.35-7.24 (m, 5H), 5.14 (dd, J=13.3, 5.1 Hz, 1H), 4.58-4.34 (m, 5H), 3.74 (s, 2H), 3.50 (t, J=6.4 Hz, 2H), 3.23-3.14 (m, 2H), 2.98-2.83 (m, 1H), 2.81-2.72 (m, 21), 2.71-2.56 (m, 3H), 2.46-2.32 (m, 1H), 2.06-2.00 (m, 1H). ESI-MS(M+H)⁺: 605.3.

### Example 2

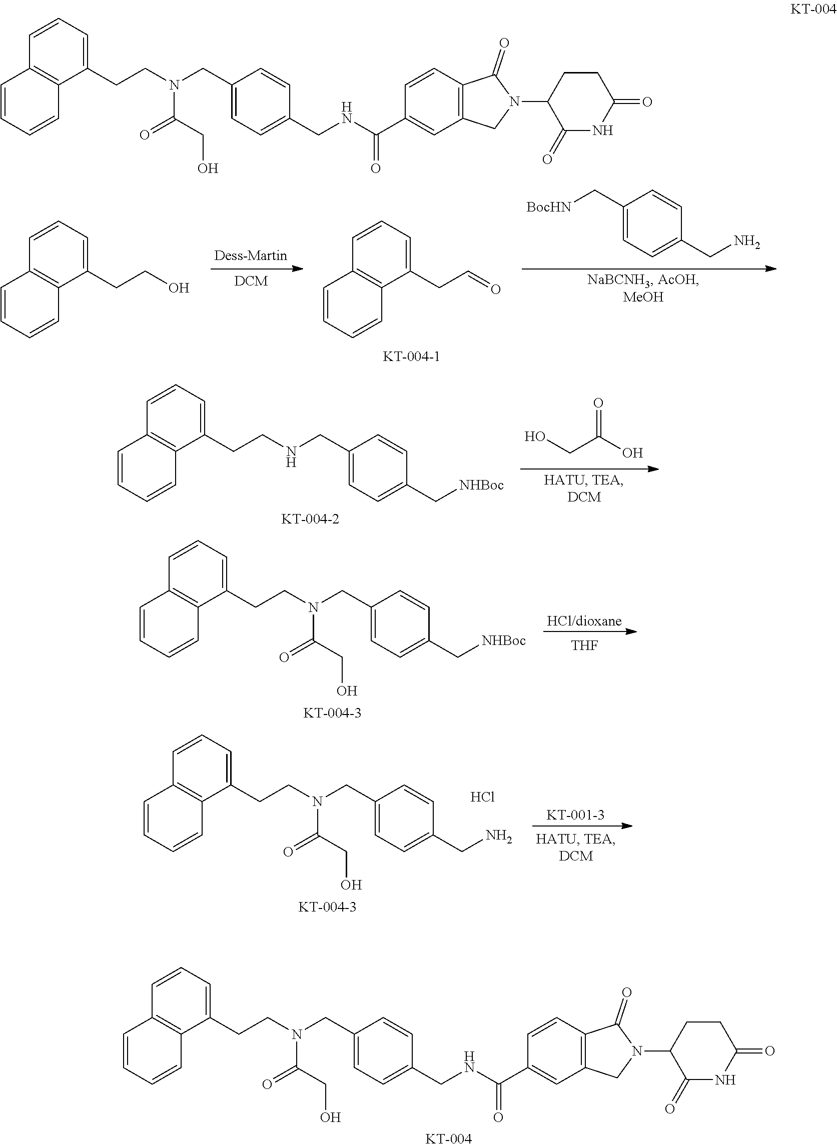

### Synthesis of compound KT-004-1

2-(1-naphthyl)ethanol (860 mg, 5 mmol) was dissolved in 18 mL DCM, to which Dess-Martin reagent (3.18 g, 7.5 mmol) was added in batches at 0° C. After stirring at room temperature for 2 h, TLC showed that the reaction was complete. The mixture was filtered directly. After addition of aqeuous NaHCO₃ (30 mL), the filtrate was extracted with dichloromethane (30 mL*3). The organic phases were combined and dried with anhydrous sodium sulfate. The organic phase was filtered, and the solvent was removed by rotary evaporation. The crude product was purified by column chromatography (petroleum ether: ethyl acetate=-100: 1-15: 1 as eluent) to give compound KT-004-1 (690 mg, 81% yield).

### Synthesis of compound KT-004-2

Compound KT-004-1 (690 mg, 4.1 mmol) and 1-(N-Boc-amuinomethyl)-4-(auinomethyl) benzene (968 mg, 4.1 mmol) were dissolved in 12 mL of methanol. NaBH₃CN (504 mg, 8 mmol) and AcOH (100 mg) were added. After stirring at room temperature for 4 h, TLC showed that the reaction was complete. After addition of aqeuous NaHCO₃ (30 mL), the mixture was extracted with dichloromethane (35 mL*3). The organic phases were combined and dried with anhydrous sodium sulfate. The organic phase was filtered, and the solvent was removed by rotary evaporation. The crude product was purified by column chromatography (dichloromethane: methanol=20: 1 as eluent) to yield compound KT-004-2 (540 mg, 34% yield).

ES1-MS(M+H)⁺: 391.2.

### Synthesis of compound KT-004-3

Compound KT-004-2 (540 mg, 1.38 mmol) and glycolic acid (106 mug, 14 mmol) were dissolved in 10 mL dichloromethane, to which HAT (630 mg, 1.66 mmol) and TEA (420 mug, 4.2 mmol) were added. After stirring at room temperature for 2 h, TLC showed that the reaction was complete. Water (30 mL) was added to quench the reaction and the aqueous phase was extracted with dichloromethane (35 mL*3). After the organic phases were combined, it was dried with anhydrous sodium sulfate. The organic phase was filtered, and the solvent was removed by rotary evaporation. The crude product was purified by column chromatography (dichloromethane: methanol=20: 1 as eluent) to yield compound KT-004-3 (406 mg, 65% yield).

EST-MS(M−H)⁺: 449.2.

### Synthesis of compound KT-004-4

Compound KT-004-3 (406 mg, 0.9 mmol) was dissolved in 4 mL of tetrahydrofuran, to which HCl/Dioxane (3 mL, 4 M) was added. After stirring at room temperature for 4 h, TLC showed that the reaction was complete. The reaction was dried by rotary evaporation to yield compound KT-004-4 (400 mg, crude), which was directly used for next step.

### Synthesis of compound KT-004

Compound KT-004-4 (400 mug, 0.89 mmol) and compound KT-001-3 (259 mg, 0.9 mmol) were dissolved in 10 mL dichloromethane. HATU (410 mg, 1.1 mmol) and triethylamine (270 mg, 2.7 mmol) were added to it. After stirring at room temperature for 1.5 h, TLC showed that the reaction was complete. Water (50 mL) was added to quench the reaction and the aqueous phase was extracted with dichloromethane (35 mL*3). After the organic phases were combined, it was dried with anhydrous sodium sulfate. The organic phase was filtered, and the solvent was removed by rotary evaporation. The crude product was purified by column chromatography (dichloromethane: methanol=15: 1 as eluent) to give a white solid compound KT-004 (127 mg, 23% yield). ¹H NMR (400 MHz, DMSO) δ 11.00 (s, 1H), 9.21 (d, J=4.2 Hz, 1H), 8.14 (d, J=7.9 Hz, 1H), 8.07 (s, 1H), 8.02-7.98 (m, 1H), 7.92-7.88 (m, 1H), 7.83-7.73 (m, 2H), 7.57-7.45 (m, 2H), 7.43-7.27 (m, 4H), 7.24-7.19 (m, 2H), 5.13 (dd, J=13.2, 5.0 Hz, 1H), 4.62 (s, 1H), 4.53-4.32 (m, 5H), 4.16 (s, 1H), 3.96 (s, 1H), 3.53-3.44 (m, 1H), 3.30-3.13 (m, 3H), 3.00-2.84 (m, H), 2.66-2.55 (m, 1H), 2.47-2.27 (m, 1H), 2.05-1.93 (m, 11H). ESi-MS(M+H)⁺: 619.3 KT-016 and KT-033 were synthesized with reference to KT-004

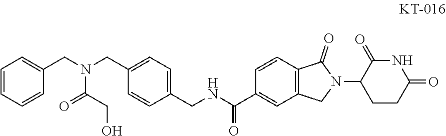 [SMILES: O=C1CCC(N2Cc3cc(C(=O)NCc4ccc(CN(Cc5ccccc5)C(=O)CO)cc4)ccc3C2=O)C(=O)N1]

¹H NMR (400 MHz, DMSO) δ 11.01 (s, 1H), 9.23 (t, J=6.0 Hz, 1H), 8.10 (s, 1H), 8.02 (d, J=8.0 Hz, 1H), 7.82 (d, J=7.9 Hz, 1H), 7.42-7.25 (m, 5H), 7.22-7,15 (m, 4H), 5.14 (did, J 13.3, 5.1 Hz, 1H), 4.75 (hr s, 1H), 4.59-4.32 (m, 8H), 4.17 (d, J=3.6 Hz, 2H), 2.99-2.84 (m, 1H), 2.66-2.54 (m, 1H), 2.47-2.32 (m, 1H), 2.07-1.97 (m, 11H). ES1-MS(M+H)⁺: 555.3.

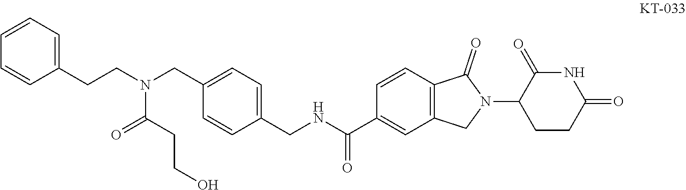 [SMILES: O=C1CCC(N2Cc3cc(C(=O)NCc4ccc(CN(CCc5ccccc5)C(=O)CCO)cc4)ccc3C2=O)C(=O)N1]

¹H NMR (400 MHz, DMSO) δ 11.00 (s, 11H), 9.20 (dd, J=13.9, 6.0 Hz, 11H), 8.09 (s, 1H), 8.00 (d, J=7.9 1z, 1H), 7.81 (d, J=7.9 Hz, 1H), 7.34-7.24 (m, 4H), 7.23-7.15 (1m, 5H), 5.14 (dd, J=13.3, 5.1 Hz, 1H), 4.56-4.35 (m, 7H), 3.69-3.57 (m, 2H), 3.42 (t, J=7.5 Hz, 2H), 2.98-2.85 (m, 1H), 2.81 (t, J=7.6 Hz, 1H), 2.78-2.69 (m, 1H), 2.66-2.54 (m, 1H), 2.48-2.34 (m, 3H), 2.07-1.96 (m, 1H). ESI-MS(M+H)—: 583.4.

### Example 3

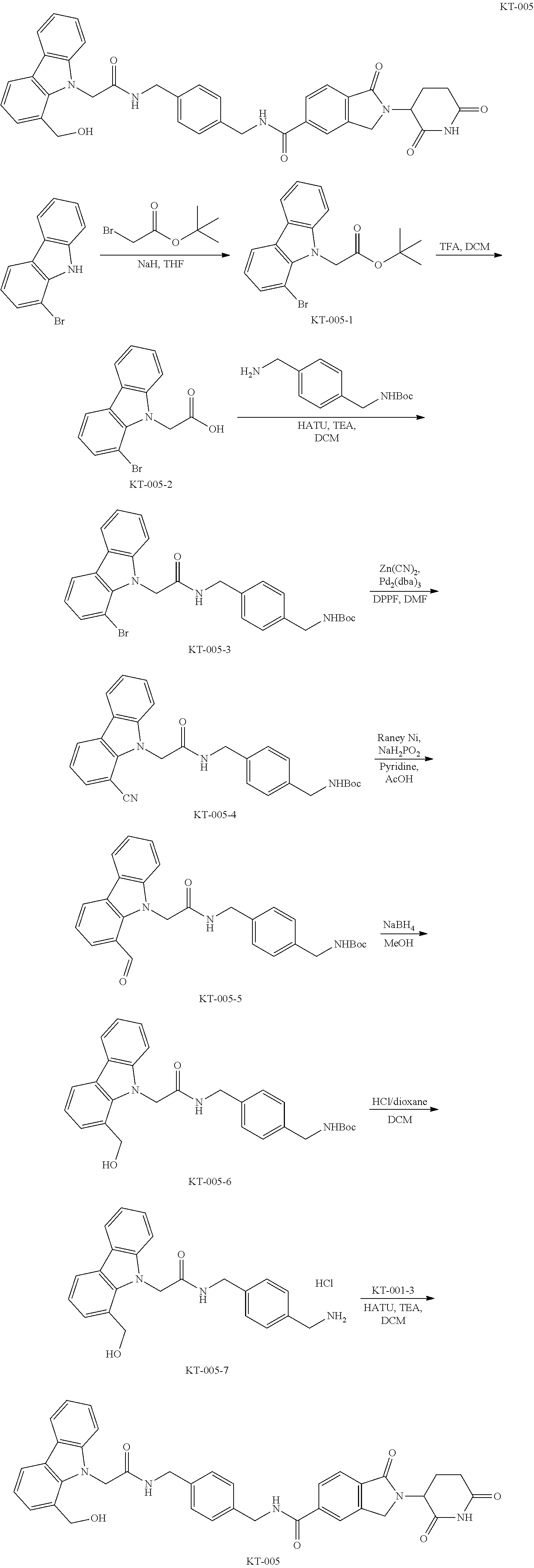

### Synthesis of compound KT-005-1

1-bromocarbazole (1.2 g, 4.9 mmol) was dissolved in 50 ml of THF. NaH (590 mg, 14.6 mmol, 60%) was added to it under an ice bath. After adding, the mixture was stirred at room temperature for 1 h. t-butyl bromoacetate (1.14 g, 5.9 mmol) was added. After stirring at room temperature overnight, TLC showed that the reaction was complete. Water (60 mL) was added to quench the reaction. The aqueous phase was extracted with ethyl acetate (80 mL*3). The organic phases were combined and washed with saturated salt water (20 mL). Then it was dried with anhydrous sodium sulfate and the solvent was removed by rotary evaporation. The crude product was purified by column chromatography (petroleum ether: ethyl acetate=10: 1 as mobile phase) yielded compound KT-005-1 (700 mg, 40% yield).

### Synthesis of compound KT-005-2

Compound KT-005-1 (700 mg, 1.9 mmol) was dissolved in 15 mL of dichloromethane, to which 5 mL of trifluoroacetic acid was added. After stirring at room temperature for 1 h, TLC showed that the reaction was complete. The reaction was dried by rotary evaporation to yield compound KT-005-2 (700 mg, crude product) which was directly used for next step.

### Synthesis of compound KT-005-3

Compound KT-005-2 (700 mg, 1.9 mmol) was dissolved in 40 mL DCM, to which 1-(N-Boc-aminomethyl) -4-(aminomethyl)benzene (560 mug, 2.4 mmol), HATU (1.1 g, 2.9 mmol) and triethylamine (800 mg, 7.9 mmol) were added. After stirring at room temperature overnight, TLC showed that the reaction was complete. After quenched with water (20 mL), the reaction was extracted with dichloromethane (80 mL*3). The organic phases were combined and washed with saturated brine (20 mL). The organic phase was dried with anhydrous sodium sulfate and the solvent was removed by rotary evaporation. The crude product was purified by column chromatography (petroleum ether: ethyl acetate=1: 1 as mobile phase) to give compound KT-005-3 (800 mg, 78% yield). ¹ H NMR (400 MHz, DMSO) δ 8.66 (t, J=6.0 Hz, 1H), 8.22-8.19 (m, 21), 7.59 (dd, J=11.2, 7.7 Hz, 2H), 7.52-7.45 (m, 11H), 7.36 (s, 1H), 7.29-7.25 (m, 1H), 7.22-7.11 (m, 5H), 5.47 (s, 2H), 4.28 (d, J=5.8 Hz, 2H), 4.09 (d, J=5.8 Hz, 2H), 139 (s, 9H).

### Synthesis of compound KT-005-4

Compound KT-005=3 (500 mg, 0.9 mmol), zinc cyanide (230 mg, 1.9 mmol), Pd₂(dba)₃ (170 mg, 0.2 mmol), and LDPPF (200 mg, 0.4 mmol) were dissolved in 18 mL. of DMF. After reacted at 120° C. overnight under nitrogen protection, TLC showed that the reaction was complete. After quenched with water (60 ml), the reaction was extracted with ethyl acetate (60 mL*3). The organic phases were combined and washed with saturated brine (20 mL). The organic phase was dried with anhydrous sodium sulfate and the solvent was removed by rotary evaporation.

The crude product was purified by column chromatography (dichloromethane: methanol=20: 1 as mobile phase) to give compound KT-005-4 (370 mg, 82% yield). H NMR (400 MHz, DMSO) δ 8.82 (t, J=5.7 Hz, 1H), 8.55 (d, J=7.7 1z, 1H), 8.29 (d, J=7.7 Hz, 1 1), 7.87 (d, J:=7.4 Hz, 1H), 7.64 (d, J=8.3 Hz, 1H), 7.57-7.53 (m, 11I), 7.41-7.32 (m, 3H), 7.24 (d, J=8.1 Hz, 2H), 7,17 (d, J=8.1 Hz, 2H), 5.43 (s, 2H), 4.31 (d, J=5.6 Hz, 2H), 4.09 (d, J=6.1 Hz, 2H), 1.36 (s, 9H).

### Synthesis of compound KT-005-5

Compound KT-005-4 (300 mg, 0.6 mol) was dissolved in a mixture of 10 mL pyridine, 3 mL AcOl and 3 mL water. sodium hypophosphite (650 mug, 6.0 mmol) and Raney nickel (1 g) were added. After reacted at room temperature overnight, TLC showed that the reaction was complete. The reaction mixture was filtered. After added to saturated sodium bicarbonate (40 mL), the filtrate was extracted with ethyl acetate (80 mL*3). The organic phases were combined and washed with saturated brine (20 ml-2). The organic phase was dried with anhydrous sodium sulfate and the solvent was removed by rotary evaporation to give compound KT-005-5 (350 mg, crude) which was directly used for next step. ESI-MS(M−H)⁺: 472.3.

### Synthesis of compound KT-005-6

Compound KT-005-5 (350 mg, 0.6 mmol) was dissolved in 25 mL of methanol, to which sodium borohydride (40 mg, 1.1 mmol) was added under ice bath. After reacted at room temperature for 2 h, TLC showed that the reaction was complete. The reaction was quenched by adding water (20 mL) and extracted with dichloromethane (60 mL*3). The organic phases were combined and washed with saturated brine (20 mL). The organic phase was dried with anhydrous sodium sulfate and the solvent was removed by rotary evaporation. The crude product was purified by column chromatography (dichloromethane: methanol=20: 1 as mobile phase) to give compound KT-005-6 (80 mg, 26% yield). ES1-MS(M−H₂O+H)⁺: 456.3.

### Synthesis of compound KT-005-7

Compound KT-005-6 (80 mg, 0.17 mmol) was dissolved in 5 mL of dichloromethane, to which HCl/dioxane (3 mL, 4 M) was added. After reacted at room temperature for 1 h, TLC showed that the reaction was complete. The solvent was removed by rotary evaporation to give compound KT-005-7 (80 mug, crude) which was directly used for next step.

### Synthesis of compound KT-005

Compound KT-005-7 was dissolved in 15 mL DCM, to which compound KT-001-3 (50 mg, 0.17 mmol), HATU (90 rug, 0.24 mmol) and triethylamine (65 mg, 0.6 mmol) were added. After reacted at room temperature overnight, TLC showed that the reaction was complete. Water (20 mL) was added to quench the reaction and the aqueous phase was extracted with dichloromethane (80 mL*3). The organic phases were combined and washed with saturated brine (20 nL). The organic phase was dried with anhydrous sodium sulfate and the solvent was removed by rotary evaporation. The crude product was purified by column chromatography (dichloromethane: methanol=10: 1 as mobile phase) to yield compound KT-005 (24 mg. 23% yield). ¹H NMR (400 MHz, DMSO) δ 11.01 (s, 1H), 9.22 (t, J=5.9 Hz, 1H), 8.71 (t, J=5.9 Hz, 1H), 8.18-8.10 (m, 2H), 8.08 (s, 1H), 8.00 (d, J=8.0 Hz, 1H), 7.82 (d, J=7.9 Hz, 1H), 7.48 (d, J=8.2 Hz, 1H), 7.45-7.40 (m, 1H), 7.33 (d, J=6.6 Hz, 1H), 7.28 (d, J=8.1 Hz, 2H), 7.24-7.18 (m, 3H), 7.17-7.12 (m, 1H), 5.55 (t, J=5.3 Hz, 1H), 5.41 (s, 2H), 5.13 (dd, J=13.3, 5.1 Hz, 1H), 4.77 (d, J=5.3 Hz, 2H), 4.55-4,37 (m, 4H), 4.28 (d, J=5.8 Hz, 2H), 2.99-2.85 (m, 1W), 2.68-2.56 (m, 1H), 2.46-2.32 (m, 1H), 2.12-1.99 (, 1H). EST-MS(M-H₂O+iH)⁺: 626.3.

### Example 4

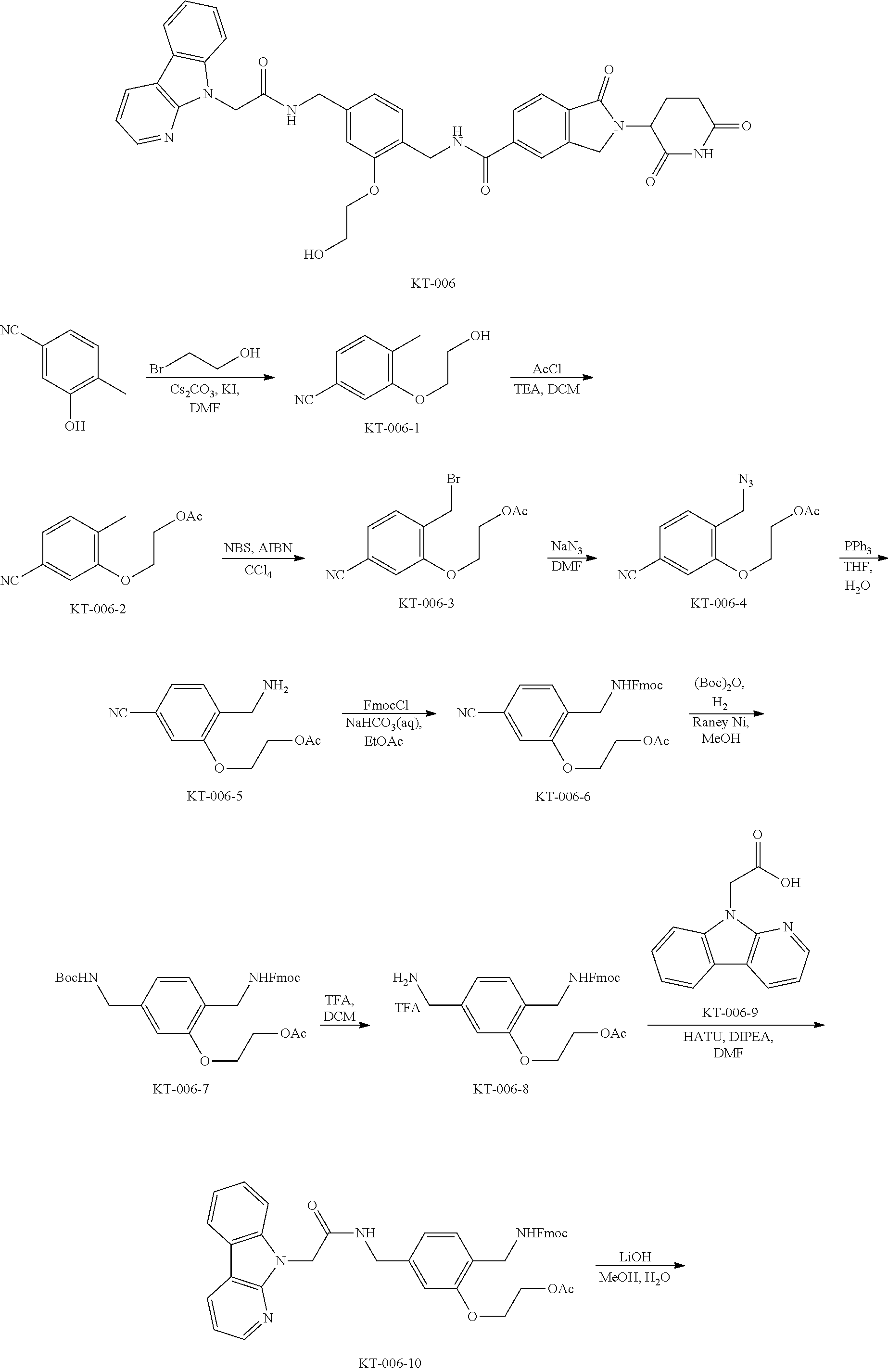

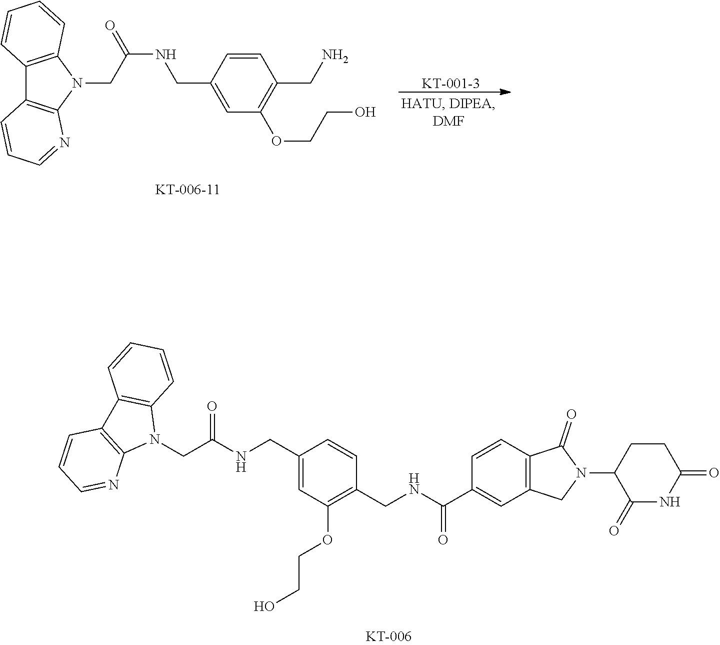 [SMILES: NCc1ccc(CNC(=O)Cn2c3ccccc3c3cccnc32)cc1OCCO ; "KT-001-3" ; O=C(Cn1c2ccccc2c2cccnc21)NCc1ccc(CNC(=O)c2ccc3c(c2)CN(C2CCC(=O)NC2=O)C3=O)c(OCCO)c1]

### Synthesis of compound KT-006-1

3-hydroxy-4-methylbenzonitrile (2.66 g, 20 mmol) was dissolved in DMF (30 ml), to which bromoethanol (3.75 g, 30 mmol) and potassium carbonate (8.28 g, 60 mmol) were added. After reacted at 80° C. for 3 h, TLC showed that the reaction was complete. The reaction solution was cooled to room temperature and water (300 ml) was added. The mixture was extracted with ethyl acetate (100 mL*2). The organic phases were combined and washed with saturated brine (50 mL*2). The organic phase was dried and the solvent was removed by rotary evaporation to yield KT-006-1 (2.55 g, 72% yield). ¹H NMR (400 MHz, DMSO) 7.37 (s, 1H), 7.34-7.29 (m, 2H), 4.88 (t, J=4.0 Hz, 1H), 4.07 (t, J=4.0 Hz, 2H), 2.23 (s, 3H).

### Synthesis of compound KT-006-2

Compound KT-006-1 (2.55 g, 14.4 mmol) was dissolved in DCM (30 ml), to which TEA (2.91 g, 28.8 mmol) was added. Acetyl chloride (1.36 g, 17.3 mmol) was added slowly under an ice bath. After addition, the mixture was warmed to room temperature. After reacted at room temperature for another 3 h, TLC showed that the reaction was complete. Ice water (100 mil) was added and the mixture was extracted with ethyl acetate (100 mL*2). The organic phases were combined and washed with saturated brine (50 mL*2). The organic phase was dried and the solvent was removed by rotary evaporation. The crude product was purified by column chromatography (petroleum ether: ethyl acetate=5: 1) to give KT-006-2 (2.65 g, 85% yield).

### Synthesis of compound KT-006-3

Compound KT-006-2 (2.65 g, 12.1 mmol) was dissolved in carbon tetrachloride (40 mi), to which NBS (2.15 g, 12.1 mmol) and AIBN (0.40 g, 2.42 mmol) were added. After reacted at 80° C. for 3 h, TLC showed that the reaction was complete. The reaction solution was dried by rotary evaporation. The crude product was purified by column chromatography (petroleum ether: ethyl acetate=4: 1) to give KT-006-3 (2.25 g, 63% yield).

### Synthesis of compound KT-006-4

Compound KT-006-3 (1.49 g, 5.0 mmol) and NaN-(0.325 g, 5.0 mmol) were dissolved in DMF (15 nil). After reacted at room temperature for 3 h, TLC showed that the reaction was complete. Water (50 ml) was added to quench the reaction. The aqueous phase was extracted with ethyl acetate (100 mL*2). The organic phases were combined and washed with saturated brine (50 mL*2). The organic phase was dried and the solvent was removed by rotary evaporation to yield compound KT-006-4 (1.3 g, 100% yield).

### Synthesis of compound KT-006-5

Compound KT-006-4 (1.3 g, 5.0 mmol) and triphenylphosphine (1.31 g, 5.0 mmol) were dissolved in THE H₂) (20 ml/5 ml). After reacted at room temperature for 5 h, TLC showed that the reaction was complete. Diluted hydrochloric acid (1 M, 50 nil) was added to quench the reaction. After the aqueous phase was extracted with ethyl acetate (50 ml*2), the aqueous phase was adjusted to p11=10 with a saturated sodium carbonate solution. The aqueous phase was then extracted with ethyl acetate (50 ml*3). The organic phase were combined and dried. The solvent was removed by rotary evaporation to yield compound KT-006-5 (1.3 g, 100% yield).

ESI-MS(M+H)⁺: 235.2.

### Synthesis of compound KT-006-6

Compound KT-006-5 (1.17 g, 5.0 mmol) was dissolved in ethyl acetate (50 ml), to which saturated sodium bicarbonate solution (20 ml) was added. FmocCl (2.1 g, 7.5 mmol) was added to it under an ice bath. After reacted at room temperature for 3 h, TLC showed that the reaction was complete. Water (50 ml) was added to quench the reaction. The aqueous phase was extracted with ethyl acetate (100 nm*2). The organic phase were combined and dried. The solvent was removed by rotary evaporation. The crude product was purified by column chromatography (petroleum ether: ethyl acetate=1: 1) to give compound KT-006-6 (1.3 g, 57%). ES1-MS(M+H)⁺: 457.2.

### Synthesis of compound KT-006-7

Compound KT-006-6 (1.3 g, 2.85 mmol) was dissolved in 40 ml methanol, to which (Boc)₂₀ (1.24 g, 5.,7 mmol) and Rany nickel (2.0 g) were added. After reacted at room temperature for 2 h under hydrogen atmosphere, TLC showed that the reaction was complete. The reaction solution was filtered and the filtrate was dried by rotary evaporation to yield compound KT-006-7 (1,15 g, 72% yield). ES1-MS(M+H)⁺: 561.2.

### Synthesis of compound KT-006-8

Compound KT-006-7 (1.15 g, 2.1 mmol) was dissolved in 20 ml dichloromethane, to which trifluoroacetic acid (4 nil) was added. After reacted at room temperature for 2 h, TLC showed that the reaction was complete. The solvent was dried by rotary evaporation to yield compound KT-006-8 (13 g, crude) which was directly used for next step.

### Synthesis of compound KT-006-10

Compound KT-006-8 (0.97 g, 2.1 mmol), compound KT-006-9 (475 mg, 2.1 mmol) and DIPEA (0.81 g, 6.3 mmol) were dissolved in DMF (20 nil). HAT U (0,96 g, 2.52 mmol) was added slowly under an ice bath. After reacted at room temperature for 2 h, TLC showed that the reaction was complete. Ice water (100 ml) was added to quench the reaction. The aqueous phase was extracted with ethyl acetate (200 mL*2). The organic phases were combined and washed with saturated brine (50 mL*2). The organic phase was dried and the solvent was removed by rotary evaporation. The crude product was purified by column chromatography (petroleum ether: ethyl acetate=1:1) to give compound KT-006-10 (1.05 g, 75% yield). ESI-MS(M+1H): 669.3.

### Synthesis of compound KT-006-11

Compound KT-006-10 (335 mg, 0.5 mmol) was dissolved in 30 mL methanol, to which an aqueous solution of lithium hydroxide (5 nil, 2 M) was slowly added under an ice bath. After reacted at room temperature for 16 h, TLC showed that the reaction was complete. Water (100 ml) was added to dilute the mixture. The aqueous phase was extracted with ethyl acetate (100 mL*3) and then the organic phases were washed with saturated salt water (50 mL*2). The organic phase was dried and the solvent was removed by rotary evaporation. The crude product was purified by column chromatography (petroleum ether: ethyl acetate=1: 1) to give compound KT-006-11 (202 mg, 100% yield).

### Synthesis of compound KT-006

Intermediate KT-006-11 (202 mg, 0.5 mmol), KT-001-3 (144 mg, 0.5 mmol) and DIPEA(194 mg, 1.5 nmol) were dissolved in 10 mL DMF. HATU (228 mg, 0.6 mmol) was added slowly under an ice bath. After reacted at room temperature for 2 h, TLC showed that the reaction was complete. Ice water (100 ml) was added to quench the reaction. The aqueous phase was extracted with ethyl acetate (200 mL*2). The organic phases were combined and washed with saturated brine (50 mL*2). The organic phase was dried and the solvent was removed by rotary evaporation. The crude product was purified by column chromatography (dichloromethane: methanol=15: 1) to give compound KT-006 (22 mg, 7% yield). “H NMR (400 MHz, DMSO) δ 11.01 (s, 11H), 8.94 (t, J=5.8 Hz, 1H1), 8.72 (t, J=5.9 Hz, 1H), 8.55 (dd, J=7.6, 1.4 Hz, 1H), 8.44 (dd, J=4.8, 1.4 Hz, 1H), 8.21 (d, J=7.7 Hz, 1H), 8.09 (s, 1H), 8.00 (d, J=8.1 Hz, 1H), 7.82 (d, J=7.9 Hz, 1H), 7.58 (d, J=8.2 Hz, 1H), 7.49 (t, J=7.7 Hz, LH), 7.32-7.23 (m, 2H), 7.16 (d, J=7.7 Hz, 1H), 6.87 (s, 1H), 6,82 (d, J=7.8 Hz, 1H), 5.20-5.10 (m, 3H), 4.94 (t, J=5.7 Hz, 1H), 4.47 (dt, J=41.6, 17.7 z, 4H), 4.28 (d, J=5.8 Hz, 2H), 3.99 (t, J=4.9 Hz, 2H), 3.76 (dd, J=10.2, 5.2 Hz, 2H), 2.99-2.85 (m, 1H), 2.65-2.56 (m, 1H), 2.47-2.36 (m, 1H), 2.06-1.95 (m, 1H). ESI-MS(M−H)⁺: 675.3.

### Example 5

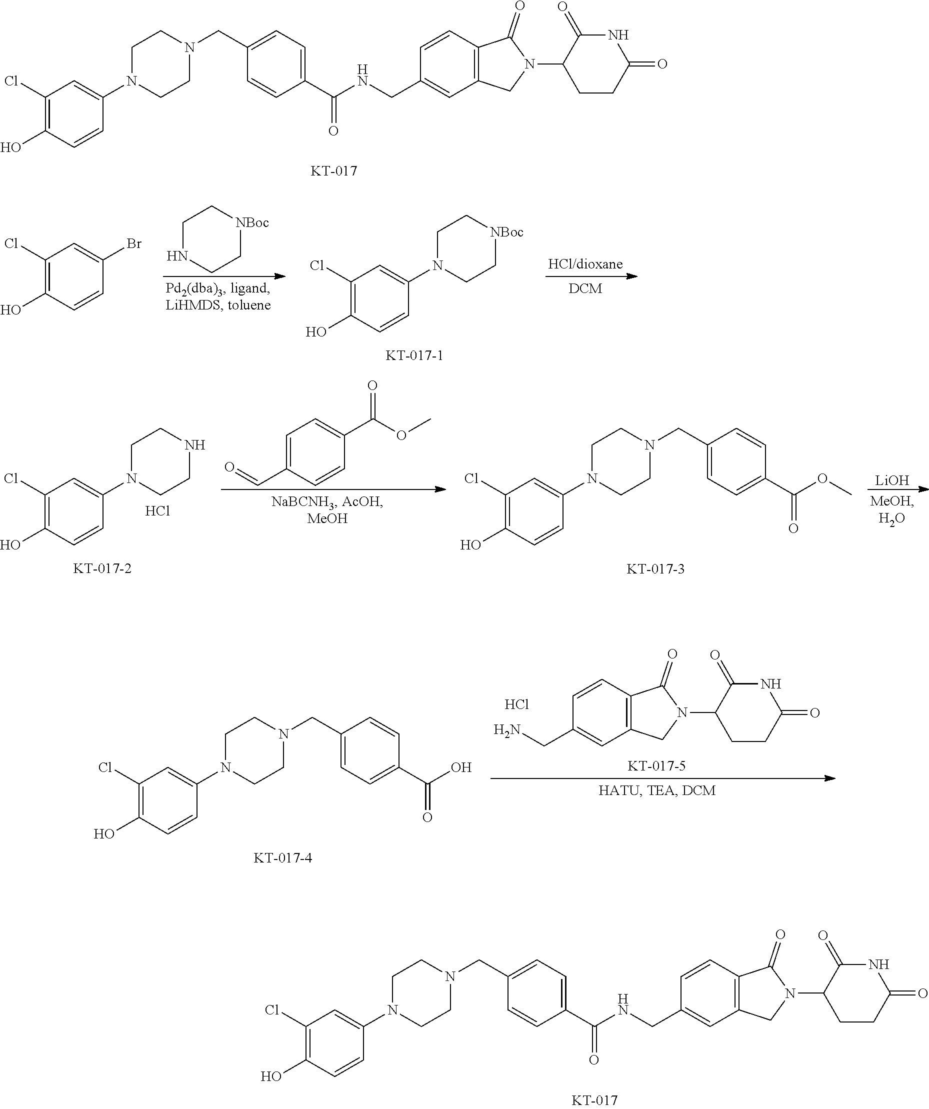

### Synthesis of compound KT-017-1

4-bromo-2-chlorophenol (600 mg, 2.9 mmol) and N-Boc-piperazine (680 mg, 3.6 mmol) were dissolved in 30 mL toluene, to which LiHMDS (6.6 mL, 1 M), Pd₂(dba)3 (120 mg, 0.15 mmol) and 2,8, 9-triisobutyl-2,5,8, 9-tetraaza-1-phosphate bicyclic [3.3.3]undecane (120 mg,0.3 mmol) were added. After reacted at 80° C. overnight under nitrogen protection, TLC showed that the reaction was complete. The reaction was quenched with water (60 ml). The aqueous phase was extracted with ethyl acetate (60 mL*3). The organic phases were combined and washed with saturated brine (20 mL). The organic phase was dried with anhydrous sodium sulfate and the solvent was removed by rotary evaporation. The crude product was purified by chromatography column (petroleum ether: ethyl acetate=4: 1 as mobile phase) to yield compound KT-017-1 (270 mg, 30% yield). ESI-MS(M+H)⁺: 313.2.

### Synthesis of compound KT-017-2

KT-017-1 (270 mg, 0.86 mmol) was dissolved in 5 mL dichloromethane, to which HCl/dioxane (5 mL, 4 M) was added. After reacted at room temperature for I h, TLC showed that the reaction was complete. The solvent was removed by rotary evaporation to give compound KT-017-2 (280 mg, crude) which was used directly for next step.

Synthesis of compound KT-017-3 Compound KT-017-2 (280 mg, crude) and methyl p-formyl benzoate (170 mug, 1.0 mmol) were dissolved in 18 mL methanol, to which a catalytic amount of acetic acid was added. After reacted at room temperature for 2 h, sodium cyanoborohydride (110 mg, 1.7 mmol) was added. After reacted at room temperature overnight, TLC showed that the reaction was complete. Water (60 mL) was added to quench the reaction and the aqueous phase was extracted with dichloromethane (60 mL*3). The organic phases were combined and washed with saturated brine (20 mL). The organic phase was dried with anhydrous sodium sulfate and the solvent was removed by rotary evaporation. The crude product was purified by column chromatography (petroleum ether: ethyl acetate=1: 1 as mobile phase) to give compound KT-017-3 (160 mg, 50% yield). ESI-MS(M+H)-: 361.2.

### Synthesis of compound KT-017-4

Compound KT-017-3 (80 mg, 0.2 mmol) was dissolved in methanol/water (12 m L/4 mL), to which lithium hydroxide (30 mug, 0.6 mmol) was added. After reacted at 60′C for 1 h, TLC showed that the reaction was complete. The reaction solution was cooled to room temperature and adjusted pH to 4 with 2 N diluted hydrochloric acid. The solvent was removed by rotary evaporation to give the compound KT-017-4 (150 mg, crude) which was used directly for next step.

### Synthesis of compound KT-017

Compounds KT-017-4 (150 mg, crude) and KT-017-5 (86 mg, 0.28 mmol) were dissolved in 20 mL. dichloromethane. HATU (130 mg, 0.35 mmol) and triethylamine (95 mg, 0.9 mmol) were added. A After reacted at room temperature overnight, TLC showed that the reaction was complete. Water (20 mL) was added to quench the reaction and the aqueous phase was extracted with DCM (80 mL*3). The organic phases were combined and washed with saturated brine (20 mL), and then dried with anhydrous sodium sulfate and concentrated. The crude product was purified by column chromatography (dichloromethane: methanol=20:1 as mobile phase) to yield compound KT-017 (7 mg, 5% yield). ^{I}-NMR (400 MHz, DMSO) δ 10.96 (s, 1H), 9.40 (br s, 1H), 9.10 (t, J=5.9 Hz, 1H), 7.88 (d, J=8.2 Hz, 2H), 7.69 (d, J=7.8 Hz, 1H), 7.54 (s, 1H), 7.47-7.41 (i, 3H), 6.88-6.80 (m, 2H), 6.75 (dd, J=8.9, 2.8 Hz, H), 5.09 (dd, J=13.3, 5.1 Hz, 1H), 4.59 (d, J=5.7 Hz, 2H), 4.37 (dd, J=53.7, 17.3 Hz, 2H), 3.57 (s, 2H), 3.04-2.96 (m, 4H), 2.94-2.85 (m, 1H), 2.64-2.56 (m, 1H), 2.49-2.47 (m, 4H), 2.43-2.31 (m, 1H), 2.04-1.97 (m, 1H). ESI-MS(M+H)⁺: 602.3.

KT-018 was synthesized according to KT-017

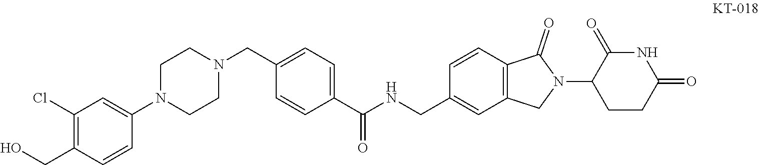 [SMILES: O=C1CCC(N2Cc3cc(CNC(=O)c4ccc(CN5CCN(c6ccc(CO)c(Cl)c6)CC5)cc4)ccc3C2=O)C(=O)N1]

¹H NMR (400 MHz, DMSO) δ 10.97 (s, 1H), 9.11 (t, J=5.9 Hz, 1H), 7.88 (d, J=8.2 Hz, 2H), 7,69 (d, J=7.8 Hz, 1H), 7.54 (s, H), 7.47-7.42 (m, 3H), 7.32 (d, J=9.2 Hz, 1H), 6.92-6.86 (m, 2H), 5.14-5.06 (m, 2H), 4.59 (d, J=5.91 z, 2H), 4.48-4.24 (m, 4H), 3.58 (s, 2H), 3.18-3.08 (m, 4H), 2.97 2.83 (m, H), 2.64-2.55 (m, 1H), 2.49-2.48 (m, 4H), 2.43-2.30 (m, 1H), 2.08-1.88 (m, 1H). ESI-MS(M+H)⁺: 616.3.

The corresponding intermediate KT-018-2 of KT-018 was synthesized as follows:

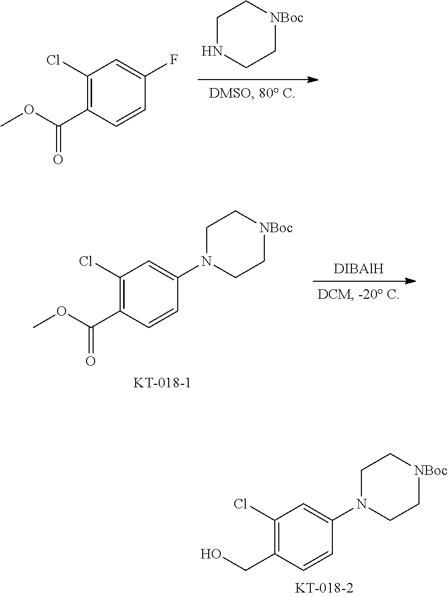

### Synthesis of compound KT-018-1

Compound 2-chloro-4-fluorobenzoate methyl ester (3.0 g, 16.0 mmol) and N-Boc piperazine (3.56 g, 19.1 mmol) were dissolved in 20 mL DMSO. A After reacted at 80° C. for 2 h, TLC showed that the reaction was complete. The reaction was quenched with water (60 mL) and the aqueous phase was extracted with ethyl acetate (60 mL*3). The organic phases were combined and washed with saturated brine (10 mL*3), and then dried with anhydrous sodium sulfate and concentrated. The crude product was purified by column chromatography (petroleum ether: ethyl acetate=5:1 as mobile phase) to give compound KT-018-1 (4.1 g, 72% yield).

### Synthesis of compound KT-018-2

Compound KT-018-1 (2.20 g, 6.21 mmol) was dissolved in 10 mL dichloromethane. Aluminum diisobutyl hydride (12 mL, 1 M, 12 mmol) was added droply to it at −20° C. After reacted at −20° C. for 2 h, TLC showed that the reaction was complete. Saturated ammonium chloride solution (60 mL) was added to quench the reaction, and the aqueous phase was extracted with dichloromethane (50 nL*3). The organic phases were combined and washed with saturated brine (10 mL*3), and then dried with anhydrous sodium sulfate and concentrated. The crude product was purified by column chromatography (petroleum ether: ethyl acetate=3:1 as mobile phase) to give compound KT-018-2 (1.4 g, 69% yield).

### Example 6

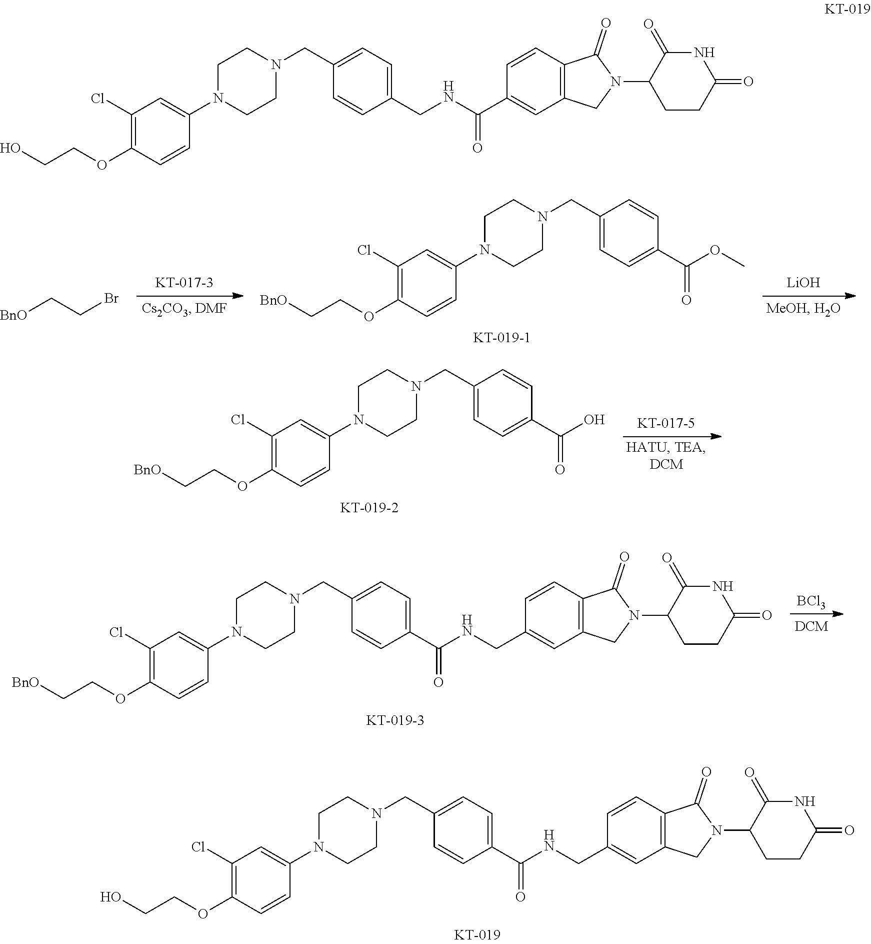

### Synthesis of compound KT-019-1

Compound KT-017-3 (80 mg, 0.2 mmol) and compound benzyl 2-bromoethyl ether (75 mg, 0.3 mol) were dissolved in 12 nL DMF, to which cesium carbonate (145 mg, 0.4 mmol) was added. After reacted at 90° C. for 2 h, TLC showed that the reaction was complete. The reaction was quenched with water (60 mL) and the aqueous phase was extracted with ethyl acetate (60 mL*3). The organic phases were combined and washed with saturated brine (10 mL*3), and then dried with anhydrous sodium sulfate and concentrated. The crude product was purified by column chromatography (petroleum ether: ethyl acetate=4: 1 as mobile phase) to give compound KT-019”-1 (85 mg, 77% yield). ESl-MS(M+H)⁺: 495.2. KT-018 was synthesized according to KT1-017

KT-019-2 and KT-019-3 were synthesized according to KT-017-4 and KT-017

### Synthesis of compound KT-019

Compound KT-019-3 (25 mg, 0.034 mmol) was dissolved in 10 mL DCM, to which BCH₃ (1 nL, 1 M) solution was added under an ice bath. After reacted at room temperature for 1 h, TLC showed that the reaction was complete. Methanol (5 mL) was added to quench the reaction and then the solvent was removed by rotary evaporation. The crude product was purified by column chromatography (dichloromethane: methanol=20: 1 as mobile phase) to yield compound KT-019 (15 mg, 68% yield). ¹H NMR (400 MHz, DMSO) δ 9.11 (t, J=6.0 Hz, 1H), 7.88 (d, J=8.1 Hz, 2H), 7.69 (d, J=7.8 Hz, 1H), 7.54 (s, 1H), 7.48-7.42 (m, 3H), 7.02 (d, J=9.0 Hz, 1H), 6.97 (d, J=2.8 Hz, 1H), 6.84 (dd, J=9.0, 2.8 Hz, 1H), 5.09 (dd, J=13.3, 5.1 Hz, 1H), 4.59 (d, J=5.8 Hz, 2H), 4.37 (dd, J=53.8, 17.3 11z, 2H), 3.96 (t, J=5.1 Hz, 2H), 3.69 (t, J=5.1 Hz, 2H), 3.57 (s, 2H), 3.10-3.00 (m, 4H), 2.95-2.85 (m, 1H), 2.64-2.54 (m, 1H), 2.49-2.48 (m, 4H), 2.43-2.30 (m, 1H) 2.04-1.97 (m, H). ESI-MS(M+H)⁺: 646.3.

### Example 7

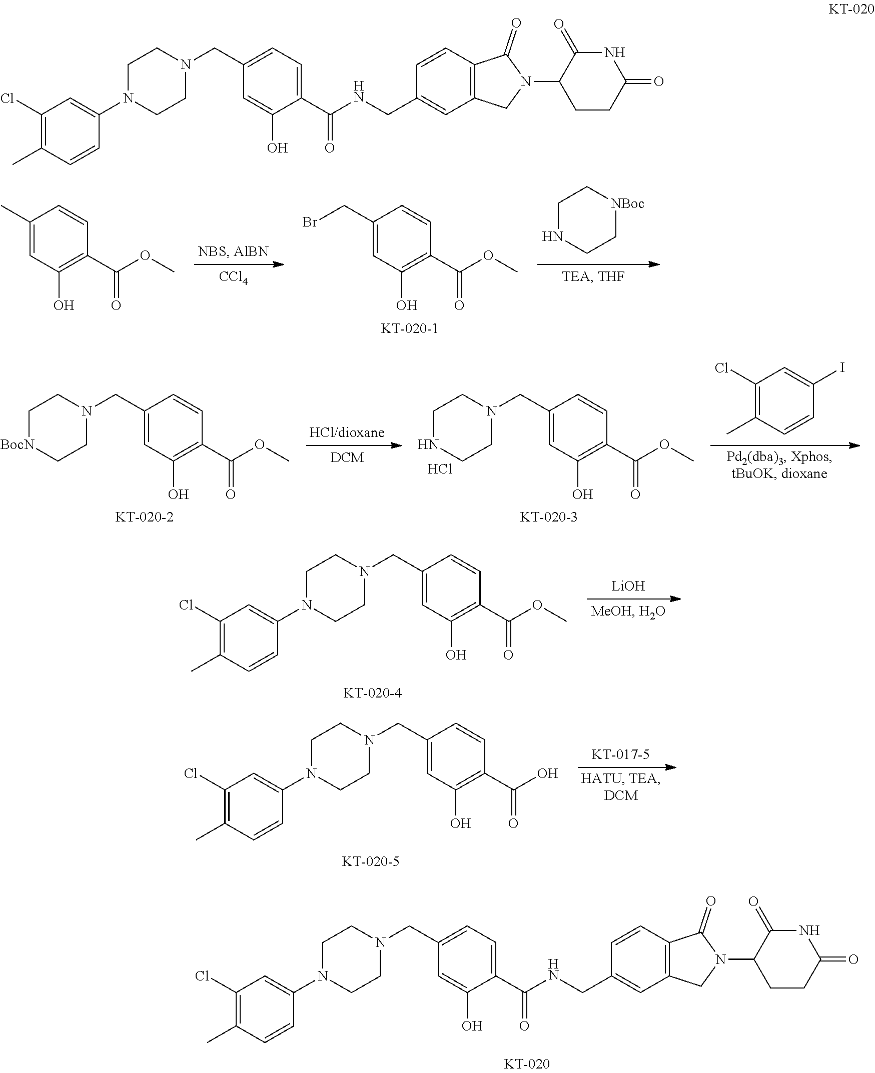

### Synthesis of compound KT-020-1

The compound 4-methylsalicylate methyl ester (500 mg, 3.0 mmol) was dissolved in 15 mL carbon tetrachloride, to which NBS (587 mg, 3.3 mmol) and AIBN (148 mg, 0.9 nmol) were added. After reacted at 80° C. overnight, TLC showed that the reaction was complete. Water (40 mL) was added to quench the reaction and the aqueous phase was extracted with dichloromethane (20 mL*3). The organic phases were combined and washed with saturated brine (10 mL*3). The organic phase was dried with anhydrous sodium sulfate and the solvent was removed by rotary evaporation to yield compound KT-020-1 (800 Ing, crude), which was directly used for next step.

### Synthesis of compound K1-020-2

Compound KT-020-1 (800 mg, crude) and N-Boc piperazine (558 mg. 3.0 mmol) were dissolved in 8 mL of tetrahydrofuran, to which triethylamine (303 mg, 3.0 mmol) was added.

After reacted at room temperature for 2 h, TLC showed that the reaction was complete. Water (40 mL) was added to quench the reaction and the aqueous phase was extracted with ethyl acetate (30 mL*3). The organic phases were combined and washed with saturated salt water (10 mL*3). The organic phase was dried with anhydrous sodium sulfate and the solvent was removed by rotary evaporation. The crude product was purified by column chromatography (petroleum ether: ethyl acetate=6: 1 as mobile phase) to yield compound KT-020-2 (544 mug, 52% yield). ESI-MS(M+H)7: 351.2.

### Synthesis of compound KT-020-3

KT1-020-2 (144 mg, 0.41 mmol) was dissolved in 5 mL dichloromethane, to which HCl/dioxane (5 mL, 4 M) was added. After reacted at room temperature for 1 h, TLC showed that the reaction was complete. The solvent was removed by rotary evaporation to give compound KT-020-3 (150 mg, crude) which was used directly for next step.

### Synthesis of compound KT-020-4

KT-020-3 (150 mg, crude) and 2-chloro-4-iodine-methylbenzene (103 mg, 0.41 mmol) were dissolved in 6 mL dioxane, to which potassium tert-butyl alcohol (92 mg, 0.82 mmol), Pd₂(dba)₃ (56 mg, 0.062 mmol) and Xphos (39 mg, 0.082 mmol) were added. After reacted at 100° C. overnight under nitrogen protection, TLC showed that the reaction was complete. Water (40 20 mL) was added to quench the reaction and the aqueous phase was extracted with ethyl acetate (40 mL*3). The organic phase were combined and washed with saturated brine (20 mL). The organic phase was dried with anhydrous sodium sulfate and concentrated. The crude product was purified by chromatography column (petroleum ether: ethyl acetate=2: 1 as mobile phase) to give compound KT-020-4 (50 mug, 80% yield). ESI-MS(M−H)⁺: 375.2.

KT1-020-5 and KT-020 were synthesized according to KT-017-4 and KT-017

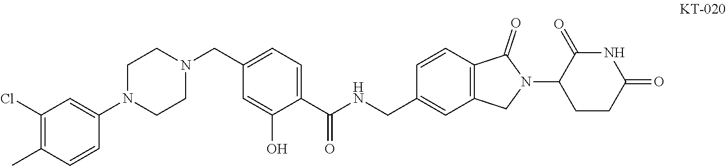 [SMILES: Cc1ccc(N2CCN(Cc3ccc(C(=O)NCc4ccc5c(c4)CN(C4CCC(=O)NC4=O)C5=O)c(O)c3)CC2)cc1Cl]

¹H NMR (400 MHz, DMSO) δ 10.97 (s, LH), 9.39 (t, J=5.9 Hz, 1H), 7.88 (d, J=8.1 Hz, LH), 7.71 (d, J=7.7 Hz, 1H), 7.56 (s, 1Hi), 7.48 (d, J=7.4 Hz, 1H1), 7.15 (d, J=8.5 1z, 1H), 6.94-6.86 (m, 3H), 6.82 (dd, J=8.4, 2.5 Hz, 1), 5.10 (dd, J=13.3, 5.1 Hz, 1H), 4.63 (d, J=6.1 Hz, 2H), 4.39 (dd, J=54.6, 17.5 Hz, 2H), 3.51 (s, 2H), 3.16-3.09 (m, 4H), 2.97-2.85 (m, H), 2.68-2.56 (m, 1H), 2.49-2.48 (m, 4H), 2.42-2.31 (m, 1H), 2.21 (s, 3H), 2.07-1.97 (m, 1H).

ESi-MS(M−H)⁺: 616.3

KT-021 was synthesized according to KT-020

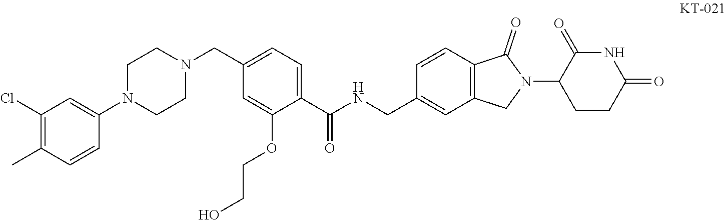 [SMILES: Cc1ccc(N2CCN(Cc3ccc(C(=O)NCc4ccc5c(c4)CN(C4CCC(=O)NC4=O)C5=O)c(OCCO)c3)CC2)cc1Cl]

¹H NMR (400 MHz, DMSO) δ 10.97 (s, H), 8.94 (t, J=6.0 Hz, 1H), 7.79 (d, J=7.9 Hz, 1H), 7.69 (d, J=7.8 Hz, 1H), 7.57 (s, 1H), 7.49 (d, J=7.9 Hz, 1H), 7.19-7.11 (m, 2H), 7.03 (d, J=8.0 Hz, 1H), 6.92 (d, J=2.4 Hz, 1H), 6.82 (dd, J=8.5, 2.5 Hz, 1H), 5.14-5.04 (m, 2H), 4.62 (d, J=6.0 Hz, 2H), 4.37 (dd, J=53.2, 17.4 Hz, 2H), 4.19 (t, J=4.7 Hz, 2H), 3.81-3.74 (m, 2H), 3.56 (s, 2H), 3.17-3.06 (m, 4H), 2.96-2.84 (m, 1H), 2.65-2.55 (m, H), 2.52-2.51 (m, 4H), 2.44-2,31 (m, 1H), 2.20 (s, 3H), 2.04-1.93 (m, 1H). ESI-MS(M+H)⁺: 660.3.

The corresponding intermediate KT-021-1 of KT-021 was synthesized as follows:

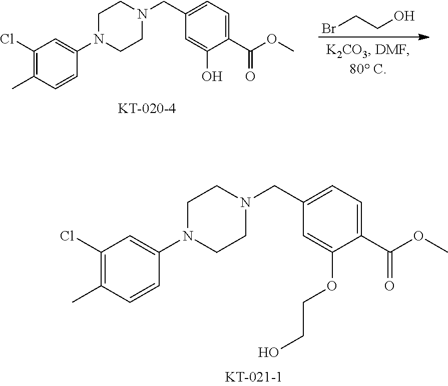

Compound KT-020-4 (96 mg, 0.26 mmol) and compound 2-bromoethanol (38 mug, 0.3 mmol) were dissolved in 5 mL DMF, to which potassium carbonate (55 mg, 0.4 mmol) was added. After reacted at 80° C. for 2 h, TLC showed that the reaction was complete. Water (20 mL) was added to quench the reaction and the aqueous phase was extracted with ethyl acetate (20 mL*3). The organic phases were combined and washed with saturated brine (10 mL*3), and then dried with anhydrous sodium sulfate and concentrated. The crude product was purified by column chromatography (petroleum ether: ethyl acetate=4: 1 as mobile phase) to give compound KT-021-1 (26 mg, 24% yield).

KT-022 was synthesized according to the KT-020

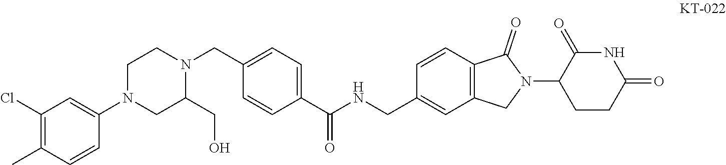 [SMILES: Cc1ccc(N2CCN(Cc3ccc(C(=O)NCc4ccc5c(c4)CN(C4CCC(=O)NC4=O)C5=O)cc3)C(CO)C2)cc1Cl]

¹H NMR (400 MHz, DMSO) δ 10.97 (s, 1H), 9.09 (t, J=6.0 Hz, 1H), 7.87 (d, J=7.9 Hz, 2H), 7.69 (d, J=7.8 Hz, 1H), 7.54 (s, 1H), 7.47-7.43 (m, 3H), 7.15 (d, J=8.5 Hz, 1H), 6.90 (s, 1H), 6.81 (dd, J=8.5, 2.1 Hz, 1H), 5.10 (dd, J=13.3, 5.1 Hz, 1H), 4.68 (s, 1H), 4.59 (d, J=5.8 Hz, 2H), 4.37 (dd, J=54.5, 17.4 Hz, 2H), 4.13 (d, J=13.8 Hz, 1H), 3.80-3.68 (m, 1H), 3.60-3.37 (m, 3H), 3.29-3.25 (m, 1H), 2.96-2.86 (m, 1H), 2.79 (t, J=10.0 Hz, 2H), 2.73-2.54 (m, 3H), 2.42-2.25 (m, 2H), 2.20 (s, 3H), 2.03-1.90 (m, 1H). ESI-MS(M+H)⁺: 630.3.

The corresponding intermediate KT-022-1 of KT-022 was synthesized as follows:

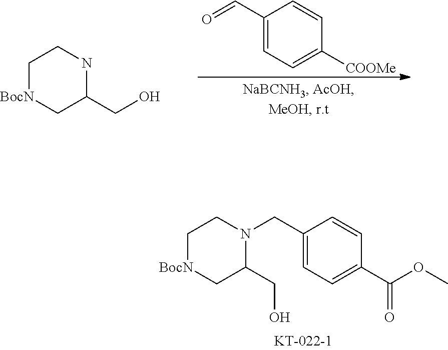 [SMILES: CC(C)(C)OC(=O)N1CCNC(CO)C1 ; COC(=O)c1ccc(C=O)cc1 ; COC(=O)c1ccc(CN2CCN(C(=O)OC(C)(C)C)CC2CO)cc1]

### Synthesis of compound KT-022-1

Compound 1-BOC-3-hydroxyethylpiperazine (1.05 g, 5.0 mmol) and methyl p-formyl benzoate (820 mg, 5.0 mmol) were dissolved in 20 mL, methanol, to which catalytic amount of acetic acid was added. After stirred for 30 min, sodium cyanoborohydride (630 mg, 10.0 mmol) was added. After reacted at room temperature overnight, TLC showed that the reaction was complete. Water (60 mL) was added to quench the reaction and the aqueous phase was extracted with dichloromethane (60 mL*3). The organic phases were combined and washed with saturated brine (20 mL), and then dried with anhydrous sodium sulfate and concentrated. The crude product was purified by column chromatography (petroleum ether: ethyl acetate=1: 1 as mobile phase) to give compound KT-022-1 (1.25 g, 69% yield).

KT-034 was synthesized according to the KT-020

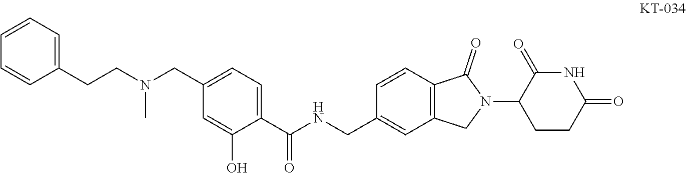 [SMILES: CN(CCc1ccccc1)Cc1ccc(C(=O)NCc2ccc3c(c2)CN(C2CCC(=O)NC2=O)C3=O)c(O)c1]

¹H NMR (400 MHz, DMSO) δ 10.97 (s, 1H), 9.38 (t, J=5.9 Hz, 1H), 7.82 (d, J=8.1 Hz, 1H), 7.69 (d, J=8.1 Hz, 1H), 7.55 (s, 1H), 7.47 (d, J=7.9 Hz, 1H), 7.28-7.24 (m, 2H), 7.20-7.15 (m, 3H), 6.86-6.76 (m, 2H), 5.08 (dd, J=13.3, 5.1 Hz, 1H), 4.61 (d, J=5.7 Hz, 2H), 4.37 (dd, J=54.7, 17.4 Hz, 2H), 3.51 (s, 2H), 2.94-2.84 (m, 1H), 2.80-2.73 (m, 2H), 2.65-2.55 (, 3H), 2.44 -2.31 (m, 1H), 2.20 (s, 3H), 2.04-1.94 (m, 1H). EST-MS(M+H1)⁺: 541.3.

The corresponding intermediate KT-034-1 of KT-034 was synthesized as follows:

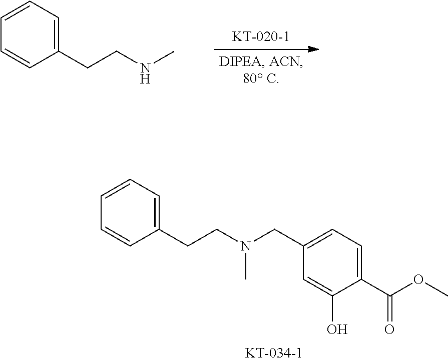

### Synthesis of compound K1-034-1

The compounds N-methyl-2-phenylethylamine (373 mg, 2.77 mmol), KT-020-1 (450 mg, 1.84 mmol) and N, N-diisopropyl ethylamine (593 mg, 4.6 mmol) were dissolved in 8 mL acetonitrile. After reacted at 80° C. overnight, TLC showed that the reaction was complete. The reaction was quenched with water (25 mL) and the aqueous phase was extracted with ethyl acetate (25 mL*3). The organic phases were combined and washed with saturated brine (10 mL*3). The organic phase was dried with anhydrous sodium sulfate and concentrated. The crude product was purified by column chromatography (petroleum ether: ethyl acetate=10: 1 as mobile phase) to give compound KT-034-1 (300 mg, 54% yield).

### Example 8

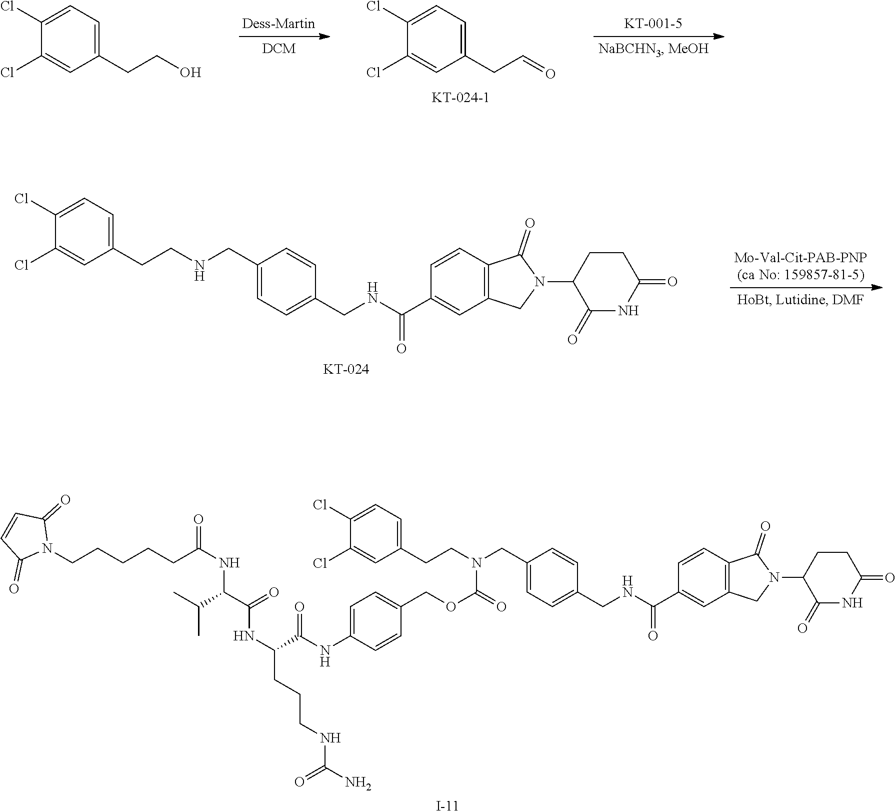

### Synthesis of compound KT-024-1

2-(3, 4-dichlorophenyl) ethanol (500 mg, 2.63 mmol) was dissolved in 12 mL DCM, to which Dess-Martin reagent (1.67 g, 3.95 mmol) was added in batches under ice bath. After reacted at room temperature for 2 h, TLC showed that the reaction was complete. The reaction solution was filtered and aqueous NaHCO₃ (30 mL) was added to the filtrate. The aqueous phase was extracted with dichloromethane (30 mL*3). The organic phases were combined and dried with anhydrous sodium sulfate, filtered, and then the solvent was removed by rotary evaporation. The crude product was purified by column chromatography (petroleum ether: ethyl acetate=100:1-15:1 as eluent) to give compound KT-024-1 (390 mg, 78% yield).

### Synthesis of compound KT-024

Compounds KT-024-1 (390 mg, 2.06 mmol), KT-001-5 (830 mg, 2.0 mmol) and acetic acid (100 mg) were dissolved in 20 mL methanol. After reacted at room temperature for 0.5 h, sodium cyanoborohydride (260 mg, 4.13 mmol) was added. After reacted at room temperature overnight, TLC showed that the reaction was complete. Saturated brine (30 mL) was added to it. The aqueous phase was extracted with dichloromethane (35 ml*3). The organic phases were combined and dried with anhydrous sodium sulfate, filtered, and then the solvent was removed by rotary evaporation. The crude product was purified by column chromatography (dichloromethane: methanol=20: 1 as eluent) to yield compound KT-024 (160 rug, yield 13%). ESI-MS(M+-H)⁺: 579.2. ¹H NMR (400 MHz, DMSO) δ 11.00 (s, 1H), 9.20 (t, J=6.0 Hz, 1H), 8.09 (s, 1H), 8.01 (d, J=8.0 Hz, 1H), 7,82 (d, J=7.9 Hz, 1H), 7.54-7.47 (m, 2H), 7.28 (s, 4H), 7.21 (dd, J=8.2, 2.0 Hz, 1H), 5.14 (dd, J=13.2, 5.1 Hz, 1H), 4.56-4.35 (m, 41), 3.73 (s, 21), 2.97-2.88 (m, 1H), 2.81-2.69 (n, 4H), 2.65-2.56 (m, 1H), 2.46-2.37 (m, 1H), 2.08-1.92 (m, 1H).

### Synthesis of compound I-11

Compounds KT-024 (160 mg, 0.28 mmol), Mc-Val-Cit-PAB-PNP (224 mg, 0.30 mmol) and dimethylpyridine (59 mg, 0.55 mmol) were dissolved in 18 mL DMF, to which HoBt(19 mg, 0.14 mmol) was added to it. After reacted for 48 h at room temperature, TLC showed that the reaction was complete. Water(50 mL) was added to quench the reaction. The precipitated solid is drained and the filted cake is rinsed with water. After the filtered cake is dried and then subjected to medium pressure preparation to give compound 1-11 (22 mg, 7% yield). ESl-MS(M+H)⁺: 1177.5. ¹H NMR (400 MHz, DMSO) δ 11.01 (s, 1H), 9.99 (s, 1H), 9.20 (t, J=5.6 Hz, 1H), 8.11-8.06 (m, 2H). 8.00 (d, J=7.9 Hz, 1H), 7.85-7.77 (m, 2H), 7.63-7.53 (m, 2H), 7.51-7.02 (m, 9H), 6.99 (s, 2H), 5.97 (t, J=5.7 Hz, 1H), 5.40 (s, 2H), 5.14 (dd, J=13.3, 5.1 Hz, 1H), 5.04-4.89 (m, 2H), 4.54-4.32 (m, 7H), 4.23-4.14 (m, 1H), 3.08-2.88 (m, 3H), 2.82-2.69 (m, 2H), 2.66-2.56 (m, 1H), 2.44-2.33 (m, 2H). 2.24-2.09 (m, 2H), 2.07-1.91 (m, 3H), 1.74-1.55 (m, 2H), 1.53-1.33 (m, 6H), 1.22-1.12 (m, 2H), 0.83 (dd, J=12.5, 6.8 Hz, 6H).

KT-025 was synthesized according to the KT-024

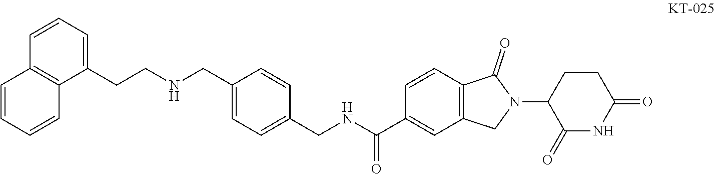 [SMILES: O=C1CCC(N2Cc3cc(C(=O)NCc4ccc(CNCCc5cccc6ccccc56)cc4)ccc3C2=O)C(=O)N1]

¹H NMR (400 MHz, DMSO) δ 11.01 (s, 1H), 9.23 (t, J=5.9 Hz, H), 8.12-7.97 (m, 3H), 7.94-7.87 (m, 1H), 7.82 (d, J=7.9 Hz, 1H), 7.78-7.75 (m, 1H), 7.54-7.25 (m, 8H), 5.14 (dd, J=13.3, 5.1 lIz, 1H), 4.59-4.34 (m, 4H), 3.83 (s, 21), 3.25-3.20 (m, 2H), 2.92-2.83 (m, 3H), 2.69-2.55 (, 1H), 2.46-2.34 (m, 1H), 2.07-1.90 (m, 1H). ESI-MS(M+H)⁺: 560.3.

### Example 9

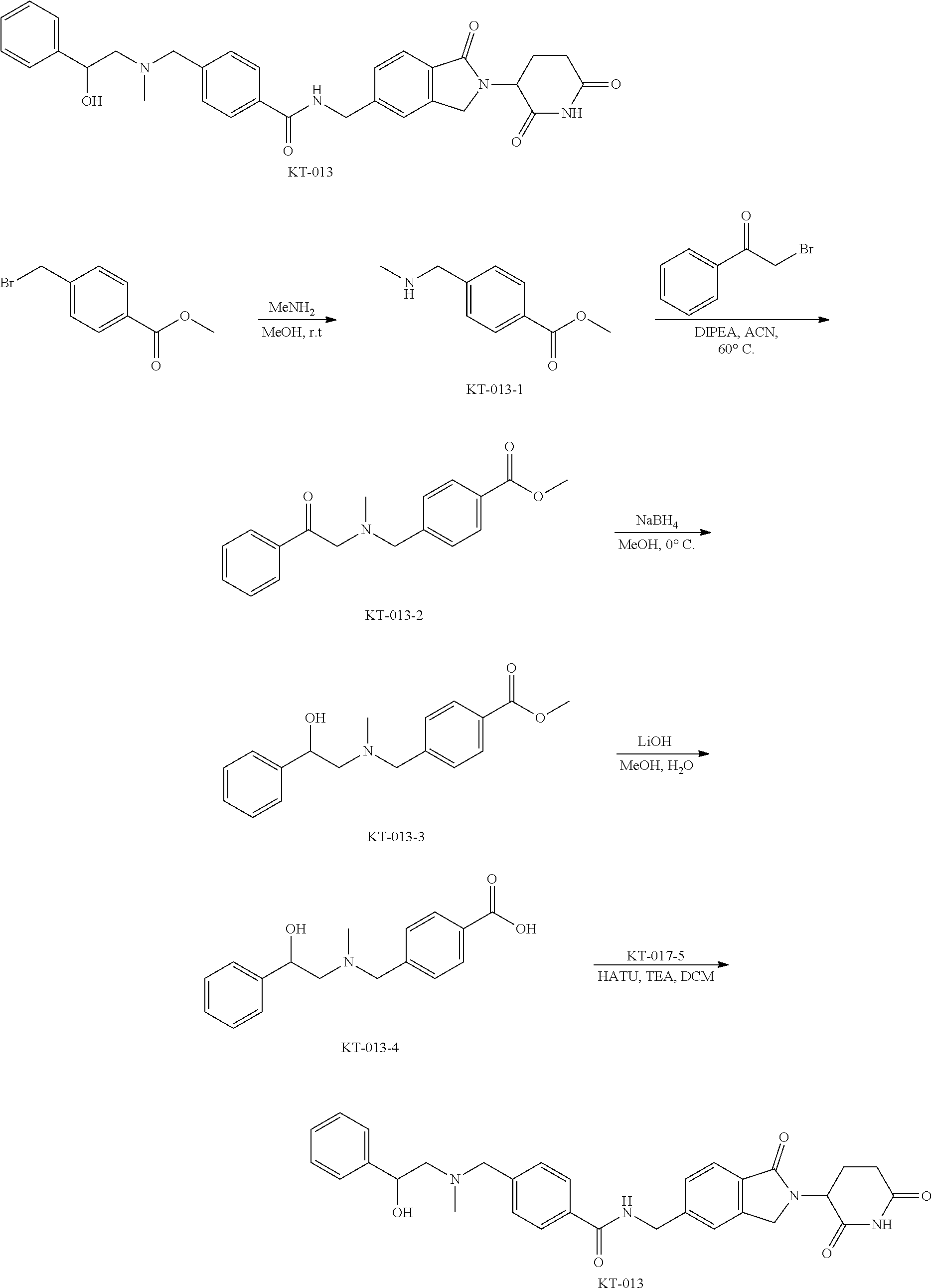

### Synthesis of compound KT-013-1

Compound methyl 4-bromomethylbenzoate (500 mg, 2.2 mmol) was dissolved in 15 mL methanol, to which a solution of methylamine (30%, 6 mL) was added. After reacted at room temperature for 1 h, TLC showed that the reaction was complete. Water (30 mL) was added. The aqueous was extracted with dichloromethane (35 ml*3). The organic phases were combined and dried with anhydrous sodium sulfate, filtered, and then the solvent was removed by rotary evaporation. The crude product was purified by column chromatography (dichloromethane: methanol=20: 1 as eluent) to yield compound KT-013-1 (230 mg, 57% yield).

### Synthesis of compound KT-013-2

Compound KT-013-1 (230 mg, 1.3 mmol) and DIPEA (660 mg, 5.1 mmol) were dissolved in 15 mL acetonitrile, to which 2-bromoacetophenone (310 mg, 15 mmol) was added. After reacted at 60° C. for 2 h, TLC showed that the reaction was complete. Water (20 mL) was added. The aqueous phase was extracted with ethyl acetate (30 mL*3). The organic phases were combined and dried with anhydrous sodium sulfate, filtered, and then the solvent was removed by rotary evaporation. The crude product was purified by column chromatography (petroleum ether: ethyl acetate=4: 1 as eluent) to yield compound KT-013-2 (250 mg, 65% yield).

### Synthesis of compound KT-013-3

Compound KT-013-2 (250 mg, 0.8 mmol) was dissolved in 15 mL methanol, to which sodium borohydride (48 mg, 1.3 mmol) was added under ice bath. After reacted at 0° C. for 2 h, TLC showed that the reaction was complete. Water (20 mL) was added. The aqueous phase was extracted with dichloromethane (30 ml*3). The organic phases were combined and dried with anhydrous sodium sulfate, filtered, and then the solvent was removed by rotary evaporation. The crude product was purified by column chromatography (petroleum ether: ethyl acetate=2: ²5 1 as eluent) to yield compound KT-013-3 (200 mg, 79% yield).

KT-013-4 and KT-013 were synthesized according to KT-017-4 and 11-017

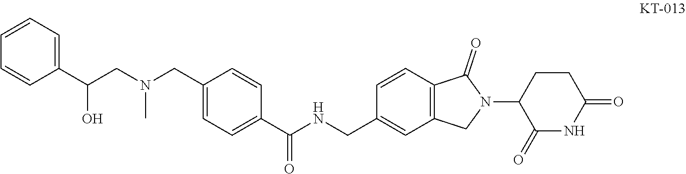 [SMILES: CN(Cc1ccc(C(=O)NCc2ccc3c(c2)CN(C2CCC(=O)NC2=O)C3=O)cc1)CC(O)c1ccccc1]

¹H NMR (400 MHz, DMSO) δ 10.97 (s, 1H), 9.09 (t, J=5.9 Hz, 1H), 7.82 (d, J=8.2 Hz, 2H), 7.69 (d, J=7.8 Hz, 1l), 7.54 (s, 1H), 7.46 (d, J=7.9 Hz, 1H), 7.36-7.27 (m, 6H), 7.26-7.19 (m, 1H), 5.10 (dd, J=13.3, 5.1 Hz, 1H), 4.74 (dd, J=7.6, 5.1 11z, 1H1), 4.58 (d, J=5.9 Hz, 2H), 4.37 (dd, J=54.6, 17.4 Hz, 2H), 3.60 (s, 2H), 2.96-2.84 (m, 1H), 2.65-2.53 (m, 2H), 2.49-2.44 (m, H), 2.43-2.32 (m, 2H), 2.22 (s, 3H), 2.05-1.95 (m, 1H). ESI-MS(M+H)⁺: 541.3.

### Example 10

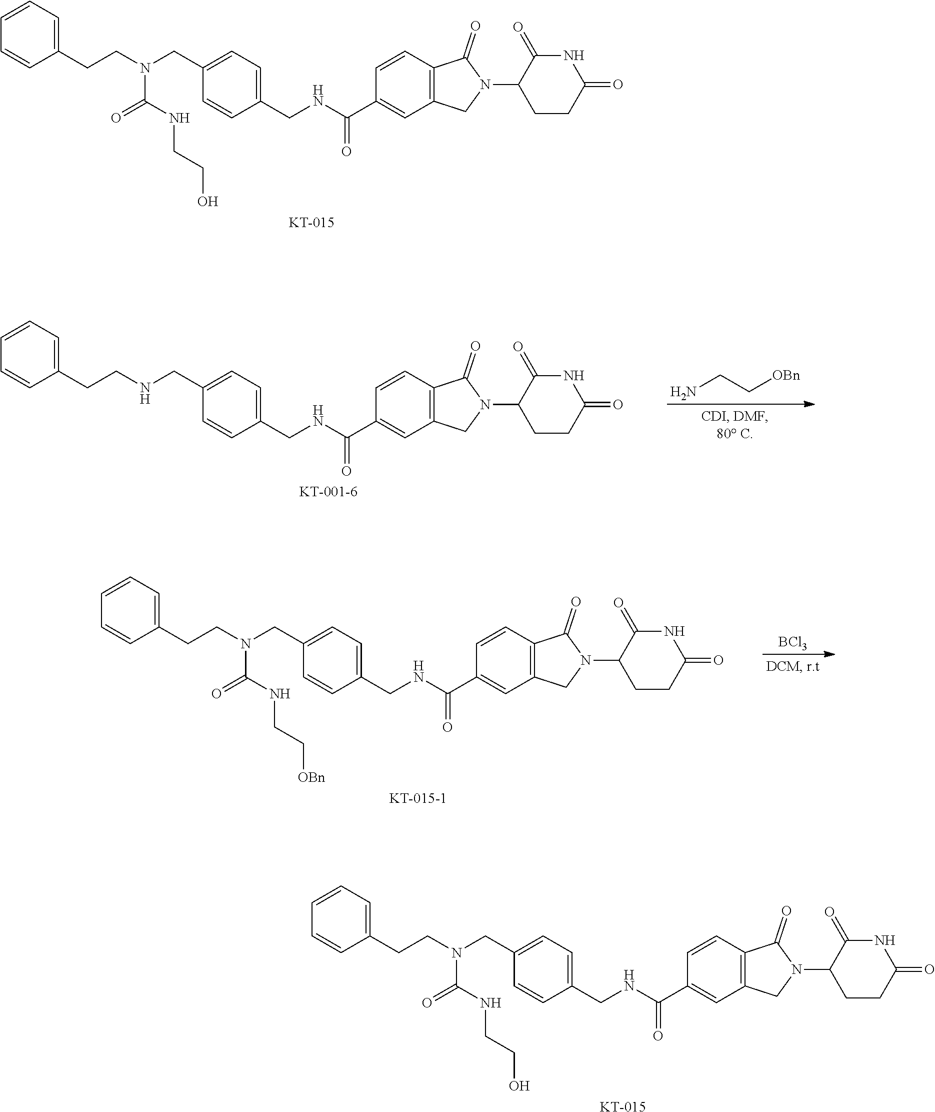

### Synthesis of compound KT-015-1

The compound 2-benzyloxy 1-ethylamine (30 mg, 0.2 mmol) was dissolved in 5 mL DMF, to which N, NV-carbonyl diimidazole (34 mg, 0.2 mL) was added. After reacted at room temperature for 2 h, KT-001-6 (50 mg, 0.1 mmol) was added. After reacted at 0° C. for another 2 h, TLC showed that the reaction was complete. Water (30 mL) was added. The aqueous phase was extracted with ethyl acetate (35 mL*3). The organic phases were combined. The organic phase was dried with anhydrous sodium sulfate, filtered, and then the solvent was removed by rotary evaporation. The crude product was purified by column chromatography (dichloromethane: methanol=20: 1 as eluent) to yield compound KT-015-1 (35 mg, 70% yield).

KT-015 was synthesized according to KT-019

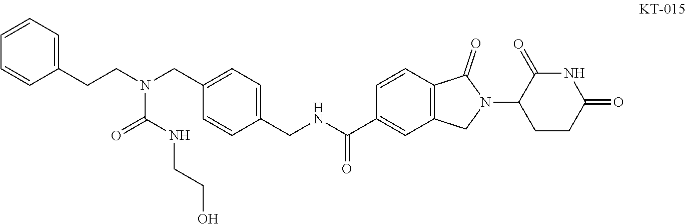 [SMILES: O=C1CCC(N2Cc3cc(C(=O)NCc4ccc(CN(CCc5ccccc5)C(=O)NCCO)cc4)ccc3C2=O)C(=O)N1]

¹H NMR (400 MHz, DMSO) δ 11.00 (s, 1H), 9.19 (t, J=5.9 Hz, H), 8.08 (s, 1H), 8.00 (d, J=8.0 Hz, 1), 7.81 (d, J=7.9 Hz, 1), 7.30-7,24 (m, 4H), 7,19-7.15 (m, 5H), 6.38 (t, J=5.5 Hz, 1H), 5.14 (dd, J=13.2, 5.1 Hz, 1H), 4.61 (t, J=5.4 Hz, 1H), 4.56-4.32 (m, 6H), 3.39 (dd, J=11.8, 6.1 Hz, 2H), 3.29-3.24 (m, 2H), 3.13 (q, J=6.1 Hz, 2H), 2.97-2.86 (m. 1H), 2.75-2.68 (m, 2H), 2.66-2.55 (m, 1H), 2.48-2.35 (m, 1H), 2.06-1.97 (i, 1H). EST-MS(M+H)⁺: 598.3.

### Example 11

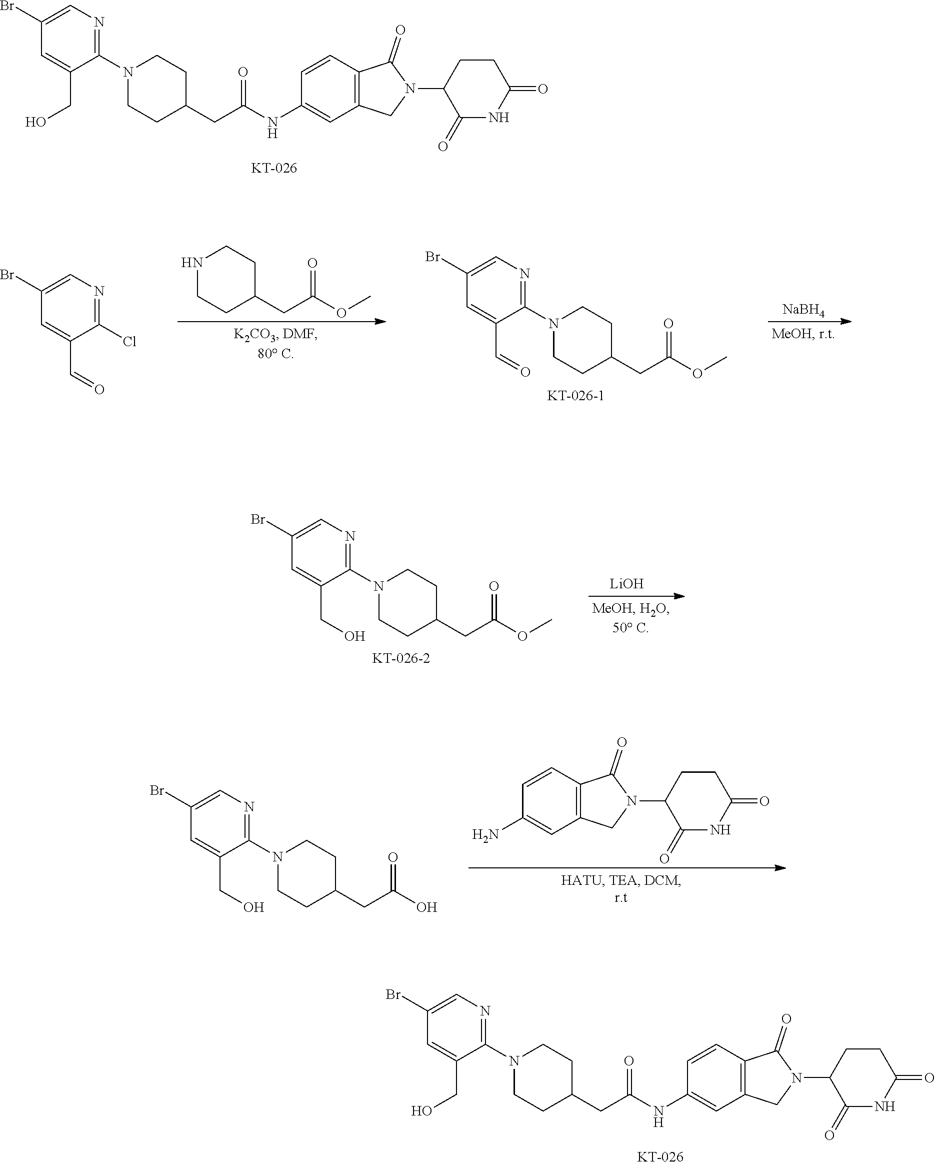

### Synthesis of compound KT-026-1

Compound 5-bromo-2-chloro-3-formylpyridine (250 mg, 1.14 mmol) was dissolved in 5 mL. DMF, to which methyl piperidine acetate (264 mug, 1.37 mL) was added. After reacted at 80° C. for 2 h, TLC showed that the reaction was complete. Water (30 mL) was added. The aqueous phase was extracted with ethyl acetate (35 mL*3), The organic phases were combined and washed in saturated brine (10 mL*3). The organic phase was dried with anhydrous sodium sulfate, filtered, and then the solvent was removed by rotary evaporation. The crude product was purified by column chromatography (petroleum ether: ethyl acetate=5: 1 as eluent) to yield compound KT-026-1 (197 ng, 50% yield).

### Synthesis of compound KT-026-2

Compound KT-026-1 (197 mg, 0.58 mmol) was dissolved in 6 mL methanol, to which sodium borohydride (33 mg, 0.87 mmol) was added under ice bath. After reacted at 0° C. for 2 h, TLC showed that the reaction was complete. Water (20 mL) was added. The aqueous phase was extracted with ethyl acetate (30 mL*3). The organic phases were combined and dried with anhydrous sodium sulfate, filtered, and then the solvent was removed by rotary evaporation. The crude product was purified by column chromatography (petroleum ether: ethyl acetate=5: 1 as eluent) to yield compound KT-026-2 (190 mg, 97% yield).

KT1-026-3 and KT-026 were synthesized according to KT-017-4 and KT-017

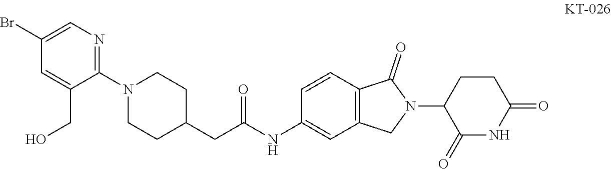 [SMILES: O=C1CCC(N2Cc3cc(NC(=O)CC4CCN(c5ncc(Br)cc5CO)CC4)ccc3C2=O)C(=O)N1]

¹H NMR (400 MHz, DMSO) δ 10.97 (s, 1H), 10.26 (s, 1H), 8.21 (d, J=2.5 Hz, 1H), 8.00 (s, 1H), 7.86 (d, J=2.4 Hz, 1H), 7.65 (d, J=8.3 Hz, 1H), 7.59 (d, J 9.6 Hz, 1H), 5.08 (dd, J=13.1, 5.0 Hz, 1H), 4.47-4.23 (m, 4H), 3.34-3.26 (m, 2H), 2.96-2.83 (m, 1H), 2.74 (t, J=11.8 Hz, 2H), 2.64-2.54 (m, 1H), 2.43-2.28 (m, 3H), 2.04-1.93 (m, 2H), 1.81-1.69 (m, 2H), 1.43-1.30 (m, 2H). ESI-MS(M+H)⁺: 570,3.

KT-028 was synthesized according to the KT-026

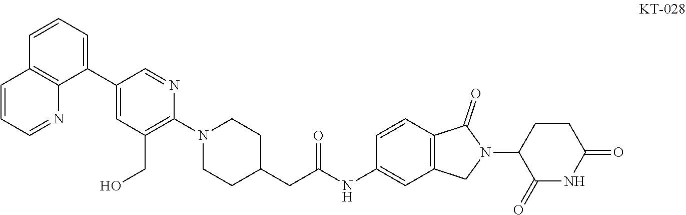 [SMILES: O=C1CCC(N2Cc3cc(NC(=O)CC4CCN(c5ncc(-c6cccc7cccnc67)cc5CO)CC4)ccc3C2=O)C(=O)N1]

¹H NMR (400 MHz, DMSO) δ 10.97 (s, 11H), 10.28 (s, 1H), 8.91 (dd, J=4.1, 1.7 Hz, 1H), 8.51-8.38 (m, 21f), 8.07 (d, J=2.2 Hz, 1H), 8.03-7.97 (m, 2H), 7.82-7.75 (m, 1H), 7.73-7.64 (m, 2H), 7.64-7.53 (m, 2H), 5.28 (t, J=5.4 Hz, 1H), 5.08 (dd, J=13.2, 5.1 Hz, 1H), 4.55 (d, J=5.1 Hz, 2H), 4.37 (dd, J=56.0, 17.3 Hz, 2H), 3.54-3.44 (m, 2H), 3.01-2.81 (m, 3H), 2.66-2.55 (in 1,H), 2.44-2.26 (m, 3H), 2.06-1.93 (m, 2H), 1.87-1.77 (m, 2H), 1.52-1.40 (n, 2H). ES1-MS(M−H)⁺: 619.3.

The corresponding intermediate KT-028, KT-028-2, were synthesized as follows:

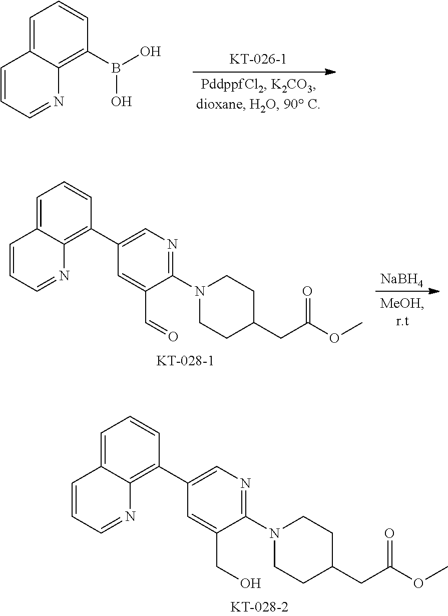 [SMILES: OB(O)c1cccc2cccnc12 ; COC(=O)CC1CCN(c2ncc(-c3cccc4cccnc34)cc2C=O)CC1 ; "NaBH4" ; COC(=O)CC1CCN(c2ncc(-c3cccc4cccnc34)cc2CO)CC1]

### Synthesis of compound KT-028-1

Compound quinoline-8-boric acid (870 mg, 5.0 mmol), KT-026-1 (1.7 g, 0.90 mmol) and potassium carbonate (2.07 g, 15.0 mmol) were dissolved in dioxane/water (20 mL/5 mL), to which Pd(dppf)CH₂ (366 mg, 0.5 mmol) was added. After reacted at 90° C. for 3 h under nitrogen protection, TLC showed that the reaction was complete. The reaction solution was filtered. The filtrate was diluted with ethyl acetate (60 mL), and the organic phase was washed with saturated brine (20 ml*3). The solvent was removed by rotary evaporation. The crude product was purified by column chromatography (petroleum ether: ethyl acetate=5: 1) to give compound KT-028-1 (300 mg, 88% yield).

### Synthesis of compound KT-028-2

Compound KT-028-1 (780 mg, 2.0 mmol) was dissolved in 40 mL methanol, to which sodium borohydride (152 mg, 4.0 mmol) was added under ice bath. After reacted at 0° C. for 2 h, TLC showed that the reaction was complete. Water (60 mL) was added. The aqueous phase was extracted with ethyl acetate (60 mL*3). The organic phases were combined and dried with anhydrous sodium sulfate, filtered, and then the solvent was removed by rotary evaporation. The crude product was purified by column chromatography (petroleum ether: ethyl acetate=1: 1 as eluent) to yield compound KT-028-2 (640 mg, 82% yield).

KT-030 was synthesized according to the KT-026

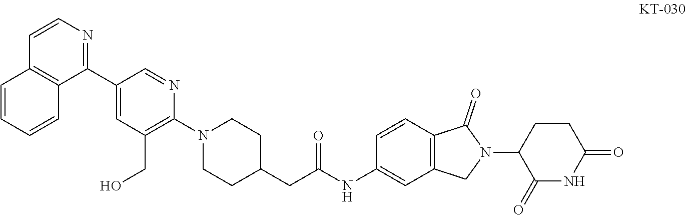 [SMILES: O=C1CCC(N2Cc3cc(NC(=O)CC4CCN(c5ncc(-c6nccc7ccccc67)cc5CO)CC4)ccc3C2=O)C(=O)N1]

¹H NMR (400 MHz, DMSO) δ 10.97 (s, 1H), 10.29 (s, 1H), 8.59 (d, J=5.6 Hz, 1H), 8.46 (d, J=2.3 Hz, 1H), 8.13-8.00 (m, 4H), 7.86-7.78 (m, 2H), 7.71-7.59 (m, 3H), 5.37 (t, J=5.4 Hz, 114), 5.08 (d d, J=13.3, 5.1 Hz, 1H), 4.56 (d, J=5.0 Hz, 2H), 4.35 (d d, J=56.0, 17.4 Hz, 2H), 3.62 -3.51 (m, 2H), 2.96-2.82 (m, 3H), 2.66-2.56 (m, 1H), 2.44-2.32 (m, 31), 2.07-1.96 (m, 2H), 1.87-1.77 (m, 2H), 1.53-1.35 (m, 2H). ESI-MS(M+H)^{y}: 619.4.

The corresponding intermediate KT-030-2 of KT-030 were synthesized as follows:

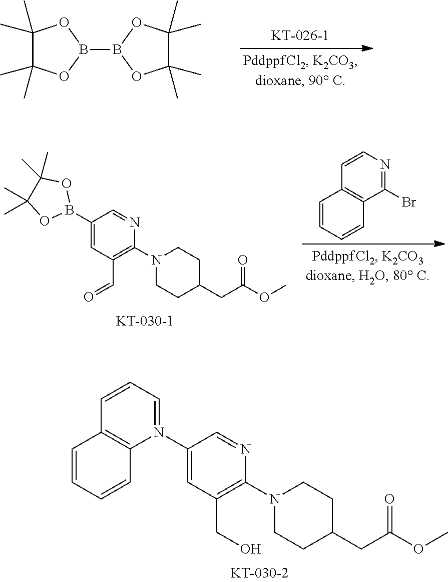

### Synthesis of compound KT-030-1

Compound pinacol borate (2.35 g, 9.26 mmol), KT-026-1 (2.1 g, 6.18 mmol) and potassium acetate (1,51 g, 15.45 mmol) were dissolved in 20 mL dioxane, to which Pd(dppf)CH₂ (454 mg, 0.62 mmol) was added. After reacted at 90° C. overnight under nitrogen protection, TLC showed that the reaction was complete. The reaction solution was filtered. The filtrate was diluted with ethyl acetate (60 mL), and the organic phase was washed with saturated brine (20 ml*3). The solvent was removed by rotary evaporation. The crude product was purified by column chromatography (petroleum ether: ethyl acetate=5:1) to give compound KT-030-1 (1.86 g, 77% yield).

### Synthesis of compound KT-030-2

1-bromoisoquinoline (290 mg, 1.4 mmol), KT-030-1 (504 mg, 1.3 mmol) and potassium carbonate (449 mg, 15.0 mmol) were dissolved in dioxane/water (4 mL/1 mL), to which Pd(dppf)CH₂ (95 mg, 0.62 mmol) was added. After reacted at 80° C. overnight under nitrogen protection, TLC showed that the reaction was complete. The reaction solution was filtered. The filtrate was diluted with ethyl acetate (60 mL), and the organic phase was washed with saturated brine (20 ml*3). The solvent was removed by rotary evaporation. The crude product was purified by column chromatography (petroleum ether: ethyl acetate=3: 1) to give compound KT-030-2 (408 mg, 81% yield).

### Example 12

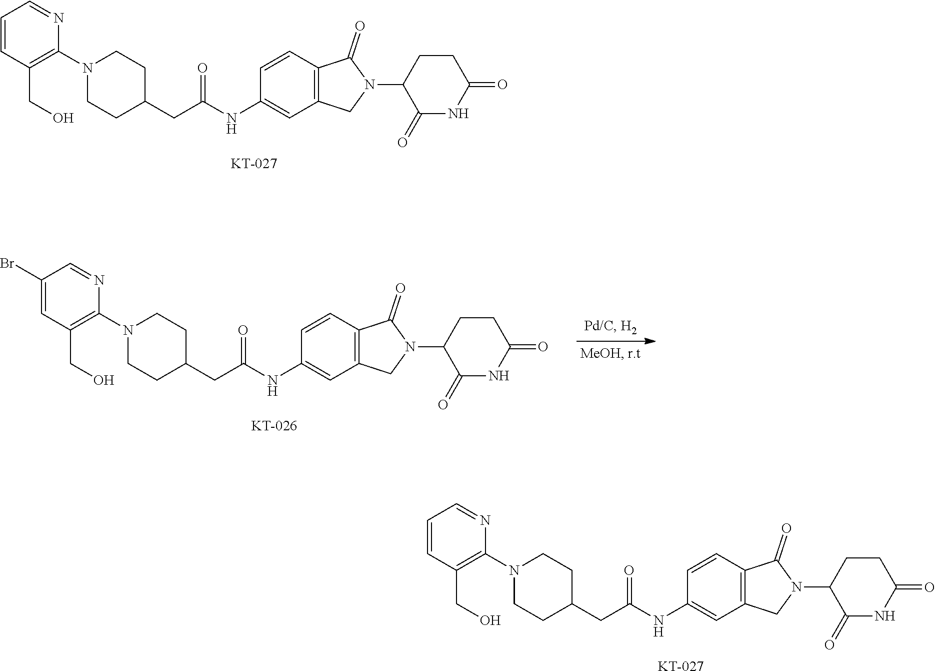

### Synthesis of compound KT1-027

Compound KT1-026 (60 mg, 0.10 mmol) and palladium carbon (5%, 10 mug) were dissolved 5in: mL methanol. After reacted at room temperature for 2 hi under hydrogen atmosphere, TLC showed that the reaction was complete. The reaction solution was filtered and the filtered cake was eluted with methanol. The filtrate was dried by rotary evaporation. The crude product was purified by column chromatography (dichiloromethane: methanol=15: 1 as eluent) to yield compound KT-=027 (8 mg, 16% yield).

¹H NMR (400 MHz, DMSO) δ 10.97 (s, 11H), 10.27 (s, 1H1), 8.12 (dd, J=4.8, 1.9 Hz, 1H1), 8.01 (s, 1H), 7.75 (d d, J=7.4, 1.8 Hz, 1H), 7.66 (d, 3=8.3 Hz, 1H), 7.60 (dd, J=8.3, 1.6 Hz, 1H), 6.98 (dd, J=7.4, 4.8 Hz, 1H), 5.25 (s, 11H), 5.08 (dd, J=13.3, 5.1 Hz, 1H1), 4.50-4.22 (m, 4H), 3.34-3.22 (m, 2H), 2.97-2.81 (m, 1H), 2.73 (t, J=11.6 Hz, 2H), 2.64-2.54 (m, 1H), 2.45-2.29 (m, 3H), 2.05-1.92 (m, 2H), 1.81-1.72 (m, 2H), 1.45-1.32 (m, 2H1). ESI-MS(M+H)⁺: 492.3.

### Example 13

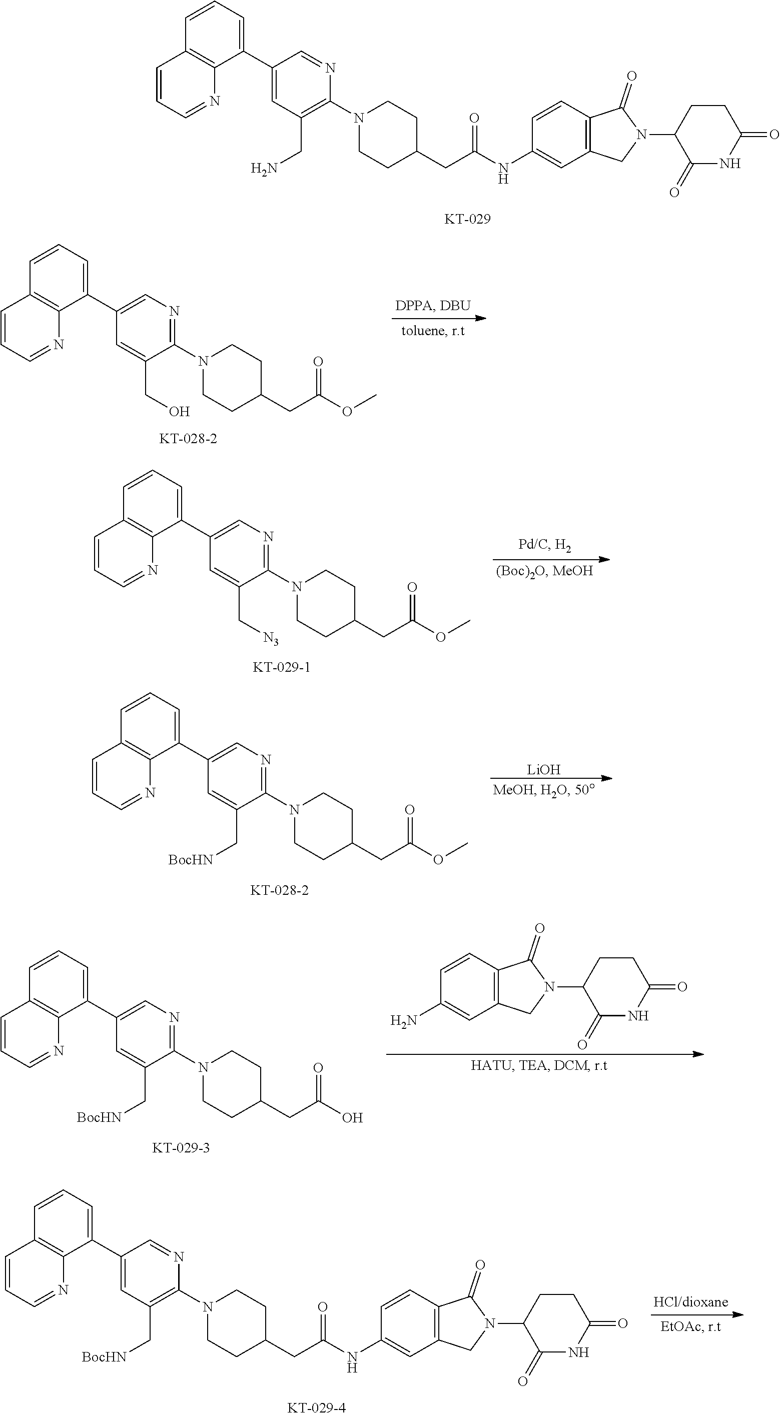

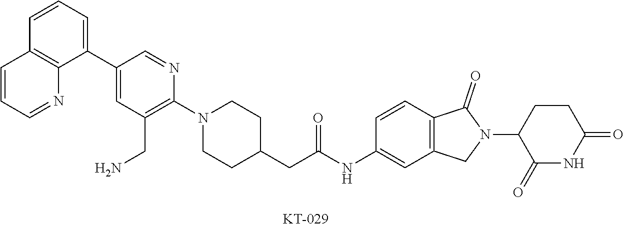 [SMILES: NCc1cc(-c2cccc3cccnc23)cnc1N1CCC(CC(=O)Nc2ccc3c(c2)CN(C2CCC(=O)NC2=O)C3=O)CC1]

### Synthesis of compound KT1-029-1

Compound KT-028-2 (640 mg, 1.63 mmol) and 1, 8-diazabicyclo [5.4.01 undecarbonyl-7-ene (380 mg, 2.5 mmol) were dissolved in 20 mL of toluene, to which diphenyl azide phosphate (550 mg, 2.0 mmol) was added. After reacted at room temperature for 3 h, TLC showed that the reaction was complete. Water (30 mL) was added. The aqueous phase was extracted with ethyl acetate (30 mL*3). The organic phases were combined and dried with anhydrous sodium sulfate, filtered, and then the solvent removed by rotary evaporation to yield the crude compound KT-029-1 (680 mg, crude) which was used directly for next step.

### Synthesis of compound KT-029-2

Compound KT-029-1 (680 mg, 1.63 mmol), di-tert-butyl dicarbonate (540 mg, 2.5 mmol) and palladium carbon (5%, 50 mg) were dissolved in 10 mL methanol. After reacted at room temperature for 5 h under hydrogen atmosphere, TLC showed that the reaction was complete. The reaction solution was filtered and the filtered cake was eluted with methanol. The filtrate was dried by rotary evaporation. The crude product was purified by column chromatography (petroleum ether: ethyl acetate=1:1 as eluent) to yield compound KT-029-2 (350 mg, 44% yield).

KT-029-3 and KT-029-4 were synthesized according to t KT-017-4 and KT-017

### Synthesis of compound KT-029

Compound KT-029-4 (108 mg, 0.15 mmol) was dissolved in 5 ml, ethyl acetate, to which HCl/dioxane (4 M, 5 mL) was added. After reacted at room temperature for 2 h, TLC showed that the reaction was complete. The mixture was dried by rotary evaporation. The crude product was purified by column chromatography (dichloromethane: methanol=10: 1 as eluent) to yield compound KT-029 (43 mg, 47% yield).

¹H NMR (400 MHz, DMSO) δ 10.33 (s, 1H), 8.91 (dd, J=4.1, 1.7 Hz, 1H), 8.50 (d, J=2.2 Hz, 1H), 8.44 (d d, J=8.3, 1.7 Hz, 1H), 8.12 (d, J=2.0 Hz, 1H), 8.05-7.96 (m, 2H), 7.81 (dd, J=7.1, 1.2 Hz, 1H), 7.74-70.64 (m, 2H), 70.64-7.54 (m, 2H), 5.07 (dd, J=13.3, 5.1 1z, 1H), 4.36 (dd, J=56.0, 17.4 Hz, 2H), 3.94 (s, 2H), 3.54-3.44 (n, 2H), 2.97-2.80 (m, 3H), 2.65-2.56 (m, 1H), 1 0 2.46-2.27 (m, 31), 2.07-1.92 (m, 2H), 1.87-1.77 (m, 2H), 1.55-1.41 (m, 2H). ESl-MS(M+H)⁺: 618.4.

### Example 14

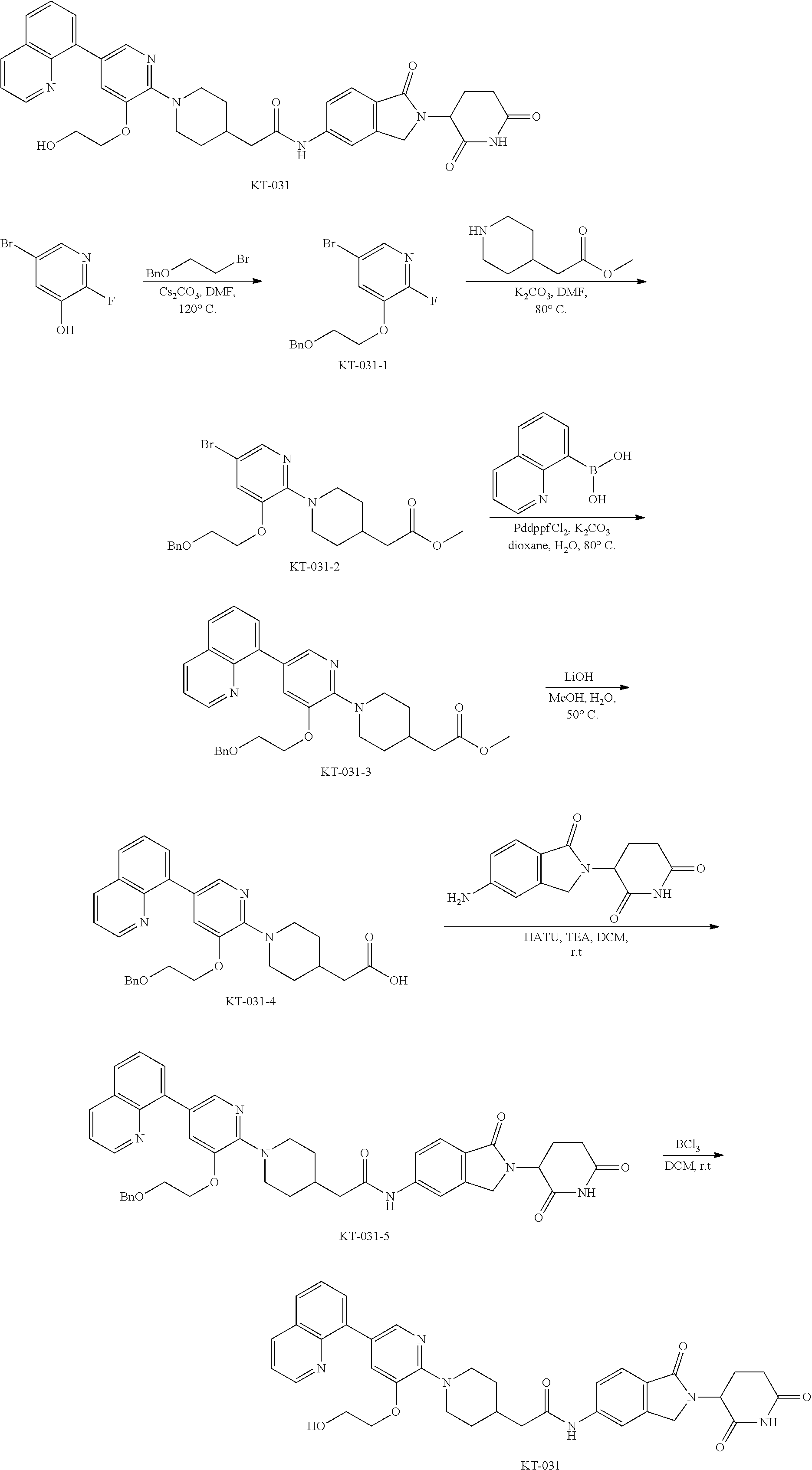

### Synthesis of compound KT-031-1

Compound 5-bromo-2-fluoro-3-hydroxypyridine (500 mg, 2.6 mmol) and compound benzyl 2-bromoethyl ether (840 mg, 0.3 mmol) were dissolved in 15 mL DMF, to which cesium carbonate (1.7 g, 5.2 mmol) was added. After reacted at 120° C. for 2 h, TLC showed that the reaction was complete. The reaction was quenched with water (60 nL). The aqueous phase was extracted with ethyl acetate (60 mL*3), The organic phases were combined and washed with saturated brine (10 mL*3). The organic phase was dried with anhydrous sodium sulfate and concentrated. The crude product was purified by column chromatography (petroleum ether: ethyl acetate=10: 1 as eluent) to give compound KT-031-1 (800 mg, 94% yield).

KT-031-2, KT-031-3, KT-031-4 and KT-031-5 were synthesized according to KT-026-1,

KT-028-1, KT-017-4 and KT-017

KT-031 was synthesized according to the KT-019

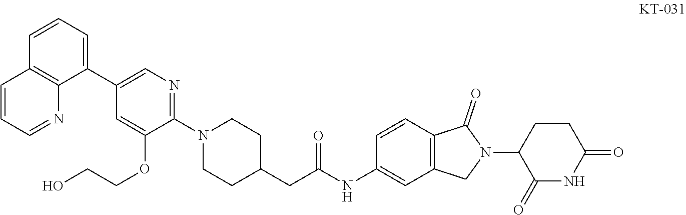 [SMILES: O=C1CCC(N2Cc3cc(NC(=O)CC4CCN(c5ncc(-c6cccc7cccnc67)cc5OCCO)CC4)ccc3C2=O)C(=O)N1]

¹H NMR (400 MHz, DMSO) δ 10.97 (s, 1H), 10.27 (s, 1H), 8.92 (dd, J=4.1, 1.8 Hz, 1H), 8.43 (dd, J=8.3, 1.7 Hz, 1H), 8.09 (d, J=1.8 Hz, 1H), 8.02-7.97 (m, 2H), 7.82 (dd, J=7.1, 1.3 Hz, 1H), 7.74-7.47 (m, 5H), 5.08 (dd, J=13.3, 5.1 Hz, 1H), 4.85 (br s, 1l), 4.36 (dd, J=56.2, 17.4 Hz, 2H), 4.17-4.08 (m, 2H), 4.06 (t, J=4.9 Hz, 2H), 3.81-3.72 (m, 2H), 2.97-2.85 (m, 1H), 2.84-2.74 (m, 2H), 2.65-2.56 (m, 1H), 2.44-2.31 (m, 3H), 2.07-1.95 (m, 2H), 1.83-1.72 (m, 1 5 2H), 1.50-1.36 (m, 2H). ESI-MS(M+H)⁺: 649.6.

### Example 15

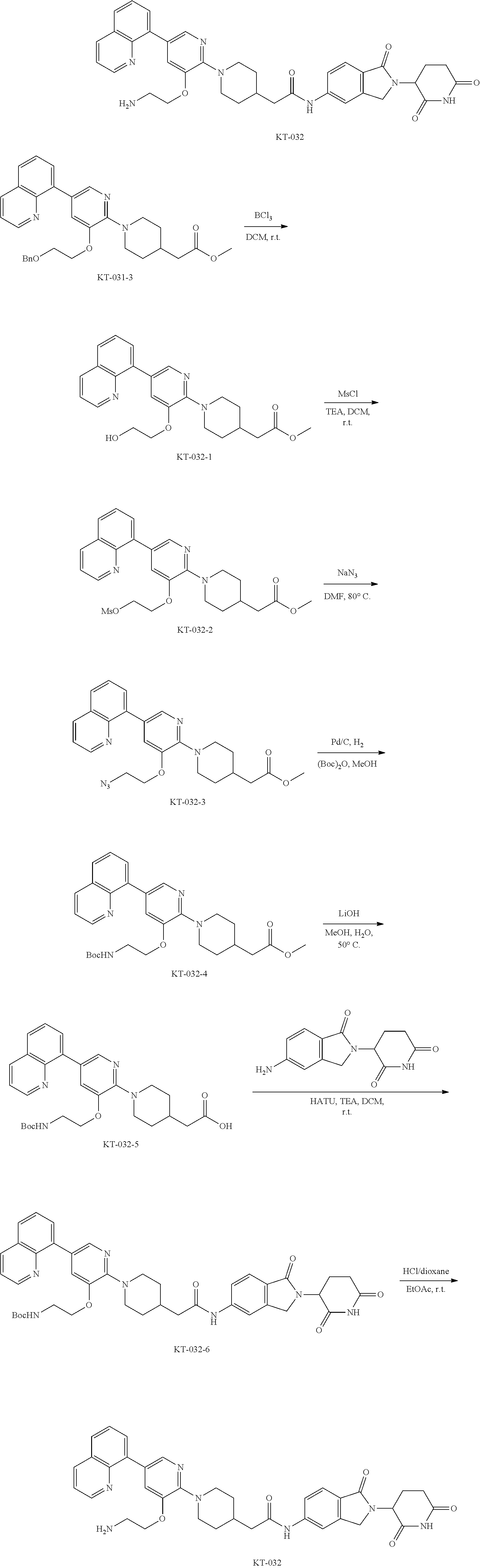

### Synthesis of compound K1-032-1

Compound KT-031-3 (2 g, 3.9 mmol) was dissolved in 40 mL dichloromethane, to which BCH₃ (11.7 mL, 1 M) solution was added under ice bath. After reacted at room temperature for 1 h, TLC showed that the reaction was complete. Methanol (10 mL) was added to quench the reaction and then the solvent was removed by rotary evaporation. The crude product was purified by column chromatography (dichloromethane: methanol=10: 1 as mobile phase) to yield compound KT-032-1 (1.5 g, 91% yield).

Synthesis of compound KT-032-2 Compound KT-032-1 (1.4 g, 3.3 mmol) and triethylamine (670 mg, 6.6 mmol) were dissolved in 20 mL dichloromethane, to which methylsulfonyl chloride (570 mg, 4.9 mmol) was added under ice bath. After reacted at room temperature for 3 h, TLC showed that the reaction was complete. The reaction was quenched by water (10 mL). The aqueous phase was extracted with dichloromethane (40 mL*3). The organic phases were combined and dried with anhydrous sodium sulfate, filtered, and then the solvent was removed by rotary evaporation. The crude product was purified by column chromatography (petroleum ether: ethyl acetate=1: 1 as mobile phase) to yield compound KT-032-2 (1.15 g, 69% yield).

### Synthesis of compound K1-032-3

Compound KT-032-2 (1.15 g, 2.3 mmol) was dissolved in 20 mL DMF, to which sodium azide (200 mg, 3 mmol) was added. After reacted at 60° C. overnight, TLC showed that the reaction was complete. The reaction was quenched by water (30 mL). The aqueous phase was extracted with ethyl acetate (30 mL*3). The organic phases were combined and washed with saturated brine (10 mL*3). The organic phase was dried with anhydrous sodium sulfate, filtered, and then the solvent was removed by rotary evaporation. The crude product was purified by column chromatography (petroleum ether: ethyl acetate=1: 1 as mobile phase) to yield compound KT-032-3 (1.03 g, 100% yield).

KT-032-4 was synthesized according to KT-029-2, KT-032-5 and KT-032-6 were synthesized according to KT-026-3 and KT-026

KT-032 was synthesized according to the KT-029

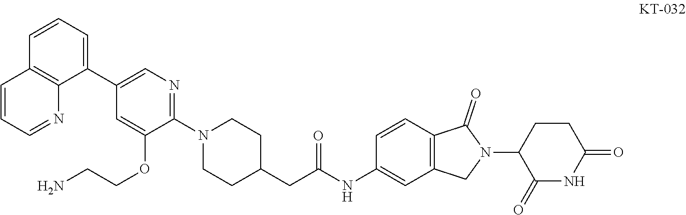 [SMILES: NCCOc1cc(-c2cccc3cccnc23)cnc1N1CCC(CC(=O)Nc2ccc3c(c2)CN(C2CCC(=O)NC2=O)C3=O)CC1]

¹H NMR (400 MHz, DMSO) δ 10.32 (s, 11H), 891 (d, J=2.6 Hz, 1H), 8.43 (d, J 7.5 Hz, 1H), 8.11 (s, 1H), 8.06-7.94 (m, 2H), 7.81 (d, J=6.9 Hz, 1H), 7,73-7.64 (m, 2H), 7.63-7.55 (m, 3H), 5.07 (dd, J=13.2, 5.1 Hz, H), 4.36 (dd, J=56.4, 17.2 Hz, 2H), 4.14-3.98 (m, 4H), 3.13-2.97 (m, 2H), 2.93-2.73 (m, 3H), 2.66-1.55 (m, 1l), 2.45-2.27 (m, 3H), 2.08-1.93 (m, 2H), 1.84-1.73 (m, 2H), 1.50-1.33 (m, 2H). ESI-MS(M+H)⁺: 648.4.

### Example 16

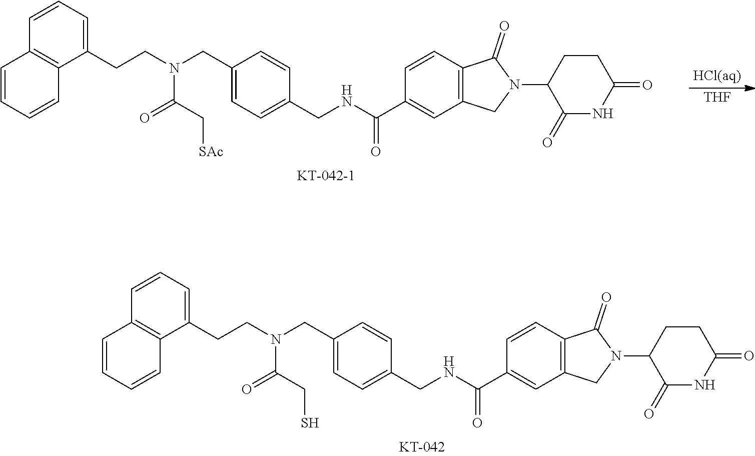

KT-042-1 was synthesized according to KT-004 (same method except that S-acetothiacetic acid was used instead of glycolic acid).

### Synthesis of compound KT-042

Compound KT-042-1 (50 mg, 0.074 mmol) was dissolved in 5 mL tetrahydrofuran, to which diluted hydrochloric acid (1 M, 1 mL) was added. After reacted at 50° C. overnight, TLC showed that the reaction was complete. Aqueous NaHCO₃ (10 mL) was added to the reaction solution. The aqueous phase was extracted with dichloromethane (10 mL*3). The organic phases were combined and dried. The solvent was removed by rotary evaporation. The crude product was purified by column chromatography (dichloromethane: methanol=15: 1 as eluent) to yield compound K1-042 (1 mg, 2% yield). ¹H NMR (400 MHz, DMSO) δ 11.02 (s, 1H), 9.23 (t, J=6.0 Hz, 1H), 8.16-7.96 (m, 3H), 7.94-7.87 (m, 1H), 7.84-7.74 (m, 2H), 7.56-7.26 (m, 6H), 7.26-7.17 (m, 2H), 5.14 (dd, J=13.3, 5.1 Hz, 1H), 4.58 (d, J=9.9 Hz, 2H), 4.53-4.32 (m, 4H), 3.57-3.43 (m, 2H), 3.33-3.28 (m, 2H), 3.22-3.14 (m, 1H), 2.98-2.77 (m, 2H), 2.65-2.56 (m, 1H), 2.43-2.36 (m, 1H), 2.05-1.95 (m, 1H). ESI-MS(M+H)⁺: 635.4.

### Implementation example 17

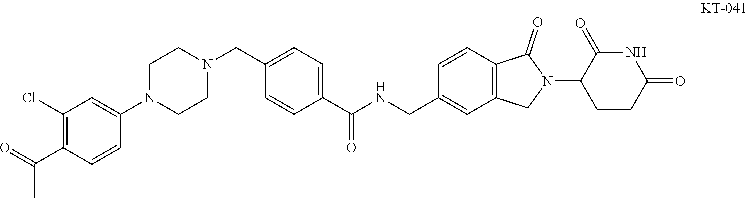 [SMILES: CC(=O)c1ccc(N2CCN(Cc3ccc(C(=O)NCc4ccc5c(c4)CN(C4CCC(=O)NC4=O)C5=O)cc3)CC2)cc1Cl]

KT-041 was synthesized according to KT-017

¹H NMR (400 MHz, DMSO) δ 10.97 (s, 1H), 9.12 (t, J=6.0 Hz, 1H), 7,88 (d, J=8.2 Hz, 2H), 7.72-7.68 (m, 2H), 7.54 (s, 1H), 7.49-7.39 (m, 31), 6.97-6.87 (m, 2H), 5.10 (dd, J=13.3, 5.1 Hz, 1H), 4.59 (d, J=5.8 Hz, 2H), 4.37 (dd, J=54.5, 17.3 Hz, 2H), 3.58 (s, 2H), 3.33-3.28 (m, 8H), 2.95-2.82 (m, 1H), 2.63-2.54 (m, 1H), 2.51 (s, 3H), 2.44-2.31 (m, 1H), 2.02-1.95 (m, 1H). ES1-MS(M+H)): 628.4.

### Biological Activity Assay

I. Anti-proliferation ability of compounds on HL60 cells
   Activity test methods:
   1. Cell plating
   HL60 cells in the logarithmic growth phase were counted and plated evenly in a 96-well transparent bottom white plate at 20000 cells per well and 100 pL per well.
   2. Drug addition to cells
   A certain amount of the compounds was weighed and diluted with DMSO to a 10 mM stock solution. The culture mediums corresponding to the cells were taken, and the drugs were diluted to the concentration required by the test, shaken gently and mixed evenly. 50 μl of the gradient dilution of the diluted compound was added to each well of the plated cells, and 50 μl of 0.1% DMSO was added to the control group. The plate was shaken gently, mixed evenly and incubated in a cell culture incubator for 3 days.
   3. CTG detection
   3.1 The CellTiter-Glo™ (CTG) reagent was taken in advance from the −20° C. refrigerator and equilibrated to room temperature in the dark.
   3.2 The 96-well plate was taken out and inspected under a microscope for growth situation.
   3.3 The bottom of the 96-well plate was tagged with white paper, and then the plate was wrapped with tin foil paper, shaken at 37° C. at 300 r for 10 mina and mixed evenly.
   3.4 50 ul of CTG detection reagent was added to each well, Luminescence signal was detected using a microplate reader, and the inhibition rate was calculated. IC50 values of the compounds were calculated by XLfit software to evaluate the proliferation inhibitory activity on 11160 cells. The activity test results were as shown in Table 1:

TABLE 1
Anti-proliferation ability of compounds against HL60 cells

| | HL60 cells | | HL60 cells |
|---|---|---|---|
| | Anti-proliferation | | Anti-proliferation |
| Compounds | activity (nM) | Compounds | activity (nM) |
| KT-001 | <3.04 | KT-004 | 0.54 |
| KT-002 | <3.04 | KT-005 | 6.26 ± 1.21 |
| KT-003 | 0.71 ± 0.04 | KT-006 | 2.19 ± 1.5 |
| KT-017 | 10.5 ± 0.4 | KT-020 | 1.36 ± 0.67 |
| KT-013 | 3.45 ± 0.52 | KT-015 | 24.2 ± 10.1 |
| KT-016 | 3.04 ± 0.84 | KT-018 | 2.17 ± 0.78 |
| KT-019 | 2.8 ± 0.1 | KT-034 | 9.9 ± 3.0 |
| KT-022 | 1.03 ± 0.19 | KT-025 | <3.04 |
| KT-026 | 23 ± 3 | KT-027 | 735 |
| KT-028 | <0.34 | KT-029 | 0.57 ± 0.1 |
| KT-030 | <0.34 | KT-031 | <0.34 |
| KT-032 | 5618 | KT-033 | 6.0 ± 1.6 |
| KT-041 | <3.04 | KT-042 | 34 ± 7 |

IL. Protein degradation experiments

### 1. Experimental Materials

Cell culture conditions: The cells in the embodiments of the invention were purchased from Shanghai Cell Bank, Chinese Academy of Sciences. Human acute promyelocytic leukemia cells (HL60 cells) were cultured in RPMI-1640 (meilunbio, MA0215)⁺20% FBS(manufacturer: Bi, product number: 04-001-1ACS)⁺1% penicillin/streptomycin (manufacturer: Hyclone, product number: SH40003.01). The cell culture conditions were 5% CO₂ and 37% humidity. When the cell coverage reached about 80%, the cells were passaged at a ratio of 1:3.

2. Cell drug treatment and protein extraction
2.1 Cell treatment

The designated cells in the exponential growth phase were digested and plated into a 6-well plate at 1 10⁶ cells per well. The cells were cultured by adding the compounds of the present invention, after 6 h of drug treatment, the protein was extracted.

2.2 Cell protein extraction

The cells were collected into 1.5 mL centrifuge tubes, and then supernatant is discarded after centrifugation at 1000 rpm for 5 min. Then the cells was washed by adding 1 mL PBS, and supernatant is discarded after centrifugation at 1000 rpm for 5 min. 100 μL of RIPA lysis buffer (Solarbio, product number: R0010) (containing 100 μM PMSF) was added to each tube, mixed thoroughly, allowed to stand on ice for 30 min and then centrifuged at 12000 rpm at 4° C. using a centrifuge for 20 min. The supernatant was used for Western blotting (WB) experiments. The sample can be stored at −80° C.

2.3 Determination of protein concentration

By using the BCA protein concentration determination kit (from Thermo Fisher, Cat. No. 23225), the BSA standard determination solution and the supernatant in 2.2 to be tested (the supernatant can be diluted and then tested) were prepared according to the table 2 below. Samples were added to the 96-well plate. Each well was supplemented with PBS to 20 μl and then 200 μl of BCA working solution (prepared according to the kit) was added respectively and mixed evenly. The plate was placed at 60′C for 10 min, and then the absorbance at 562 nim was detected. After the reading was recorded, a standard curve was plotted using concentration gradients of the standards, and the absorbance of the samples was substituted to calculate the protein concentration of the samples.

TABLE 2
Preparation of protein quantitative standards

| | Number | | | | | | | | |
|---|---|---|---|---|---|---|---|---|---|
| | 0 | 1 | 2 | 3 | 4 | 5 | 6 | 7 | 8 |
| | 1 mg/ml BSA | | | | | | | 5 mg/ml BSA | |
| | standard solution(μl) | | | | | | | standard solution (μl) | |
| BSA standard | 0 | 0.5 | 2.5 | 5.0 | 10 | 15 | 20 | 6 | 8 |
| solution (μl) | | | | | | | | | |
| PBS solution(μl) | 20 | 19.5 | 17.5 | 15 | 10 | 5 | 0 | 14 | 12 |
| The final | 0 | 25 | 125 | 250 | 500 | 750 | 1000 | 1500 | 2000 |
| concentration | | | | | | | | | |
| of BSA (μg/ml) | | | | | | | | | |

| | |
|---|---|
| The total | 20 μl |
| volume μl | |

3 Standard procedure for Western bloting experiments

3.1 Protein denaturation: RIPA protein buffer and 5x Loading Buffer (SDS, glycerol, bromophenol blue, TRIS) (purchased from Solarbio, Cat. No. P1040) were added, and the protein samples were denatured at 100° C. for 5 min.

3.2 Sample loading and electrophoresis: 10% 15-well precast gel (Genscript, Cat. No. M00666) and the corresponding electrophoresis buffer (Genscript, Cat. No. M00138) were used. The same mass of protein sample and protein ladder (purchased from thermo, Cat. No. 26617) were loaded into each well, and electrophoresis at 200 V was performed for 30 min.

3.3 Blocking: The gel was excised to remove the excess part. Transferring to PVDF membrane (Millipore, Cat. No. ISEQ00010) was performed by the wet transfer method (before use, it was necessary to activate the PVDF membrane with methanol for 1 min) at 300 mA for 2 h, during which a lot of heat was generated, and thus cooling with an ice box was required.

3.4 Blocking: After the transferring, the PVDF membrane was placed in 5% (w/w) skim milk and blocked with shaking at room temperature for 1 h.

3.5 Incubating with primary antibody: The PVDF membrane was cut according to the molecular weight indicated on the marker and placed in e-myc (purchased from abeam, Cat. No. ab32072), GSPT1 (purchased from abeam, Cat. No. ab234433), GAPDH (purchased from CST, Cat. No. #97166) primary antibodies. The antibodies were diluted with TBST buffer (a solution prepared from TRIS, KCL and NaCl and adjusted to pH 7.4 with hydrochloric acid, and Tween 20 was added) at a ratio of 1: 1000, and blocked overnight on a shaker at 4° C. 3.6 Incubating with secondary antibody: After incubated with the primary antibodies, the PVDF membrane was washed 3 times with TBST on a shaker, 10 min each time. After washed, the membrane was respectively placed in anti-mouse IgG, HRP-linked antibody (purchased from CST, Cat. No. #7076) or anti-rabbit IgG, HRP-linked antibody (purchased from CST, Cat. No. #7074) and incubated on a shaker at room temperature for 1 h.

3.7 Washing membrane and exposure: After incubated with the secondary antibodies, the membrane was washed 3 times with TBST on a shaker, 10 minutes each time. After washed, the membrane was subjected to enhanced ehemiluminescence (ECL) for visualizing the intensity of chemoluminescence.

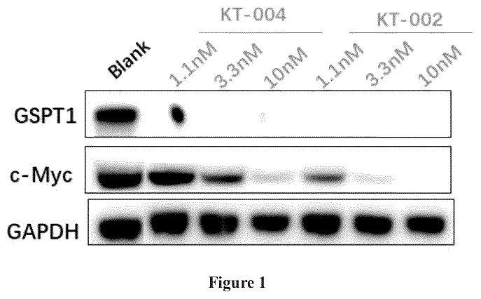

According to the above method, the ability to degrade proteins in the above cells was tested respectively, and the results were as shown in FIG. 1. Among them, Blank groups represent the DMSO blank groups without adding tested compounds. FIG. 1 shows KT-004 and KT -002 completely degrade e-Myc and GSPT1 protein at lower concentration (about 3 μm to 10 nmn) in HL60 cells.

Summary of technical effect: the invention of compounds have excellent degradation ability to tumorigenicity protein in a wide variety of tumor cells, such as e-Myc and GSPT1 in HL60 cells; Also the compounds can degrade other tumor cells contained in e-Myc, N-Myc, GSPT1, CK1αa, IKZF (1/213), AR, and AR-V7, any one or more of the protein, Which can be used to prevent, alleviate or cure any of the above (such as e-Myc) or a variety of protein e-Myc high expression related diseases, such as cancer, cardiovascular disease, the prevention and treatment of viral infections and other diseases. The compounds have precise and significant effects on degrading c-Myc, N-Mve, GSPT1, CK1α, IKZF (11213), AR, and AR-V7 proteins.

The content of the present invention merely exemplifies some specific embodiments set forth thereby, and the technical features recited in one or more technical solutions may be combined with any one or more technical solutions, wherein the technical solutions obtained by combination also fall within the scope of protection of the present application, as if the technical solutions obtained by combination have been specifically recited in the content disclosed by the present invention.

1. A compound represented by formula (I), and a pharmaceutically acceptable salt, solvent compound, stereoisomer, isotope and prodrug thereof: 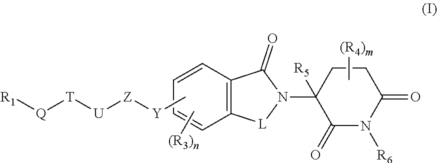
Wherein,
R₁ is selected from: Rₐ(CH₂)ₐ—, Rₐ(CH₂)ₐC(═O)—, Rₐ(CH₂)ₐNHC(═O)—, Rₐ(CH₂)ₐOC(═O)—or Rₐ(CH₂)ₐS(═O)₂—, wherein the “CH₂” is optionally substituted with one or more R₉, or is optionally substituted with CH₂CH₂—, and the “NH” is optionally substituted with R₁₄.
Q is selected from: —NR₂—, —O—, 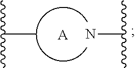
R₂ is selected from: hydrogen, R_{b}—, R_{b}C(═O)—, R_{b}S(═O)₂—, R₁₄;
Ring A represents an optionally substituted hererocycloalkyl containing at least one N atom as a heteroatom, and is linked to T or R₁ through the N atom, and ring A is alternately substituted with one or more groups selected from R₉;
Rₐ, R_{b} is selected from: C₁-C₈ alkyl, C₃-C₁₀ cycloalkyl, C₃-C₁₀ bridged cyclic group, —NR₁₁R₁₂, C₃-C₁₀ heterocyclyl optionally containing O, S, SO₂, N or NHC(═O)R₂₂, aryl, heteroaryl, fused arylcycloalkyl, fused arylheterocyclyl, fused heteroarylcycloalkyl, fused heteroarylheterocyclyl, aryl-aryl, aryl-heteroaryl, heteroaryl-aryl, heteroaryl-heteroaryl, aryl-alkyl, heteroaryl-alkyl, aryl-cycloalkyl, aryl-heterocyclyl, cycloalkyl-heterocyclyl or heterocyclyl-heterocyclyl, wherein a group selected from one of —O—, —S—, —C(═O)—, —S(═O)—, —S(═O)₂—, —NH—, —NHC(═O)—, —NHC(═O)NH— or —NHS(═O)₂— may be inserted between any two C—C of the C₁-C₈ alkyl, and Rₐ may be optionally substituted with one or more R₉, R_{b} may be optionally substituted with one or more R₁₀;
T, U and Z are each independently selected from: chemical bond, N, O, carbonyl, C₁-C₆ alkylene, C₃-CIO cycloalkylene, arylene, heteroarylene or heterocyclylene, wherein the alkylene, cycloalkylene, arylene, heteroarylene or heterocyclylene may be optionally substituted with one or more R₉;
Y is selected from: chemical bond, —C(═O)NH(CH₂)_{b}—, —NHC(═O)_{b}(CH₂)_{b}—, —O(CH₂)_{b}—, —NR₂(CH₂)_{b}—, wherein the “CH₂” is optionally substituted with one or more R₉, or is optionally substituted with —CH₂CH₂—, and the “NH” is optionally substituted with R₁₄;
L is selected from —(CR₇R₈)0— or —C(═O)—;
R₃-R₅ and R₇-R₉ are each independently selected from: hydrogen, R₁₃, halogen, cyano, amino, hydroxyl, sulfhydryl, nitro, —R₂₁N(R₂₂)R₂₂, —R₂₁C(═O)R₂₂, —R₂₁NHC(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxy, C₁-C₆ alkylthio, C₁-C₆ alkylamino, di(C₁-C₆ alkyl)amino, C₃-C₈ cycloalkyl, C₂-C₈ alkenyl, C₂-C₈ alkynyl, aryl, 5- to 6-membered heteroaryl containing 1-3 heteroatoms or 3-to 10-membered heterocyclyl containing 1-3 heteroatoms, wherein the alkyl, alkoxy, alkylamino, alkylthio, cycloalkyl, aryl, heteroaryl or heterocyclyl is optionally substituted with the group selected from halogen, cyano, C₁-C₃ alkyl or C₁-C₃ alkoxy; when there are multiple R₃-R₅ and R₇-R₉, any two adjacent ones may be combined to form a ring;
R₁₀ is selected from: hydrogen, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxy, C₁-C₆ alkylthio, C₁-C₆ alkylamino, di(C₁-C₆ alkyl)amino, C₃-C₈ cycloalkyl group, C₂-C₈ alkenyl, C₂-C₈ alkynyl, aryl, 5- to 6-membered heteroaryl containing 1-3 heteroatoms or 3- to 10-membered heterocyclyl containing 1-3 heteroatoms, wherein the alkyl, alkoxy, alkylamino, alkylthio, cycloalkyl, aryl, heteroaryl or heterocyclyl is optionally substituted with the group selected from halogen, cyano, C₁-C₃ alkyl or C₁-C₃ alkoxy; When R₁₀ is more than one, any two adjacent ones may be combined to form a ring;
R₁₃ is selected from: hydroxyl, sulfhydryl, amino, Rₙ, —ORₙ, —C(═O)Rₙ, —OC(═O)Rₙ, —NRₘRₙ, —C(═O)NRₘRₙ, —NRₘ—C(═O)Rₙ, —NRₙ—C(═O)Rₘ, —S(═O)₂Rₙ, —S(═O)₂NRₘRₙ, —NRₘ S(═O)₂Rₙ, —NRₙ—S(═O)₂Rₘ;
R₁₄ is selected from: Rₙ, —C(═O)Rₙ, —C(═O)ORₙ, —C(═O)NRₘRₙ, —S(═O)₂Rₙ, —S(═O)₂NRₘRₙ;
Rₙis selected from: C₁-C₈ alkyl substituted with hydroxyl, sulfhydryl, or amino group, wherein a group selected from one of —O—, —S—, —C(═O)—, —C(═O)O—, —OC(═O)O—, —S(═O)——S(═O)₂—, —NH—, —NHC(═O)—, —NHC(═O)NH—, —NHS(═O)₂— may be inserted between any two C—C of the C₁-C₈ alkyl; C₁-C₈ alkyl is each optionally substituted with 1-3 groups selected from halogen, cyano, amino, nitro or C₁-C₃ alkoxy;
Rₘ is independently selected from: hydrogen, C₁-C₈alkyl, wherein a group selected from one of —O—, —S—, —C(═O)—, —C(═O)O—, —OC(═O)O—, —S(═O)—, —S(═O)₂—, —NH—, —NHC(═O)—, —NHC(═O)NH—, —NHS(═O)₂— may be inserted between any two C—C of the C₁-C₈ alkyl, C₁-C₈alkyl is each optionally substituted with 1-3 groups selected from halogen, hydroxyl, cyano, amino, nitro or C₁-C₃ alkoxy;
R₆ is selected from: hydrogen, C₁-C₆ alkyl, C₃-C₈ cycloalkyl, C₂-C₈ alkenyl, C₂-C₈ alkynyl, aryl, 5- to 6-membered heteroaryl containing 1-3 heteroatoms or 3- to 10-membered heterocyclyl containing 1-3 heteroatoms, wherein the alkyl, cycloalkyl, aryl, heteroaryl or heterocyclyl is optionally substituted with 1-3 groups each selected from halogen, hydroxyl, cyano, amino, nitro, —C(═O)OR₂₃, —OC(═O)R₂₃, —NHC(═O)R₂₃, —C(═O)NHR₂₃, C₁-C₃ alkyl, C₁-C₃ alkoxy or —OP(═O)(OM)₂;
M is independently selected from: hydrogen or C₁-C₄ alkyl;
R₁₃ and R₁₂ are each independently selected from: hydrogen, C₁-C₄ alkyl, aryl, aryl-alkyl;
R₂₁ is selected from: chemical bond, C₁-C₄ alkylene;
R₂₂ and R₂₃ are each independently selected from: hydrogen, C₁-C₄ alkyl, aryl, aryl-alkyl, and the C₁-C₄ alkyl, aryl, aryl-alkyl are optionally substituted with halogen, hydroxyl, amino;
a is selected from: 0, 1, 2, 3, 4 or 5;
b is selected from: 0, 1, 2, 3, 4 or 5;
n is selected from: 0, 1, 2 or 3;
m is selected from: 0, 1, 2, 3 or 4;
o is selected from: 1 or 2;
Not following compounds are in formula (I): 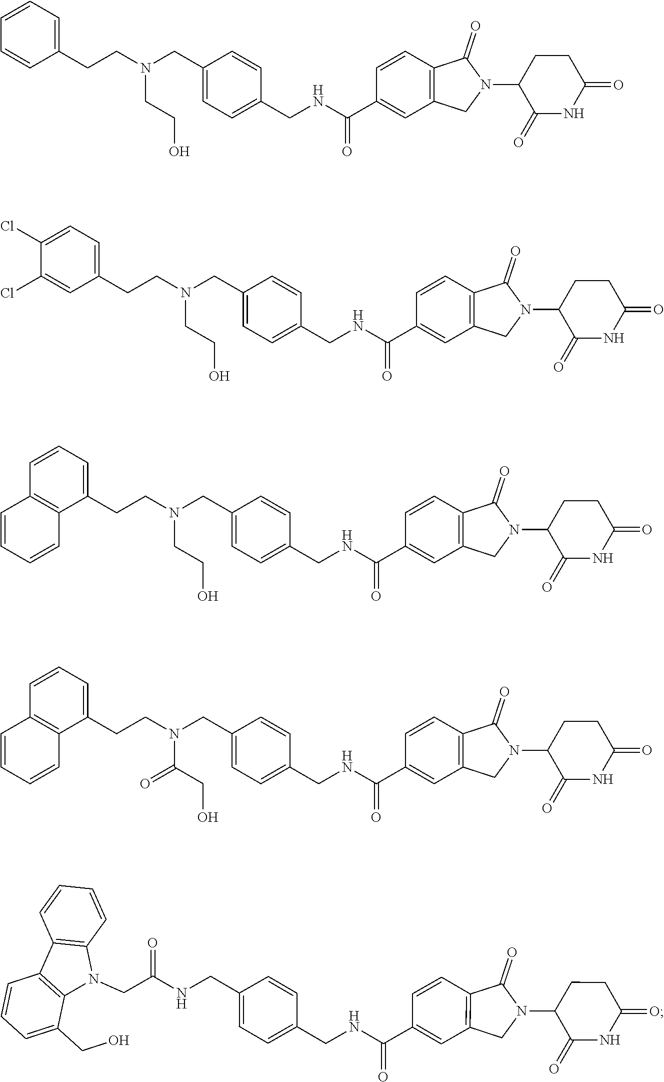 [SMILES: O=C1CCC(N2Cc3cc(C(=O)NCc4ccc(CN(CCO)CCc5ccccc5)cc4)ccc3C2=O)C(=O)N1 ; O=C1CCC(N2Cc3cc(C(=O)NCc4ccc(CN(CCO)CCc5ccc(Cl)c(Cl)c5)cc4)ccc3C2=O)C(=O)N1 ; O=C1CCC(N2Cc3cc(C(=O)NCc4ccc(CN(CCO)CCc5cccc6ccccc56)cc4)ccc3C2=O)C(=O)N1 ; O=C1CCC(N2Cc3cc(C(=O)NCc4ccc(CN(CCc5cccc6ccccc56)C(=O)CO)cc4)ccc3C2=O)C(=O)N1 ; O=C(Cn1c2ccccc2c2cccc(CO)c21)NCc1ccc(CNC(=O)c2ccc3c(c2)CN(C2CCC(=O)NC2=O)C3=O)cc1]
Preferably, the compound shown in formula (I) comprises at least one group selected from R₁₃ or R₁₄.

2. The compound and the pharmaceutically acceptable salt, solvent compound, stereoisomer, isotope and prodrug thereof according to claim 1, wherein, R₁ is selected from: RₐC(═O)—, RₐCH₂C(═O)—, RₐCH₂CH₂C(═O)—, Rₐ—, RₐCH₂—, RₐCH₂CH₂—, RₐNHC(═O)—, RₐCH₂NHC(═O)—, RₐCH₂CH₂NHC(═O)—, RₐOC(═O)—, RₐCH₂OC(═O)—, RₐCH₂CH₂OC(═O)—, RₐS(═O)₂—, RₐCH₂S(═O)₂—, RₐCH₂CH₂S(═O)₂—, wherein CH₂ is optionally substituted with one or more R₉, or optionally substituted with —CH₂CH₂—; wherein NH is optionally substituted with R₁₄;
preferably, R₁ is selected from: RₐC(═O)—, RₐCHR₉C(═O)—, RₐCHR₉CH₂C(═O)—, RₐCHCHR₉C(═O)—, Rₐ—, RₐCHR₉—, RₐCHR₉CH₂—, RₐCHCHR₉—, RₐNR₄C(═O)—, RₐCHR₉NHC(═O)—, RₐCHNR₄C(═O)—, RₐCHR₉CH₂NHC(═O)—, RₐCH₂CHR₉NHC(═O)—, RₐCH₂CH₂NR₄C(═O)—, RₐOC(═O)—, RₐCHR₉OC(═O)—, RₐCHR₉CH₂OC(═O)—, RₐCH₂CHR₉OC(═O)—, RₐCHR₉S(═O)₂—, RₐCHR₉CH₂S(═O)₂—, RₐCH₂CHR₉S(═O)₂—;
preferably, R₉ is selected from: hydrogen, C₁-C₄ alkyl, —R₂₁N(R₂₂)R₂₂, —R₂₁C(═O)R₂₂, R₁₃;
R₁₃ is selected from: hydroxyl, sulfhydryl, amino, Rₙ, —OR.;
R₁₄ is selected from: Rₙ, —C(═O)R.;
Rₙ is selected from: hydroxyl, sulfhydryl or amino substituted C₁-C₈ alkyl; Preferably, the Rₙ is selected from: hydroxyl, sulfhydryl or amino substituted C₁-C₄ alkyl; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH.

3. (canceled)

4. (canceled)

5. The compound and the pharmaceutically acceptable salt, solvent compound, stereoisomer, isotope and prodrug thereof according to claim 1, wherein, Rₐ is selected from: phenyl, naphthyl, carbazolyl, 1-aza-carbazolyl, 2-aza-carbazolyl, 1,8-diaza-carbazolyl, indolyl, 7-aza-indole, 2,3-dihydroindolyl, 2,3-dihydro-7-aza-indole, phenoxazinyl, fluorenyl, quinolinyl, isoquinolinyl, nalididinyl, tetrahydronalididinyl, tetrahydroquinolinyl, pyrimidinyl, pyridinyl, quinolinyl-pyridyl, triazolyl, bicyclo[1.1.1]pentyl, norborneyl, adamantane; Preferably, the Rₐ is optionally substituted with one or more R₉, wherein R₉ is selected from hydrogen, R₁₃, halogen, cyano, C₁-C₆ alkyl, C₁-C₆ haloalkyl, C₁-C₆ alkoxyl, phenyl, naphthyl;
preferably, Rₐ is selected from: phenyl, naphthalen-1-yl, naphthalen-2-yl, carbazole-9-yl, 1-aza-carbazole-9-yl, 2-aza-carbazole-9-yl, 1,8-diaza-carbazole-9-yl, indole-1-yl, 2, 3-dihydro-indole-1-yl, 7-aza-indole-1-yl, 2, 3-dihydro-7-aza-indole-1-yl, phenoxazine-10-yl, fluorene-9-yl, quinoline-4-yl, quinoline-5-yl, quinoline-8-yl, isoquinoline-1-yl, isoquinoline-4-yl, isoquinoline-5-yl, isoquinoline-8-yl, 1,2,3,4-tetrahydro-1,8-naphthyridine-1-yl, 1,2,3,4-tertrahydro-quinoline-1-yl, pyrimidine-2-yl, pyrimidine-4-yl, pyrimidine-5-yl, pyridine-2-yl, pyridine-3-yl, pyridine-4-yl, 5-(8-quinoline-yl)-pyridine-2-yl, 1,2,3-triazol-1-yl, 1,2,4-triazol-1-yl, 1,3,4-triazol-1-yl, bicyclo[1.1.11pentane-1-yl, norborne-1-yl, adamantane-1-yl; Preferably, Rₐis optionally substituted with one or more R₉, wherein R₉ is selected from: R₁₃, F, Cl, Br, cyano, methyl, ethyl, trifluoromethyl, methoxyl, ethoxy, phenyl, naphthalen-1-yl, naphthalen-2-yl; or
Rₐ is selected from: isoquinoline-pyridyl; Preferably, the Rₐ is optionally substituted with one or more R₉, wherein R₉ is selected from hydrogen, R₁₃, halogen, cyano, C₁-C₆ alkyl, C₁-C₆ haloalkyl, C₁-C₆ alkoxyl, phenyl, naphthyl;
preferably, Rₐ is selected from: 5-(1-isoquinoline-yl)-pyridine-2-yl; Preferably, Rₐ is optionally substituted with one or more R₉, wherein R₉ is selected from: R₁₃, F, Cl, Br, cyano, methyl, ethyl, trifluoromethyl, methoxyl, ethoxy, phenyl, naphthalen-1-yl, naphthalen-2-yl;
preferably, R₁₃ is selected from: hydroxyl, sulfydryl, amino, Rₙ, —ORₙ, —OC(═O)Rₙ, —NRₘRₙ, —NRₘ—C(═O)Rₙ, —NRₙ—C(═O)Rₘ, —NRₘ—S(═O)₂Rₙ, —NRₙ—S(═O)₂Rₘ; Preferably, R₁₃ is selected from: hydroxyl, Rₙ, —ORₙ, —NRₘRₙ, —NRₙ—C(═O)Rₘ, —NRₙ—S(═O)₂Rₘ;
wherein Rₙ is selected from: C₁-C₈ alkyl substituted with hydroxyl, sulfhydryl or amino; more preferably, Rₙ is selected from: C₁-C₄ alkyl substituted with hydroxyl, sulfhydryl or amino; Preferably, Rₙis selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH:
Rₘ is selected from: hydrogen, —C₁-C₄ alkyl;
preferably, R₉ is selected from: F, Cl, Br, cyano, methyl, ethyl, trifluoromethyl, methoxyl, ethoxyl, hydroxyl, sulfhydryl, amino, —C(═O)CH₃, C(═O)CH₂CH₃, C(═O)CH₂CH₂CH₃, —CH₂CH₂N(CH₃)₂, —CH₂OH, —CH₂CH₂OH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —OCH₂OH, —OCH₂CH₂OH, —OCH₂CH₂CH₂OH, —OCH₂NH₂, —OCH₂CH₂NH₂, —OCH₂CH₂CH₂NH₂, —OCH₂SH, —OCH₂CH₂SH, —OCH₂CH₂CH₂SH, —NHCH₂OH, —NHCH₂CH₂OH, —NHCH₂CH₂CH₂OH, —NHCH₂NH₂, —NHCH₂CH₂NH₂, —NHCH₂CH₂CH₂NH₂, —NHCH₂SH, —NHCH₂CH₂SH, —NHCH₂CH₂CH₂SH, —N(CH₃)CH₂OH, —N(CH₃)CH₂CH₂OH, —N(CH₃)CH₂CH₂CH₂OH, —N(CH₃)CH₂NH₂, —N(CH₃)CH₂CH₂NH₂, —N(CH₃)CH₂CH₂CH₂NH₂, —N(CH₃)CH₂SH, —N(CH₃)CH₂CH₂SH, —N(CH₃)CH₂CH₂CH₂SH, —N(COCH₃)CH₂OH, —N(COCH₃)CH₂CH₂OH, —N(COCH₃)CH₂CH₂CH₂OH, —N(COCH₃)CH₂NH₂, —N(COCH₃)CH₂CH₂NH₂, —N(COCH₃)CH₂CH₂CH₂NH₂, —N(COCH₃)CH₂SH, —N(COCH₃)CH₂CH₂SH, —N(COCH₃)CH₂CH₂CH₂SH, —N(SO₂CH₃)CH₂OH, —N(SO₂CH₃)CH₂CH₂OH, —N(SO₂CH₃)CH₂CH₂CH₂OH, —N(SO₂CH₃)CH₂NH₂, —N(SO₂CH₃)CH₂CH₂NH₂, —N(SO₂CH₃)CH₂CH₂CH₂NH₂, —N(SO₂CH₃)CH₂SH, —N(SO₂CH₃)CH₂CH₂SH, —N(SO₂CH₃)CH₂CH₂CH₂SH; or
Rₐ is selected from the following groups: 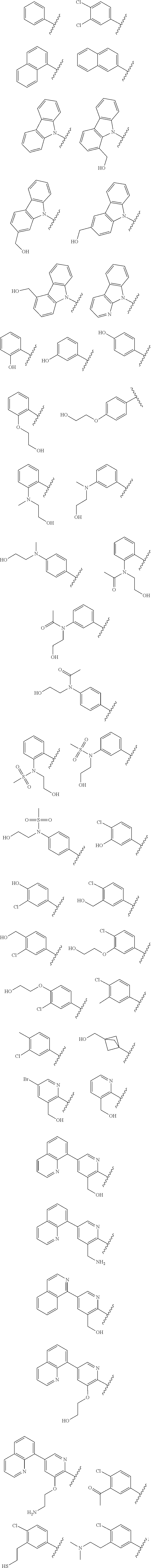
or
Rₐ, is selected from the following groups:

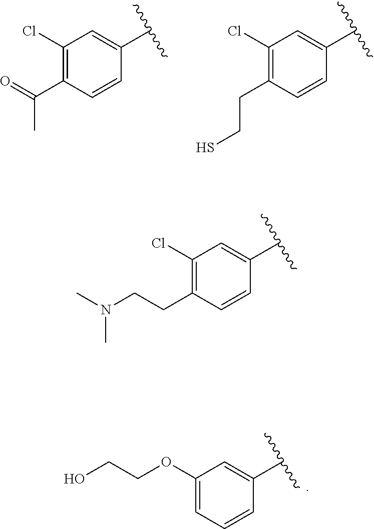 [SMILES: *c1ccc(C(C)=O)c(Cl)c1 ; *c1ccc(CCS)c(Cl)c1 ; *c1ccc(CCN(C)C)c(Cl)c1 ; *c1cccc(OCCO)c1]

6-12. (canceled)

13. The compound and the pharmaceutically acceptable salt, solvent compound, stereoisomer, isotope and prodrug thereof according to claim 1, wherein, R₂ is selected from: hydrogen, R₁₄, C₁-C₈alkyl, C₃-C₁₀ cycloalkyl, aryl, —C(═O)C₃-C₁₀ cycloalkyl, —C(═O)C₁-C₈alkyl or —S(═O)₂C₁-C₈alkyl, wherein C₁-C₈ alkyl, C₃-CIO cycloalkyl, aryl, and the cycloalkyl or alkyl portion of the —C(═O)C₃-C₁₀ cycloalkyl, —C(═O)C₁-C₈alkyl or —S(═O)₂C₁-C₈alkyl is optionally replaced substituted with one or more R₉, wherein R₉ is selected from:
hydrogen, halogen, amino, cyano, carboxyl, C₁-C₆ alkyl, C₁-C₆ alkoxy or aryl; Preferably, R₂ is selected from: R₁₄;
preferably, R₁₄ is selected from: Rₙ, —C(═O)Rₙ, —C(═O)ORₙ, —C(═O)NRₘRₙ, —S(═O)₂Rₙ, —S(═O)₂NRₘR₁;
wherein Rₙ is selected from: C₁-C₈ alkyl substituted with hydroxyl, sulfhydryl or amino; Preferably, the Rₙ is selected from: C₁-C₄ alkyl substituted with hydroxyl, sulfhydryl or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH:
wherein Rₘ is selected from: hydrogen, —C₁-C₄ alkyl;
preferably, R₂ is selected from: hydrogen, methyl, ethyl, —SO₂CH₃, —COCH₃, —CO— isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthyl ethyl, —CH₂OH, —CH₂CH₂OH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —C(═O)CH₂OH, —C(═O)CH₂CH₂OH, —C(═O)CH₂CH₂CH₂OH, —C(═O)CH₂NH₂, —C(═O)CH₂CH₂NH₂, —C(═O)CH₂CH₂CH₂NH₂, —C(═O)CH₂SH, —C(═O)CH₂CH₂SH, —C(═O)CH₂CH₂CH₂SH, —C(═O)OCH₂OH, —C(═O)OCH₂CH₂OH, —C(═O)OCH₂CH₂CH₂OH, —C(═O)OCH₂NH₂, —C(═O)OCH₂CH₂NH₂, —C(═O)OCH₂CH₂CH₂NH₂, —C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂CH₂CH₂OH, —C(═O)NHCH₂NH₂, —C(═O)NHCH₂CH₂NH₂, —C(═O)NHCH₂CH₂CH₂NH₂, —C(═O)NHCH₂SH, —C(═O)NHCH₂CH₂SH, —C(═O)NHCH₂CH₂CH₂SH, —S(═O)₂CH₂OH, —S(═O)₂CH₂CH₂OH, —S(═O)₂CH₂CH₂CH₂OH, —S(═O)₂CH₂NH₂, —S(═O)₂CH₂CH₂NH₂, —S(═O)₂CH₂CH₂CH₂NH₂, —S(═O)₂CH₂SH, —S(═O)₂CH₂CH₂SH, —S(═O)₂CH₂CH₂CH₂SH, —S(═O)₂NHCH₂OH, —S(═O)₂NHCH₂CH₂OH, —S(═O)₂NHCH₂CH₂CH₂OH, —S(═O)₂NHCH₂NH₂, —S(═O)₂NHCH₂CH₂NH₂, —S(═O)₂NHCH₂CH₂CH₂NH₂, —S(═O)₂NHCH₂SH, —S(═O)₂NHCH₂CH₂SH, —S(═O)₂NHCH₂CH₂CH₂SH.

14. (canceled)

15. (canceled)

16. The compound and the pharmaceutically acceptable salt, solvent compound, stereoisomer, isotope and prodrug thereof according to claim 1, wherein, 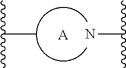
is selected from one of the following structures: 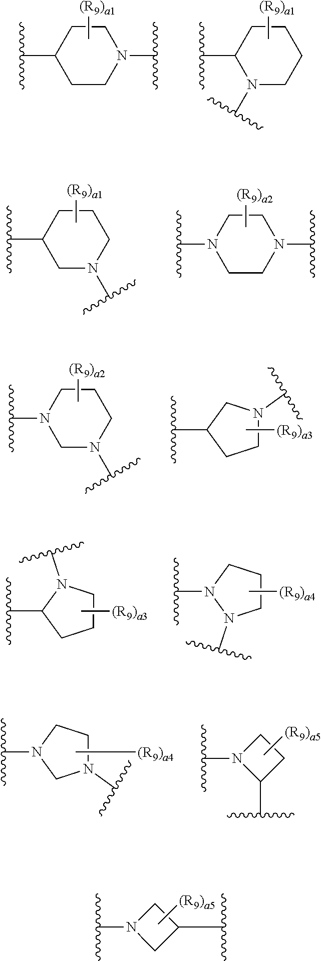
Wherein R₉ is independently selected from: hydrogen, R₁₃, halogen, cyano, nitro, C₁-C₆ alkyl, C₁-C₆ haloalkyl, C₁-C₆ alkoxyl;
a1 is independently selected from: 0, 1, 2, 3, 4, 5, 6, 7, 8, 9; a2 is independently selected from: 0, 1, 2, 3, 4, 5, 6, 7, 8; a3 is independently selected from: 0, 1, 2, 3, 4, 5, 6, 7; a4 is independently selected from: 0, 1, 2, 3, 4, 5, 6, 7; a5 is independently selected from: 0, 1, 2, 3, 4, 5;
preferably, R₁₃ is selected from: hydroxyl, sulfydryl, amino, Rₙ, —ORₙ, —OC(═O)Rₙ, —NRₘRₙ, —NRₘ—C(═O)Rₙ, —NRₙ—C(═O)Rₘ, —NRₘ—S(═O)₂Rₙ, —NRₙ—S(═O)₂Rₘ;
wherein Rₙ is selected from: C₁-C₈ alkyl substituted with hydroxyl, sulfhydryl or amino; more preferably, Rₙ is selected from: C₁-C₄ alkyl substituted with hydroxyl, sulfhydryl or amino; Preferably, Rₙis selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, or —CH₂CH₂CH₂CH₂SH:
wherein Rₘ is selected from: hydrogen, —C₁-C₄ alkyl; or preferably, 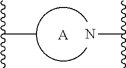
is selected from one of the following structures:

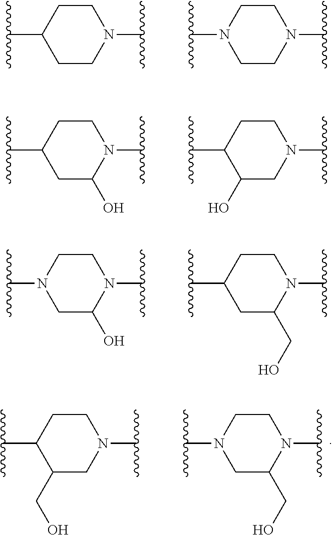

17. (canceled)

18. (canceled)

19. The compound and the pharmaceutically acceptable salt, solvent compound, stereoisomer, isotope and prodrug thereof according to claim 1, wherein, T and Z are independently selected from: chemical bond, carbonyl, C₁-C₆ alkylene or C₃-CIO cyclohexene, wherein C₁-C₆ alkylene, C₃-CIO cyclohexene is optionally substituted with one or more R₉;
preferably, T and Z are independently selected from: chemical bond, carbonyl, methylene, 1,2-ethylidene, 1,1-cyclopropylene, or 2,2-propylene, wherein methylene, 1,2-ethylidene, 1,1-cyclopropylene, 2,2-propylene is optionally substituted with one or more R₉;
preferably, R₉ is selected from: hydrogen, hydroxyl, sulfhydryl, amino, —R₂₁N(R₂₂)R₂₂—R₂₁C(═O)R₂₂, R₁₃, C₁-C₆ alkyl or C₃-C₈ cycloalkyl;
wherein R₁₃ is selected from: hydroxyl, sulfhydryl, amino, Rₙ, —OR.;
wherein Rₙ is selected from: C₁-C₈ alkyl substituted with hydroxyl, sulfhydryl or amino; Preferably, the Rₙ is selected from: C₁-C₄ alkyl substituted with hydroxyl, sulfhydryl or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH.

20. (canceled)

21. (canceled)

22. The compound and the pharmaceutically acceptable salt, solvent compound, stereoisomer, isotope and prodrug thereof according to claim 1, wherein, U is selected from: C₁-C₆ alkylene, C₃—CHO cycloalkylene, arylene, or heteroarylene, wherein alkylene, cycloalkylene, arylene, or heteroarylene is optionally substituted with one or more R₉; Preferably, U is selected from: C₂-C₆ alkylene, C₅-C₆ cycloalkylene, C₆-C₁₀ alkylene, 5-6 membered monocyclic heteroarylene, wherein alkylene, cycloalkylene, arylene, or heteroarylene is optionally substituted with one or more R₉, or
U is selected from: 1,2-ethylidene, 1,3-propylidene, 1,4-butylidene, 1,5- pentylidene, 1,6-hexylidene, 1,3-cyclopentylene, 1,3-cyclohexylidene, 1,4-cyclohexylidene, 1,2-phenylene, 1, 3-phenylene, 1,4-phenylene, 2,5-pyridinylene, 2,5-pyrimidylene, 2,5-thiazolylene or 2,4-oxazolylene, wherein 1,2-ethylidene, 1,3-propylidene, 1,4-butylidene, 1,5-pentylidene, 1,6-hexylidene, 1,3-cyclopentylene, 1,3-cyclohexylidene, 1,4-cyclohexylidene, 1,2-phenylene, 1, 3-phenylene, 1,4-phenylene, 2,5-pyridinylene, 2,5-pyrimidylene, 2,5-thiazolylene or 2,4-oxazolylene is optionally substituted with one or more R₉;
preferably, R₉ is selected from: hydrogen, amino, hydroxyl, sulfhydryl, —R₂₁N(R₂₂)R₂₂, —R₂₁C(═O)R₂₂, R₁₃, halogen, C₁-C₆ alkyl, C₁-C₆ haloalkyl, C₁-C₆ alkoxyl;
wherein R₁₃ is selected from: hydroxyl, amino, sulfhydryl, Rₙ, —ORₙ, —OC(═O)Rₙ, —NRₘRₙ, —NRₘ C(═O)Rₙ, —NRC(═O)Rₘ, —NRₘ S(═O)₂Rₙ, —N(═O)₂Rₘ;
wherein Rₙ is selected from: C₁-C₈ alkyl substituted with hydroxyl, sulfhydryl or amino; Preferably, the Rₙ is selected from: C₁-C₄ alkyl substituted with hydroxyl, sulfhydryl or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH:
wherein Rₘ is selected from: hydrogen, —C₁-C₄ alkyl;
preferably, R₉ is selected from: F, Cl, Br, cyano, methyl, ethyl, trifluoromethyl, methoxyl, ethoxyl, hydroxyl, sulfhydryl, amino, —C(═O)CH₃, C(═O)CH₂CH₃, C(═O)CH₂CH₂CH₃, —CH₂CH₂N(CH₃)₂, —CH₂OH, —CH₂CH₂OH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —OCH₂OH, —OCH₂CH₂OH, —OCH₂CH₂CH₂OH, —OCH₂NH₂, —OCH₂CH₂NH₂, —OCH₂CH₂CH₂NH₂, —OCH₂SH, —OCH₂CH₂SH, —OCH₂CH₂CH₂SH, —NHCH₂OH, —NHCH₂CH₂OH, —NHCH₂CH₂CH₂OH, —NHCH₂NH₂, —NHCH₂CH₂NH₂, —NHCH₂CH₂CH₂NH₂, —NHCH₂SH, —NHCH₂CH₂SH, —NHCH₂CH₂CH₂SH, —N(CH₃)CH₂OH, —N(CH₃)CH₂CH₂OH, —N(CH₃)CH₂CH₂CH₂OH, —N(CH₃)CH₂NH₂, —N(CH₃)CH₂CH₂NH₂, —N(CH₃)CH₂CH₂CH₂NH₂, —N(CH₃)CH₂SH, —N(CH₃)CH₂CH₂SH, —N(CH₃)CH₂CH₂CH₂SH, —N(COCH₃)CH₂OH, —N(COCH₃)CH₂CH₂OH, —N(COCH₃)CH₂CH₂CH₂OH, —N(COCH₃)CH₂NH₂, —N(COCH₃)CH₂CH₂NH₂, —N(COCH₃)CH₂CH₂CH₂NH₂, —N(COCH₃)CH₂SH, —N(COCH₃)CH₂CH₂SH, —N(COCH₃)CH₂CH₂CH₂SH, —N(SO₂CH₃)CH₂OH, —N(SO₂CH₃)CH₂CH₂OH, —N(SO₂CH₃)CH₂CH₂CH₂OH, —N(SO₂CH₃)CH₂NH₂, —N(SO₂CH₃)CH₂CH₂NH₂, —N(SO₂CH₃)CH₂CH₂CH₂NH₂, —N(SO₂CH₃)CH₂SH, —N(SO₂CH₃)CH₂CH₂SH, —N(SO₂CH₃)CH₂CH₂CH₂SH; or
U is selected from the following groups:

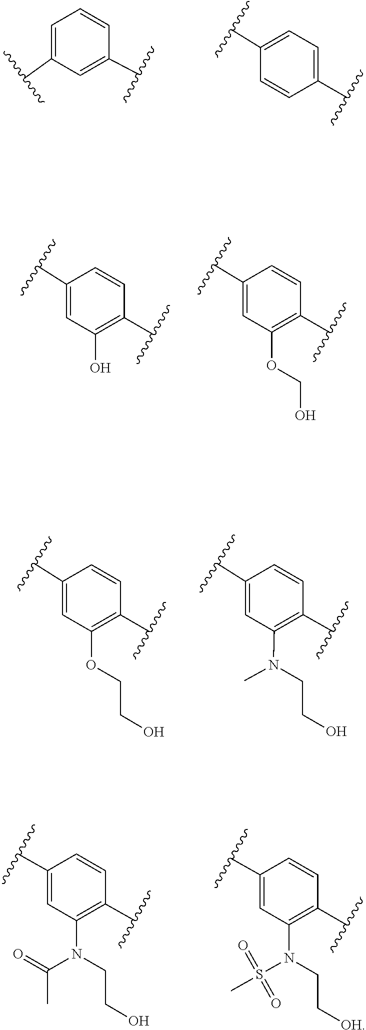

23-26. (canceled)

27. The compound and the pharmaceutically acceptable salt, solvent compound, stereoisomer, isotope and prodrug thereof according to claim 1, wherein, -T-U-Z-together form a group selected from the following groups: 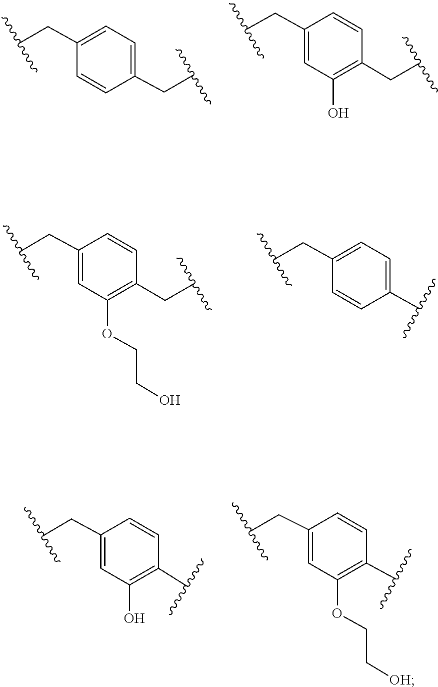 [SMILES: *Cc1ccc(C*)cc1 ; *Cc1ccc(C*)c(O)c1 ; *Cc1ccc(C*)c(OCCO)c1 ; *Cc1ccc(*)cc1 ; *Cc1ccc(*)c(O)c1 ; *Cc1ccc(*)c(OCCO)c1]
-T-U-Z- together form a group selected from the following groups: C₁ alkylene, 1,2-ethylidene, 1,3-propylidene, 1,4-butylidene, 1,5- pentylidene, 1,6-hexylidene.

28. (canceled)

29. The compound and the pharmaceutically acceptable salt, solvent compound, stereoisomer, isotope and prodrug thereof according to claim 1, wherein, Y is selected from: chemical bond, —C(═O)NH—, —C(═O)NHCH₂—, —C(═O)NHCH₂CH₂— -C(═O)NHCH₂CH₂CH₂—, —NHC(═O)—, —NHC(═O)CH₂—, —NHC(═O)CH₂CH₂—, —NHC(═O)CH₂CH₂CH₂—, —O—, —OCH₂—, —OCH₂CH₂—, —OCH₂CH₂CH₂—, —NR₂—, —NR₂CH₂— —NR₂CH₂CH₂—, —NR₂CH₂CH₂CH₂; or
Y is selected from: chemical bond, —NR₄C(═O)—, —C(═O)NR₄—, —C(═O)NR₄CH₂—, —C(═O)NHCHR₉—, —NR₄C(═O)CH₂—, —NHC(═O)CHR₉—, —O—, —OCHR₉—, —OCHR₉CH₂—, —OCH₂CHR₉—, —NR₂—, —NR₂CH₂—, —NHCHR₉—;
R₉ is selected from: hydrogen, C₁-C₄ alkyl, —R₂₁N(R₂₂)R₂₂, —R₂₁C(═O)R₂₂, R₁₃;
R₁₃ is selected from: hydroxyl, sulfhydryl, amino, Rₙ, —ORₙ;
R₁₄ is selected from: Rₙ, —C(═O)Rₙ;
R₂ is selected from: hydrogen, R₁₄, C₁-C₈alkyl, C₃—CHO cycloalkyl, aryl, —C(═O)C₃-C₁₀ cycloalkyl, —C(═O)C₁-C₈alkyl or —S(═O)₂C₁-C₈alkyl, wherein C₁-C₈alkyl, C₃—CHO cycloalkyl, ary, and the cycloalkyl or alkyl portion of the —C(═O)C₃—CHO cycloalkyl, —C(═O)C₁-C₈alkyl or —S(═O)₂C₁-C₈alkyl is optionally substituted with one or more R₉, and the R₉ is selected from:
hydrogen, halogen, amino, cyano, carboxyl, C₁-C₆ alkyl, C₁-C₆ alkoxyl or aryl; wherein R₁₄ is selected from: Rₙ, —C(═O)Rₙ, —C(═O)ORₙ, —C(═O)NRₘRₙ, —S(═O)₂Rₙ, —S(═O)₂NRₘRₙ;
Rₙ is selected from: C₁-C₈ alkyl substituted with hydroxyl, sulfhydryl or amino; Preferably, Rₙ is selected from: C₁-C₄ alkyl substituted with hydroxyl, sulfhydryl or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH:
wherein Rₘ is selected from: hydrogen, —C₁-C₄ alkyl;
preferably, R₂ is selected from: hydrogen, methyl, ethyl, —SO₂CH₃, —COCH₃, —CO— isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthyl ethyl, —CH₂OH, —CH₂CH₂OH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —C(═O)CH₂OH, —C(═O)CH₂CH₂OH, —C(═O)CH₂CH₂CH₂OH, —C(═O)CH₂NH₂, —C(═O)CH₂CH₂NH₂, —C(═O)CH₂CH₂CH₂NH₂, —C(═O)CH₂SH, —C(═O)CH₂CH₂SH, —C(═O)CH₂CH₂CH₂SH, —C(═O)OCH₂OH, —C(═O)OCH₂CH₂OH, —C(═O)OCH₂CH₂CH₂OH, —C(═O)OCH₂NH₂, —C(═O)OCH₂CH₂NH₂, —C(═O)OCH₂CH₂CH₂NH₂, —C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂CH₂CH₂OH, —C(═O)NHCH₂NH₂, —C(═O)NHCH₂CH₂NH₂, —C(═O)NHCH₂CH₂CH₂NH₂, —C(═O)NHCH₂SH, —C(═O)NHCH₂CH₂SH, —C(═O)NHCH₂CH₂CH₂SH, —S(═O)₂CH₂OH, —S(═O)₂CH₂CH₂OH, —S(═O)₂CH₂CH₂CH₂OH, —S(═O)₂CH₂NH₂, —S(═O)₂CH₂CH₂NH₂, —S(═O)₂CH₂CH₂CH₂NH₂, —S(═O)₂CH₂SH, —S(═O)₂CH₂CH₂SH, —S(═O)₂CH₂CH₂CH₂SH, —S(═O)₂NHCH₂OH, —S(═O)₂NHCH₂CH₂OH, —S(═O)₂NHCH₂CH₂CH₂OH, —S(═O)₂NHCH₂NH₂, —S(═O)₂NHCH₂CH₂NH₂, —S(═O)₂NHCH₂CH₂CH₂NH₂, —S(═O)₂NHCH₂SH, —S(═O)₂NHCH₂CH₂SH, —S(═O)₂NHCH₂CH₂CH₂SH.

30. (canceled)

31. (canceled)

32. The compound of claim 1 and pharmaceutically acceptable salt, solvent compound, stereoisomer, isotope and prodrug thereof, wherein L is selected from: —CH₂—, —CH₂CH₂—, C(═O)—, —CH(OH)—; or
R₃-R₅ is independently selected from: hydrogen, R₁₃, halogen, cyano, amino, C₁-C₆ alkyl, C₁-C₆ alkoxy, C₁-C₆ alkylthio, C₁-C₆ alkylamino, di(C₁-C₆ alkyl)amino, C₃-C₈ cycloalkyl, wherein the alkyl, alkoxy, alkylamino, alkylthio, or cycloalkyl is optionally substituted with 1-3 groups each selected from halogen, cyano, C₁-C₃ alkyl or C₁-C₃ alkoxy; when there are multiple R₃-R₅, any two adjacent ones may be combined to form a ring; or
R₆ is selected from: hydrogen, C₁-C₆ alkyl, C₃-C₈ cycloalkyl, wherein alkyl or cycloalkyl is optionally substituted with 1-3 groups each selected from halogen, hydroxyl, cyano, amino, —C(═O)OR₂₃, —OC(═O)R₂₃, —NHC(═O)R₂₃, —C(═O)NHR₂₃ or —OP(═O)(OM)₂; Preferably, R₆ is selected from: hydrogen, C₁-C₄ alkyl, C₁-C₄ haloalkyl, —C₁-C₄ alkyleneOC(═O)R₂₃, —C₁-C₄ alkylene C(═O)OR₂₃, —C₁-C₄ alkyleneOP(═O)(OH)₂; wherein R₂₃ is selected from: hydrogen, C₁-C₄ alkyl, and the C₁-C₄ alkyl is optionally substituted with 1-3 groups selected from hydroxyl and amino, respectively.

33. (canceled)

34. (canceled)

35. The compound and the pharmaceutically acceptable salt, solvent compound, stereoisomer, isotope and prodrug thereof according to claim 1, wherein, the compound is a compound represented by formula (I-1) or formula (I-2): 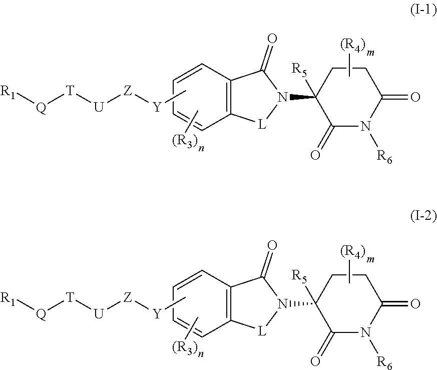
Wherein, R₁, R₃—R₆, Q, T, U, Z, Y, L, n, m are defined as above; or
the compound is a compound represented by formula (II) or (III): 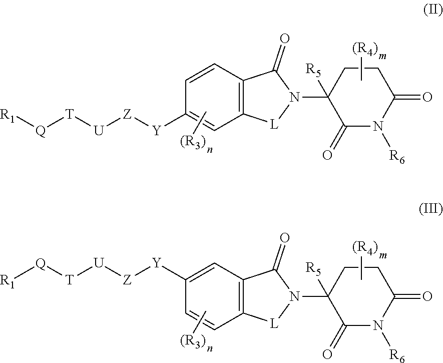
wherein R₁, R₃—R₆, Q, T, U, Z, Y, L, n, m are defined as above; or
the compound is a compound represented by formula (IV) or Formula (V): 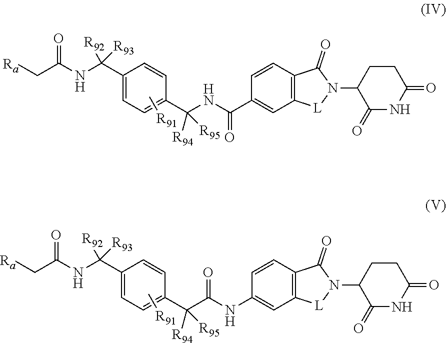
wherein NH of RₐCH₂C(═O)NH—, —C(R₉₄)(R₉₅)NHC(═O)—, —C(R₉₄)(R₉₅)C(═O)NH—, is optionally substituted with R₁₄;
Rₐ, R_{b4} and L are defined as above.
R₉₁-R₉₅ is independently selected from: hydrogen, R₁₃, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxyl, C₁-C₆ alkylthio, C₁-C₆ alkylamino, di(C₁-C₆alkyl)amino, C₃-C₈ cycloalkyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, cyano group, C₁-C₃ alkyl or C₁-C₃ alkoxyl; Any two adjacent ones in R₉₂-R₉₅ may be combined to form a ring;
preferably, R₉₁ is selected from: hydrogen, R₁₃, halogen, cyano, amino, C₁-C₆ alkyl, C₁-C₆ alkoxyl, C₁-C₆ alkylthio, C₁-C₆ alkylamino, di(C₁-C₆ alkyl)amino, C₃-C₈ cycloalkyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with 1-3 groups selected from halogen, cyano group, C₁-C₃ alkyl or C₁-C₃ alkoxyl; Preferably, R₉₁ is selected from: hydrogen, halogen, C₁-C₆ alkyl, C₁-C₆ haloalkyl, C₁-C₆ alkoxyl, hydroxyl, Rₙ, —ORₙ, —OC(═O)Rₙ, —NRₘRₙ, —NRₘ—C(═O)Rₙ, —NRₙ-C(═O)Rₘ, —NRₘ—S(═O)₂Rₙ, —NRₙ—S(═O)₂Rₘ; More preferably, R₉₁ is selected from: hydrogen, halogen, C₁-C₆ alkyl, C₁-C₆ haloalkyl, C₁-C₆ alkoxyl, hydroxyl, Rₙ, —ORₙ, —NRₘRₙ, —NRₙ-C(═O)Rₘ, —NRₙ—S(═O)₂Rₘ;
wherein R₉₂—R₉₅ is independently selected from: hydrogen, R₁₃, C₁-C₆ alkyl, or any two adjacent groups in R₉₂—R₉₅ can be combined to form a cycloalkyl group; Preferably, R₉₂—R₉₅ is independently selected from: hydrogen, methyl, ethyl, hydroxyl, Rₙ, —ORₙ, or R₉₂ and R₉₃ together form —CH₂CH₂—, or R₉₄ and R₉₅ together form —CH₂CH₂—;
preferably, R₉₁ is selected from R₁₃, or one of R₉₁—R₉₅ is selected from R₁₃.

36-39. (canceled)

40. The compound and the pharmaceutically acceptable salt, solvent compound, stereoisomer, isotope and prodrug thereof according to claim 1, wherein, the compound is a compound represented by formula (IV′) or formula (V′): 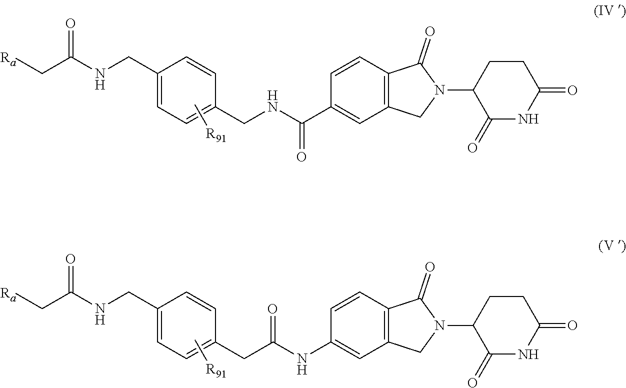
Wherein, Rₐ and R₉₁ are defined as above;
Preferably, R₉₁ is selected from: hydroxyl, Rₙ, —ORₙ, —NRₘRₙ, —NRₙ—C(═O)Rₘ, —NRₙ—S(═O)₂Rₘ; or
the compound is a compound represented by formula (VI) or formula (VII): 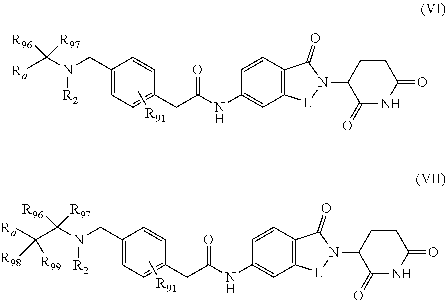
wherein each NH of —CH₂C(═O)NH— is substituted with R₁₄;
Rₐ, R₂, R₉₁, R₁₄, L are defined as above;
R₉₆-R₉₉ is independently selected from: hydrogen, R₁₃, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxyl, C₁-C₆ alkylthio, C₁-C₆ alkylamino, di(C₁-C₆ alkyl)amino, C₃-C₈ cycloalkyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, cyano group, C₁-C₃ alkyl or C₁-C₃ alkoxyl; Any two adjacent ones in R₉₆-R₉₉ may be combined to form a ring.
Preferably, R₉₆—R₉₉ is independently selected from: hydrogen, R₁₃, C₁-C₆ alkyl group, or any two adjacent groups in R₉₆—R₉₉ can be combined to form a cycloalkyl group; More preferably, R₉₆—R₉₉ is independently selected from: hydrogen, methyl, ethyl, hydroxyl, Rₙ, —ORₙ, or R₉₆ and R₉₇ together form —CH₂CH₂—, or R₉₈ and R₉₉ together form —CH₂CH₂—, respectively;
preferably, R₂ is selected from: hydrogen, R₁₄, C₁-C₈alkyl, C₃-CIO cycloalkyl, —C(═O)C₃-C₁₀ cycloalkyl, —C(═O)C₁-C₈alkyl or —S(═O)₂C₁-C₈alkyl, wherein C₁-C₈alkyl, C₃-CIO cycloalkyl and the cycloalkyl or alkyl portion of the —C(═O)C₃-C₁₀ cycloalkyl, —C(═O)C₁-C₈alkyl or —S(═O)₂C₁-C₈alkyl is optionally substituted with one or more R₉, wherein R₉ is selected from: hydrogen, halogen, amino, cyano, carboxyl, C₁-C₆ alkyl, C₁-C₆alkoxyl or aryl;
wherein R₁₄ is selected from: Rₙ, —C(═O)Rₙ, —C(═O)ORₙ, —C(═O)NRₘRₙ, —S(═O)₂Rₙ, —S(═O)₂NRₘR;
wherein Rₙ is selected from: C₁-C₈alkyl substituted with hydroxyl, sulfhydryl or amino; Preferably, Rₙ is selected from: C₁-C₄ alkyl substituted with hydroxyl, sulfhydryl or amino; More preferably, Rₙis selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH:
wherein Rₘ is selected from: hydrogen, —C₁-C₄ alkyl;
preferably, R₂ is selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthyl ethyl, —CH₂OH, —CH₂CH₂OH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —C(═O)CH₂OH, —C(═O)CH₂CH₂OH, —C(═O)CH₂CH₂CH₂OH, —C(═O)CH₂NH₂, —C(═O)CH₂CH₂NH₂, —C(═O)CH₂CH₂CH₂NH₂, —C(═O)OCH₂OH, —C(═O)OCH₂CH₂OH, —C(═O)OCH₂CH₂CH₂OH, —C(═O)OCH₂NH₂, —C(═O)OCH₂CH₂NH₂, —C(═O)OCH₂CH₂CH₂NH₂, —C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂CH₂CH₂OH, —C(═O)NHCH₂NH₂, —C(═O)NHCH₂CH₂NH₂, —C(═O)NHCH₂CH₂CH₂NH₂, —C(═O)NHCH₂SH, —C(═O)NHCH₂CH₂SH, —C(═O)NHCH₂CH₂CH₂SH, —S(═O)₂CH₂OH, —S(═O)₂CH₂CH₂OH, —S(═O)₂CH₂CH₂CH₂OH, —S(═O)₂CH₂NH₂, —S(═O)₂CH₂CH₂NH₂, —S(═O)₂CH₂CH₂CH₂NH₂, —S(═O)₂CH₂SH, —S(═O)₂CH₂CH₂SH, —S(═O)₂CH₂CH₂CH₂SH, —S(═O)₂NHCH₂OH, —S(═O)₂NHCH₂CH₂OH, —S(═O)₂NHCH₂CH₂CH₂OH, —S(═O)₂NHCH₂NH₂, —S(═O)₂NHCH₂CH₂NH₂, —S(═O)₂NHCH₂CH₂CH₂NH₂, —S(═O)₂NHCH₂SH, —S(═O)₂NHCH₂CH₂SH, —S(═O)₂NHCH₂CH₂CH₂SH:
preferably, R₂ is selected from R₁₄; R₉₁ is selected from R₁₃, or one of R₉₆—R₉₉ is selected from R₁₃.

41-44. (canceled)

45. The compound and the pharmaceutically acceptable salt, solvent compound, stereoisomer, isotope and prodrug thereof according to claim 1, wherein, the compound is a compound represented by formula (VI′) or formula (VII′): 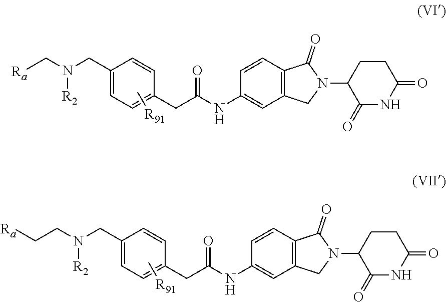
Wherein, Rₐ, R₂, R₉₁ is defined as above;
Preferably, R₂ is selected from: Rₙ, —C(═O)Rₙ, —C(═O)ORₙ, —C(═O)NRₘRₙ.;
Preferably, R₉₁ is selected from: hydroxyl, Rₙ, —ORₙ, —NRₘRₙ, —NRₙ—C(═O)Rₘ, —NRₙ-S(═O)₂Rₘ.or
the compound is a compound represented by formula (VIII): 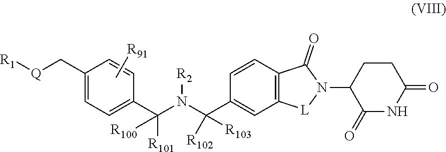
wherein, R₁, Q, R₂, R₉₁, L is defined as above;
R₁₀₀-R₁₀₃ is independently selected from: hydrogen, R₁₃, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxyl, C₁-C₆ alkylthio, C₁-C₆ alkylamino, di(C₁-C₆ alkyl)amino, C₃-C₈ cycloalkyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, cyano group, C₁-C₃ alkyl or C₁-C₃ alkoxyl; Any two adjacent ones in R₁₀₀-R₁₀₃ may be combined to form a ring;
Preferably, the R₁₀₀-R₁₀₃ is independently selected from: hydrogen, R₁₃, C₁-C₆ alkyl group, or any two adjacent groups in R₁₀₀-R₁₀₃ can be combined to form a cycloalkyl group; More preferably, R₁₀₀-R₁₀₃ is independently selected from: hydrogen, methyl, ethyl, hydroxyl, Rₙ, —ORₙ, or R₁₀₀ and R₁₀₀ together form —CH₂CH₂—, or R₁₀₂ and R₁₀₃ together form —CH₂CH₂—, respectively;
preferably, R₂ is selected from: hydrogen, R₁₄, C₁-C₈ alkyl, C₃-CIO cycloalkyl, aryl, —C(═O)C₃-C₁O cycloalkyl, —C(═O)C₁-C₈ alkyl or —S(═O)₂C₁-C₈alkyl, wherein C₁-C₈ alkyl, C₃-CIO cycloalkyl and the cycloalkyl or alkyl portion of —C(═O)C₃-C₁₀ cycloalkyl, —C(═O)C₁-C₈ alkyl or —S(═O)₂C₁-C₈alkyl is optionally substituted with one or more R₉, wherein R₉ is selected from: hydrogen, halogen, amino, cyano, carboxyl, C₁-C₆ alkyl, C₁-C₆alkoxyl or aryl;
wherein R₁₄ is selected from: Rₙ, —C(═O)Rₙ, —C(═O)ORₙ, —C(═O)NRₘRₙ, —S(═O)₂Rₙ, —S(═O)₂NRₘR;
wherein Rₙ is selected from: C₁-C₈alkyl substituted with hydroxyl, sulfhydryl or amino; Preferably, Rₙ is selected from: C₁-C₄ alkyl substituted with hydroxyl, sulfhydryl or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH:
wherein Rₘ is selected from: hydrogen, —C₁-C₄ alkyl;
preferably, R₂ is selected from: hydrogen, methyl, ethyl, —SO₂CH₃, —COCH₃, —CO-isopropyl, —CO-cyclopropyl, isopropyl, cyclopropyl, 2-methoxyethyl, 2-cyanoethyl, phenyl, naphthyl, benzyl, 2-phenyl ethyl, 1-naphthyl methyl, 2-naphthyl ethyl, —CH₂OH, —CH₂CH₂OH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —C(═O)CH₂OH, —C(═O)CH₂CH₂OH, —C(═O)CH₂CH₂CH₂OH, —C(═O)CH₂NH₂, —C(═O)CH₂CH₂NH₂, —C(═O)CH₂CH₂CH₂NH₂, —C(═O)CH₂SH, —C(═O)CH₂CH₂SH, —C(═O)CH₂CH₂CH₂SH, —C(═O)OCH₂OH, —C(═O)OCH₂CH₂OH, —C(═O)OCH₂CH₂CH₂OH, —C(═O)OCH₂NH₂, —C(═O)OCH₂CH₂NH₂, —C(═O)OCH₂CH₂CH₂NH₂, —C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂CH₂CH₂OH, —C(═O)NHCH₂NH₂, —C(═O)NHCH₂CH₂NH₂, —C(═O)NHCH₂CH₂CH₂NH₂, —C(═O)NHCH₂SH, —C(═O)NHCH₂CH₂SH, —C(═O)NHCH₂CH₂CH₂SH, —S(═O)₂CH₂OH, —S(═O)₂CH₂CH₂OH, —S(═O)₂CH₂CH₂CH₂OH, —S(═O)₂CH₂NH₂, —S(═O)₂CH₂CH₂NH₂, —S(═O)₂CH₂CH₂CH₂NH₂, —S(═O)₂CH₂SH, —S(═O)₂CH₂CH₂SH, —S(═O)₂CH₂CH₂CH₂SH, —S(═O)₂NHCH₂OH, —S(═O)₂NHCH₂CH₂OH, —S(═O)₂NHCH₂CH₂CH₂OH, —S(═O)₂NHCH₂NH₂, —S(═O)₂NHCH₂CH₂NH₂, —S(═O)₂NHCH₂CH₂CH₂NH₂, —S(═O)₂NHCH₂SH, —S(═O)₂NHCH₂CH₂SH, —S(═O)₂NHCH₂CH₂CH₂SH: preferably R₂ is selected from hydrogen, methyl, ethyl, isopropyl, cyclopropyl, benzyl, —CH₂OH, —CH₂CH₂OH, —CH₂CH₂CH₂OH, —CH₂NH₂, —CH₂CH₂NH₂, —CH₂CH₂CH₂NH₂, —CH₂SH, —CH₂CH₂SH, —CH₂CH₂CH₂SH, —CH₂CH₂CH₂CH₂SH, —C(═O)CH₂OH, —C(═O)CH₂CH₂OH, —C(═O)CH₂CH₂CH₂OH, —C(═O)CH₂ NH₂, —C(═O)CH₂CH₂NH₂, —C(═O)CH₂CH₂CH₂NH₂, —C(═O)CH₂SH, —C(═O)CH₂CH₂SH, —C(═O)CH₂CH₂CH₂SH, —C(═O)OCH₂OH, —C(═O)OCH₂CH₂OH, —C(═O)OCH₂CH₂CH₂OH, —C(═O)OCH₂NH₂, —C(═O)OCH₂CH₂NH₂, —C(═O)OCH₂CH₂CH₂NH₂, —C(═O)OCH₂SH, —C(═O)OCH₂CH₂SH, —C(═O)OCH₂CH₂CH₂SH, —C(═O)NHCH₂OH, —C(═O)NHCH₂CH₂OH, —C(═O)NHCH₂CH₂CH₂OH, —C(═O)NHCH₂NH₂, —C(═O)NHCH₂CH₂NH₂, —C(═O)NHCH₂CH₂CH₂NH₂, —C(═O)NHCH₂SH, —C(═O)NHCH₂CH₂SH, —C(═O)NHCH₂CH₂CH₂SH:
preferably, R₂ is selected from R₁₄, or R₉₁ is selected from R₁₃, or one of the R₁₀₀-R₁₀₃ is selected from R₁₃.

46-49. (canceled)

50. The compound and the pharmaceutically acceptable salt, solvent compound, stereoisomer, isotope and prodrug thereof according to claim 1, wherein, the compound is a compound represented by formula (VIII′): 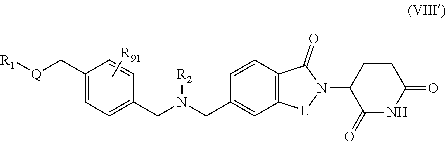
Wherein, R₁, Q, R₂, R₉₁ is defined as above;
Preferably, R₂ is selected from: Rₙ, —C(═O)Rₙ, —C(═O)ORₙ, —C(═O)NRₘRₙ.;
Preferably, R₉₁ is selected from: hydroxyl, Rₙ, —ORₙ, —NRₘRₙ, —NRₙ—C(═O)Rₘ, —NRₙ—S(═O)₂Rₘ.or
the compound is a compound represented by formula (XI): 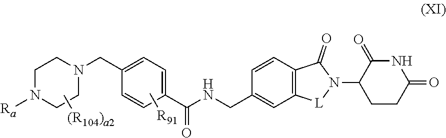
wherein NH of —C(═O)NHCH₂— is optionally substituted with R₁₄;
Rₐ, R_{b4}, R₉₁, a2 is defined as above;
R₁₀₄ is selected from: hydrogen, R₁₃, halogen, cyano, amino, nitro, —R₂₁C(═O)R₂₂, —R₂₁NHC(═O)R₂₂, —R₂₁C(═O)NHR₂₂, —R₂₁C(═O)OR₂₂, —R₂₁OC(═O)R₂₂, —R₂₁S(═O)₂R₂₂, —R₂₁S(═O)₂NHR₂₂, C₁-C₆ alkyl, C₁-C₆ alkoxyl, C₁-C₆ alkylthio, C₁-C₆ alkylamino, di(C₁-C₆alkyl)amino, C₃-C₈ cycloalkyl, C₂-C₈ alkenyl, C₂-C₈ alkynyl, aryl, 5-6 membered heteroaryl containing 1-3 heteroatoms or 3-10 membered heterocyclyl containing 1-3 heteroatoms, wherein alkyl, alkoxyl, alkylamino, alkylthio, cycloalkyl, aryl, heteroaryl, heterocyclyl is optionally substituted with halogen, cyano group, C₁-C₃ alkyl or C₁-C₃ alkoxyl;
preferably, R₁₀₄ is selected from: hydrogen, R₁₃, halogen, C₁-C₆ alkyl, C₁-C₆ haloalkyl; More preferably, the R₁₀₄ is selected from: hydrogen, F, Cl, Br, methyl, ethyl, trifluoromethyl, hydroxyl, Rₙ, —OR.;
preferably, Rₙ is selected from: C₁-C₈ alkyl substituted with hydroxyl, sulfhydryl or amino; Preferably, Rₙ is selected from: C₁-C₄ alkyl substituted with hydroxyl, sulfhydryl or amino; More preferably, Rₙ is selected from: —CH₂OH, —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH:
preferably, R₉₁ is selected from R₁₃, or one of R₁₀₄ is selected from R₁₃; or
the compound is a compound represented by formula (XI′): 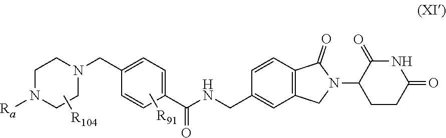
wherein, Rₐ, R₉₁, R₁₀₄ is defined as above;
preferably, R₉₁ is selected from: hydroxyl, Rₙ, —ORₙ, —NRₘRₙ, —NRₙ—C(═O)Rₘ, —NRₙ—S(═O)₂Rₘ;
preferably, R₁₀₄ is selected from: hydroxyl, Rₙ, —ORₙ.

51-54. (canceled)

55. The compound and the pharmaceutically acceptable salt, solvent compound, stereoisomer, isotope and prodrug thereof according to claim 1, wherein,
R₁ is selected from RₐC(═O)—, RₐCHR₉C(═O)—, RₐCHR₉CH₂C(═O)—, RₐCHCHR₉C(═O)—Rₐ—, RₐCHR₉—RₐCHR₉CH₂—RₐCHCHR₉—; the R₉ is selected from hydroxyl, R₁₃; the R₁₃ is selected from hydroxyl, sulfhydryl, amino, Rₙ; the Rₙis selected from C₁-C₄ alkyl substituted with hydroxyl, sulfhydryl or amino;
Rₐ is selected from phenyl, naphthalen-1-yl, naphthalen-2-yl, carbazole-9-yl, 1-aza-carbazole-9-yl, 2-aza-carbazole-9-yl, 1,8-diaza-carbazole-9-yl, indole-1-yl, 2, 3-dihydro-indole-1-yl, 7-aza-indole-1-yl, 2, 3-dihydro-7-aza-indole-1-yl, phenoxazine-10-yl, fluorene-9-yl, quinoline-4-yl, quinoline-5-yl, quinoline-8-yl, isoquinoline-1-yl, isoquinoline-4-yl, isoquinoline-5-yl, isoquinoline-8-yl, 1,2,3,4-tetrahydro-1,8-naphthyridine-1-yl, 1,2,3,4-tertrahydro-quinoline-1-yl, pyrimidine-2-yl, pyrimidine-4-yl, pyrimidine-5-yl, pyridine-2-yl, pyridine-3-yl, pyridine-4-yl, 5-(8-quinoline-yl)-pyridine-2-yl, 1,2,3-triazol-1-yl, 1,2,4-triazol-l-yl, 1,3,4-triazol-1-yl, bicyclo[1.1.1]pentane-1-yl, norborne-l-yl, adamantane-1-yl; or Rₐ is selected from 5-(1-isoquinoline-yl)-pyridine-2-yl;
Rₐ is optionally substituted with one or more R₉, and the R₉ is selected from: R₁₃, F, Cl, Br, cyano, methyl, ethyl, trifluoromethyl, methoxyl, ethoxy, phenyl, naphthalen-1-yl, naphthalen-2-yl; R₁₃ is selected from hydroxyl, sulfhydryl, amino, Rₙ, —ORₙ, —OC(═O)Rₙ, —NRₘRₙ, —NRₘ C(═O)Rₙ, —NRₙ—C(═O)Rₘ, —NRₘ—S(═O)₂Rₙ, —NRₙ—S(═O)₂Rₘ; the Rₙis selected from C₁-C₄ alkyl substituted with hydroxyl, sulfhydryl or amino; the Rₘ is selected from hydrogen, —C₁-C₄ alkyl;
Q is selected from: —NR₂—, 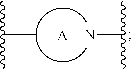
R₂ is selected from: hydrogen, R₁₄, C₁-C₈ alkyl; R₁₄ is selected from: Rₙ, —C(═O)Rₙ, —C(═O)ORₙ, —C(═O)NRₘRₙ, —S(═O)₂Rₙ, —S(═O)₂NRₘRₙ; Rₙis selected from: —CH₂OH , —CH₂CH₂OH, —CH₂CH₂CH₂OH, —CH₂CH₂CH₂CH₂OH, —CH₂NH₂, —CH₂CH₂NH₂, —CH₂CH₂CH₂NH₂, —CH₂CH₂CH₂CH₂NH₂, —CH₂SH, —CH₂CH₂SH, —CH₂CH₂CH₂SH, —CH₂CH₂CH₂CH₂SH; Rₘ is selected from hydrogen, —C₁-C₄ alkyl; 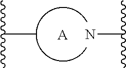
is selected from one of the following structures: 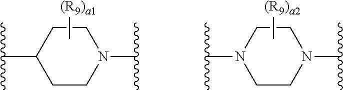
wherein, each R₉ is independently selected from: hydrogen, R₁₃; R₁₃ is selected from hydroxyl, sulfhydryl, amino, Rₙ; Rₙis selected from C₁-C₄ alkyl substituted with hydroxyl, sulfhydryl or amino;
each a1 is independently selected from: 0, 1, 2, 3, 4, 5, 6, 7, 8, 9; each a2 is independently selected from: 0, 1, 2, 3, 4, 5, 6, 7, 8;
T, Z is independently selected from: chemical bond, methylene, 1,2-ethylidene;
U is selected from: C₁-C₆ alkylene, 1,4-phenylene; the 1,4-phenylene is optionally substituted with one or more R₉, and the R₉ is selected from: hydrogen, amino, hydroxyl, sulfhydryl, R₁₃; R₁₃ is selected from Rₙ, —ORₙ, —OC(═O)Rₙ, —NRₘRₙ, —NRₘ—C(═O)Rₙ, —NRₙ—C(═O)Rₘ, —NRₘ—S(═O)₂Rₙ, —NRₙ—S(═O)₂Rₘ; the Rₙis selected from C₁-C₄ alkyl substituted with hydroxyl, sulfhydryl or amino; the Rₘ is selected from hydrogen, —C₁-C₄ alkyl;
Y is selected from: chemical bond, —C(═O)NH(CH₂)_{b}—, —NHC(═O)(CH₂)_{b}—, —NR₂(CH₂)_{b}—;
L is selected from: —(CR₇R₈)o-, —C(═O)—; the R₇-R₈ is independently selected from: hydrogen;
R₃-R₅ is independently selected from: hydrogen;
R₆ is selected from: hydrogen;
b is selected from:0, 1, 2, 3, 4 or 5;
n is selected from:0, 1, 2, or 3;
m is selected from:0, 1, 2, 3 or 4;
o is selected from: 1.

56. The compound and the pharmaceutically acceptable salt, solvent compound, stereoisomer, isotope and prodrug thereof according to claim 1, wherein, R₁ is selected from: RₐC(═O)—, RₐCHR₉C(═O)—, RₐCHR₉CH₂C(═O)—, RₐCHCHR₉C(═O)—, Rₐ—, RₐCHR₉—, RₐCHR₉CH₂—, RₐCHCHR₉—; R₉ is selected from: hydrogen, R₁₃; R₁₃ is selected from: hydroxyl, sulfhydryl, amino, Rₙ; Rₙis selected from C₁-C₄ alkyl substituted with hydroxyl, sulfhydryl or amino;
Rₐ is selected from the following groups: 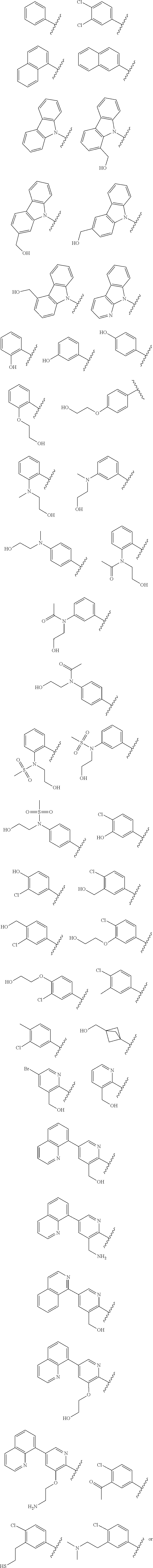 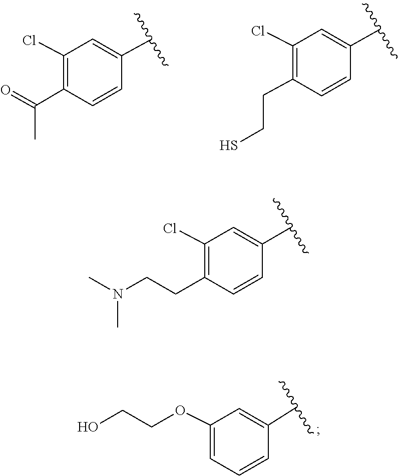 [SMILES: *c1ccc(C(C)=O)c(Cl)c1 ; *c1ccc(CCS)c(Cl)c1 ; *c1ccc(CCN(C)C)c(Cl)c1 ; *c1cccc(OCCO)c1]
Q is selected from: —NR₂— 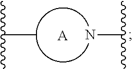
R₂ is selected from: hydrogen, R₁₄, C₁-C₈ alkyl; R₁₄ is selected from: R₂₁, —C(═O)R₁₁, —C(═O)ORₙ, —C(═O)NRₘRₙ, —S(═O)₂R₁₁, —S(═O)₂NRₘRₙ; R₁₁ is selected from: —CH₂OH, —CH₂CH₂OH, —CH₂CH₂CH₂OH, —CH₂CH₂CH₂CH₂OH, —CH₂NH₂, —CH₂CH₂NH₂,-CH₂CH₂CH₂NH₂, —CH₂CH₂CH₂CH₂NH₂, —CH₂SH, —CH₂CH₂SH, —CH₂CH₂CH₂SH,-CH₂CH₂CH₂CH₂SH; Rₘ is selected from: hydrogen. —C₁-C₄ alkyl; 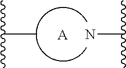
is selected from one of the following structures: 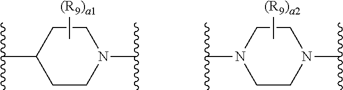
wherein, each R₉ is independently selected from: hydrogen, R₁₃; R₁₃ is selected from hydroxyl, sulfhydryl, amino, Rₙ; Rₙis selected from C₁-C₄ alkyl substituted with hydroxyl, sulfhydryl or amino;
each a1 is independently selected from: 0, 1, 2, 3, 4, 5, 6, 7, 8, 9; each a2 is independently selected from: 0, 1, 2, 3, 4, 5, 6, 7, 8;
T, Z is independently selected from: chemical bond, methylene, 1,2-ethylidene;
U is selected from: C₁-C₆ alkylene, 1,4-phenylene; the 1,4-phenylene is optionally substituted with one or more R₉, and the R₉ is selected from: hydrogen, amino, hydroxyl, sulfhydryl, R₁₃; R₁₃ is selected from Rₙ, —ORₙ, —OC(═O)Rₙ, —NRₘRₙ, —NRₘC(═O)Rₙ, —NRₙ—C(═O)Rₘ, —NRₘ—S(═O)₂Rₙ, —NRₙ—S(═O)₂Rₘ; the Rₙis selected from C₁-C₄ alkyl substituted with hydroxyl, sulfhydryl or amino; the Rₘ is selected from hydrogen, —C₁-C₄ alkyl;
Y is selected from: chemical bond, —C(═O)NH(CH₂)_{b}—, —NHC(═O)(CH₂)_{b}—, —NR₂(CH₂)_{b}—;
L is selected from: —(CR₇R₈)o-, —C(═O)—; the R₇-R₈ is independently selected from: hydrogen;
R₃-R₅ is independently selected from: hydrogen;
R₆ is selected from: hydrogen;
b is selected from:0, 1, 2, 3, 4 or 5;
n is selected from:0, 1, 2, or 3;
m is selected from:0, 1, 2, 3 or 4;
o is selected from: 1.

57. The compound and the pharmaceutically acceptable salt, solvent compound, stereoisomer, isotope and prodrug thereof according to claim 1, wherein, the compound has the following structures:

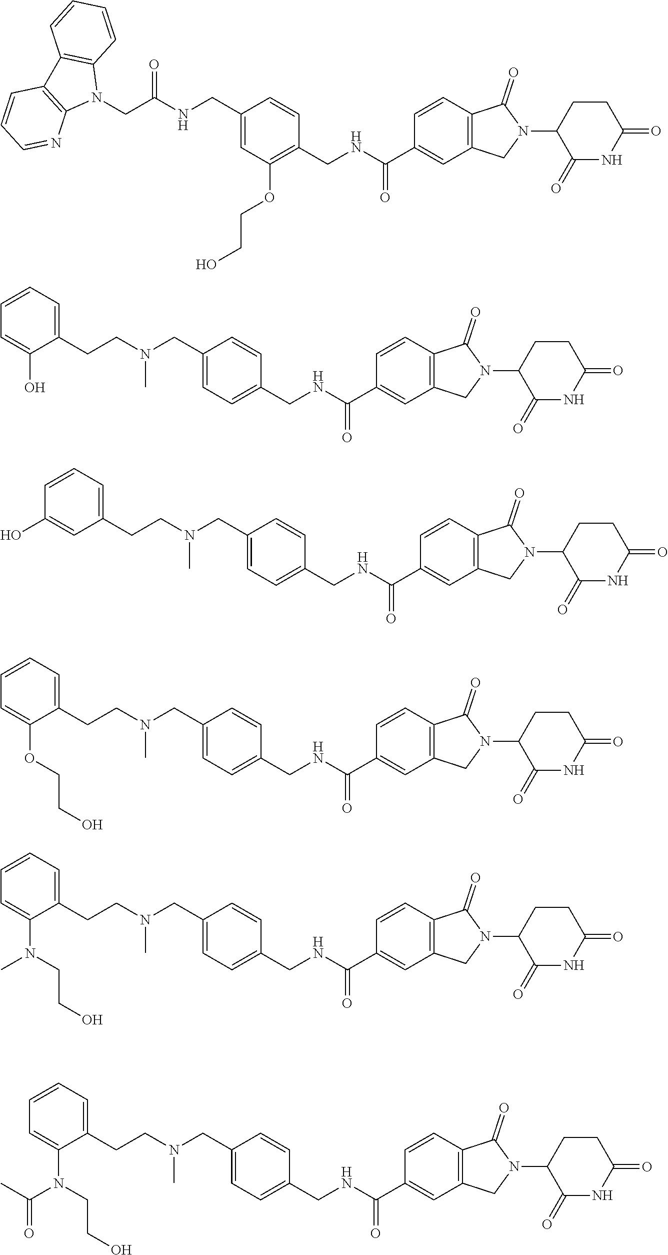 [SMILES: O=C(Cn1c2ccccc2c2cccnc21)NCc1ccc(CNC(=O)c2ccc3c(c2)CN(C2CCC(=O)NC2=O)C3=O)c(OCCO)c1 ; CN(CCc1ccccc1O)Cc1ccc(CNC(=O)c2ccc3c(c2)CN(C2CCC(=O)NC2=O)C3=O)cc1 ; CN(CCc1cccc(O)c1)Cc1ccc(CNC(=O)c2ccc3c(c2)CN(C2CCC(=O)NC2=O)C3=O)cc1 ; CN(CCc1ccccc1OCCO)Cc1ccc(CNC(=O)c2ccc3c(c2)CN(C2CCC(=O)NC2=O)C3=O)cc1 ; CN(CCc1ccccc1N(C)CCO)Cc1ccc(CNC(=O)c2ccc3c(c2)CN(C2CCC(=O)NC2=O)C3=O)cc1 ; CC(=O)N(CCO)c1ccccc1CCN(C)Cc1ccc(CNC(=O)c2ccc3c(c2)CN(C2CCC(=O)NC2=O)C3=O)cc1]

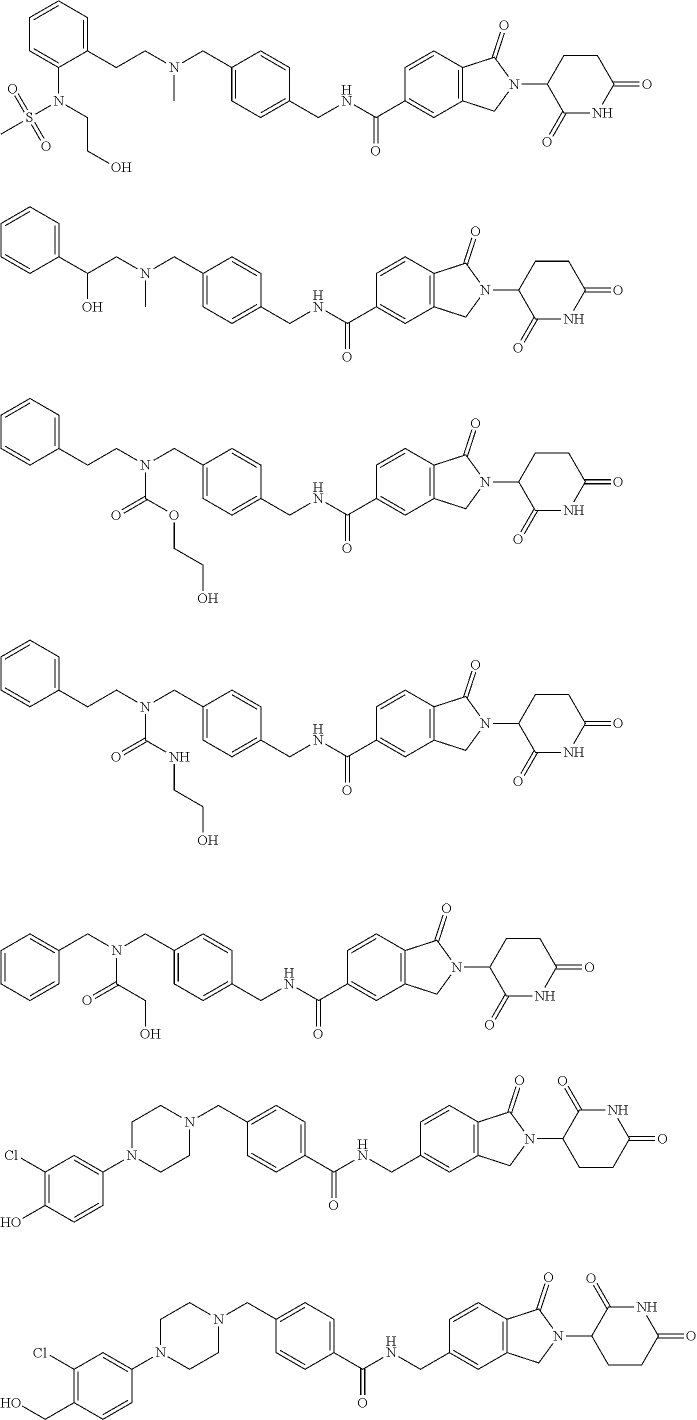

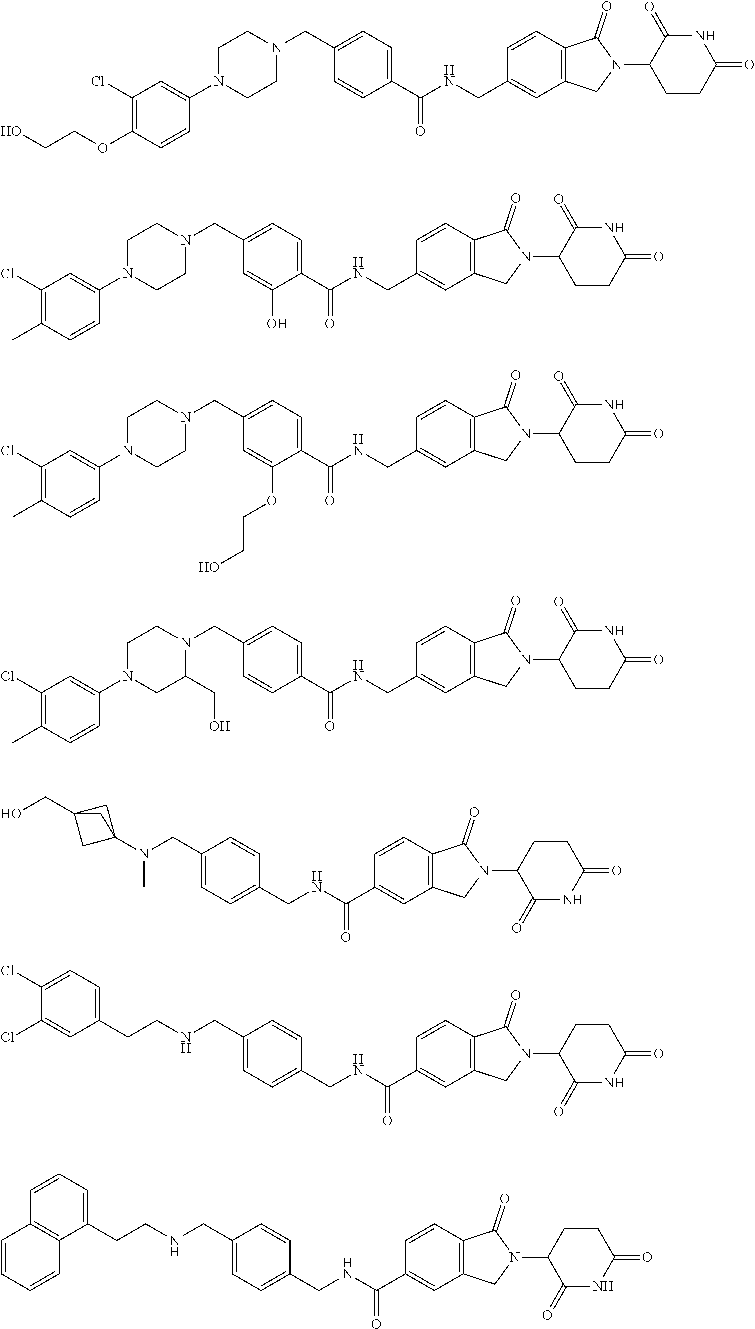

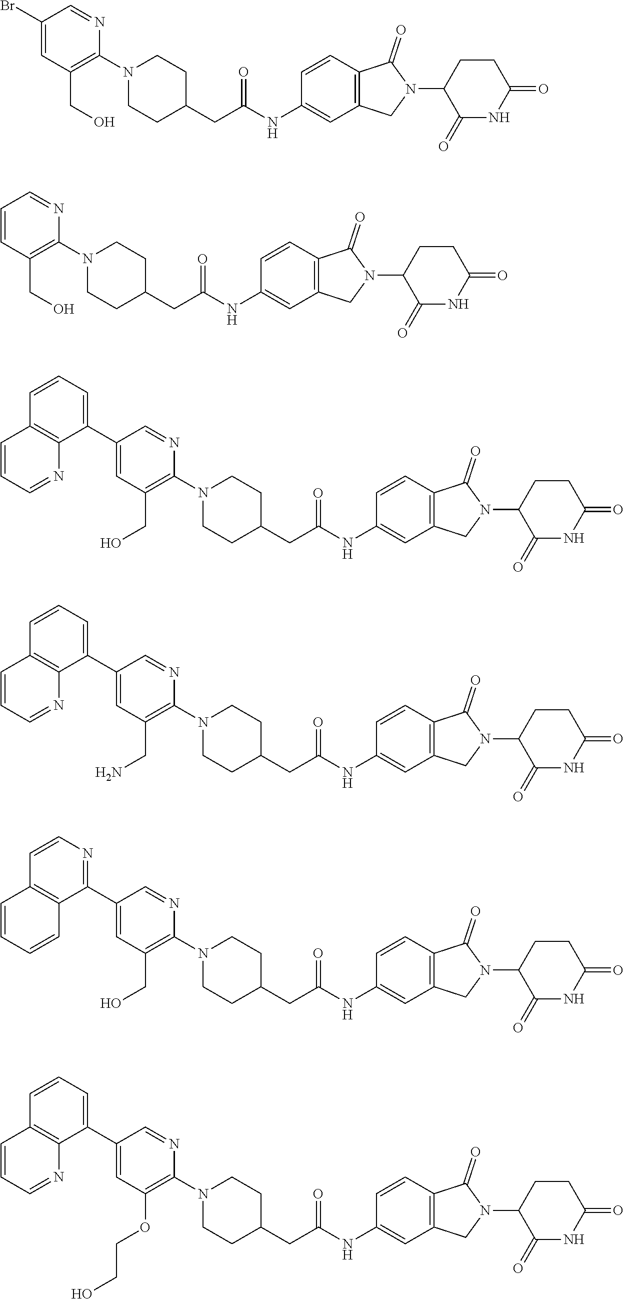 [SMILES: O=C1CCC(N2Cc3cc(NC(=O)CC4CCN(c5ncc(Br)cc5CO)CC4)ccc3C2=O)C(=O)N1 ; O=C1CCC(N2Cc3cc(NC(=O)CC4CCN(c5ncccc5CO)CC4)ccc3C2=O)C(=O)N1 ; O=C1CCC(N2Cc3cc(NC(=O)CC4CCN(c5ncc(-c6cccc7cccnc67)cc5CO)CC4)ccc3C2=O)C(=O)N1 ; NCc1cc(-c2cccc3cccnc23)cnc1N1CCC(CC(=O)Nc2ccc3c(c2)CN(C2CCC(=O)NC2=O)C3=O)CC1 ; O=C1CCC(N2Cc3cc(NC(=O)CC4CCN(c5ncc(-c6nccc7ccccc67)cc5CO)CC4)ccc3C2=O)C(=O)N1 ; O=C1CCC(N2Cc3cc(NC(=O)CC4CCN(c5ncc(-c6cccc7cccnc67)cc5OCCO)CC4)ccc3C2=O)C(=O)N1]

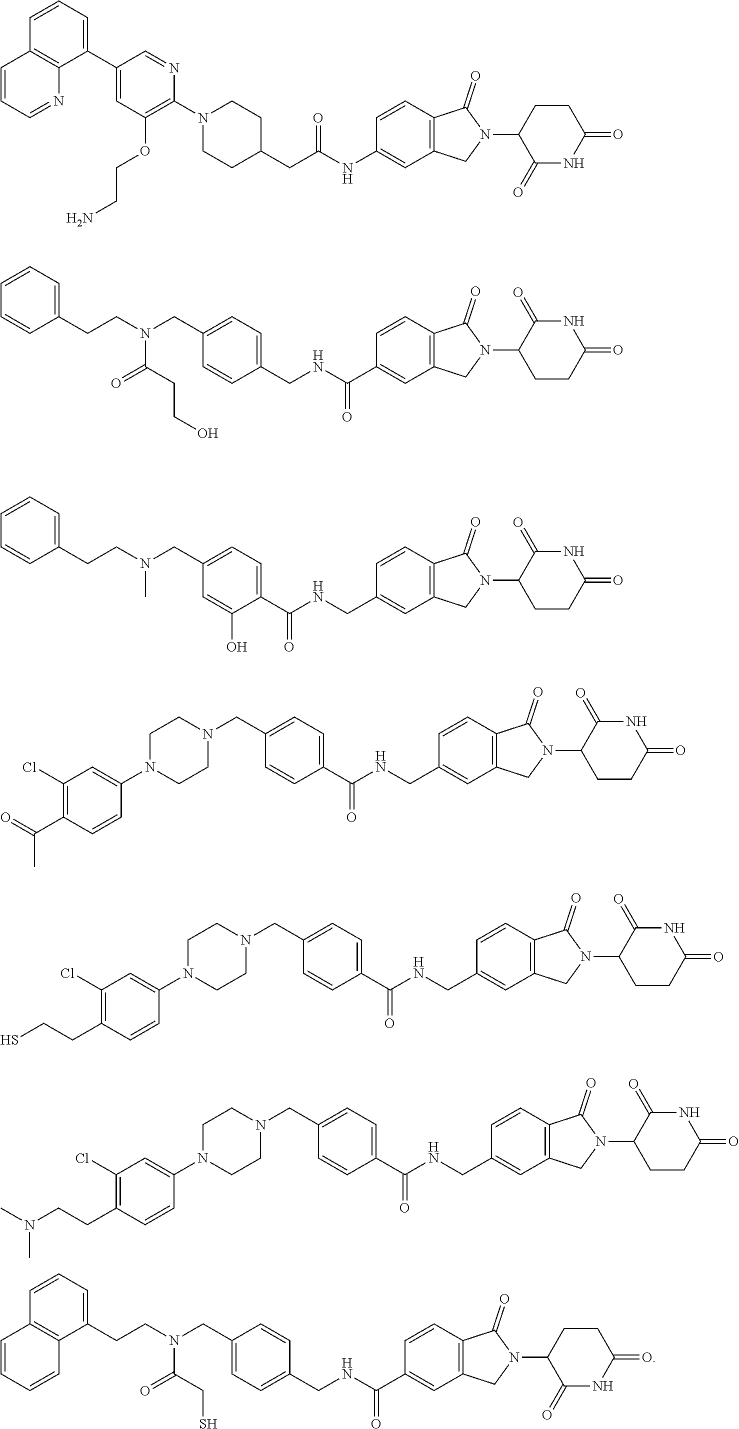

58. A pharmaceutical composition, wherein the pharmaceutical composition comprises a pharmaceutically acceptable carrier, and the compound and the pharmaceutically acceptable salt, solvent compound, stereoisomer, isotope and prodrug thereof according to claim 1;
preferably, the pharmaceutical composition may further comprise an MYC inhibitor, a DNA methyltransferase inhibitor or a Bcl-2 selective inhibitor;
preferably, the MYC inhibitor is any one or more of OMO-103, APTO-253, PLX-51107, DCR-M1711, Oncomyc-NG, INX-3280, PU-27, GSK-3179106, cholesterol butyrate and NSC-165563; the DNA methyltransferase inhibitor is any one or more of 5-azacytidine, RG108, SGI-1027, GSK3685032, CM272, Bobcat339 hydrochloride, Decitabine (NSC 127716), Thioguanine (NSC 752), 2′-Deoxy-5-Fluorocytidine, Procainamide HCl or Zebularine (NSC 309132); the Bcl-2 selective inhibitor is any one or more of Venetoclax (ABT-199), S55746, BDA-366, Obatoclax Mesylate (GX15-070), HA14-1 or APG-2575 (CAS No. 2180923-05-9); or
preferably, the pharmaceutical composition comprises a Bcl-2 selective inhibitor, and the Bcl-2 selective inhibitor is Venetoclax (ABT-199), Obatoclax Mesylate (GX15-070) or APG-2575 (CAS No. 2180923-05-9).

59-62. (canceled)

63. A method for treating a disease related to dysregulation of proteins comprising any one of or at least two of c-Myc, N-myc, GSPT1, CK1αa, IKZF (1/2/3), AR and AR-V7, comprising administering the compound and the pharmaceutically acceptable salt, solvent compound, stereoisomer, isotope and prodrug thereof according to claim 1 to a subject in need thereof;
preferably, the dysregulation of proteins is selected from an overexpression of proteins; or
preferably, the disease related to dysregulation of proteins is selected from cancer, a cardiovascular and cerebrovascular disease, and a viral infection-related disease;
preferably, the cancer is selected from leukemia, lymphoma, malignant glioma, medulloblastoma, melanoma, multiple myeloma, myelodysplastic syndrome, liver cancer, lung cancer, kidney cancer, pancreatic cancer, oral cancer, gastric cancer, esophageal cancer, laryngeal cancer, nasopharyngeal cancer, skin cancer, breast cancer, colon cancer, rectal cancer, cervical cancer, ovarian cancer, prostate cancer, rhabdomyosarcoma, osteoblastic sarcoma or chondrosarcoma; the viral infection-related disease is selected from HIV, hepatitis B, hepatitis C, hepatitis A, influenza, epidemic encephalitis B or herpes; the leukemia includes chronic lymphocytic leukemia (CLL), chronic granulocytic leukemia (CML), acute myeloid leukemia (AML) or acute non-lymphocytic leukemia (ANLL).

64-67. (canceled)

68. A method for treating acute myeloid leukemia (AML),
comprising administering the compound and the pharmaceutically acceptable salt, solvent compound, stereoisomer, isotope and prodrug thereof according to claim 1;
preferably, the pharmaceutical composition comprises a Bcl-2 selective inhibitor, and the Bcl-2 selective inhibitor is Venetoclax (ABT-199), Obatoclax Mesylate (GX15-070) or APG-2575 (CAS No. 2180923-05-9).

69. (canceled)

70. (canceled)
